(19) United States
(12) Patent Application Publication
(10) Pub. No.: US 2025/0215615 A1
(43) Pub. Date: Jul. 3, 2025

(54) METHOD FOR EVALUATING DNA-ENCODED LIBRARY

(71) Applicant: Nissan Chemical Corporation, Tokyo (JP)

(72) Inventors: Masatoshi NIWA, Funabashi (JP); Munefumi TOKUGAWA, Funabashi (JP); Jun HAYASHIDA, Shiraoka (JP)

(73) Assignee: Nissan Chemical Corporation, Tokyo (JP)

(21) Appl. No.: 18/713,086

(22) PCT Filed: Nov. 24, 2022
(86) PCT No.: PCT/JP2022/043405

§ 371 Date: May 23, 2024

(30) Foreign Application Priority Data
Nov. 24, 2021 (JP) 2021-190592

(51) Int. Cl. C40B 30/04; C12Q 1/6876

(52) U.S. Cl. CPC C40B 30/04; C12Q 1/6876

(57) ABSTRACT

The invention provides a method of inducing a DNA-encoded library (DEL) comprising cleavable site(s) in the DNA strand to a cross linker-modified double-stranded DEL and evaluating the DEL. Both the merits of the hairpin-stranded DEL and the double-stranded DEL are achieved in the invention by introducing the cleavable site(s) such as deoxyuridine into the DNA strand. The invention further provides a technique for screening a compound in which both “simple DEL synthesis method” and “expansion and improvement of the DEL evaluation method” are achieved by easily inducing the compound into a cross linker-modified DEL.

### TECHNICAL FIELD

The present invention relates to a method of evaluating a DNA-encoded library.

### BACKGROUND ART

A compound library is a group of compound derivatives in which compounds having a possibility to have a specific activity, such as a drug candidate compound, are systematically collected. The compound library is, in many cases, synthesized based on synthetic techniques and methodologies of combinatorial chemistry. The combinatorial chemistry is a field of experimental methods for efficiently synthesizing a series of compound libraries enumerated and designed based on the combinatorics with a wide variety of compounds by a systematic synthetic route and research relating thereto.

DNA-encoded library is one kind of compound library based on the combinatorial chemistry. Hereinafter, the DNA-encoded library is appropriately abbreviated to as DEL. In DEL, a DNA tag is added to each compound in the library. The sequence of the DNA tag is designed so that each structure in each compound can be identified and functions as a label for the compound (Patent Documents 1 to 3).

A plurality of compounds having potential for pharmaceutical development have been found by screening using DEL. The screening using DEL is carried out, for example, as follows (Non-Patent Documents 1 to 3).
1) Immobilize a target protein on an immobilization carrier,
2) Contact a target with DEL,
3) Wash and remove DEL having low affinity with the target,
4) Denature the target protein and elute DEL having high affinity, and
5) Amplify a DNA sequence comprised in the eluted DEL and identify the sequence.

However, the screening method as described above has several problems. First, since it is necessary to immobilize the target protein, depending on the protein, the steric structure will change after the immobilization. In such a case, the compound obtained by screening does not bind to a desired non-immobilized target protein, and a target adaptation range of the DEL is limited (Patent Documents 4 and 5, and Non-Patent Documents 4 and 5). In addition, in the above screening method, a binder having high affinity can be recovered, but a binder having medium affinity (for example, a Kd value is on the order of M) has low kinetic stability of a complex with a target protein, and is difficult to recover after washing and removal. As is well-known to those skilled in the art, such a binder having medium affinity is also useful as a hit compound serving as a starting point for drug discovery, and is also useful as structure-activity relationship information (Non-Patent Document 6).

As an approach to solve the above problems, a plurality of screening methods using a cross linker-modified DEL have recently been reported. As will be shown below, several different methods have been reported, all of which are common in that targets in proximity to an affinity library molecule are reacted with a cross linker to form a covalent bond. The method is useful because it allows screening for DEL without immobilizing the target protein (or before immobilizing the target protein) (Patent Documents 4 and 5, and Non-Patent Documents 4, 5, and 7 to 9).

Here, a DNA strand structure of the DEL will be described. The DNA strand structure of the conventionally known DEL is represented by two kinds, i.e. a hairpin strand and a double strand.

Hereinafter, the outline of a double strand DEL and a hairpin strand DEL and the merit and the demerit thereof are described.

### (1) Hairpin Strand DEL

A DEL using a hairpin-stranded DNA is a single-stranded structure in which two complementary DNA strands are linked, and synthesized by using a hairpin type DNA having functional groups for introducing various building blocks as a starting material (head piece) (Patent Document 3, and Non-Patent Document 1 and 2).

### (A) Merits

### (a) Short DNA Tags can be Used.

In many cases, this method uses a relatively short double-stranded DNA tag with about 9 to 13-mer having a sticky end with 2-mer, and the double-stranded DNA tag is introduced by a ligation reaction by a DNA ligase. Using such a short DNA tag becomes possible because the hairpin-stranded DNA strongly forms a duplex in the molecule and the DNA site other than the sticky end does not interfere with the DNA tag. Using a short double-stranded DNA tag has some merits in the DEL synthesis. Cost for synthesizing the DNA tag is low is mentioned as one merit. Also, as another merit, using a shorter DNA tag suppresses the overall length of the DEL short when the same number of reaction cycles are encoded. That is, if a larger number of cycles is encoded, the overall length of the DEL can be suppressed to a range in which the DNA sequence can be efficiently read by the next-generation sequencer. In fact, in Non-Patent Document 3, construction of the DEL using a hairpin-stranded DNA encoding 6 cycle reactions has been achieved by using a hairpin-stranded DNA.

### (b) Chemical Stability is High

Different from the double strand, in the hairpin strand, even when a duplex structure is melted during the reaction under heating, the duplex in the original molecule is reformed without generating strand exchange under the subsequent reannealing conditions. Accordingly, DEL using a hairpin-stranded DNA has a merit that it can be used under a wider range of chemical conditions (Non-Patent Document 2). Also, in general, as for nucleic acid strands, if the chain length is the same, the hairpin strand forms a stronger duplex than the double strand (Tm value is high). Accordingly, under various chemical conditions at the time of introducing the building blocks, each chemical structure of the hairpin-stranded DNA, particularly the structure of the base portion, should resist the structural conversion as compared with the double strand.

### (B) Demerit

The hairpin-stranded DNA forms a duplex in the molecule, and it is difficult to newly form a double strand with another oligonucleotide chain. Therefore, it is difficult to convert it into a cross linker-modified double-stranded DEL by adding a new cross linker-modified oligonucleotide.

### (2) Double-Stranded DEL

A DEL using a double-stranded DNA is synthesized using a single-stranded DNA (single-stranded DNA that is not a hairpin strand) or double-stranded DNA having a functional group(s) for introducing various building blocks as a starting material (head piece).

### (A) Demerit

Contrary to the DEL using the hairpin-stranded DNA, in many cases, relatively long single-stranded or double-stranded DNA tags with about 20 to 30-mer having 4 to 10-mer sticky end have been used (Patent Document 2 and Non-Patent Document 10) and a DEL encoding about 3 cycles reaction is common.

### (B) Merit

The double-stranded DNA can be converted into a single-stranded DNA by denaturation, or a strand exchange reaction can be carried out for it, which is advantageous in that it can be converted into a DNA structure suitable for various purposes. Therefore, conversion to a cross linker-modified double-stranded DEL can be carried out by adding a new cross linker-modified single-stranded oligonucleotide (Non-Patent Documents 7, 8, and 11).

Like this, although the hairpin-stranded DNA and the double-stranded DNA each have merits at the time of synthesis of DEL and evaluation, no technique that can achieve both merits has been known.

As an example of a report on screening using a cross linker-modified DEL, the following is known.

In Non-Patent Documents 7 and 8, a single-stranded DEL having a library molecule at the 5′ end is synthesized, a double strand with a DNA having a photoreactive cross linker at the 3′ end are formed, and screening is carried out to obtain a binder having medium affinity. On the other hand, in this method, since the DNA linked to the photoreactive cross linker does not contain a coding sequence, when the DNA is exposed to strong separation or elution conditions for removaling a non-specific binder, etc., there is a possibility that the double strand is separated and a sequence encoding a desired structure cannot be obtained. In addition, since the single-stranded DEL is used, the merits of the hairpin-stranded DEL cannot be utilized in synthesis.

Xiaoyu Li et al. report a screening method using a cross linker-modified DEL (Patent Document 5, and Non-Patent Documents 4, 5, and 12). In Non-Patent Document 5, a single-stranded DEL having a library molecule at the 3′ end is synthesized, and a double strand are formed with a short-stranded DNA having a photoreactive cross linker at the 5′ end and to make a photoreactive cross linker-modified DEL. Merits of this method that the coding sequence is covalently linked to the target by an elongation reaction with DNA polymerase since a photoreactive cross linker is present at the 5′ end are included. Therefore, strong separation and elution conditions can be included in the screening. However, a method for simply synthesizing a single-stranded DEL having a library molecule at the 3′ end has not been reported. In addition, similarly, the method does not take the merits of the hairpin-stranded DEL in synthesis.

Patent Document 4 describes a hairpin-stranded DEL having a linking site with a cross linker. Although this method can solve the problems as in Non-Patent Document 5 described above, it has another problem that library synthesis needs to be carried out without impairing the functional group for cross linker linking and that usable reactions and/or library molecular structures can be limited.

Patent Document 6 describes synthesis of a double-stranded DEL crosslinked by a reversible covalent bond (which is considered to have properties equivalent to those of a hairpin-stranded DEL from the viewpoint of duplex forming ability, etc.) and conversion to a cross linker-modified DEL. As a reversible covalent bond, a covalent bond by [2+2] photocyclization between a special base such as cyanovinylcarbazole and a pyrimidine base is disclosed. However, it is known that the photocyclized pyrimidine base has lost aromaticity, and such a pyrimidine base having lost aromaticity is chemically unstable and decomposed under basic conditions (Non-Patent Document 13). Therefore, in this method, usable reactions are limited during DEL synthesis, and the library molecular structure that can be constructed is also limited.

As described above, there is no known technique which has merits in terms of synthesis at the same level as the conventional hairpin-stranded DEL and is easily adaptable to screening with a cross linker-modified DEL.

### PRIOR ART DOCUMENTS

### Patent Documents

Patent Document 1: WO 93/20243
Patent Document 2: WO 2004/039825
Patent Document 3: WO 2005/058479
Patent Document 4: WO 2019/149198
Patent Document 5: WO 2019/157692
Patent Document 6: CN 112760720

### Non-Patent Documents

Non-Patent Document 1: Nature Chemical Biology, 2009, vol. 5, pp. 647-654
Non-Patent Document 2: A Handbook for DNA-Encoded Chemistry, Edited by Robert A. Goodnow, Jr., John Wiley & Sons, Inc.
Non-Patent Document 3: Progress in Medicinal Chemistry, 2020, vol. 59, pp. 181-249, Chapter 4—An overview of DNA-encoded libraries: A versatile tool for drug discovery
Non-Patent Document 4: Angewandte Chemie International Edition, 2014, vol. 53, pp. 10056-10059
Non-Patent Document 5: ACS Combinatorial Science, 2019, vol. 21, pp. 345-349
Non-Patent Document 6: Chem Biochem, 2019, vol. 20, pp. 955-962
Non-Patent Document 7: ACS Combinatorial Science, 2020, vol. 22, pp. 204-212
Non-Patent Document 8: Chemistry A European Journal, 2021, vol. 27, pp. 7160-7167
Non-Patent Document 9: Accounts of Chemical Research, 2021, vol. 54, pp. 3491-3503
Non-Patent Document 10: Nature Chemistry, 2018, vol. 10, pp. 441-448
Non-Patent Document 11: Annual Review of Biochemistry, 2018, vol. 87, pp. 479-502
Non-Patent Document 12: Bioconjugate Chemistry, 2017, vol. 28, pp. 2293-2301
Non-Patent Document 13: Journal of the American Chemical Society, 2014, vol. 136, pp. 12938-12946

### SUMMARY OF THE INVENTION

### Problems to be Solved by the Invention

The present invention provides a method of inducing a DEL containing a cleavable site in a DNA strand to a cross linker-modified double-stranded DEL and evaluating the DEL.

### Means to Solve the Problems

As one of chemistry of a nucleic acid such as DNA, there is a technology to cleavage the nucleic acid. For example, when deoxyuridine is introduced into a DNA strand, it can be selectively cleaved by USER® enzyme.

The present inventors have found that, as a result of earnest studies, both the merits of hairpin-stranded DNA and double-stranded DNA can be obtained, for example, by introducing a cleavable site such as deoxyuridine to the DNA strand, and have solved the above problems by easily inducing to a cross linker-modified DEL.

Accordingly, the present invention is as follows.
[1]A method of evaluating a cross linker-modified double-stranded DNA-encoded library (DEL) induced from a hairpin type DEL having “selectively cleavable site(s)”, comprised of the following steps of:
   (1) contacting the DEL with a biological target under conditions suitable for binding at least one library molecule of the DEL to the biological target;
   (2) crosslinking a cross linker of the library molecule bound to the biological target with a biological target;
   (3) separating a complex of the crosslinked library molecule and the biological target from a non-crosslinked library molecule;
   (4) identifying sequences of oligonucleotides that the library molecule in the recovered complex have; and
   (5) using the sequences determined in (4) to identify a structure of one or more compounds that bind to the biological target.
[2] The method according to [1], wherein the cross linker of the cross linker-modified double-stranded DEL is linked to an oligonucleotide having a coding sequence via a covalent bond.
[3] The method according to [1] or [2], wherein the cross linker of the cross linker-modified double-stranded DEL binds to the 5′ end of the oligonucleotide directly or via a bifunctional spacer.
[4] The method according to any of [1] to [3], wherein the induction to the cross linker-modified double-stranded DEL includes the following steps of:
   (i) cleaving at least one “selectively cleavable site” of the hairpin type DEL having the “selectively cleavable site(s)” to convert into a double-stranded DEL;
   (ii) removing an oligonucleotide to which a library molecule is not bound from the double-stranded DEL obtained in (i) to convert into a single-stranded DEL; and
   (iii) forming a double strand of the single-stranded DEL obtained in (ii) and the cross linker-modified DNA to induce to the cross linker-modified double-stranded DEL.
[5] The method according to any of [1] to [3], wherein the induction to the cross linker-modified double-stranded DEL includes the following steps of:
   (i) cleaving at least one “selectively cleavable site” of the hairpin type DEL having the “selectively cleavable site(s)” to convert into a double-stranded DEL;
   (ii) removing an oligonucleotide to which a library molecule is not bound from the double-stranded DEL obtained in (i) to convert into a single-stranded DEL;
   (iii) forming a double strand of the single-stranded DEL obtained in (ii) and a DNA having a reactive group for cross linker modification; and
   (iv) reacting the reactive group for cross linker modification with a cross linker unit to induce to the cross linker-modified double-stranded DEL.
[6] The method according to any of [1] to [3], wherein the induction to the cross linker-modified double-stranded DEL includes the following steps of:
   (i) cleaving at least one “selectively cleavable site” of the hairpin type DEL having the “selectively cleavable site(s)” to convert into a double-stranded DEL;
   (ii) removing an oligonucleotide to which a library molecule is not bound from the double-stranded DEL obtained in (i) to convert into a single-stranded DEL; and
   (iii) adding a cross linker-modified primer to the single-stranded DEL obtained in (ii) and elongating the added primer to induce to a cross linker-modified double-stranded DEL.
[7] The method according to any of [1] to [3], wherein the induction to the cross linker-modified double-stranded DEL includes the following steps of:
   (i) cleaving at least one “selectively cleavable site” of the hairpin type DEL having the “selectively cleavable site(s)” to convert into a double-stranded DEL;
   (ii) removing an oligonucleotide to which a library molecule is not bound from the double-stranded DEL obtained in (i) to convert into a single-stranded DEL;
   (iii) adding a modified primer having a reactive group for cross linker modification to the single-stranded DEL obtained in (ii) and elongating the added primer to induce to a double-stranded DEL having a reactive group for cross linker modification; and
   (iv) reacting the reactive group for cross linker modification with a cross linker unit to induce to a cross linker-modified double-stranded DEL.
[8] The method according to any of [1] to [3], wherein the induction to the cross linker-modified double-stranded DEL includes the following steps of:
   (i) cleaving at least one “selectively cleavable site” of the hairpin type DEL having the “selectively cleavable site(s)” to convert into a double-stranded DEL; and
   (ii) adding a cross linker-modified primer to the double-stranded DEL obtained in (i) and elongating the added primer to induce to a cross linker-modified double-stranded DEL.
[9] The method according to any of [1] to [3], wherein the induction to the cross linker-modified double-stranded DEL includes the following steps of:
   (i) cleaving at least one “selectively cleavable site” of the hairpin type DEL having the “selectively cleavable site(s)” to convert into a double-stranded DEL; and
   (ii) adding a modified primer having a reactive group for cross linker modification to the double-stranded DEL obtained in (i), elongating the added primer, and reacting a reactive group for cross linker modification with a cross linker unit to induce to a cross linker-modified double-stranded DEL.
[10] The method according to [6] or [8], wherein
   (I) the hairpin type DEL in which at least one “selectively cleavable site” is present in a 3′ direction from a site where the library molecule is bound is used, and
   (II) the cross linker-modified primer in which the cross linker binds to the 5′ end of the oligonucleotide directly or via the bifunctional spacer is used.
[11] The method according to [7] or [9], wherein
   (I) the hairpin type DEL in which at least one “selectively cleavable site” is present in a 3′ direction from a site where the library molecule is bound is used, and
   (II) the modified primer having the reactive group for cross linker modification in which the reactive group for cross linker modification binds to the 5′ end of the oligonucleotide directly or via the bifunctional spacer is used.
[12] The method according to any of [4] to [7], wherein in the step (ii), an oligonucleotide to which a library molecule is not bound has a functional molecule and is removed by a treatment according to a function of the functional molecule.
[13] The method according to [12], wherein the functional molecule is biotin.
[14] The method according to any of [4] to [7], wherein in the step (ii), removing the oligonucleotide to which the library molecule is not bound is degradation by exonuclease.
[15] The method according to [14], wherein the exonuclease is lambda exonuclease.
[16] The method according to any of [1] to [15], wherein the cross linker comprises at least one of an azide group, a diazirine group, a sulfonyl fluoride group, a diazo group, a cinnamoyl group, or an acrylate group.
[17] The method according to any of [1] to [15], wherein the cross linker comprises at least one of an azide group, a diazirine group, or a sulfonyl fluoride group.
[18] The method according to any of [1] to [15], wherein the cross linker comprises a structure of any of the formulae (AA) to (AE):

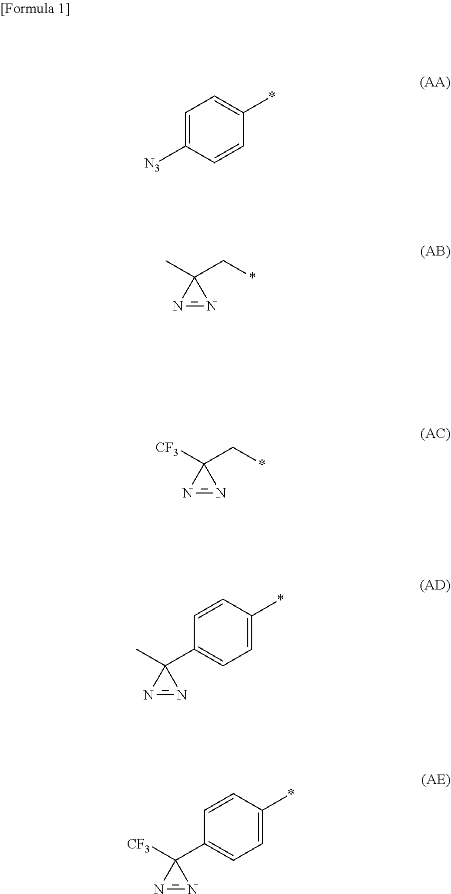

wherein * means a binding site with the 5′ end of the double-stranded DEL or with the bifunctional spacer side binding to the 5′ end of the double-stranded DEL.
[19] The method according to any of [1] to [15], wherein the cross linker is a structure of any of the formulae (AA) to (AE):

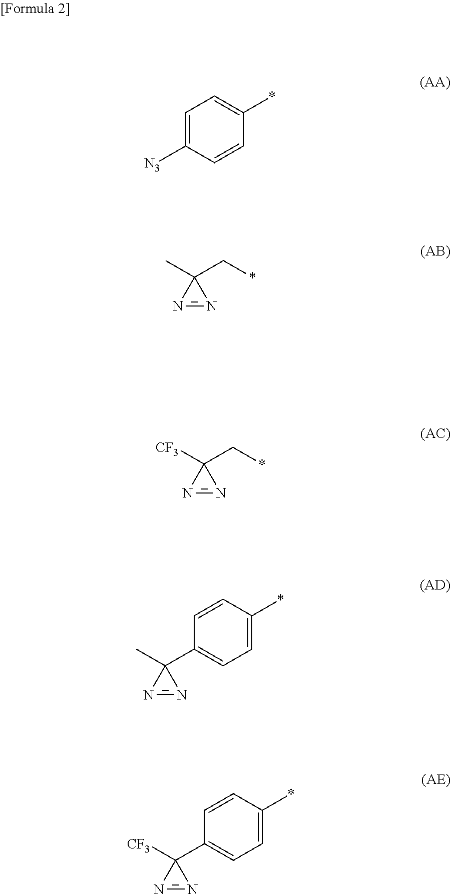

wherein * means a binding site with the 5′ end of the double-stranded DEL or with the bifunctional spacer side binding to the 5′ end of the double-stranded DEL.
[20] The method according to any of [1] to [15], wherein the cross linker comprises a structure of either formula (BA) or (BB):

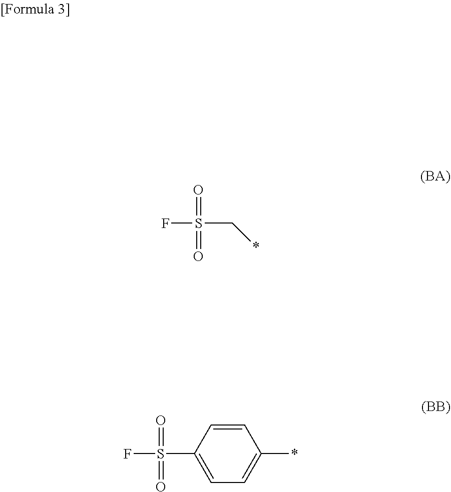 [SMILES: *CS(=O)(=O)F ; *c1ccc(S(=O)(=O)F)cc1]

wherein * means a binding site with the 5′ end of the double-stranded DEL or with the bifunctional spacer side binding to the 5′ end of the double-stranded DEL.
[21] The method according to any of [1] to [15], wherein the cross linker is a structure of either formula (BA) or (BB):

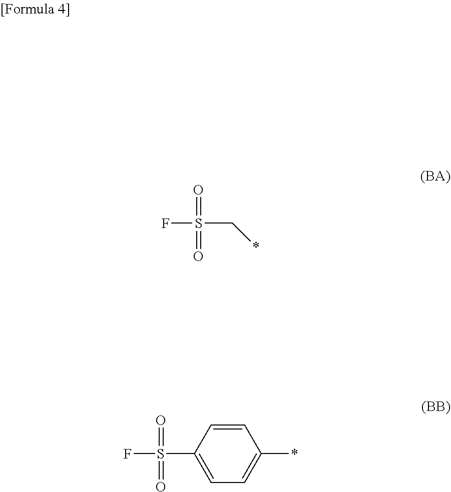 [SMILES: *CS(=O)(=O)F ; *c1ccc(S(=O)(=O)F)cc1]

wherein * means a binding site with the 5′ end of the double-stranded DEL or with the bifunctional spacer side binding to the 5′ end of the double-stranded DEL.
[22] The method according to [5], [7], [9], or [11], wherein the reactive group for cross linker modification is a reactive group for the click reaction.
[23] The method according to [5], [7], [9], or [11], wherein the reactive group for cross linker modification is an alkynyl group, an alkenyl group, an azide group, or a tetrazinyl group.
[24] The method according to [5], [7], [9], or [11], wherein the reactive group for cross linker modification is a structure of any of the formulae (CA) to (CL):

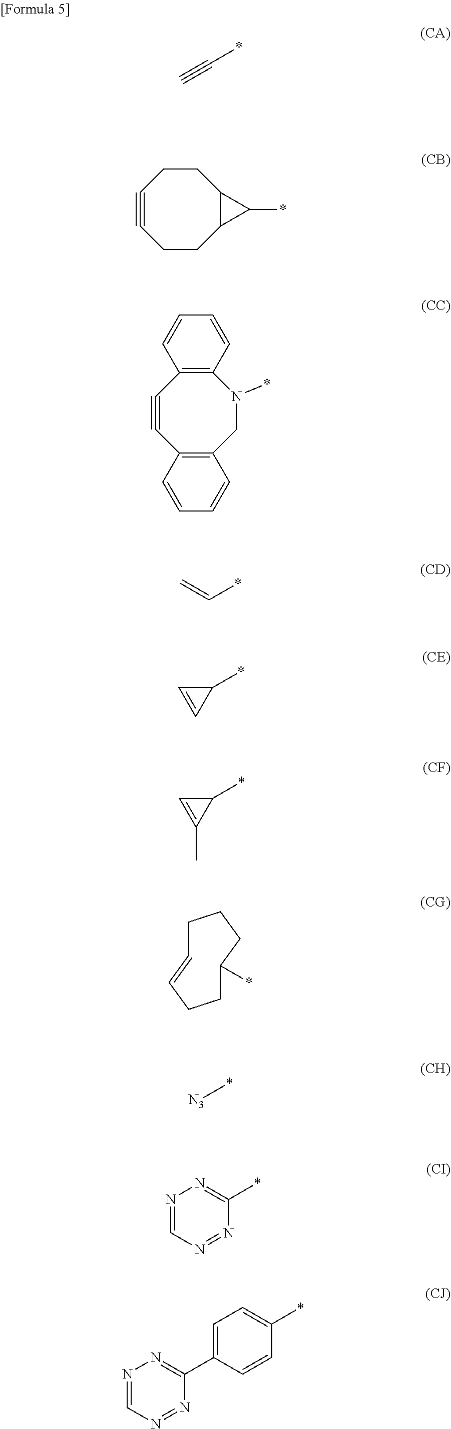

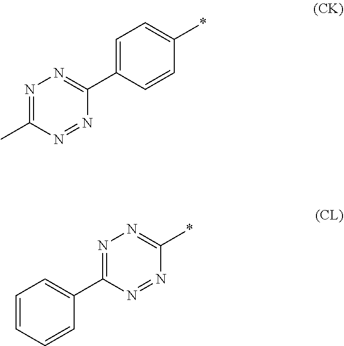 [SMILES: *c1ccc(-c2nnc(C)nn2)cc1 ; *c1nnc(-c2ccccc2)nn1]

wherein * means a binding site with the 5′ end of the double-stranded DEL or with the bifunctional spacer side binding to the 5′ end of the double-stranded DEL.
[25] The method according to any of [1] to [19], wherein the step of “crosslinking the cross linker of the library molecule bound to the biological target with the biological target” in (2) is a step of “crosslinking the cross linker of the library molecule bound to the biological target with the biological target by light irradiation”.
[26] The method according to [25], wherein the light irradiation condition is light irradiation at a wavelength of 250 to 500 nm.
[27] The method according to [25], wherein the light irradiation condition is light irradiation at a wavelength of 365 nm.
[28] The method according to any of [25] to [27], wherein the light irradiation condition is light irradiation for 10 seconds to 180 minutes.
[29] The method according to any of [25] to [27], wherein the light irradiation condition is light irradiation for 30 seconds to 30 minutes.
[30] The method according to any of [1] to [24], wherein the step of “crosslinking the cross linker of the library molecule bound to the biological target with the biological target” in (2) is a step of “crosslinking the cross linker of the library molecule bound to the biological target with the biological target by incubation”.
[31] The method according to any of [1] to [30], wherein the step of “separating the complex of the crosslinked library molecule and the biological target from the non-crosslinked library molecule” in (3) is a step of “separating the complex of the crosslinked library molecule and the biological target from a non-crosslinked library molecule by electrophoresis”.
[32] The method according to [31], wherein the electrophoresis is gel electrophoresis.
[33] The method according to [31], wherein the electrophoresis is capillary electrophoresis.
[34] The method according to any of [1] to [30], wherein the step of “separating the complex of the crosslinked library molecule and the biological target from the non-crosslinked library molecule” in (3) is a step of “separating the complex of the crosslinked library molecule and the biological target by immobilizing the biological target on an immobilization carrier, and removing the non-crosslinked library molecule by washing”.
[35] The method according to any of [1] to [34], wherein the hairpin type DEL having the “selectively cleavable site(s)” is the DEL represented by the formula (I):

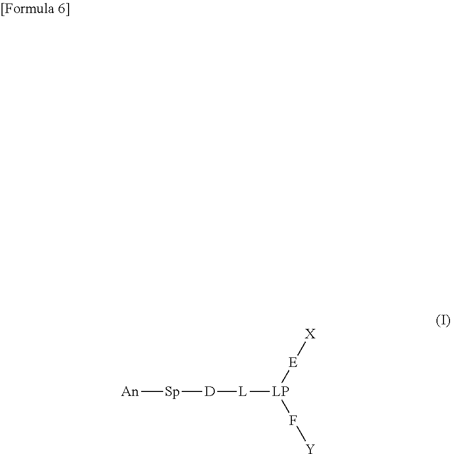

wherein
   X and Y are oligonucleotide chains,
   E and F are each independently an oligomer composed of nucleotides or nucleic acid analogues,
   provided that E and F comprise base sequences complementary to each other and they form a duplex oligonucleotide,
   LP is a loop site,
   L is a linker,
   D is a divalent group derived from a reactive functional group,
   Sp is a bonding or a bifunctional spacer,
   An is a partial structure comprised of at least one building block,
   X and Y have a sequence capable of forming a duplex at least a part thereof,
   X binds to E at the 5′ end,
   Y binds to F at the 3′ end, and
   at least any one site of E, F, or LP has at least one selectively cleavable site.
[36] The method according to [35], wherein the hairpin type DEL having the “selectively cleavable site(s)” is the DEL represented by the formula (III):

An-Sp-C-Bn (III)
wherein
   An and Sp represent the same meanings as defined in [35],
   Bn represents a double-stranded oligonucleotide tag formed by an oligonucleotide chain X and an oligonucleotide chain Y,
   C is the formula (I):

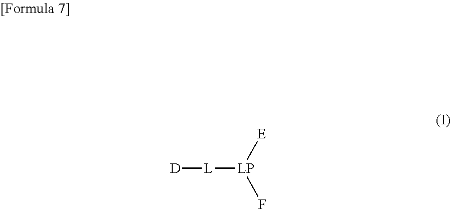

wherein E, LP, L, D, and F represent the same meanings as defined in [35], provided that D binds to An directly or via the bifunctional spacer, and E and F respectively bind to the corresponding terminal side of the double-stranded oligonucleotide tag Bn.
[37] The method according to [35] or [36], wherein An is the same as defined in [35] and is a partial structure constructed by n building blocks α1 to αn (n is an integer of 1 to 10), and
   Bn is a double-stranded oligonucleotide tag formed by an oligonucleotide chain X and an oligonucleotide chain Y and is a partial structure comprising an oligonucleotide which comprises a base sequence capable of identifying the structure of An.
[38] The method according to any of [35] to [37], wherein LP is a loop site represented by (LP1)p-LS-(LP2)q and
   LS is a partial structure selected from a compound group described in the following (A) to (C):
      (A) a nucleotide,
      (B) a nucleic acid analogue, and
      (C) a C1 to 14 trivalent group optionally having a substituent(s);
   LP1 is each a partial structure independently or differently selected with the number of p from a compound group described in the following (1) and (2):
      (1) a nucleotide, and
      (2) a nucleic acid analogue;
   LP2 is each a partial structure independently or differently selected with the number of q from the compound group described in the following (1) and (2):
      (1) a nucleotide, and
      (2) a nucleic acid analogue; and
   the total number of p and q is 0 to 40.
[39] The method according to [38], wherein the total number of p and q is 2 to 20.
[40] The method according to [38], wherein the total number of p and q is 2 to 10.
[41] The method according to [38], wherein the total number of p and q is 2 to 7.
[42] The method according to [38], wherein the total number of p and q is 0.
[43] The method according to any of [38] to [42], wherein LP1, LP2, and LS are each a structure independently or differently selected from the following structures:
   (A) a nucleotide
   or
   (B) a nucleic acid analogue satisfying the following (B11) to (B15):
      (B11) the nucleic acid analogue has phosphoric acid (or a corresponding site thereto) and a hydroxyl group (or a corresponding site thereto),
      (B12) the nucleic acid analogue is comprised of carbon, hydrogen, oxygen, nitrogen, phosphorus or sulfur,
      (B13) the nucleic acid analogue has a molecular weight from 142 to 1,500,
      (B14) the nucleic acid analogue has the number of the atoms between the residues from 3 to 30, and
      (B15) the nucleic acid analogue has the bonding mode of the atoms between the residues in which all bonds are single bonds or in which one or two double bonds are comprised and the remaining are single bonds.
[44] The method according to any of [38] to [43], wherein LP1, LP2, and LS are each a structure independently or differently selected from the following structures:
   (A) a nucleotide
   or
   (B) a nucleic acid analogue satisfying the following (B21) to (B25)
      (B21) the nucleic acid analogue has phosphoric acid and a hydroxyl group,
      (B22) the nucleic acid analogue is comprised of carbon, hydrogen, oxygen, nitrogen, or phosphorus,
      (B23) the nucleic acid analogue has a molecular weight from 142 to 1,000,
      (B24) the nucleic acid analogue has the number of the atoms between the residues from 3 to 15, and
      (B25) the nucleic acid analogue has the bonding mode of the atoms between the residues in which all bonds are single bonds.
[45] The method according to any of [38] to [44], wherein LP1, LP2, and LS are each a structure independently or differently selected from the following structures:
   (A) a nucleotide
   or
   (B) a nucleic acid analogue satisfying the following (B31) to (B35)
      (B31) the nucleic acid analogue has phosphoric acid and a hydroxyl group,
      (B32) the nucleic acid analogue is comprised of carbon, hydrogen, oxygen, nitrogen, or phosphorus,
      (B33) the nucleic acid analogue has a molecular weight from 142 to 700,
      (B34) the nucleic acid analogue has the number of the atoms between the residues from 4 to 7, and
      (B35) the nucleic acid analogue has the bonding mode of the atoms between the residues in which all bonds are single bonds.
[46] The method according to any of [38] to [45], wherein LP1 and LP2 are each any of the following:
   (B41) d-Spacer,
   (B5) a polyalkylene glycol phosphoric acid ester.
[47] The method according to any of [38] to [46], wherein LP1 and LP2 are each diethylene glycol phosphoric acid ester or triethylene glycol phosphoric acid ester.
[48] The method according to any of [38] to [47], wherein LP1 and LP2 are each triethylene glycol phosphoric acid ester.
[49] The method according to any of [38] to [46], wherein LP1 and LP2 are each d-Spacer.
[50] The method according to any of [38] to [45], wherein LP1 and LP2 are each a nucleotide.
[51] The method according to any of [38] to [50], wherein LS is any of the formula (a) to the formula (g):

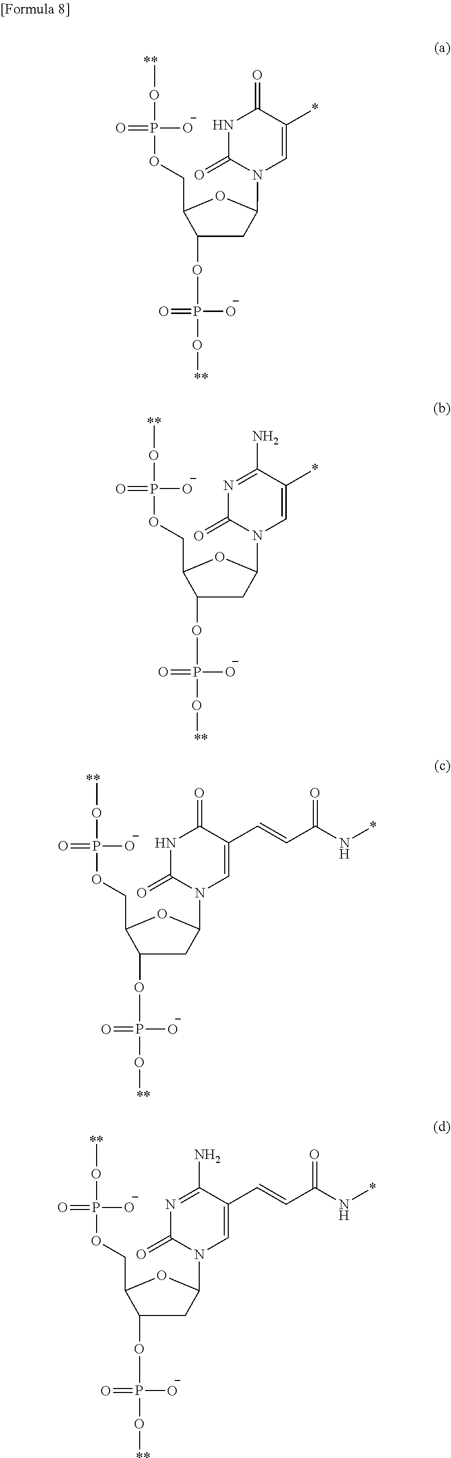

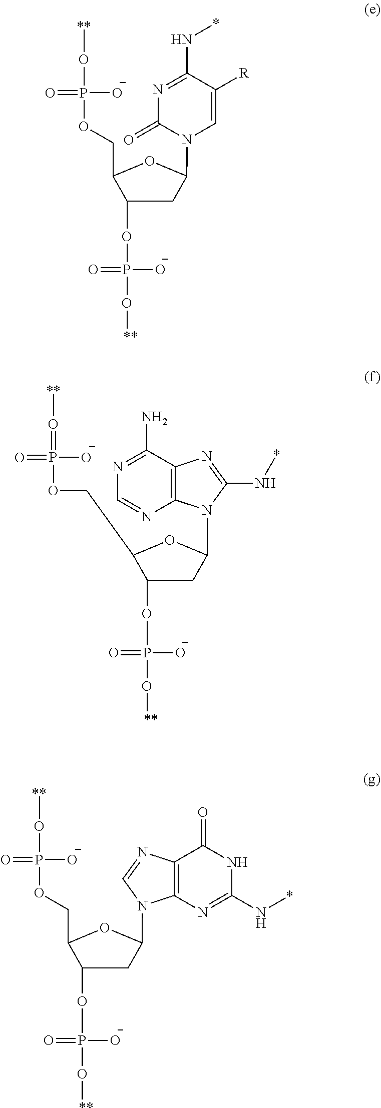

wherein * means a binding site with the linker, ** means a binding site with LP1 or LP2, and R is a hydrogen atom or a methyl group.
[52] The method according to any of [38] to [50], wherein LS is the formula (h):

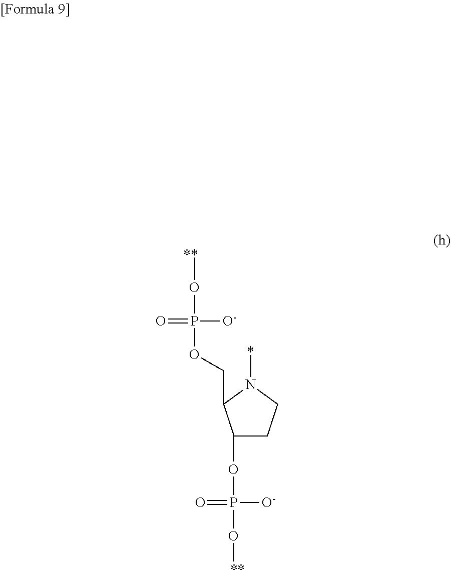

wherein * means a binding site with the linker, and ** means a binding site with LP1 or LP2.
[53] The method according to any of [38] to [50], wherein LS is a polyalkylene glycol phosphoric acid ester.
[54] The method according to any of [38] to [50], wherein LS is any of the formula (i) to the formula (k):

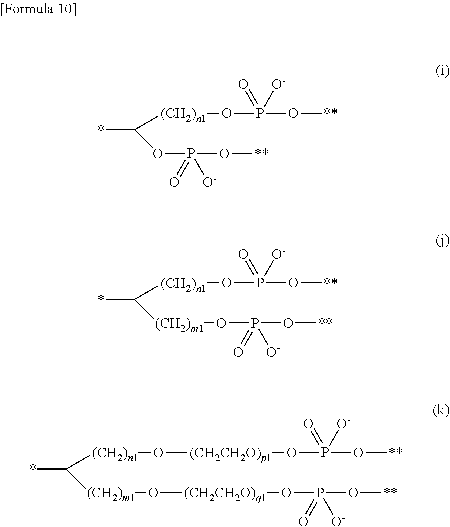

wherein n1, m1, p1, and q1 are each independently an integer of 1 to 20, * means a binding site with the linker, and ** means a binding site with LP1 or LP2.
[55] The method according to any of [38] to [50], wherein LS is the formula (1):

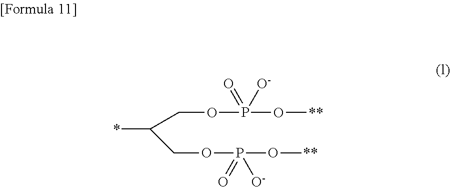

wherein * means a binding site with the linker, and ** means a binding site with LP1 or LP2.
[56] The method according to any of [38] to [50], wherein LS is any of (B42), (B43), or (B44):
   (B42) Amino C6 dT
   (B43) mdC (TEG-Amino)
   (B44) Uni-Link® Amino Modifier.
[57] The method according to any of [38] to [50], wherein LS is a nucleotide.
[58] The method according to any of [38] to [42] and [46] to [50], wherein LS is (C) the C1 to 14 trivalent group optionally having a substituent(s), wherein (C) is any of the following structures:
   (1) a C1 to 10 aliphatic hydrocarbon optionally having a substituent(s) and may be replaced with 1 to 3 hetero atoms,
   (2) a C6 to 14 aromatic hydrocarbon optionally having a substituent(s),
   (3) a C2 to 9 aromatic heterocyclic ring optionally having a substituent(s), or
   (4) a C2 to 9 non-aromatic heterocyclic ring optionally having a substituent(s).
[59] The method according to any of [38] to [42] and [46] to [50], wherein LS is (C) the C1 to 14 trivalent group optionally having a substituent(s), wherein (C) is any of the following structures:
   (1) a C1 to 6 aliphatic hydrocarbon optionally having a substituent(s),
   (2) a C6 to 10 aromatic hydrocarbon optionally having a substituent(s), or
   (3) a C2 to 5 aromatic heterocyclic ring optionally having a substituent(s).
[60] The method according to any of [38] to [42] and [46] to [50], wherein LS is (C) the C1 to 14 trivalent group optionally having a substituent(s), wherein (C) is any of the following structures:
   (1) a C1 to 6 aliphatic hydrocarbon,
   (2) benzene, or
   (3) a C2 to 5 nitrogen-containing aromatic heterocyclic ring,
   wherein (1) to (3) are unsubstituted, or may be substituted with 1 to 3 substituents independently or differently selected from a substituent group ST1, wherein the substituent group ST1 is a group comprised of a C1 to 6 alkyl group, a C1 to 6 alkoxy group, a fluorine atom, and a chlorine atom, provided that when the substituent group ST1 is substituted to the aliphatic hydrocarbon, an alkyl group is not selected from the substituent group ST1.
[61] The method according to any of [38] to [42] and [46] to [50], wherein LS is (C) the C1 to 14 trivalent group optionally having a substituent(s), wherein (C) is any of the following structures:
   (1) a C1 to 6 alkyl group, or
   (2) benzene which is unsubstituted or substituted with one or two C1 to 3 alkyl group(s) or C1 to 3 alkoxy group(s).
[62] The method according to any of [38] to [42] and [46] to [50], wherein LS is (C) the C1 to 14 trivalent group optionally having a substituent(s), wherein (C) is the following structure:
   (1) a C1 to 6 alkyl group.
[63] The method according to any of [35] to [62], wherein E and F are each independently an oligomer composed of nucleotides or nucleic acid analogues, and a chain length of E and F is each 3 to 40.
[64] The method according to any of [35] to [63], wherein E and F are each independently an oligomer composed of nucleotides or nucleic acid analogues, and a chain length of E and F is each 4 to 30.
[65] The method according to any of [35] to [64], wherein E and F are each independently an oligomer composed of nucleotides or nucleic acid analogues, and a chain length of E and F is each 6 to 25.
[66] The method according to any of [35] to [65], wherein E and F are each independently an oligomer composed of nucleotides or nucleic acid analogues,
   E and F comprise base sequences complementary to each other and they form a duplex oligonucleotide, and
   the duplex oligonucleotide of E and F has a protruding end.
[67] The method according to [66], wherein a protruded portion of the protruding end has a length of 2 bases or more.
[68] The method according to any of [35] to [65], wherein
   E and F are each independently an oligomer composed of nucleotides or nucleic acid analogues,
   E and F comprise base sequences complementary to each other and they form a duplex oligonucleotide, and
   the duplex oligonucleotide of E and F has a blunt end.
[69] The method according to any of [35] to [68], wherein chain lengths of the base sequences complementary to each other comprised in E and F are each 3 bases or more.
[70] The method according to any of [35] to [69], wherein chain lengths of the base sequences complementary to each other comprised in E and F are each 4 bases or more.
[71] The method according to any of [35] to [70], wherein chain lengths of the base sequences complementary to each other comprised in E and F are each 6 bases or more.
[72] The method according to any of [35] to [71], wherein E and F are each independently an oligomer composed of a nucleotide.
[73] The method according to any of [35] to [72], wherein the nucleotide is a ribonucleotide or a deoxyribonucleotide.
[74] The method according to any of [35] to [73], wherein the nucleotide is a deoxyribonucleotide.
[75] The method according to any of [35] to [74], wherein the nucleotide is deoxyadenosine, deoxyguanosine, thymidine, or deoxycytidine.
[76] The method according to any of [35] to [71], wherein E and F are each independently an oligomer composed of nucleic acid analogues.
[77] The method according to any of [35] to [76], wherein L is
   (1) a C1 to 20 aliphatic hydrocarbon optionally having a substituent(s) and may be replaced with 1 to 3 hetero atoms, or
   (2) a C6 to 14 aromatic hydrocarbon optionally having a substituent(s).
[78] The method according to any of [35] to [77], wherein L is a C1 to 6 aliphatic hydrocarbon optionally having a substituent(s), a C1 to 6 aliphatic hydrocarbon which may be replaced with one or two oxygen atoms, or a C6 to 10 aromatic hydrocarbon optionally having a substituent(s).
[79] The method according to any of [35] to [78], wherein L is a C1 to 6 aliphatic hydrocarbon which can be substituted with the substituent group ST1 or benzene which can be substituted with the substituent group ST1, wherein the substituent group ST1 is a group comprised of a C1 to 6 alkyl group, a C1 to 6 alkoxy group, a fluorine atom, and a chlorine atom, provided that when the substituent group ST1 is substituted to the aliphatic hydrocarbon, an alkyl group is not selected from the substituent group ST1.
[80] The method according to any of [35] to [79], wherein L is a C1 to 6 alkyl group, or a benzene which is unsubstituted or substituted with one or two C1 to 3 alkyl group(s) or C1 to 3 alkoxy group(s).
[81] The method according to any of [35] to [80], wherein L is a C1 to 6 alkyl group.
[82] The method according to any of [35] to [81], wherein the reactive functional group for D is a reactive functional group which can constitute
   a C—C, amino, ether, carbonyl, amide, ester, urea, sulfide, disulfide, sulfoxide, sulfonamide, or sulfonyl bond.
[83] The method according to any of [35] to [82], wherein the reactive functional group for D is a C1 hydrocarbon having a leaving group, an amino group, a hydroxyl group, a precursor of a carbonyl group, a thiol group, or an aldehyde group.
[84] The method according to any of [35] to [83], wherein the reactive functional group for D is a C1 hydrocarbon having a halogen atom(s), a C1 hydrocarbon having a sulfonic acid-based leaving group, an amino group, a hydroxyl group, a carboxyl group, a halogenated carboxyl group, a thiol group, or an aldehyde group.
[85] The method according to any of [35] to [84], wherein the reactive functional group for D is —CH₂Cl, —CH₂Br, —CH₂OSO₂CH₃, —CH₂OSO₂CF₃, an amino group, a hydroxyl group, or a carboxy group.
[86] The method according to any of [35] to [85], wherein the reactive functional group for D is a primary amino group.
[87] The method according to any of [35] to [86], wherein the selectively cleavable site(s) is/are deoxyribonucleoside which is not any one of deoxyadenosine, deoxyguanosine, thymidine, and deoxycytidine.
[88] The method according to any of [35] to [87], wherein the selectively cleavable site(s) is/are deoxyuridine, bromodeoxyuridine, deoxyinosine, 8-hydroxydeoxyguanosine, 3-methyl-2′-deoxyadenosine, N6-etheno-2′-deoxyadenosine, 7-methyl-2′-deoxyguanosine, 2′-deoxyxanthosine, or 5,6-dihydroxy-5,6 dihydrodeoxy-thymidine.
[89] The method according to any of [35] to [88], wherein the selectively cleavable site(s) is/are deoxyuridine or deoxyinosine.
[90] The method according to any of [35] to [89], wherein the selectively cleavable site(s) is/are deoxyuridine
[91] The method according to any of [35] to [89], wherein the selectively cleavable site(s) is/are deoxyinosine.
[92] The method according to any of [35] to [86], wherein the selectively cleavable site(s) is/are a phosphodiester bond at the second from deoxyinosine in the 3′ direction.
[93] The method according to any of [35] to [86], wherein the selectively cleavable site(s) is/are ribonucleoside.
[94] The method according to any of [35] to [93], wherein the selectively cleavable site is one selectively cleavable site.
[95] The method according to any of [35] to [93], wherein at least one cleavable site is comprised in E or (LP1)p, and at least one cleavable site is comprised in F or (LP2)q.
[96] The method according to [95], wherein the cleavable site comprised in E or (LP1)p and the cleavable site comprised in F or (LP2)q can be cleaved under different conditions.
[97] The method according to any of [35] to [96], wherein An is a partial structure constructed by n building blocks α1 to αn, wherein n is an integer of 1 to 10.
[98] The method according to any of [35] to [97], wherein An is a low molecular weight organic compound.
[99] The method according to any of [35] to [98], wherein the building block for An is a compound having a molecular weight of 500 or less.
[100] The method according to any of [35] to [99], wherein the building block for An is a compound having a molecular weight of 300 or less.
[101] The method according to any of [35] to [100], wherein the building block for An is a compound having a molecular weight of 150 or less.
[102] The method according to any of [35] to [101], wherein An is an organic compound composed of an element selected alone or differently from the element group consisting of H, B, C, N, O, Si, P, S, F, Cl, Br, and I.
[103] The method according to any of [35] to [102], wherein An is a low molecular weight organic compound having a substituent selected alone or differently from a substituent group consisting of an aryl group, a non-aromatic cyclyl group, a heteroaryl group, and a non-aromatic heterocyclyl group.
[104] The method according to any of [35] to [103], wherein An has a molecular weight of 5,000 or less.
[105] The method according to any of [35] to [104], wherein An has a molecular weight of 800 or less.
[106] The method according to any of [35] to [105], wherein An has a molecular weight of 500 or less.
[107] The method according to any of [35] to [97], wherein An is a polypeptide.
[108] The method according to any of [35] to [107], wherein Sp is a bifunctional spacer.
[109] The method according to any of [1] to [107], wherein the bifunctional spacers are each SpD-SpL-SpX, wherein
   SpD is a divalent group derived from a reactive group capable of constituting a C—C, amino, ether, carbonyl, amide, ester, urea, sulfide, disulfide, sulfoxide, sulfonamide, or sulfonyl bond,
   SpL is polyalkylene glycol, polyethylene, a C1 to 20 aliphatic hydrocarbon which may be optionally replaced with a hetero atom(s), a peptide, an oligonucleotide, or a combination thereof, and
   SpX is a divalent group derived from a reactive group which forms an amino, carbonyl, amide, ester, urea, or sulfonamide bond.
[110] The method according to any of [1] to [107], wherein the bifunctional spacers are each SpD-SpL-SpX, wherein
   SpD is a divalent group derived from a primary amino group,
   SpL is polyethylene glycol or polyethylene, and
   SpX is a divalent group derived from a carboxy group.
[111] The method according to any of [35] to [110], wherein the oligonucleotide chain X and the oligonucleotide chain Y are sequences capable of forming a duplex.
[112] The method according to any of [35] to [111], wherein the oligonucleotide chain X and the oligonucleotide chain Y comprise complementary base sequences.
[113] The method according to any of [35] to [112], wherein the oligonucleotide chain X and the oligonucleotide chain Y are each having a length of 1 to 200 bases.
[114] The method according to any of [35] to [113], wherein the oligonucleotide chain X and the oligonucleotide chain Y are each having a length of 3 to 150 bases.
[115] The method according to any of [35] to [114], wherein the oligonucleotide chain X and the oligonucleotide chain Y are each having a length of 30 to 150 bases.
[116] The method according to any of [35] to [115], wherein the oligonucleotide chain X and the oligonucleotide chain Y have a blunt end.
[117] The method according to any of [35] to [115], wherein the oligonucleotide chain X and the oligonucleotide chain Y have a protruding end.
[118] The method according to [117], wherein a protruded portion of the protruding end has a length of 1 to 30 bases.
[119] The method according to [117] or [118], wherein a protruded portion of the protruding end has a length of 2 to 5 bases.
[120] The method according to any of [117] to [119], wherein the oligonucleotide chain X and the oligonucleotide chain Y have a protruding end and a specific molecular recognition sequence is further bonded to the protruding end.
[121] The method according to any of [35] to [120], wherein a functional molecule is bound to any one of X and Y.
[122] The method according to any of [35] to [120], wherein biotin is bound to any one of X and Y.
[123] The method according to any of [35] to [107], wherein Sp is a bond.

### Effects of the Invention

The present invention provides a method of inducing a DEL containing a cleavable site in a DNA strand to a cross linker-modified double-stranded DEL and evaluating the DEL. That is, it provides a technique for screening a compound including both “simple DEL synthesis method” and “expansion and improvement of the DEL evaluation method” as compared with the conventional one. Therefore, according to the present invention, an opportunity to acquire a hit compound useful in the development of drugs, agrochemicals, and medical materials is expanded.

### BRIEF DESCRIPTION OF THE DRAWINGS

FIG. 1 shows an exemplary method of producing a DEL of Form 1. Using a head piece which contains a first oligonucleotide chain containing a cleavable site in a DNA strand, a loop site, and a second oligonucleotide chain as a starting material, binding of building blocks and a double-stranded ligation of the oligonucleotide tag corresponding to the building blocks are repeated (three times in FIG. 1) and further, if desired, double-stranded ligation of the oligonucleotide tag containing the primer region is carried out to accomplish the production of the DEL.

FIG. 2 shows an exemplary method of using a DEL of Form 1. For a DEL containing a cleavable site in a first oligonucleotide chain of a head piece, the cleavable site is cleaved using a cleaving means such as an enzyme to induce to a double-stranded oligonucleotide which is not bound by the loop site, which enables PCR to be carried out with high efficiency.

FIG. 3 shows an exemplary method of using a DEL of Form 2. For a DEL containing a cleavable site in a second oligonucleotide chain of a head piece, the cleavable site is cleaved using a cleaving means such as an enzyme to induce to a double-stranded oligonucleotide which is not bound by the loop site, which enables PCR to be carried out with high efficiency.

FIG. 4 shows an exemplary method of using a DEL of Form 3. For a DEL containing cleavable sites in a first oligonucleotide chain and a second oligonucleotide chain of a head piece, both the cleavable sites are cleaved using a cleaving means such as an enzyme to induce to a double-stranded oligonucleotide which is not bound by the loop site, which enables PCR to be carried out with high efficiency.

FIG. 5 shows an exemplary method of using a DEL of Form 4. For a DEL containing two kinds of cleavable sites different from each other in a first oligonucleotide chain and a second oligonucleotide chain of a head piece, one of the first oligonucleotide chain or the second oligonucleotide chain can be selectively cleaved by selecting the cleavage conditions,

FIG. 6 shows an exemplary method of using a DEL of Form 5. The cleavable site is provided near the terminal of the DNA tag and if desired, the site is cleaved to form a new protruding end. By using the protruding end as a sticky end, a desired nucleic acid sequence, for example, UMIs (a specific molecule identification sequence), etc., can be ligated and a new function can be imparted.

FIG. 7 shows an exemplary method of using a DEL of Form 6. In this invention, a cleavable site can be used in combination with a modifying group or a functional molecule, and for example, it is possible to prepare a DEL in which a hairpin-stranded DNA is converted into a single-stranded DNA. For example, to the synthesized DEL compound, (A) a double-stranded oligonucleotide chain having a functional molecule (for example, biotin) at the 3′ end is ligated, (B) a cleavable site is cleaved, and (C) a treatment depending on a function of the functional molecule is applied. For example, when the functional molecule is biotin, the oligonucleotide chain to which biotin is bound is selectively removed from the system by using streptavidin beads having biotin affinity, etc. Then, it is possible to obtain a DEL having a single-stranded DNA.

FIG. 8 shows an exemplary method of using a DEL obtained in Form 6. For the DEL having the single-stranded DNA obtained in Form 6, a double strand is formed with a modified oligonucleotide having a desired functional site (for example, a cross linker-modified DNA, such as a photoreactive cross linker), which can impart a new function.

FIG. 9 shows an exemplary method of using a DEL of Form 7. In this invention, a cross linker can be introduced using a cleavable site. For the synthesized DEL compound, (A) the cleavable site is cleaved, (B) a modified primer is imparted, and (C) based on the imparted primer, a cross linker-modified double-stranded DEL compound can be synthesized. The cross linker-modified double-stranded DEL compound can markedly improve detection sensitivity in screening of the DEL library (see Non-Patent Documents 7 and 11, etc.).

FIG. 10 is a graph representing a conversion rate of the cleavage reaction at each incubation time when the cleavage reaction of a partial structure of a hairpin type DEL containing deoxyuridine (10 kinds of U-DEL1-sh, U-DEL2-sh, U-DEL3-sh, U-DEL4-sh, U-DEL5-HP, U-DEL6-HP, U-DEL7-HP, U-DEL8-HP, U-DEL9-HP, and U-DEL10—HP) by USER® enzyme was evaluated in Example 1.

FIG. 11 is a schematic drawing showing synthetic procedure of various kinds of hairpin DEL (U-DEL1, U-DEL2, U-DEL4, U-DEL7, U-DEL8, U-DEL9, U-DEL10, H-DEL, U-DEL5, U-DEL11, U-DEL12, U-DEL13, I-DEL1, I-DEL2, I-DEL3, R-DEL1, and BIO-DEL) in Examples 2, 3, 4, 5, and 7. Head pieces corresponding to each are used as starting materials and a hairpin DEL synthesis is accomplished by two-step double-stranded ligation with a double-stranded oligonucleotide Pr_TAG and CP.

FIG. 12 is a graph showing each sample amount which shows the Ct value measured by the real-time PCR of 8 kinds of hairpin DELs (U-DEL1, U-DEL2, U-DEL4, U-DEL7, U-DEL8, U-DEL9, U-DEL10, and H-DEL) and a double-stranded DEL (DS-DEL) in Example 2. Samples in which various kinds of DEL are treated by USER® enzyme are indicated to as “USER(+)” and untreated samples are indicated as “USER(−)”. Cleavable hairpin DELs containing deoxyuridine (U-DEL1, U-DEL2, U-DEL4, U-DEL7, U-DEL8, U-DEL9, and U-DEL10) show the same Ct values as the Ct value of the double-stranded DEL (DS-DEL) after the treatment with USER® enzyme.

FIG. 13 is an image of a gel obtained by modified polyacrylamide gel electrophoresis showing the progress of the cleavage reaction of 6 kinds of hairpin DELs containing deoxyuridines (U-DEL5, U-DEL7, U-DEL9, U-DEL11, U-DEL12, and U-DEL13) by USER® enzyme in Example 3. The numbers in this figure indicate the numbers of each lane.

FIG. 14 is an image of a gel obtained by modified polyacrylamide gel electrophoresis showing the progress of the cleavage reaction of 4 kinds of hairpin DELs containing deoxyinosines (I-DEL1, I-DEL2, I-DEL3, and I-DEL4) by endonuclease V in Example 4. The numbers in this figure indicate the numbers of each lane.

FIG. 15 is an image of a gel obtained by modified polyacrylamide gel electrophoresis showing the progress of the cleavage reaction of hairpin DEL containing ribonucleoside (R-DEL1) by RNaseHII in Example 5. The numbers in this figure indicate the numbers of each lane.

FIG. 16 is a schematic drawing showing a procedure for synthesizing a model library comprising 3×3×3 (27) kinds of compounds using U-DEL9-HP as a starting material. In Example 6, synthesis of the model library is accomplished by 3 times (Cycles A, B, and C) of split-and-pool steps using U-DEL9-HP as a starting material. In each cycle, a ligation reaction of a double-stranded oligonucleotide tag and a chemical reaction for introducing building blocks are included.

FIG. 17 is an image of a gel obtained by agarose gel electrophoresis showing the progress of a ligation reaction in each cycle in a model library synthesis of Example 6. The numbers in the figure indicate the numbers of each lane.

FIG. 18A is a chromatograph obtained from the sample after the completion of Cycle C in a model library synthesis of Example 6. FIG. 18B is a result of deconvolution of MS spectrum obtained by the sample after the completion of Cycle C in a model library synthesis of Example 6.

FIG. 19 is an image of a gel obtained by modified polyacrylamide gel electrophoresis showing the progress of the cleavage reaction of a model library by USER® enzyme in Example 6. The numbers in this figure indicate the numbers of each lane.

FIG. 20 is an image of a gel obtained by modified polyacrylamide gel electrophoresis showing the progress of the cleavage reaction of 5 kinds of DEL compounds having biotin at 3′ end (“AAZ-BIO-DEL”, “SABA-BIO-DEL”, “ClSABA-BIO-DEL”, “mSABA-BIO-DEL”, and “Amino-BIO-DEL”) by USER® enzyme in Example 7. The numbers in this figure indicate the numbers of each lane.

FIG. 21 is an image of a gel obtained by polyacrylamide gel electrophoresis showing the result of conducting a primer elongation reaction using DEL compounds having a single-stranded DNA (“SS-AAZ-DEL”, “SS-SABA-DEL”, “SS-ClSABA-DEL”, “SS-mSABA-DEL”, and “SS-Amino-DEL”) and a photoreactive cross linker-modified primer “PXL-Pr” in Example 7. The numbers in this figure indicate the numbers of each lane.

FIG. 22 is a graph showing a Ct value and a ΔCt value (difference from a Ct value of the negative control) obtained by measuring a recovery amount by real-time PCR to compare the binder recovery efficiencies of various kinds of photoreactive cross linker-modified double-stranded DELs depending on the presence or absence of a photocrosslinking reaction in Example 8. The sample without UV radiation is indicated as “UV(−)” and the sample with UV radiation is indicated as “UV(+)”. In addition, the “solution S” is indicated as “S” and the “solution E” is indicated as “E”. Each notation in the graph corresponds to each sample as follows.

| Notation | |
|---|---|
| in graph: | Sample |
| AAZ: | “PXL-DS-AAZ-DEL” |
| SABA: | “PXL-DS-SABA-DEL” |
| ClSABA: | “PXL-DS-ClSABA-DEL” |
| mSABA: | “PXL-DS-mSABA-DEL” |
| Amino: | “PXL-DS-Amino-DEL” |

FIG. 23 is an image of a gel obtained by polyacrylamide gel electrophoresis showing the result of conducting a primer elongation reaction using DEL compounds having a single-stranded DNA (“SS-AAZ-DEL”, “SS-SABA-DEL”, “SS-ClSABA-DEL”, “SS-mSABA-DEL”, and “SS-Amino-DEL”) and a photoreactive cross linker-modified primer “PXL-Pr2” in Example 10. The numbers in this figure indicate the numbers of each lane.

FIG. 24 is an image of a gel obtained by polyacrylamide gel electrophoresis showing the result of conducting a primer elongation reaction using DEL compounds having a single-stranded DNA (“SS-AAZ-DEL3”, “SS-SABA-DEL3”, “SS-ClSABA-DEL3”, “SS-mSABA-DEL3”, and “SS-Amino-DEL3”) and a photoreactive cross linker-modified primer “PXL-Pr3” in Example 10. The numbers in this figure indicate the numbers of each lane.

FIG. 25 is a graph showing a ΔCt value (difference from the negative control) calculated using a Ct value measured by real-time PCR to compare the binder recovery efficiencies of various kinds of photoreactive cross linker-modified double-stranded DELs having different linker structures depending on the presence or absence of a photocrosslinking reaction in Example 11. The sample without UV radiation is indicated as “UV(−)” and the sample with UV radiation is indicated as “UV(+)”. In addition, each notation in the graph corresponds to each sample as follows.

| Notation | |
|---|---|
| in graph: | sample |
| mSABA-DEL2: | “PXL-DS-mSABA-DEL2” |
| mSABA-DEL3: | “PXL-DS-mSABA-DEL3” |

FIG. 26 is a graph showing a ΔCt value (difference from the negative control) calculated using a Ct value measured by real-time PCR to compare the binder recovery efficiencies of various kinds of “photoreactive cross linker-modified double-stranded DELs having a covalent bond between a cross linker and a coding sequence” with various kinds of “photoreactive cross linker-modified double-stranded DELs without a covalent bond between a cross linker and a coding sequence” in Example 12. Each bar in the graph corresponds to each sample in order from the left as follows.
Leftmost bar: “PXL-DS-SABA-DEL3” without UV radiation
Second bar from the left: “PXL-DS-ClSABA-DEL3” without UV radiation
Third bar from the left: “PXL-DS-mSABA-DEL3” without UV radiation
Fourth bar from the left: “PXL-DS-SABA-DEL3” with UV radiation
Fifth bar from the left: “PXL-DS-ClSABA-DEL3” with UV radiation
Sixth bar from the left: “PXL-DS-mSABA-DEL3” with UV radiation
Seventh bar from the left: “PXL-DS-SABA-DEL4” without UV radiation
Eighth bar from the left: “PXL-DS-ClSABA-DEL4” without UV radiation
Ninth bar from the left: “PXL-DS-mSABA-DEL4” without UV radiation
Tenth bar from the left: “PXL-DS-SABA-DEL4” with UV radiation
Eleventh bar from the left: “PXL-DS-ClSABA-DEL4” with UV radiation
Rightmost bar: “PXL-DS-mSABA-DEL4” with UV radiation

FIG. 27 is a graph showing a ΔCt value (difference from the negative control) calculated using a Ct value measured by real-time PCR to compare the binder recovery efficiencies of various kinds of “photoreactive cross linker-modified double-stranded DELs having a covalent bond between a cross linker and a coding sequence” with various kinds of “photoreactive cross linker-modified double-stranded DELs without a covalent bond between a cross linker and a coding sequence” in Example 13. Each bar in the graph corresponds to each sample in order from the left as follows.
Leftmost bar: “PXL-DS-SABA-DEL3” without UV radiation
Second bar from the left: “PXL-DS-ClSABA-DEL3” without UV radiation
Third bar from the left: “PXL-DS-mSABA-DEL3” without UV radiation
Fourth bar from the left: “PXL-DS-SABA-DEL3” with UV radiation
Fifth bar from the left: “PXL-DS-ClSABA-DEL3” with UV radiation
Sixth bar from the left: “PXL-DS-mSABA-DEL3” with UV radiation
Seventh bar from the left: “PXL-DS-SABA-DEL4” without UV radiation
Eighth bar from the left: “PXL-DS-ClSABA-DEL4” without UV radiation
Ninth bar from the left: “PXL-DS-mSABA-DEL4” without UV radiation
Tenth bar from the left: “PXL-DS-SABA-DEL4” with UV radiation
Eleventh bar from the left: “PXL-DS-ClSABA-DEL4” with UV radiation Rightmost bar: “PXL-DS-mSABA-DEL4” with UV radiation

FIG. 28 is an image of a gel obtained by modified polyacrylamide gel electrophoresis showing the progress of the cleavage reaction of a hairpin DEL compound (“mSABA-DEL5”) by USER® enzyme in Example 14. The numbers in this figure indicate the numbers of each lane.

FIG. 29 is an image of a gel obtained by polyacrylamide gel electrophoresis showing the result of conducting a primer elongation reaction using a DEL compound having a single-stranded DNA (“SS-mSABA-DEL5”) and a photoreactive cross linker-modified primer “PXL-Pr5” in Example 14. The numbers in this figure indicate the numbers of each lane.

FIG. 30 is an image of a gel obtained by polyacrylamide gel electrophoresis showing the result of conducting a primer elongation reaction using a DEL compound “SS-mSABA-DEL” having a single-stranded DNA and cross linker-modified primers “TPD-Pr”, and SS-CISABA-DEL” and “ACA-Pr” in Example 15. The numbers in this figure indicate the numbers of each lane.

FIG. 31 is an image of a gel obtained by polyacrylamide gel electrophoresis showing the result of conducting a primer elongation reaction using a DEL compound having a single-stranded DNA (“SS-mSABA-DEL”) and a reactive group-modified primer for cross linker modification “BCN-Pr” in Example 16. The numbers in this figure indicate the numbers of each lane.

FIG. 32 is an image of a gel obtained by polyacrylamide gel electrophoresis showing the result of conducting a primer elongation reaction using a single-stranded DEL model library, a photoreactive cross linker-modified primer “PXL-Pr”, and a reactive group-modified primer for cross linker modification “BCN-Pr” in Example 17. The numbers in this figure indicate the numbers of each lane.

### EMBODIMENTS TO CARRY OUT THE INVENTION

As mentioned above and a concept well known to those skilled in the art, in the present invention, a compound library means a group of compound derivatives in which compounds possibly having a specific activity such as a drug candidate compound are systematically collected. The compound library is, in many cases, synthesized based on the synthetic techniques and methodologies of combinatorial chemistry. The combinatorial chemistry is a field of experimental methods for efficiently synthesizing a series of compound libraries enumerated and designed based on the combinatorics with a wide variety of compounds by a systematic synthetic route and research relating thereto.

As mentioned above and well known to those skilled in the art, there is a DNA-encoded library as one kind of compound library based on the combinatorial chemistry. The DNA-encoded library is appropriately abbreviated as DEL. Also, DEL is essentially synonymous with a DNA-encoded compound library.

In the present invention, the DNA-encoded library means a library in which a tag of DNA is added to each compound in the library. In the tag of DNA, a sequence is so designed that each structure in each compound can be identified and functions as a label of the compound.

Nucleotides are, in general, understood as substances in which a phosphate group is bound to a nucleoside. Whereas nucleotides and nucleosides are terms well known to those skilled in the art, nucleosides are, as one general embodiment, understood as materials in which a nucleic acid base, such as a purine base or a pyrimidine base, is attached to the 1-position of a sugar such as a pentose via a glycoside bonding. Nucleosides and nucleotides are also units that constitute nucleic acids such as DNA and RNA.

Also, a nucleic acid is a well-known concept for those skilled in the art, and as a general embodiment, it is understood as a polymer of nucleotides.

As one embodiment, the nucleic acid according to the present invention is a polymer composed of nucleotides and nucleic acid analogues mentioned later.

Also, in the present specification, in addition to a nucleic acid polymer composed of nucleotide and nucleic acid analogues, a nucleic acid monomer, such as nucleotides and nucleic acid analogues, may be also simply referred to as a nucleic acid. The latter usage is also a usage according to the common technical knowledge and can be understood by those skilled in the art according to the context as appropriate.

Nucleotides in a broad sense include, in addition to natural nucleotides (original nucleotides), artificial nucleotides (various kinds of nucleic acid analogues). Nucleotides in a broad sense in the present invention include the following embodiments.
(A) Nucleotides of natural nucleosides
   (Examples of the nucleosides include adenosine, thymidine, guanosine, cytidine, uridine, deoxyadenosine, deoxyuridine, deoxyguanosine, deoxycytidine, inosine, and diaminopurine deoxyriboside.)
(B) Nucleotide of nucleoside having analogue of nucleic acid base
   (Examples of the nucleoside having an analogue of nucleic acid base include 2-aminoadenosine, 2-thiothymidine, pyrrolopyrimidine deoxyriboside, 3-methyladenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, 6-O-methylguanosine, and 2-thiocytidine.)
(C) Nucleotides having intercalated nucleic acid base
(D) Unnatural nucleotides having ribose or 2′-deoxyribose
(E) Nucleotides having modified sugar in sugar moiety
   (Examples of the modified sugar include modified ribose, modified 2′-deoxyribose, 2′-O-methylribose, 2′-fluororibose, D-threoninol, arabinose, hexose, anhydrohexytol, altritol, and mannitol.)
(F) Nucleic acid analogues
   (Examples of the nucleic acid analogues include nucleic acids in which oxygen in cyclohexanyl nucleic acid, cyclohexenyl nucleic acid, morpholinonucleic acid (PMO), locked nucleic acid (LNA), glycol nucleic acid (GNA), threose nucleic acid (TNA), serinol nucleic acid (SNA), acyclic threoninol nucleic acid (aTNA) or ribose is replaced.)
   Hereinafter, each nucleic acid analogue will be explained in detail.
(F1) PMO
   PMO is a nucleic acid analogue having a morpholine ring in the sugar moiety and a phosphorodiamidate structure without electric charge in the phosphoric acid diester site.
(F2) LNA
   LNA is a nucleic acid analogue having a crosslinked structure in the sugar moiety and the most typical examples are those in which 2′-hydroxyl of ribose is crosslinked by a C1 to 6 alkylene or C1 to 6 heteroalkylene on the 4′-carbon of the same ribose sugar. Examples of the crosslinked structure include methylene, propylene, ether, or an amino-crosslinked structure.
   Typical LNA includes 2′,4′-BNA (2′-O,4′-C-methano-crosslinked nucleic acid).
(F3) GNA
   Glycol nucleic acid is also called GNA. Examples include R-GNA and S-GNA. In this case, ribose is replaced by a glycol unit(s) bonded to the phosphodiester bond.
(F4) TNA
   Threose nucleic acid is also called TNA. In this case, ribose is replaced by α-L-threofuranosyl-(3′→2′).
(F5) SNA
   Serinol nucleic acid is also called SNA. In this case, ribose is replaced by a serinol unit(s) bonded to the phosphodiester bond.
(F6) aTNA
   Acyclic threoninol nucleic acid is also called aTNA. Examples include D-aTNA and L-aTNA. In this case, ribose is replaced by a threoninol unit(s) bonded to the phosphodiester bond.
(F7) Sugar in which oxygen in ribose is replaced
   Specific examples include those in which oxygen is replaced with S, Se, or alkylene (examples include methylene and ethylene).
(G) Nucleotide in which the skeleton is modified
   (Examples of the nucleotide in which the skeleton is modified include a peptide nucleic acid (the peptide nucleic acid is also called PNA. In this case, 2-aminoethyl-glycine linkage is replaced with ribose and phosphodiester skeleton.).)
(H) Nucleotide in which phosphate group is modified
   (Examples of the nucleotide in which phosphate group is modified include phosphorothioate, 5′-N-phosphoroamidite, phosphoroselenate, boranophosphoric acid, boranophosphate, hydrogen phosphonate, phosphoramidate, phosphorodiamidate, alkyl or aryl phosphonate, phosphotriester, crosslinked phosphoramidate, crosslinked phosphorothioate, crosslinked methylene-phosphonate, and the like.)
   Oligonucleotide, oligonucleotide chain, a double-stranded oligonucleotide, a double-stranded oligonucleotide chain, and a double-stranded DNA of the present invention in the following explanation is the nucleotide as defined above.

In the present invention, when it is described as a nucleotide without any particular limitation, it means a natural nucleotide. The natural nucleotide is a term well known to those skilled in the art and is not particularly limited as long as it is essentially naturally existing nucleotide. As one embodiment, the natural nucleotide in the present invention is the nucleotide described in the above (A).

### (Nucleic Acid Analogue)

Nucleic acid analogue is a term well known to those skilled in the art and the structure of the nucleic acid analogue in the present invention is not limited as long as it has the effect of the present invention

As one embodiment, the nucleic acid analogue is a compound of the embodiments of the above (B) to (H).

As one embodiment, the nucleic acid analogue in the present invention is a compound having a phosphoric acid-corresponding site and a hydroxyl group-corresponding site in the nucleic acid monomer. The nucleic acid analogue is more preferably a compound having a phosphoric acid site and a hydroxyl group.

As one embodiment, the nucleic acid analogue in the present invention is a compound that can be utilized as a monomer in a nucleic acid synthesizer. Whereas it is well known for those skilled in the art, in the nucleic acid synthesizer, a nucleic acid oligomer can be synthesized by converting phosphoric acid (or a corresponding site thereto) of the nucleic acid analogue into a phosphoramidite and utilizing it as a monomer in which a hydroxyl group (or a corresponding site thereto) is protected by a protective group.

The partial structure other than phosphoric acid site (or a corresponding site thereto) and a hydroxyl group (or a corresponding site thereto) in the nucleic acid analogue can be said to be a nucleic acid analogue residue. The structure of the nucleic acid analogue residue is not limited as long as it has the effect of the present invention, but when the characteristics of the respective structures of the natural nucleic acids (deoxyadenosine, thymidine, deoxycytidine, and deoxyguanosine) are confirmed as a reference, the characteristics include that the molecular weight is from about 322 (thymidine monophosphate) to about 347 (deoxyguanosine monophosphate) and the number of the atoms from oxygen atom of the hydroxyl group at the 3′ position and the phosphorus atom at the 5′ position constituting the nucleic acid strand (including the oxygen atom and the phosphorus atom; hereinafter also referred to as the number of atoms between the residues) is 6. As the nucleic acid analogue that can be used for a nucleic acid synthesizer, the following are known.

| | |
|---|---|
| Amino C6 dT | molecular weight: 476, number |
| | of residue atoms: 6 |
| mdC (TEG-Amino) | molecular weight: 526, number |
| | of residue atoms: 6 |
| Uni-Link ® Amino | molecular weight: 227, number |
| Modifier (see Nucleic | of residue atoms: 6 |
| Acid Research, 1992, | |
| vol. 20, pp. 6253-6259) | |
| d-Spacer | molecular weight: 198, number |
| | of residue atoms: 6 |
| Triethylene glycol | molecular weight: 230, number |
| phosphoric acid ester | of residue atoms: 11 |
| (Spacer9) | |

As a reference, the structure of each nucleic acid analogue is described below.

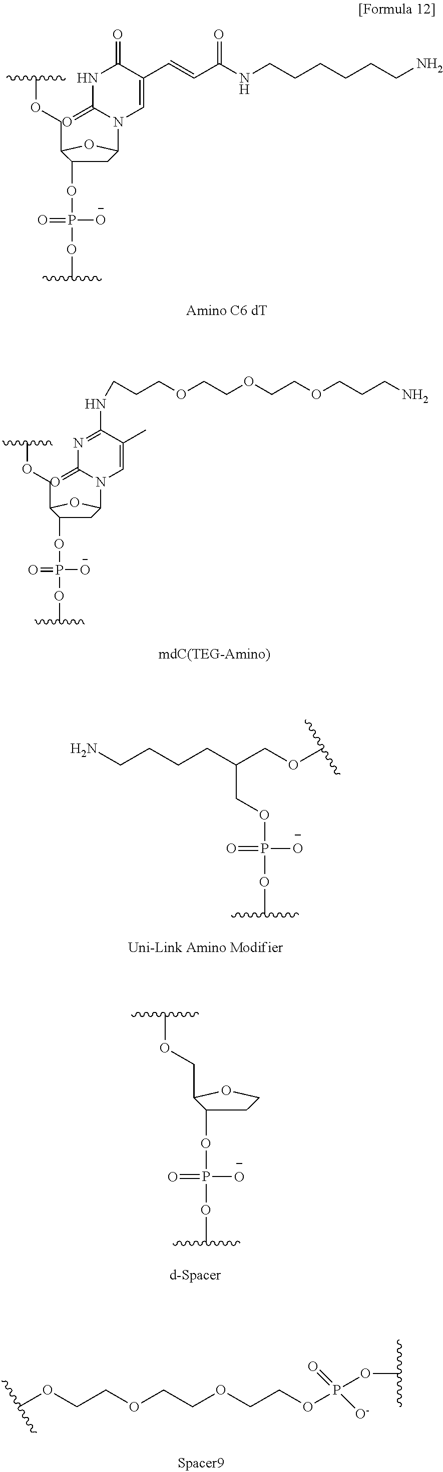 [SMILES: *OCC1OC(n2cc(/C=C/C(=O)NCCCCCCN)c(=O)[nH]c2=O)CC1OP(=O)([O-])O* ; *OCC1OC(n2cc(C)c(NCCCOCCOCCOCCCN)nc2=O)CC1OP(=O)([O-])O* ; *OCC(CCCCN)COP(=O)([O-])O* ; *OCC1OCCC1OP(=O)([O-])O* ; *OCCOCCOCCOP(=O)([O-])O*]

Accordingly, as one embodiment, the nucleic acid analogue is a compound (B1) characterized by the following:
(B11) It has phosphoric acid (or a corresponding site thereto) and a hydroxyl group (or a corresponding site thereto);
(B12) It is comprised of carbon, hydrogen, oxygen, nitrogen, phosphorus, or sulfur;
(B13) The molecular weight is from 142 to 1,500;
(B14) The number of atoms between the residues is from 5 to 30; and
(B15) It has the bonding mode of the atoms between the residues in which all bonds are single bonds or in which one or two double bonds are comprised and the remaining are single bonds.

As one embodiment, the nucleic acid analogue is a compound (B2) characterized by the following:
(B21) It has phosphoric acid and a hydroxyl group;
(B22) It is comprised of carbon, hydrogen, oxygen, nitrogen, or phosphorus;
(B23) The molecular weight is from 142 to 1,000;
(B24) The number of atoms between residues is from 5 to 20; and
(B25) It has the bonding mode of the atoms between the residues in which all bonds are single bonds.

As one embodiment, the nucleic acid analogue is a compound (B3) characterized by the following:
(B31) It has phosphoric acid and a hydroxyl group;
(B32) It is comprised of carbon, hydrogen, oxygen, nitrogen, or phosphorus;
(B33) The molecular weight is from 142 to 700;
(B34) The number of atoms between residues is from 5 to 12; and
(B35) It has the bonding mode of the atoms between the residues in which all bonds are single bonds.

As one embodiment, the nucleic acid analogue is a following compound (B41), (B42), (B43), (B44), (B5), (B51), or (B52).
(B41) d-Spacer
(B42) Amino C6 dT
(B43) mdC (TEG-Amino)
(B44) Uni-Link® Amino Modifier
(B5) Polyalkylene glycol phosphoric acid ester
(B51) Diethylene glycol phosphoric acid ester or triethylene glycol phosphoric acid ester
(B52) triethylene glycol phosphoric acid ester

In the present invention, an oligonucleotide and oligonucleotide chain mean a polymer of a nucleotide having one or more nucleotides at the 5′ end and the 3′ end and at the internal positions between the 5′ end and the 3′ end.

Mutually complementary base sequence means a sequence of nucleotides which can form the so-called complementary base pairs that form a fixed pair of adenine and thymine (or uracil), or guanine and cytosine between two oligonucleotides of nucleic acids and are linked by hydrogen bonds. Formation of the complementary base pairs is also called hybridization.

The complementary base pairs are a concept generally called “Watson-Crick type base pairs” and “natural type base pairs”. However, the base pairs may be Watson-Crick type, Hoogsteen type base pairs, or base pairs by other hydrogen bond motif (for example, diaminopurine and T, 5-methyl C and G, 2-thiothymine and A, 6-hydroxypurine and C, pseudoisocytosine and G) formation, etc. As long as two oligonucleotides are sequences that can form a double strand and can be used for the purpose of the present invention, there is no limitation on the sequence of “mutually complementary base sequence” and there is no limitation on the homology between the two sequences. The homology is preferably, in a more preferable order, 99% or more, 98% or more, 95% or more, 90% or more, 85% or more, 80% or more, 70% or more, 60% or more, or 50% or more.

Whereas it is repeated again, to hybridize in the present invention means an act to form a double strand by oligonucleotides or oligonucleotide chains containing mutually complementary base sequences and a phenomenon to form a duplex by oligonucleotides or oligonucleotide chains containing complementary sequences.

The duplex in the present invention means a state that two nucleic acid strands form complementary base pairs (are hybridized). The two nucleic acid strands may be derived from two nucleic acid strands or may be derived from two nucleic acid sequences in one nucleic acid strand molecule.

In the present invention, the double-stranded oligonucleotide and the double-stranded oligonucleotide chain mean a secondary structure formed by two or more different oligonucleotide chains being hybridized. The chain lengths of the two oligonucleotides may be different and may have regions that are not hybridized.

The region in which the two chains are hybridized is a duplex.

In the present invention, the double-stranded DNA means a secondary structure formed by two different DNA strands being hybridized. The chain lengths of the respective DNA strands may be different and may have regions that are not hybridized. The DNA strands are not limited to naturally existing deoxyribonucleotides and mean all oligonucleotide chains that can be amplified by DNA polymerase.

In the present invention, “forming a duplex” may form a duplex under standard conditions for handling oligonucleotides, for example, at a temperature of 4 to 40° C., an aqueous solvent and a pH of 4 to 10. For example, even if there is a case where no duplex is formed by a specific solvent and conditions, the nucleic acid is a nucleic acid that forms a duplex if the nucleic acid forms a duplex under standard conditions.

In the present invention, the Tm value refers to a temperature at which half of the DNA molecules are annealed with the complementary strand.

In the present invention, the blunt end means that both terminals of the double-stranded oligonucleotide are paired without protruding.

In the present invention, the protruding end means that, among the terminals of the double-stranded oligonucleotide, one of the strands has a protruded portion. The protruded portion of the protruding end can be of any length, and the length is preferably 1 to 50 bases, more preferably 1 to 30 bases, further preferably 1 to 15 bases, and most preferably 2 to 6 bases. In a specific embodiment, it is possible that the protruded portion can be used as a hybridizing region for carrying out ligation of the sticky end.

PCR means a polymerase chain reaction. PCR is means for amplifying the oligonucleotide chains and is a technique well known to those skilled in the art. When the outline of the process of PCR is explained, in PCR, (1) the double-stranded oligonucleotide chain to be amplified is dissociated into two single strands by heat treatment, etc., and (2) after adjusting the temperature suitable for the enzymatic reaction, strands complementary to the respective single strands are synthesized by an enzyme (DNA polymerase, etc.) existing in the reaction system. That is, one double-stranded oligonucleotide can be amplified to be two. In PCR, oligonucleotide chains can be amplified with high efficiency by repeating the processes (1) and (2) by adjusting the temperature.

In the present invention, the primer means an oligonucleotide that is annealed to an oligonucleotide chain which becomes a template and can be elongated by a polymerase in a template-dependent manner.

In the present invention, the primer sequence for PCR means a sequence of a portion in the oligonucleotide chain to which the primer is annealed and is preferably a sequence suitable for PCR as known in the art and is preferably present at the terminal of the oligonucleotide chain.

In the present invention, the nick means a portion in the double-stranded oligonucleotide chain in which a linkage between the nucleotides is lacking and the oligonucleotide chain is broken. The 5′ side of this lacking portion may have a phosphoric acid group or may not have a phosphoric acid group.

In the present invention, the gap means a portion in the double-stranded oligonucleotide chain in which one or more consecutive nucleotides are deleted and the oligonucleotide chains are separated. The 5′ side of the deleted portion may have a phosphoric acid group or may not have a phosphoric acid group.

In the present invention, the hairpin strand is a single-stranded structure in which two complementary nucleic acid strands are linked and the characteristics of the hairpin strand and the hairpin strand DEL are as described above. The terms “hairpin site”, “hairpin structure”, and “hairpin type” used in the present invention are understood as terms derived from the hairpin having the same concept as the above-mentioned “hairpin strand”.

In the present invention, the nucleic acid ligation reaction and ligation mean a reaction in which the terminals of nucleic acids are linked to each other.

The nucleic acid ligation reaction by an enzyme and enzymatic ligation means a reaction in which the terminals of nucleic acids are linked to each other using an enzyme.

An enzyme that can be used in the nucleic acid ligation reaction is, for example, DNA ligase, RNA ligase, DNA polymerase, RNA polymerase, or topoisomerase.

As one embodiment, DNA ligase is an enzyme that ligates the terminals of DNA strands with a phosphoric acid diester bond. As one embodiment, DNA ligase is understood as a ligase belonging to EC number: 6.5.1.1 or 6.5.1.2. DNA ligase is also called polydeoxyribonucleotide synthase or polynucleotide ligase, etc. Examples of DNA ligase include DNA ligases I, II, III, and IV, and T4 DNA ligase, etc.

As one embodiment, RNA ligase is an enzyme that ligates the terminals of RNA strands with a phosphoric acid diester bond. As one embodiment, RNA ligase is understood as a ligase belonging to EC number: 6.5.1.3. Also, as one embodiment, RNA ligase belongs to the lineage of poly(ribonucleotide): poly(ribonucleotide) ligase. RNA ligase is also called as polyribonucleotide synthase or polyribonucleotide ligase.

In the present invention, the chemical ligation means a reaction in which the terminals of the nucleic acids are bound to each other without using an enzyme.

In the chemical ligation, a ligating portion is formed by reacting the terminals of the nucleic acids having a functional group which becomes a pair of the chemical reaction. The functional group which becomes a pair of the chemical reaction is, for example, a pair of an alkynyl group which may be substituted and an azide group which may be substituted, a pair of a diene which may be substituted having a 471 electron system (for example, a 1,3-unsaturated compound which may be substituted, for example, it includes 1,3-butadiene, 1-methoxy-3-trimethylsilyloxy-1,3-butadiene, cyclopentadiene, cyclohexadiene, or furan, each of which may be substituted) and a dienophile which may be substituted or a heterodienophile which may be substituted (for example, it includes an alkenyl group which may be substituted or an alkynyl group which may be substituted) having a 2π electron system, a pair of an amino group which may be substituted and a carboxylic acid group, a pair of a phosphorothioate group and an iodo group (for example, it includes a phosphorothioate group at the 3′ end and an iodo group at the 5′ end), or a pair of a phosphoric acid group and a hydroxy group (for example, it includes a pair of a phosphoric acid group at the 5′ end and a hydroxy group at the 3′ end, or a pair of a hydroxy group at the 5′ end and a phosphoric acid group at the 3′ end).

The chemical ligation is a concept well known to those skilled in the art and those skilled in the art can appropriately achieve chemical ligation based on the common general technical knowledge. In addition to the above, it can be also referred to Artificial DNA; PNA & XNA, 2014, vol. 5, e27896; Current Opinion in Chemical Biology, 2015, vol. 26, pp. 80-88, etc.

In the present invention, “selectively cleavable” means that, in a certain compound, only a specific site can be selectively cleaved under predetermined conditions without changing the other molecular structures of the compound.

In the present invention, a “selectively cleavable site” means, in a certain compound, a site that can be selectively cleaved under predetermined conditions.

As one embodiment, a preferred structure of the “selectively cleavable site” in the present invention is a “selectively cleavable nucleic acid”. The site may be a site that is comprised of a plurality of nucleic acids and can be successfully cleaved by a specific sequence, or may be a site comprised of a single nucleic acid. When the cleavable site is a nucleic acid, it is preferable in the viewpoints that (1) established producing methods, such as a nucleic acid synthesizer, can be utilized so that the production efficiency is good, (2) in the reaction conditions for constructing the building blocks of DEL, it is essential that the nucleic acid at the DNA tag portion is not decomposed, so that when the cleavable site is nucleic acid, it does not decompose as well, etc.

More preferred structure of the above-mentioned “selectively cleavable nucleic acid” is nucleic acid containing a nucleotide which is not comprised in the sequence of the DNA tag of DEL. If the cleavable site is a nucleotide which is not comprised in the sequence of the DNA tag, it is possible to utilize it without limiting the sequence of the DNA tag to avoid the cleavage of the DNA tag portion.

As the nucleic acid used for the sequence of the DNA tag, deoxyadenosine, deoxyguanosine, thymidine, and deoxycytidine are preferable. Therefore, a preferred structure of the selectively cleavable site is a nucleic acid that is neither deoxyadenosine, deoxyguanosine, thymidine, nor deoxycytidine.

Examples of the “selectively cleavable site” include a “nucleotide having a cleavable base”. For example, in the “nucleotide having a cleavable base” in DEL, the N-glycoside bond between the base portion and the sugar portion is cleaved by the action of DNA glycosylase to leave an abasic site. The phosphodiester bond adjacent to the abasic site is cleaved by chemical condition change (for example, temperature rise, basic hydrolysis, etc.) or an enzyme having depurine/depyrimidine (AP) endonuclease activity or AP lyase activity (for example, endonuclease III, endonuclease IV, endonuclease V, endonuclease VI, endonuclease VII, endonuclease VIII, APEl (human-derived AP endonuclease), Fpg (formamide pyridine-DNA glycosylase), etc.) to form a gap with one base unit or a nick.

Examples of the “nucleotide having the cleavable base” include deoxyuridine, bromodeoxyuridine, deoxyinosine, 8-hydroxydeoxyguanosine, 3-methyl-2′-deoxyadenosine, N6-etheno-2′-deoxyadenosine, 7-methyl-2′-deoxyguanosine, 2′-deoxyxanthosine, 5,6-dihydroxydeoxythymidine, etc. Nucleotides having other cleavable bases are obvious to those of skill in the art. By incorporating these “nucleotides having the cleavable base” into DEL and using a DNA glycosylase that specifically recognizes the structure, the DEL is selectively debased.

In the present invention, the DNA glycosylase refers to an enzyme which is an optional enzyme having glycosylase activity, recognizes an optional nucleic acid base portion in the oligonucleotide, cleaves the N-glycoside bond between the base portion and the sugar portion, and creates an abasic site. For example, Examples include uracil DNA glycosylase (recognizing deoxyuridine), alkyladenine DNA glycosylase (recognizing 3-methyl-2′-deoxyadenosine, 7-methyl-2′-deoxyguanosine, and deoxyinosine), Fpg (recognizing 8-hydroxydeoxyguanosine), endonuclease VIII (recognizing decomposed pyrimidine base such as 5,6-dihydroxydeoxythymidine or uracil glycol), SUMG1 (abbreviation of single-strand selective uracil DNA glycosylase, which recognizes deoxyuridine), etc.

In the present invention, more preferable examples of the “selectively cleavable site include deoxyinosine and deoxyuridine.

In the present invention, particularly preferable examples of the “selectively cleavable site” include deoxyuridine.

As one embodiment, the “selectively cleavable site” in the present invention is preferably cleaved using an enzyme. The enzyme generally has high substrate specificity and does not recognize the DNA tag portion of DEL and the compound portion constructed by a plurality of building blocks as a substrate and recognizes only the “selectively cleavable site” and acts so that it is preferable. Also, the cleavage using the above-mentioned enzyme may be achieved by changing the structure of the “selectively cleavable site” by the enzyme and then changing the chemical conditions. Examples of the enzyme include glycosylase and nuclease.

In the present invention, the glycosylase is an enzyme having a function of hydrolyzing a glycoside bond (a covalent bond formed by dehydration condensation of a sugar molecule and another organic compound). Among them, the DNA glycosylase is an enzyme that recognizes the nucleic acid base portion in the oligonucleotide, as mentioned above, and hydrolyzes the glycoside bond.

In the present invention, the nuclease is an enzyme having a function of hydrolyzing a phosphodiester bond between the sugar and the phosphoric acid of the nucleic acid. The nuclease includes AP endonuclease, nicking endonuclease, and ribonuclease, for example.

The AP endonuclease cleaves a phosphodiester bond adjacent to the abasic site formed by the action of an optional DNA glycosylase as mentioned above. Therefore, in the present invention, it is preferable to use DNA glycosylase and AP endonuclease in combination.

The nicking endonuclease (for example, Nb.BbvCI, Nb.BsmI, Nb.BsrDI, etc.) recognizes a specific DNA sequence and generates a nick in which a phosphodiester bond is cleaved only one strand among the double strand. Also, the endonuclease V can generate a nick in which the second phosphodiester bond in the 3′ direction from the deoxyinosine is cleaved, which is useful for carrying out the present invention.

The ribonuclease is an enzyme that decomposes RNA. In the present invention, the ribonucleoside is used as the “selectively cleavable site” and it can be utilized by allowing ribonuclease to act on it. RNaseHII, which is a kind of the ribonuclease, can generate a nick in which the phosphodiester bond on the 5′ side of the ribonucleotide incorporated into the DNA sequence is cleaved, which is useful for carrying out the present invention.

In the present invention, USER® means “Uracil-Specific Excision Reagent” Enzyme. The USER is an endonuclease cocktail that removes uracil including uracil DNA glycosylase (UDG) and endonuclease VIII. The USER removes uracil in the double-stranded DNA to generate a one base gap to cleave the DNA strand. In the process of the USER, UDG firstly removes an uracil base to produce an abasic site. Subsequently, the endonuclease decomposes a phosphodiester bond to liberate a deoxyribose having no base to produce a one base gap.

In the explanation of the present specification, USER® enzyme and USER® Enzyme are USER® in the above-mentioned definition.

In the present invention, the exonuclease is an enzyme having a function of sequentially hydrolyzing phosphodiester bonds from the 5′ end or 3′ end of nucleic acids. The exonuclease includes lambda exonuclease, exonuclease III, and T7 exonuclease, for example.

The lambda exonuclease is an enzyme that decomposes DNA whose 5′ end in the double-stranded DNA is phosphorylated. In the present invention, it is useful in the scene where a double-stranded DEL is converted into a single-stranded DEL.

In the present invention, the building block is a portion that has a functional group and can constitute a part of a compound, which may be in the form of a compound.

In the present invention, the base sequence which can identify the respective building blocks means a specific base sequence designed to correspond to the structures of the respective building blocks. To design a sequence means, for example, to assign the nucleic acid base sequence to each structure such as to assign the nucleic acid base sequence AAA to the building block structure A, the nucleic acid base sequence TTT to the structure B, and the nucleic acid base sequence CGC to the structure C. The sequence can be freely designed as long as the object of the present invention is achieved. For example, an optional number of base sequences can be assigned to one building block.

In the present invention, the oligonucleotide tag is a partial structure comprising an oligonucleotide which comprises a base sequence capable of identifying the structure of the partial structure constructed by the building blocks. In the present invention, the oligonucleotide tag may be an oligonucleotide corresponding to each building block, or may be a longer chain oligonucleotide containing an oligonucleotide corresponding to a plurality of building blocks.

The nucleotide constituting the oligonucleotide tag of the present invention is not limited as long as it can accomplish the effect of the present invention and, in the viewpoint of easiness of amplification by PCR and analysis by a sequencer, it is desirably a nucleotide suitable for these operations. Examples of such a preferable nucleotide include a nucleotide having the above-mentioned natural nucleic acid base as the base portion and having the above-mentioned ribose or 2′-deoxyribose as the sugar portion and more preferable examples include deoxyadenosine, thymidine, deoxycytidine, and deoxyguanosine.

### (Head Piece)

In the present invention, the head piece means a starting compound for producing a compound library such as DEL. The structure of the head piece according to the present invention is not limited as long as it can accomplish the object of the present invention, but in the most typical embodiment, it has at least one site to which the building block can be linked and at least one site to which the oligonucleotide tag can be ligated and further contains at least one selectively cleavable site in the structure.

As described later, the DNA tag is preferably a double-stranded oligonucleotide chain and the site to which the oligonucleotide tag can be ligated is preferably two.

As one embodiment, the head piece is a compound shown in the following schematic drawing.

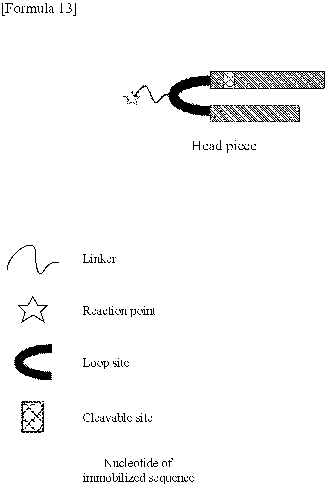

As one embodiment, the head piece is desirably to be chemically stable.

In addition, as one embodiment, the head piece preferably has a structure in which the DNA tag and the building block can be arranged in an appropriate space.

As one embodiment, it is preferable that the head piece has appropriate flexibility.

Here, more appropriate spatial arrangement and flexibility (structural characteristics of the head piece) will be explained. Here, the structural characteristics of the head piece to be explained may be achieved by the head piece alone or may be achieved by coupling the head piece with a bifunctional spacer.

As one embodiment, the preferred structural characteristics of the head piece are structural characteristics that the head piece or the DNA tag does not inhibit the forming reaction of the building block and conversely the head piece or the building block does not inhibit the elongation reaction of the DNA tag.

As one embodiment, the preferred structural characteristics of the head piece are structural characteristics that the head piece or the DNA tag portion does not affect the interaction between the building block compound (library compound) and the target (target protein, etc.).

As one embodiment, the preferred structural characteristics of the head piece are structural characteristics that the DNA tag and the building block site are oriented on opposite sides (for example, 90 degrees or more on the opposite side).

As one embodiment, the preferred structural characteristics of the head piece are structural characteristics that the loop site and the building block of the head piece are separated from several atoms to a dozen atoms in terms of the skeleton of the organic compound.

As one embodiment, the head piece preferably has an appropriate affinity with the DNA tag portion and the building block portion. The appropriate affinity means, for example, chemical reactivity and stability so that a bond can be formed, maintained, and cleaved under desired conditions for carrying out the present invention.

In the present invention, the bifunctional spacer means a spacer portion having at least two reactive groups that enables binding between the building block site and the head piece.

In the explanation of the present invention, the terms “head piece”, “head piece compound”, and “compound for the head piece” are terms referring to the same conceptual compound.

In the explanation of the present invention, a “compound used as a head piece” can be understood essentially the same as “use of a compound as a head piece” from the viewpoint of use and can be understood essentially the same as the “method of using the compound as a head piece” from the viewpoint of a method. The same applies to the compound library.

Hereinafter, a structure of the preferred head piece is explained but the structure of the head piece is not limited as long as the effects of the present invention are achieved.

As one embodiment, the head piece is comprised of
(D) a reactive functional group having at least one site that can be linked directly or linked indirectly via a bifunctional spacer to the building block,
(L) a linker elongating from the reactive functional group,
(E) a first oligonucleotide chain having one binding site that can be linked to one of the strands of the oligonucleotide tag,
(F) a second oligonucleotide chain having one binding site that can be linked to the other strand of the oligonucleotide tag, and
(LP) a loop site that that can be linked to the above-mentioned linker and two oligonucleotide chains,
wherein at least any one site of E, F, or LP has at least one selectively cleavable site.

As one embodiment, the head piece is a compound represented by the following formula (I).

The compound represented by

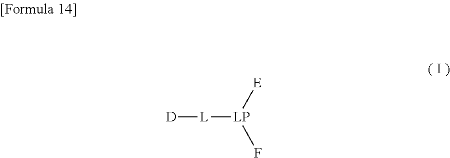

wherein E and F are each independently an oligomer composed of nucleotides or nucleic acid analogues,
provided that E and F comprise base sequences complementary to each other and they form a duplex oligonucleotide,
LP is a loop site,
L is a linker, and
D is a reactive functional group,
wherein at least any one site of E, F, or LP has at least one selectively cleavable site.

In the present invention, among the loop sites, the partial structure of the site that binds to the linker may be sometimes referred to as a linking site or (LS).

Also, in the present invention, E-LP-F may be sometimes collectively referred to as a hairpin site.

### (First and Second Oligonucleotide Chains)

Hereinafter, preferred embodiments of the first oligonucleotide chain (E) and the second oligonucleotide chain (F) will be explained.

It is preferable that the first oligonucleotide chain (E) and the second oligonucleotide chain (F) form a duplex in the molecule via the loop site (LP) and the head piece form a hairpin structure. The chain length preferable for the formation of the duplex in the molecule is 3 bases or more, more preferably 4 bases or more, and further preferably 6 bases or more.

The chain length of E and F is, as one embodiment, each 3 to 40, respectively.

The chain length of E and F is, as one embodiment, each 4 to 40, respectively.

The chain length of E and F is, as one embodiment, each 6 to 25, respectively.

The site to which the oligonucleotide tag is linked preferably has a structure suitable for enzymatic ligation or chemical ligation. As one embodiment, the ligation between the head piece and the oligonucleotide tag is carried out by double-stranded ligation using an enzyme. In that case, it is preferable that the first and the second oligonucleotide chains form a protruding end for ligation. The above-mentioned chain length of the protruding end is preferably 2 bases or more, more preferably 2 to 10 bases, and further preferably 2 to 5 bases. Accordingly, it is preferable that one of the first and the second oligonucleotide chains is longer than the other chain by the chain length of the protruding end. Also, for ligation with the DNA ligase, among the first and the second oligonucleotide chains, it is preferable that the 5′ end of the chain having the 5′ end of the head piece is phosphorylated.

In addition, the first and the second oligonucleotide chains may contain a part or whole of the primer binding sequence for PCR. The appropriate chain length for the primer binding sequence is 17 to 25 bases.

### (Linker)

Hereinafter, preferred embodiments of the linker (L) will be explained.

The linker is, as mentioned above, a site that elongates from the reactive functional group and binds to the linking site. Typically, the linker is a divalent group (-L-) derived from the following embodiments.

As one embodiment, the linker is the following embodiment (L1).
(L1) C1 to 20 aliphatic hydrocarbon optionally having a substituent(s) and may be replaced with 1 to 3 hetero atoms, or (2) C6 to 14 aromatic hydrocarbon optionally having a substituent(s).

As other embodiments, L is the following embodiment (L2), (L3), (L4), or (L5).
(L2)
C1 to 6 aliphatic hydrocarbon optionally having a substituent(s), C1 to 6 aliphatic hydrocarbon which may be replaced with one or two oxygen atoms, or C6 to 10 aromatic hydrocarbon optionally having a substituent(s).
(L3)
C1 to 6 aliphatic hydrocarbon which can be substituted with the substituent group ST1, or benzene which can be substituted with the substituent group ST1. Here, the substituent group ST1 is a group comprised of a C1 to 6 alkyl group, a C1 to 6 alkoxy group, a fluorine atom, and a chlorine atom. Provided that when the substituent group ST1 is substituted to the aliphatic hydrocarbon, an alkyl group is not selected from the substituent group ST1.
(L4)
C1 to 6 alkyl, or benzene which is unsubstituted or substituted with one or two C1 to 3 alkyl group(s) or C1 to 3 alkoxy group(s).
(L5)
C1 to 6 alkyl.

### (Reactive Functional Group)

Hereinafter, preferred embodiments of (D) the reactive functional group will be explained.

As described above, the reactive functional group has at least one site that can be linked directly or linked indirectly via the bifunctional spacer to the building block and is a site that binds to the linker group. Typically, the reactive functional group becomes a monovalent group (D-) in the head piece and it becomes a “divalent group derived from the reactive functional group” (-D-) based on the above-mentioned (D-) in the DEL.

For example, when D is an amino group, the specific structure of (D-) is (R—HN—) (R is a substituent explained below). For example, it reacts with an activated carboxy group, a reactive sulfonyl group, or an isocyanate group to form an amide bond, a sulfonamide bond, or urea bond, respectively. At that time, the specific structure of (-D-) is (—NR—).

R is not limited as long as the effects of the present invention are accomplished but in the following embodiments of (D1) to (D5), R is preferably (1) a hydrogen atom, or (2) a C1 to 6 alkyl group which is unsubstituted or substituted with 1 to 3 substituents selected solely or different from a substituent group consisting of a C1 to 6 alkoxy group, a fluorine atom, and a chlorine atom.

R is more preferably a hydrogen atom or a C1 to 3 alkyl group, and further preferably a hydrogen atom.

Also, for example, when (D-) is a methylene group having a leaving group (X—), the specific structure of (D-) is (X—CH₂—) and, for example, it reacts with a nucleophilic reagent such as an amino group, a hydroxy group, or a thiol group to form a carbon-nitrogen bond, a carbon-oxygen bond or a carbon-sulfur bond. At that time, the specific structure of (-D-) is (—CH₂—). Also, for example, when (D-) is an aldehyde group, the specific structure of (-D-) is (HOC—). The aldehyde group forms a carbon-nitrogen bond, for example, by the reductive amination reaction with an amino group, and at that time, (-D-) is —CH₂—; and forms a carbon-carbon double bond, for example, by the reaction with a phosphorus-iride group, and at that time, (-D-) is —CH═; and forms a carbon-carbon triple bond, for example, by the reaction with an α-diazophosphonate group, and at that time, (-D-) is —C—.

As one embodiment, the site (D-) is the following embodiment (D1).
(D1)
Functional groups capable of constituting C—C, amino, ether, carbonyl, amide, ester, urea, sulfide, disulfide, sulfoxide, sulfonamide, or sulfonyl bond. (Although it is literal, in this case, (-D-) is C—C, amino, ether, carbonyl, amide, ester, urea, sulfide, disulfide, sulfoxide, sulfonamide, or sulfonyl bond.)

As other embodiments, (D-) is the following embodiment (D2), (D3), (D4), or (D5).
(D2)
C1 hydrocarbon having a leaving group, a precursor of amino group, hydroxyl group, or carbonyl group, a thiol group, or an aldehyde group.

In this case, (-D-) can be —(C1 hydrocarbon)-, —NR—, —O—, —(C═O)—, —S—, —CH₂—, —CH═, —C≡, and the like.
(D3)
C1 hydrocarbon having a halogen atom(s), C1 hydrocarbon having a sulfonic acid-based leaving group, an amino group, a hydroxyl group, a carboxy group, a halogenated carboxy group, a thiol group, or an aldehyde group.

In this case, (-D-) can be —(C1 hydrocarbon)-, —NR—, —O—, —(C═O)—, —S—, —CH₂—, —CH=, —C≡, and the like
(D4)
—CH₂Cl, —CH₂Br, —CH₂OSO₂CH₃, —CH₂OSO₂CF₃, an amino group, a hydroxyl group, or a carboxy group.

In this case, (-D-) can be —CH₂—, —NR—, —O—, or —(C═O)—, respectively.
(D5)
Primary amino group.

In this case, (-D-) is —NH—.

Hereinafter, preferred embodiments of the loop site (LP) will be explained.

The loop site (LP) is preferably so designed that the first oligonucleotide chain (E) and the second oligonucleotide chain (F) form a duplex in the molecule and the head piece can form a hairpin structure. That is, the loop site (LP) preferably has a chain length that makes the loop structure thermodynamically stable and flexibility of bonding.

Accordingly, as one embodiment, the loop site (LP) is as follows.
LP is a loop site represented by (LP1)p-LS-(LP2)q,
LS is a partial structure selected from the compound group described in the following (A) to (C),
   (A) a nucleotide,
   (B) a nucleic acid analogue, and
   (C) a C1 to 14 trivalent group optionally having a substituent(s);
LP1 is each a partial structure independently or differently selected with the number of p from the compound group described in the following (1) and (2),
   (1) a nucleotide, and
   (2) a nucleic acid analogue;
LP2 is each a partial structure independently or differently selected with the number of q from the compound group described in the following (1) and (2),
   (1) a nucleotide, and
   (2) a nucleic acid analogue;
wherein the total number of p and q is 0 to 40.

Further preferred embodiments of the loop site are as explained above.

Hereinafter, the structure of the loop site will be further supplemented.

Here, the nucleotide is the natural nucleotide of the above-mentioned explanation and the nucleic acid analogues is as the above-mentioned explanation.

Here, LP1 is each a partial structure independently or differently selected with the number of p from the compound group described in the following (1) and (2), and LP2 is each partial structure selected solely or differently from the compound groups described in the following (1) and (2) with the number of q.
(1) a nucleotide, and
(2) a nucleic acid analogue

Selected solely or differently with the number of q is that, for example, when p is 4, LP1 can be selected solely or differently from the compound group described in (1) and (2), like AATG, ATCG, TC (d-Spacer) G or A (d-Spacer) (d-Spacer) C. The same applies to LP2.

Also, the loop site may contain a part or whole of the primer bond sequence for PCR.

### (As for LS)

As one embodiment, LS is (A) a nucleotide or (B) a nucleic acid analogue.

When LS is (A) a nucleotide or (B) a nucleic acid analogue, the loop site is a nucleic acid oligomer. The nucleic acid oligomer according to the present invention refers to an oligomer in which the nucleotide or the nucleic acid analogues is linked as a monomer. The oligomer can be also said to be a chain state compound.

Accordingly, the nucleic acid oligomer according to the present invention is any of an oligonucleotide chain, a nucleic acid analogue chain, or a mixed chain of a nucleotide and a nucleic acid analogue.

When LS is (A) a nucleotide or (B) a nucleic acid analogue, the loop site is a nucleic acid oligomer. In this case, the head piece can be produced by a nucleic acid synthesizer, which is markedly preferable in practice.

When LS is (A) a nucleotide or (B) a nucleic acid analogue, in the production of the head piece, as one embodiment, a monomer for nucleic acid synthesis in which the linker site (L) and the reactive functional group site (D) are bound to LS is prepared and then a nucleic acid oligomer can be synthesized.

Examples of such a monomer for nucleic acid synthesis include the above-mentioned Amino C6 dT, mdC (TEG-Amino), Uni-Link® Amino Modifier, and the like.

In the case of this embodiment, for example, among the structures of mdC (TEG-Amino), which is the monomer, the nucleotide portion corresponds to the linking site (LS), and the side chain portion elongating from the base corresponds to the linker site (L) and the reactive functional group site (D).

In the preparation, the reactive functional group (D) may be protected by a protective group.

In such a case, as one embodiment, the nucleic acid analogue is the following compound (B6).
(B6) A compound in which the above-mentioned (-L-D) is bound to the base portion of a nucleotide.

As one embodiment, the nucleic acid analogue is the following compound (B61), (B62), (B63), (B64), or (B65).
(B61) (B6) in which (-L-D) is (-L1-D1)
(B62) (B6) in which (-L-D) is (-L2-D2).
(B63) (B6) in which (-L-D) is (-L3-D3).
(B64) (B6) in which (-L-D) is (-L4-D4).
(B65) The compound described in any of (B61) to (B64) in which (-D) is (-D5).

When LS is (A) a nucleotide or (B) a nucleic acid analogue, in the production of the head piece, as one embodiment, a nucleic acid oligomer is firstly synthesized, and then the above-mentioned linker site (L) and the reactive functional group site (D) can be bound to it.

In such a case, it is preferable to put the “specific nucleic acid analogue” to which the linker site binds into the hairpin site (nucleic acid analogues oligomer) as the linking site (LS). Examples of the “specific nucleic acid analogue” include the above-mentioned Amino C6 dT, mdC (TEG-Amino), and Uni-Link® Amino Modifier.

In the case of this embodiment, for example, mdC (TEG-Amino) itself corresponds to a linking site (LS), and additional sites that further bind from the chain on the base side correspond to the linker site (L) and the reactive functional group site (D).

### (As for p and q)

As mentioned above, it is preferable that the chain length of the above-mentioned loop site is the one so that the first oligonucleotide chain (E) and the second oligonucleotide chain (F) form a duplex in the molecule and the head piece forms the hairpin structure.

As one embodiment, the total number of p and q is 1 to 40.

As one embodiment, the total number of p and q is 2 to 20.

As one embodiment, the total number of p and q is 2 to 10.

As one embodiment, the total number of p and q is 2 to 7.

As one embodiment, the loop site of the present invention is comprised of
(A) a nucleotide and the following nucleic acid analogue (B41), (B42), (B43), (B44), or (B52).
(B41) d-Spacer
(B42) Amino C6 dT
(B43) mdC (TEG-Amino)
(B44) Uni-Link® Amino Modifier
(B52) triethylene glycol phosphoric acid ester

As one embodiment, LS is preferably B42, B43, or B44.

Also, as one embodiment, LP1 and LP2 are preferably A, B41, or B52.

As one embodiment, the loop site is a nucleic acid oligomer according to the sequences described in the following (X1) to (X9).
(X1) A-B41-B42-B41-A
(X2) A-B41-B43-B41-A
(X3) A-B41-B44-B41-A
(X4) B41-B41-B42-B41-B41
(X5) B41-B41-B43-B41-B41
(X6) B41-B41-B44-B41-B41
(X7) B52-B42-B52
(X8) B52-B43-B52
(X9) A52-A44-A52

In the above-mentioned head piece, the number of the cleavable sites is preferably within 5 and more preferably 1 to 2.

In the above-mentioned head piece, when the cleavable sites are two or more, it is preferable that at least one cleavable site is in the first oligonucleotide chain or between the first oligonucleotide chain and the linker binding site and at least one cleavable site is in the second oligonucleotide chain or between the second oligonucleotide chain and the linker binding site.

As one embodiment, in the above-mentioned head piece, the position of the cleavable site is preferably within 20 bases, more preferably within 10 bases, and further preferably within 3 bases, starting from the binding portion between the loop site and the first oligonucleotide chain or the second oligonucleotide chain.

As one aspect, the present invention provides appropriate conditions in a method of inducing a DEL containing a cleavable site in a DNA strand to a cross linker-modified double-stranded DEL and evaluating it.

As one embodiment, in the above-mentioned head piece, the position of the cleavable site is present in the 3′ direction and is preferably within 20 bases, more preferably within 10 bases, further preferably within 3 bases, and most preferably within 1 base, starting from the binding portion between the loop site and the first oligonucleotide chain or the second oligonucleotide chain.

Although this is the explanation just in case, the preferred embodiment of the “selectively cleavable site” and, for example, the preferred embodiment of E, F or LP, etc., is different concept, respectively. That is, even if the position of the “selectively cleavable site” is included in E, the preferred embodiment of E does not necessarily apply to the “selectively cleavable site”.

As one embodiment, the compound constituting the DEL of the present invention is a compound represented by the following formula (II).

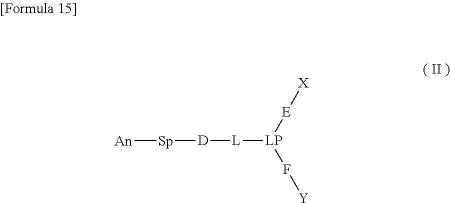

wherein
X and Y are oligonucleotide chains,
E and F are each independently an oligomer composed of nucleotides or nucleic acid analogues,
provided that E and F comprise base sequences complementary to each other and they form a duplex oligonucleotide,
LP is a loop site,
L is a linker,
D is a divalent group derived from a reactive functional group,
Sp is a bonding or a bifunctional spacer,
An is a partial structure comprised of at least one building block,
X and Y have a sequence capable of forming a duplex at least a part thereof,
X binds to E at the 5′ end,
Y binds to F at the 3′ end, and
at least any one site of E, F, or LP has at least one selectively cleavable site.

As one embodiment, preferred embodiments of E, F, LP, L, and D in the above-mentioned compound represented by the formula (II) are the same as the preferred embodiments of E, F, LP, L, and D explained with respect to the above-mentioned formula (I).

Preferred embodiments of X, Y, Sp, and An will be explained separately.

### (Bifunctional Spacer)

As described above, the bifunctional spacer is a spacer portion having at least two reactive groups that enables binding between the partial structure An of the compound library and the head piece. As one embodiment, the bifunctional spacer is SpD-SpL-SpX.

SpX is a reactive group that forms a covalent bond with the reactive functional group of the head piece.

SpD is a reactive group that forms a covalent bond with the partial structure An of the compound library.

SpL is a chemically inactive spacing portion

Similar to the reactive functional group (D), the reactive group (SpX) is a monovalent group (-SpX) in the bifunctional spacer substance itself (in the state of the reagent before binding to the head piece) and is a “divalent group derived from a reactive group” (-SpX-) based on the above-mentioned (-SpX) in the DEL (in the state of being bound to the head piece).

Also, similarly, the reactive group (SpD) is a monovalent group (SpD-) in the state before binding to An and is a “divalent group derived from a reactive group” (-SpD-) based on the above-mentioned (SpD-) in the DEL (in the state of being bound to An).

A preferred embodiment of SpX is a reactive group that forms an amino, carbonyl, amide, ester, urea, or sulfonamide bond. As one embodiment, SpX is a structure of the following (SpX1), (SpX2), or (SpX3), which is a reactive group suitable when the reactive functional group of the head piece is an amino group.
(SpX1): a carboxy group, a halogenated carboxy group, an aldehyde group, or a halogenated sulfonyl group
(SpX2): a carboxy group or a halogenated sulfonyl group
(SpX3): a carboxy group

A preferred embodiment of SpD is the same as the above-mentioned D.

As one embodiment, SpD is the above-mentioned (D1), (D2), (D3), (D4), or (D5).

A preferred embodiment of SpL is the following embodiments.

As one embodiment, SpL is the above-mentioned (L1), (L2), (L3), (L4), or (L5).

As one embodiment, SpL is the following (SpL1), (SpL2), or (SpL3).
(SpL1) Polyalkylene glycol, polyethylene, C1 to 20 aliphatic hydrocarbon which may be optionally replaced with a hetero atom(s), peptide, oligonucleotide, or a combination thereof.
(SpL2) Polyalkylene glycol, polyethylene, C1 to 10 aliphatic hydrocarbon, or peptide
(SpL3) Polyethylene glycol or polyethylene

As one embodiment, the bifunctional spacer is as follows.
(Sp1): (D4)-(SpL1)-(SpX1)
(Sp2): (D4)-(SpL2)-(SpX2)
(Sp3): (D4)-(SpL3)-(SpX3)
(Sp4): (D5)-(SpL1)-(SpX1)
(Sp5): (D5)-(SpL2)-(SpX2)
(Sp6): (D5)-(SpL3)-(SpX3)

As one embodiment, (Sp-D-L) portion of the compound constituting the DEL is so constituted as (SpDL1), (SpDL2), (SpDL3) (SpDL4), (SpDL5), (SpDL6), (SpDL7), (SpDL8), (SpDL9), or (SpDL10).
(SpDL1): (D4)-(L1)
(SpDL2): (D5)-(L1)
(SpDL3): (D4)-(L2)
(SpDL4): (D5)-(L2)
(SpDL5): (Sp1)-(D5)-(L5)
(SpDL6): (Sp2)-(D5)-(L5)
(SpDL7): (Sp3)-(D5)-(L5)
(SpDL8): (Sp4)-(D5)-(L5)
(SpDL9): (Sp5)-(D5)-(L5)
(SpDL1O): (Sp6)-(D5)-(L5)
In (SpDL1), (SpDL2), (SpDL3), and (SpDL4), Sp means a bond.

In carrying out the present invention, it is advantageous if the head piece can be synthesized by a nucleic acid synthesizer. In this practice, as mentioned above, as one embodiment, a monomer for nucleic acid synthesis in which the linker site (L) and the reactive functional group site (D) are bound to LS can be prepared, and then a nucleic acid oligomer can be synthesized. Examples of such a monomer for nucleic acid synthesis include the above-mentioned Amino C6 dT, mdC (TEG-Amino), Uni-Link® Amino Modifier, and the like.

On the other hand, when the above-mentioned commercially available monomer for nucleic acid synthesis or a nucleic acid analogue that can be used in a nucleic acid synthesizer is used, there is a possibility that the length of the linker site is limited. In such a case, as one embodiment, by introducing an appropriate bifunctional spacer, it becomes possible to adjust the distance between the head piece and An, which is advantageous in carrying out the invention.

In the explanation of the present invention, “of C1 to C6” or “C1 to 6” in the terms such as a “C1 to C6 alkyl group” or a “C1 to 6 alkyl group” means that the number of carbon atom(s) is 1 to 6. Similarly, when m and n are integers and there are expression “of Cm to Cn” or “Cm to n”, the expression means that the number of carbon atom(s) is m to n. Accordingly, a “C1 to C6 alkyl group” or a “C1 to 6 alkyl group” means an alkyl group of which the number of carbon atom(s) is 1 to 6, and a “C1 to C6 alkylene” or a “C1 to 6 alkylene” means an alkylene of which the number of carbon atom(s) is 1 to 6.

In the present invention, the “C1 to 6 alkyl” means a linear or branched alkyl group of which the number of carbon atom(s) is 1 to 6. Specific examples include methyl, ethyl, propyl, isopropyl, butyl, isobutyl, sec-butyl, tert-butyl, pentyl, hexyl, and the like.

In the present invention, the “C1 to 3 alkyl” means a linear or branched alkyl group of which the number of carbon atom(s) is 1 to 3. Specific examples are methyl, ethyl, propyl, and isopropyl.

In the present invention, the “C1 to 6 alkoxy” means a linear or branched alkoxy of which the number of carbon atom(s) is 1 to 6. Specific examples include methoxy, ethoxy, propoxy, isopropoxy, butoxy, isobutoxy, sec-butoxy, tert-butoxy, pentyloxy, hexyloxy, and the like.

In the present invention, the “C1 to 3 alkoxy” means a linear or branched alkoxy of which the number of carbon atom(s) is 1 to 3. Specific examples are methoxy, ethoxy, propoxy, and isopropoxy.

In the present invention, the “hydrocarbon” means a linear, branched, or cyclic saturated or unsaturated compound comprised of carbon atom and hydrogen atom only.

In the present invention, the “aliphatic hydrocarbon” means a non-aromatic material among the hydrocarbons. The “aliphatic hydrocarbon” may be linear, branched, or cyclic, or may be saturated or unsaturated. Specific examples of the structure include alkyl, alkenyl, alkynyl, cycloalkyl, or cycloalkenyl, or a structure by a combination thereof.

In the present invention, the “C1 to 20 aliphatic hydrocarbon” means an aliphatic hydrocarbon of which the number of carbon atom(s) is 1 to 20.

In the present invention, the “C1 to 10 aliphatic hydrocarbon” means an aliphatic hydrocarbon of which the number of carbon atom(s) is 1 to 10.

In the present invention, the “C1 to 6 aliphatic hydrocarbon” means an aliphatic hydrocarbon of which the number of carbon atom(s) is 1 to 6.

In the present invention, the “aromatic hydrocarbon” means an aromatic one among the hydrocarbons.

In the present invention, the “C6 to 14 aromatic hydrocarbon” means an aromatic hydrocarbon of which the number of carbon atoms is 6 to 14. Specific examples include benzene, naphthalene, and anthracene.

In the present invention, the “C6 to 10 aromatic hydrocarbon” means an aromatic hydrocarbon of which the number of carbon atoms is 6 to 10. Specific examples are benzene or naphthalene.

The aromatic heterocyclic ring in the present invention is an aromatic heterocyclic ring having an element(s) selected solely or differently from the group consisting of nitrogen, oxygen, and sulfur as a hetero atom(s) in the cyclic structure.

As one embodiment, the aromatic heterocyclic ring is a “C1 to 9 aromatic heterocyclic ring” of which the number of carbon atom(s) is 1 to 9, and as one embodiment, the “C1 to 9 aromatic heterocyclic ring” is a “5 to 10-membered aromatic heterocyclic ring”.

As one embodiment, the aromatic heterocyclic ring is a “C1 to 5 aromatic heterocyclic ring” of which the number of carbon atom(s) is 1 to 5, and as one embodiment, the “C1 to 5 aromatic heterocyclic ring” is a “5 to 10-membered aromatic heterocyclic ring”.

As one embodiment, the aromatic heterocyclic ring is a “C2 to 9 aromatic heterocyclic ring” of which the number of carbon atoms is 2 to 9, and as one embodiment, the “C2 to 9 aromatic heterocyclic ring” is a “5 to 10-membered aromatic heterocyclic ring”.

As one embodiment, the aromatic heterocyclic ring is a “C2 to 5 aromatic heterocyclic ring” of which the number of carbon atoms is 2 to 5, and as one embodiment, the “C2 to 5 aromatic heterocyclic ring” is a “5 to 6-membered aromatic heterocyclic ring”.

The nitrogen-containing aromatic heterocyclic ring in the present invention is an aromatic heterocyclic ring having nitrogen in the cyclic structure as a hetero atom.

As one embodiment, the nitrogen-containing aromatic heterocyclic ring is a “C1 to 5 nitrogen-containing aromatic heterocyclic ring” of which the number of carbon atom(s) is 1 to 5, and as one embodiment, the “C1 to 5 nitrogen-containing aromatic heterocyclic ring” is a “5 to 6-membered aromatic heterocyclic ring”.

As one embodiment, the nitrogen-containing aromatic heterocyclic ring is a “C2 to 5 nitrogen-containing aromatic heterocyclic ring” of which the number of carbon atoms is 2 to 5, and as one embodiment, the “C2 to 5 nitrogen-containing aromatic heterocyclic ring” is a “5 to 6-membered aromatic heterocyclic ring”.

The non-aromatic heterocyclic ring in the present invention is a non-aromatic heterocyclic ring having an element(s) selected solely or differently from the group consisting of nitrogen, oxygen, and sulfur as a hetero atom(s) in the cyclic structure.

The non-aromatic heterocyclic ring may contain a partially-unsaturated bond.

As one embodiment, the non-aromatic heterocyclic ring is a “C2 to 9 non-aromatic heterocyclic ring” of which the number of carbon atoms is 2 to 9, and as one embodiment, the “C2 to 9 non-aromatic heterocyclic ring” is a “5 to 10-membered non-aromatic heterocyclic ring”.

In the present invention, the “trivalent group of C1 to 14” means a trivalent group derived from a compound of which the number of carbon atom(s) is 1 to 14. As long as the effect of the present invention is achieved, the structure is not limited.

In the present invention, when there is a description that “may be replaced with a hetero atom(s)”, the hetero atom means an atom other than carbon and hydrogen.

The hetero atom is preferably an oxygen atom, a nitrogen atom, a silicon atom, a phosphorus atom, or a sulfur atom, and more preferably an oxygen atom, a nitrogen atom, or a sulfur atom.

Accordingly, for example, when propyl (—CH₂—CH₂—CH₃) is mentioned as an example of the hydrocarbon, the “propyl which may be replaced with a hetero atom(s)” is a concept including structures such as an ether ((—CH₂—O—CH₃) or (—O—CH₂—CH₃)) in which methylene (—CH₂—) in the alkyl is replaced with oxygen, and an amine ((—CH₂—NH—CH₃) or (—NH—CH₂—CH₃)) in which it is replaced with nitrogen.

In the present invention, when there is a description that “may have a substituent(s)”, the substituent is not limited as long as it achieves the object of the present invention.

The substituent is preferably a C1 to 6 alkyl group, a C1 to 6 alkoxy group, an amino group, a hydroxy group, a nitro group, a cyano group, an oxo group, or a halogen atom.

The substituent is more preferably a C1 to 6 alkyl group, a C1 to 6 alkoxy group, a fluorine atom, or a chlorine atom.

In the present invention, the polypeptide and the peptide mean a compound or a partial structure formed by connecting amino acids. The amino acid is a general term for organic compounds having both functional groups of an amino group and a carboxy group. The amino acid that constitutes the polypeptide and peptide according to the present invention is not particularly limited and includes a modified amino acid, etc. In accordance with general usage in the field of life science, in the present invention, proline (classified as imino acid) is also included in the amino acids. The amino acid that constitutes the polypeptide and the peptide according to the present invention is preferably a amino acid and more preferably an “amino acid that “constitutes a protein”.

The halogen atom in the present invention includes a fluorine atom, a chlorine atom, a bromine atom, and an iodine atom.

A C—C, amino, ether, carbonyl, amide, ester, urea, sulfide, disulfide, sulfoxide, sulfonamide, and sulfonyl bond are chemical bonds having chemical structures understood by their respective names. Those skilled in the art understand that, for example, an ether bond is a bond that can generally be represented by “—O—” and a carbonyl bond is a bond that can generally be represented by “—C(═O)—”. An amino, amide, and urea bond have a hydrogen atom or other substituent(s) on the nitrogen atom, but the structure on the nitrogen atom is not limited as long as it has the effect of the present invention. The above-mentioned substituent(s) on the nitrogen atom is/are preferably a C1 to 6 alkyl group(s) or a hydrogen atom(s), and more preferably a hydrogen atom(s). Also, it may be needless to say, the C—C bond means a carbon-carbon bond. The C—C bond includes a single bond, a double bond, and a triple bond. As one embodiment, in the steps a and/or c in the production method according to the present invention, a bond appropriately selected from the above-mentioned 11 kinds is constructed. These 11 kinds of bonds are particularly basic bonding modes in organic chemistry and the reactions for constructing them are also well known to those skilled in the art. Accordingly, in designing and constructing the partial structure An of the compound library according to the present invention, those skilled in the art can combine these 11 kinds of bonds appropriately and use them.

The organic compound composed of an element selected alone or differently from the element group consisting of H, B, C, N, O, Si, P, S, F, Cl, Br, and I is an organic compound constructed by the bond of the above-mentioned 12 kinds of elements.

As one embodiment, the partial structure An of the compound library according to the present invention is constructed by the above-mentioned 12 kinds of elements. These 12 kinds of elements are particularly basic elements in organic compounds and the reactions for constructing them are also well known to those skilled in the art. Accordingly, in designing and constructing the partial structure An of the compound library according to the present invention, those skilled in the art can combine these 12 kinds of elements appropriately and used them.

A low molecular weight organic compound having a substituent(s) selected alone or differently from a substituent group consisting of an aryl group, a non-aromatic cyclyl group, a heteroaryl group, and a non-aromatic heterocyclyl group is a low molecular weight organic compound having a chemical structure understood by each name. The low molecular weight compound is a concept well known to those skilled in the art and examples of the preferred molecular weight of the low molecular weight compound in the present invention will be mentioned separately.

The aryl group in the present invention is preferably a C6 to 10 aryl group and more preferably a phenyl group.

The non-aromatic cyclyl group in the present invention is preferably a 5-membered to 8-membered non-aromatic cyclyl group and more preferably a 5-membered or 6-membered non-aromatic cyclyl group. The non-aromatic cyclyl group may contain a partially-unsaturated bond.

The heteroaryl group and the non-aromatic heterocyclyl group in the present invention are groups having an element selected alone or differently from the group consisting of nitrogen, oxygen, and sulfur as a hetero atom(s) in the cyclic structure. The heteroaryl group and the non-aromatic heterocyclyl group in the present invention is preferably a 5-membered to 8-membered group and more preferably a 5-membered or 6-membered group, and the non-aromatic heterocyclyl group may contain a partially-unsaturated bond.

As one embodiment, the partial structure An of the compound library according to the present invention has the above-mentioned 4 kinds of groups. These 4 kinds of groups are particularly basic partial structures in organic compounds and reactions for constructing them in the compounds are also well known to those skilled in the art. Accordingly, in designing and constructing the partial structure An of the compound library according to the present invention, those skilled in the art can appropriately combine and use these 4 kinds of groups.

The above-mentioned preferred embodiments, that is, a compound library constructed by the 11 kinds of bonds, the 12 kinds of elements and/or the 4 kinds of groups, have particular core value. Accordingly, those skilled in the art would understand that compound libraries constructed without these preferred embodiments would generally limit their use, and in many cases, would limit their commercial value.

The synthesis history of An means a general record of operations carried out until An is synthesized, and in particular, it means the structure of the building blocks the order thereof used until An is synthesized. For example, when reaction is carried out using respective different building block and/or different reaction conditions in two or more separate reaction vessels, an oligonucleotide chain having a previously determined sequence is ligated to the products in the respective reaction vessels before and after the reaction, whereby the synthesis history is imparted as sequence information of the oligonucleotide. By repeating such an operation until An is constructed, an oligonucleotide of Bn having the synthesis history of An is constructed

The split and pool synthesis is a synthetic method developed by Geisen et al., as a combinatorial chemical constructing method of a peptide library utilizing a solid-phase synthetic method in the early days of combinatorial chemistry. The split and pool synthesis is also called a split-mix method, etc.

In accordance with the above, when the synthesis of a peptide library utilizing a solid-phase synthetic method is explained as an example. In the split and pool synthesis, each step of increasing the terminal of the peptide, without cutting out the sample from the solid-phase carrier to which amino acids are peptide-bonded, N kinds of carriers are once mixed and homogenized, and then they are divided into equal parts and the terminal by the next N kinds of amino acids is increased.

That is, one kind of peptide chain is formed for each carrier, and when all 20 kinds of natural amino acids are applied at each stage, a peptide library combinable with all peptides having specific lengths is to be constructed.

If this peptide library is to be screened by antigen presentation or receptor binding, an assay can be carried out by utilizing a peptide on a solid-phase carrier using an ELISA method, etc. That is, it is not necessary to cut out the peptide of the sample from the carrier, and the carrier particles that have reacted in the assay are picked up (for example, the carrier particles of about 0.1 mm that are fluorescently labeled are picked up by an optical microscope). Then, the objective peptide sequence can be determined by the peptide of the particles using an instrument analyzer (peptide analyzer, etc.), or the peptide sequence that is indirectly becomes a candidate for screening can be determined by other combinatorial chemical identification method (for example, tag method), etc.

Further, in the production method according to the present invention, the following case will be explained where v kinds of structures when m is 2 and w kinds when m is 3 are synthesized by the split and pool synthesis. In the explanation, the steps are repeated in the order of (c) and (d).

(m=2)

In the step of m=2, to A1-Sp-C-B1, α2 is added in the step (c) and β2 in the step (d), respectively, to produce A2-Sp-C-B2.

Here, α2 (α2 (a-v)) with v kinds of structures and v kinds of β2 (β2 (a-v)) corresponding thereto are prepared and the steps (c) and (d) are each carried out for each structure, and then v kinds of A2-Sp-C-B2 (A2(a)-Sp-C-B2(a), A2(b)-Sp-C-B2(b) . . . A2(v)-Sp-C-B2(v): that is, A2(a-v)-Sp-C-B2(a-v)) can be obtained. In the split and pool synthesis, v kinds of A2-Sp-C-B2 are mixed and then divided into the number of w. Division means, most specifically, that it is subdivided into reaction vessels with the number of w.

(m=3)

In the step of m=3, to A2-Sp-C-B2, α3 is added in the step (c) and β3 in the step (d), respectively, to produce A3-Sp-C-B3.

Here, α3 (α3 (a-w)) with w kinds of structures and w kinds of β3 (β2 (a-w)) corresponding thereto are prepared, and to (A2 (a-v)-Sp-C-B2 (a-v) mixture) with the number of w, the steps (c) and (d) are each carried out. Then, through the steps of n=2 and 3, (v×w) kinds of A3-Sp-C-B3 is to be efficiently synthesized by (v+w) times of syntheses.

### (Biological Evaluation)

When the obtained products with the number of w are mixed, a mixture of (v×w) kinds of A3-Sp-C-B3 compound library is obtained. For example, if a binding test of a drug receptor is carried out to this mixture, screening of (v×w) kinds of compounds can be carried out at one time. By washing away the compounds that did not bind to the drug receptor, only the bound compounds can be isolated. In a DEL like the present invention, the DNA of the isolated A3-Sp-C-B3 compound is amplified to an amount that can be sequenced and the structure of A3 can be grasped from the sequence information.

A compound library, building blocks, split and pool, etc., are terms well known to those skilled in the art in fields such as combinatorial chemistry and can be carried out in a timely manner with reference to the following Literature, etc.
(1) Takashi Takahashi, Takayuki Doi “Combinatorial chemistry”, Journal of The Society of Synthetic Organic Chemistry, 2002, vol. 60, pp. 426-433
(2) Edited by Combinatorial Chemistry Study Group, “Combinatorial Chemistry”, Kagaku-Dojin Publishing

A DNA-encoded library (or DEL) is a compound library comprising a group of compounds labeled with DNA or oligonucleotides having substantially the same function as DNA (DNA-encoded compound). By the split and pool synthesis as mentioned above, the structure or synthesis history of each compound is imparted to the labeled DNA as sequence information. From such characteristics, the DNA-encoded library is screened in the form of a mixture of 10² to 10²⁰ kinds of compounds and the DNA sequences contained in the obtained compounds are identified by techniques known in the art (for example, use of next-generation sequencers and/or use of microarrays), whereby it is possible to identify the structure of the compound. As one embodiment of the above-mentioned screening method, a method can be selected where a target such as a protein is contacted with a DNA-encoded library and a compound bound to the target is selected.

A “biological target” is a term well known to those skilled in the art, and as one embodiment, in the present invention, the “biological target” is a biological substance group that can be a target in the development of a drug, etc., represented by medical and agrochemical drugs, including, for example, an enzyme (for example, kinase, phosphatase, methylase, demethylase, protease, and DNA repair enzyme), a protein involved in protein-protein interaction (for example, a ligand for receptor), receptor target (for example, GPCR), ion channel, cell, bacteria, virus, parasite DNA, RNA, prion, and sugar.

“Biological activity evaluation” is a term well known to those skilled in the art, and as one embodiment, in the present invention, the “biological activity evaluation” is to evaluate the presence or absence, or strength of the biological activity possessed by a compound (for example, an ability to bind to a biological target, inhibitory function of enzyme activity, promotion function of enzyme activity, etc.). As specific examples of the biological activity evaluation, the above-mentioned Patent Documents 2 and 3, Non-Patent Documents 1 to 6, etc., can be also referred to.

“Functionality evaluation” is a term well known to those skilled in the art, and as one embodiment, in the present invention, the “functionality evaluation” is to evaluate the presence or absence, or strength of a specific function possessed by a compound (for example, binding ability, biological activity, luminescence property, etc.).

The present invention provides a plurality of methods having several advantages with respect to DEL and a method for producing DEL by using a DNA strand having a cleavable site. Forms 1 to 7 will be described in detail below.

### Form 1

The present invention provides a DEL using the above-mentioned “hairpin type head piece having a cleavable site”.

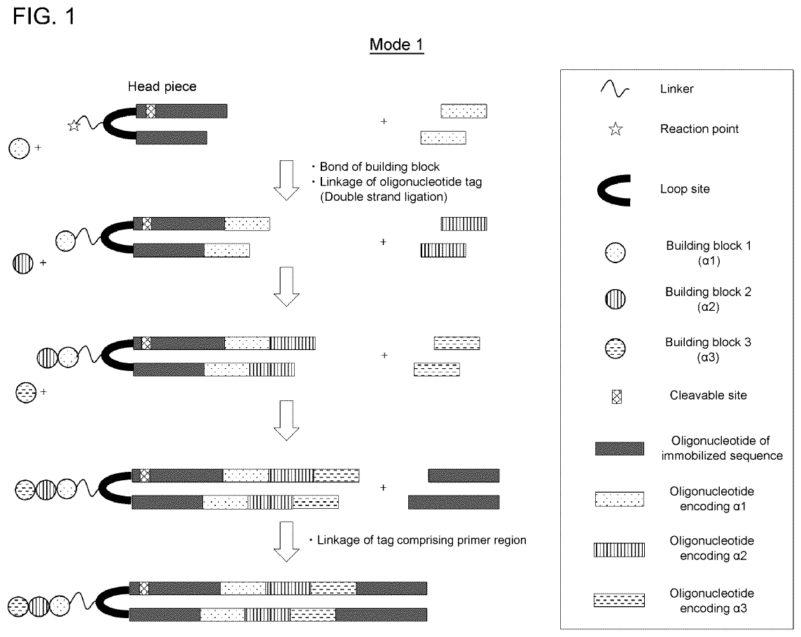

As exemplified in FIG. 1, in Form 1, a head piece which contains a first oligonucleotide chain containing a cleavable site in a DNA strand, a loop site, and a second oligonucleotide chain is used as a starting material. Linking of building blocks and double strand ligation of an oligonucleotide tag corresponding to the building blocks are repeated (three times in FIG. 1) and further, if desired, a double strand ligation of an oligonucleotide tag containing a primer region is carried out, whereby production of a DEL is achieved.

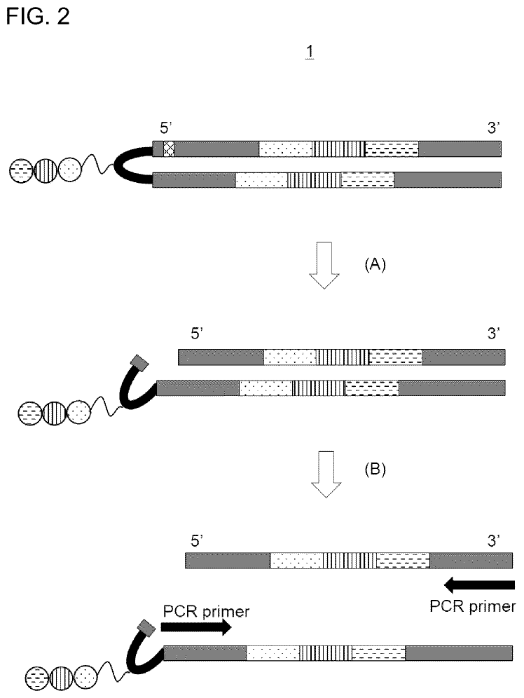

As exemplified in FIG. 2, in Form 1, to a DEL containing the cleavable site in the first oligonucleotide chain of the head piece, the cleavable site is cleaved using a cleaving means such as an enzyme and induced to a double strand oligonucleotide that is not bound at the loop site, whereby PCR can be carried out with high efficiency.

### (With Regard to Form 2)

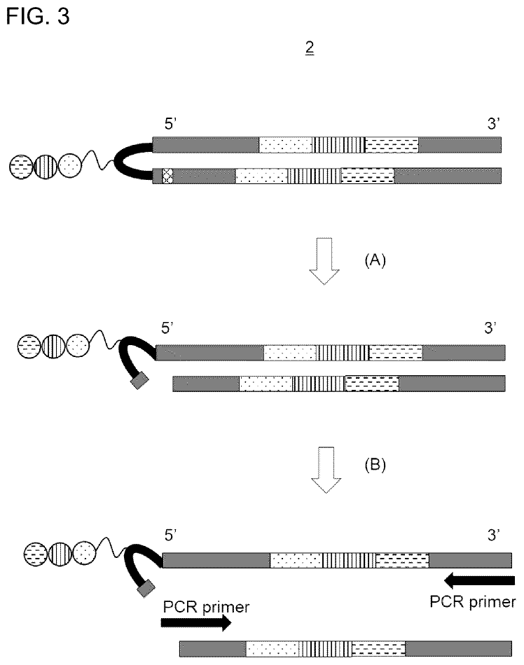

As exemplified in FIG. 3, in the DEL using the “hairpin type head piece having a cleavable site”, the cleavable site may exist in the second oligonucleotide chain. The characteristics of Form 2 are the same as those of Form 1 except for the cleavable site.

### (With Regard to Form 3)

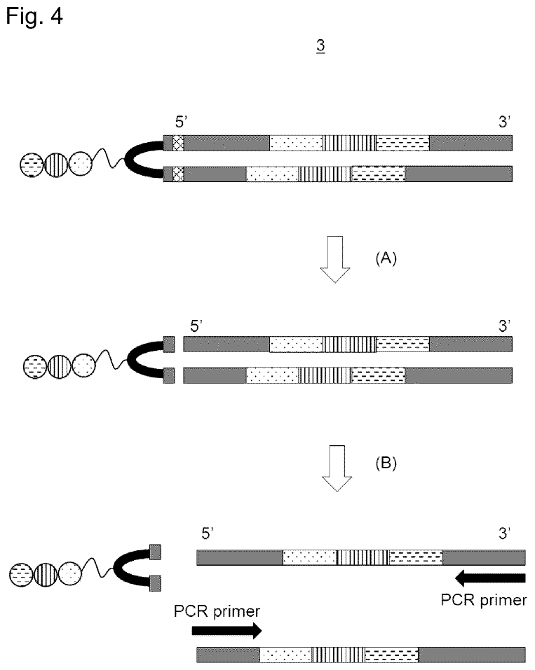

As exemplified in FIG. 4, in the DEL using the “hairpin type head piece having a cleavable site”, the cleavable site may exist in both the first and second oligonucleotide chains. In this embodiments, the loop sites are cleaved from both of the oligonucleotide chains, whereby it is expected to further improve PCR efficiency.

### (With Regard to Form 4)

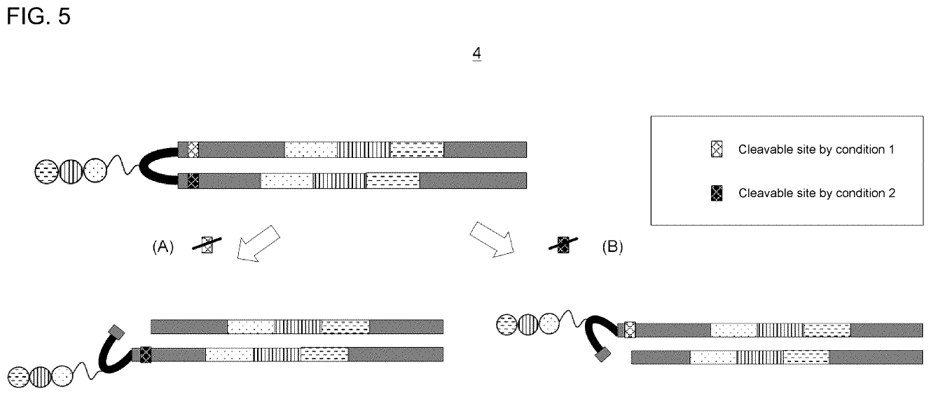

As exemplified in FIG. 5, in the present invention, the cleavable site may exist in both the first oligonucleotide chain (E) and the second oligonucleotide chain (F), and further the structures of the cleavable sites may be different. In such a case, by utilizing the difference in characteristics of the two (or more) cleavable sites, the cleaving site can be controlled.

For example, deoxyuridine may be used as the cleavable site in the first oligonucleotide chain (E) and deoxyinosine may be used as the cleavable site in the second oligonucleotide chain (F).

In this case, when the USER enzyme is used, deoxyuridine in the first oligonucleotide chain (E) can be selectively cleaved.

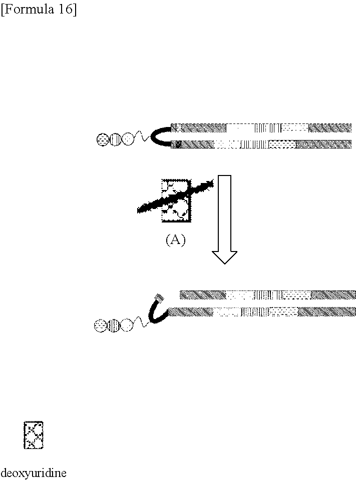

On the other hand, when alkyladenine DNA glycosylase and endonuclease VIII are used, in the second oligonucleotide chain (F), the cleavage site originating from deoxyinosine can be selectively cleaved.

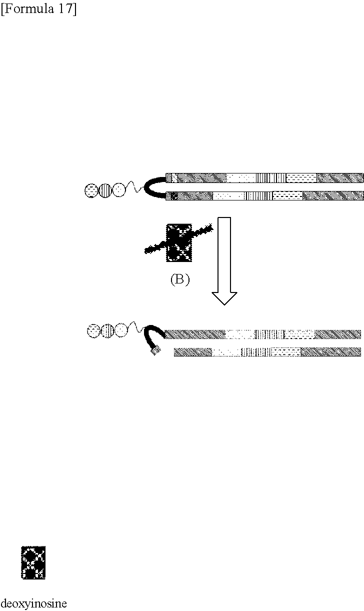

Like this, by selecting the cleavage site as desired, a wider range of modifications of the DEL becomes possible and a wider means can be applied to the evaluation thereafter.

can be expected.

### (With Regard to Form 5)

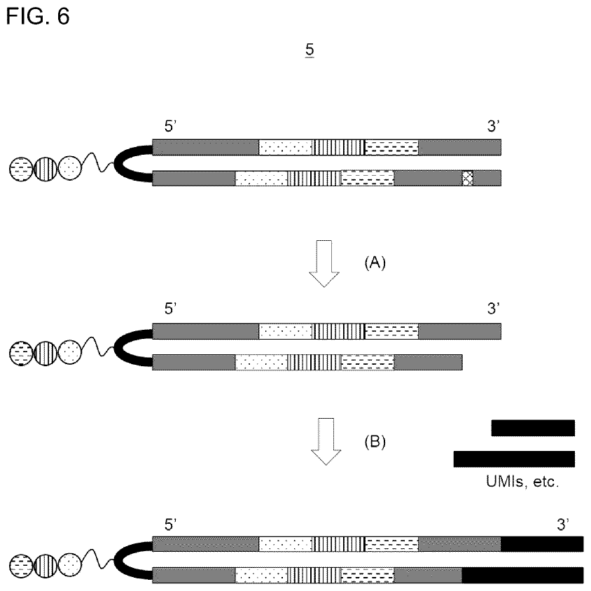

As exemplified in FIG. 6, in the present invention, the cleavable site may be provided at the DNA tag portion (for example, oligonucleotide chain (Y)). The cleavable site is provided near the terminal of the DNA tag, and if desired, the site is cleaved to form a new protruding end.

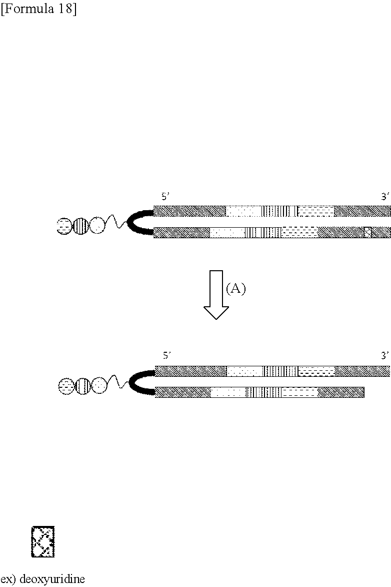

The protruding end is utilized as a sticky end and a desired nucleic acid sequence, for example, UMIs (a specific molecule identification sequence), etc., can be ligated.

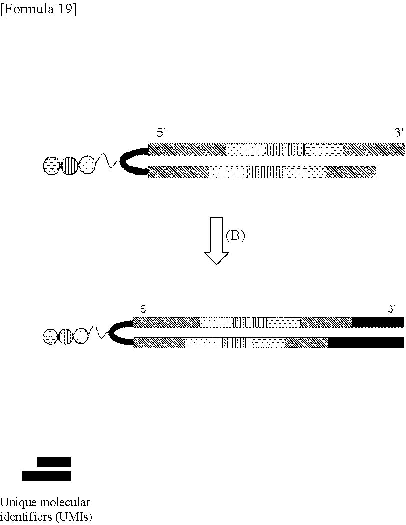

After the biological evaluation, to the selected DEL compound, the UMIs region is imparted as mentioned above, and by subjecting to DNA sequencing, it is possible to carry out the analysis in which amplification bias by PCR is reduced

Like this, in the present invention, by having selectively cleavable site(s) in the nucleic acid sequence, it is possible to impart unconventional properties in the aspect of production and use of the DEL compound.

Here, UMIs (a specific molecule identification sequence) is a molecular identifier that gives individual DNA sequence to each DNA molecule by imparting it to the DNA comprised in a certain sample (see Nature Method, 2012, vol. 9, pp. 72-74). By providing such a molecular identifier before PCR amplification, when the number of DNA molecules having a specific sequence in the sample is quantified, it is possible to identify PCR duplication (sequences derived from the same molecule) and quantification in reducing PCR amplification bias is possible.

### (With Regard to Form 6)

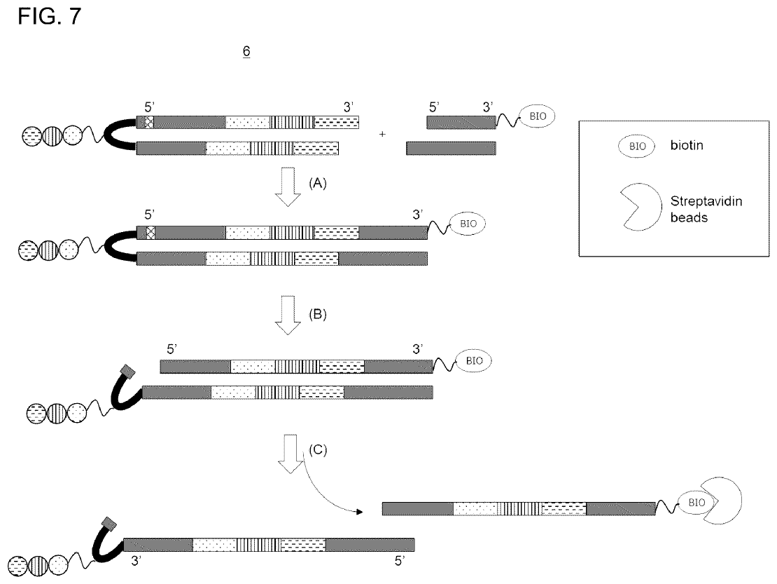

As exemplified in FIG. 7, in the present invention, a cleavable site can be used in combination with a modifying group or a functional molecule. For example, it is possible to prepare a DEL in which a hairpin-stranded DNA is converted into a single-stranded DNA.

In accordance with FIG. 7, a DEL compound using a head piece having a cleavable site in an E portion is mentioned as an example.
(Step A) A double-stranded oligonucleotide chain having a modifying group which is removable with a solid-phase-carrier (for example, biotin) at the 3′ end is ligated to the synthesized DEL compound.
(Step B) The cleavable site is cleaved.
(Step C) A treatment according to the function of the modifying group is applied. For example, in the case of biotin, by using streptavidin beads, etc., having biotin affinity, the oligonucleotide chain to which biotin is bound is selectively removed from the system, whereby it is possible to obtain a DEL having a single-stranded DNA.

Here, the functional molecule is a molecule having a specific chemical or biological function (for example, solubility, photoreactivity, substrate-specific reactivity, target protein degradation-inducing property), and by imparting it to a DEL, it is possible to carry out evaluation or purification of the DEL depending on the function.

Here, biotin means all biotins that bind to avidin and includes not only vitamin B7 but also, for example, desthiobiotin.

As one aspect, the present invention provides appropriate conditions in a method of inducing a DEL containing a cleavable site in a DNA strand to a cross linker-modified double-stranded DEL and evaluating it.

As another method of preparing a DEL in which a hairpin-stranded DNA is converted into a single-stranded DNA, a method using the following exonuclease can also be mentioned.
(Step A) A hairpin type having “selectively cleavable site(s)” is cleaved using an enzyme which cleaves the 5′ end of the cleavage site in a phosphorylated state after a cleavage reaction, for example, USER® Enzyme, etc.
(Step B) For example, one oligonucleotide chain whose 5′ end is phosphorylated is decomposed and removed by a treatment with lambda exonuclease, whereby, a DEL having a single-stranded DNA (single-stranded DEL) is obtained.

The single-stranded DEL obtained above is preferably a single-stranded DEL having a library molecule in the 3′ direction of the oligonucleotide chain. The single-stranded DEL can be subjected to a primer elongation reaction using a cross linker-modified primer having a cross linker at the 5′ end. By this method, it is possible to easily synthesize a “cross linker-modified double-stranded DEL” in which a cross linker is linked to an oligonucleotide having a coding sequence via a covalent bond.

In the case of acquiring the single-stranded DEL having the library molecules in the 3′ direction, the “selectively cleavable site” of the hairpin type DEL as a starting material is present in the 3′ direction from the site to which the library molecule is bound.

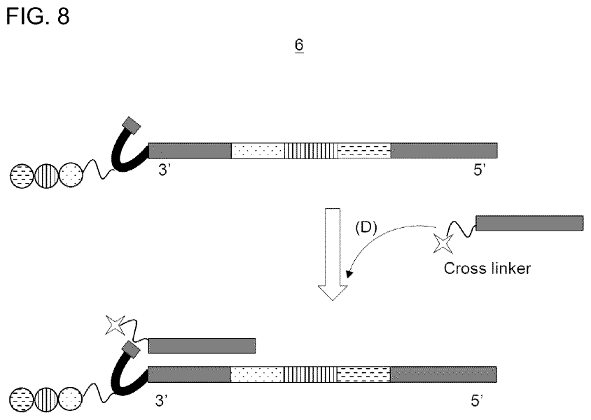

As exemplified in FIG. 8, a DEL having a single-stranded DNA is formed by forming a double strand with a modified oligonucleotide having a desired functional site (for example, a cross linker-modified DNA such as photoreactive cross linker or a cross linker-modified primer such as a photoreactive cross linker), whereby it is possible to impart a new function.

Furthermore, when the cross linker-modified primer is used, an optionally added primer may be elongated to induce to a cross linker-modified double-stranded DEL compound. Such a cross linker-modified double-stranded DEL compound is useful in the present invention because a covalent bond is formed between the biological target and the coding sequence after screening.

### (With Regard to Form 7)

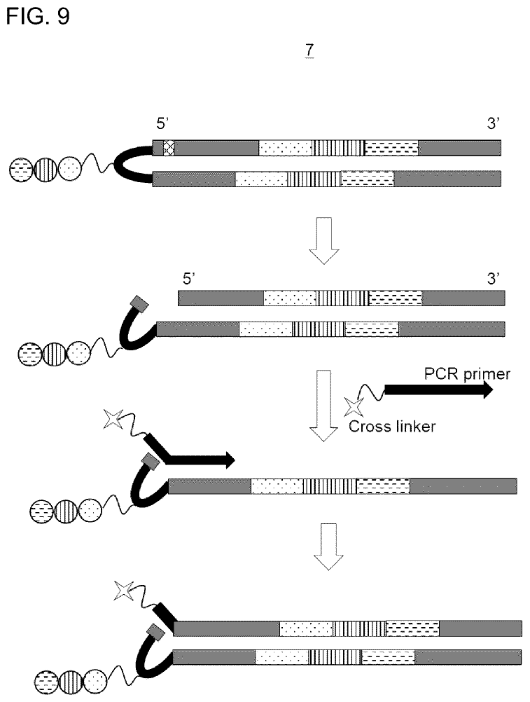

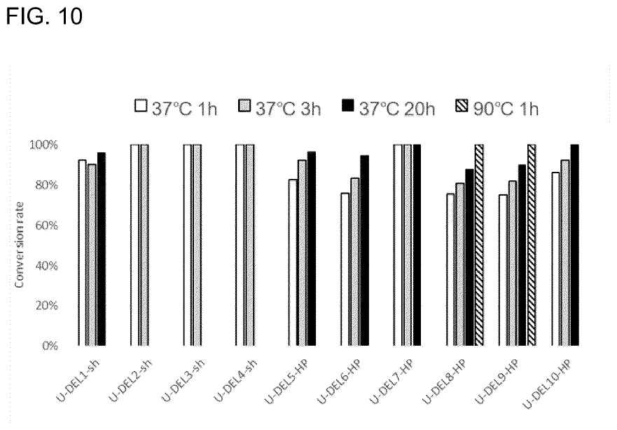

As exemplified in FIG. 9, in the present invention, a cross linker can be introduced by utilizing a cleavable site.

In accordance with FIG. 9, a DEL compound using a head piece having a cleavable site in an E portion is mentioned as an example.
(Step A) A cleavable site is cleaved with respect to the synthesized DEL compound.
(Step B) A modified primer having a desired functional site (for example, a cross linker-modified primer such as a photo-reactive cross linker) is imparted.
(Step C) The imparted primer is elongated to synthesize a cross linker-modified double-stranded DEL compound.

In the scene of DEL evaluation, when the building block compound (library low molecular weight compound) binds to the target protein, the cross linker-modified double-stranded DEL compound can further bind the cross linker to the target protein, whereby detection sensitivity can be markedly improved (refer to Non-Patent Documents 7 and 11, etc.). In practice of the DEL technique for evaluating a large number of library compounds, it is extremely useful to enhance the affinity of the library compounds and to improve the detection sensitivity.

The present invention is to provide a novel and highly efficient method of producing a cross linker-modified double-stranded DEL compound, which is extremely useful.

As one aspect, the present invention provides appropriate conditions in a method of inducing a DEL containing a cleavable site in a DNA strand to a cross linker-modified double-stranded DEL and evaluating it.

As one embodiment, in the cross linker-modified double-stranded DEL compound, the cross linker is preferably linked to an oligonucleotide having a coding sequence via a covalent bond. Such a “cross linker-modified double-stranded DEL compound” is significantly useful because a covalent bond is formed between a target and a coding sequence after screening, and the compound is resistant to separation and elution conditions stronger than the conventional ones for removal of a non-specific binder, etc.

### (Cross Linker)

As one embodiment, in the present invention, the “cross linker” means a reactive group having reactivity capable of forming a covalent bond by a reaction with a biological target such as a protein or a nucleic acid molecule. For example, cross linkers as described in Thermo Scientific Crosslinking Technical Handbook are known.

The cross linker used in the present invention is preferably a reactive group comprising at least one of an azide group, a diazirine group, a sulfonyl fluoride group, a diazo group, a cinnamoyl group, or an acrylate group, and more preferably a reactive group comprising at least one of an azide group, a diazirine group, or a sulfonyl fluoride group.

As one embodiment, in the present invention, “having a cross linker” and “cross linker modification” mean having a partial structure comprising a cross linker as a substituent.

As one preferred embodiment, in the “cross linker-modified double-stranded DEL”, the “cross linker-modified DNA”, and the “cross linker-modified primer” bind directly or via a bifunctional spacer to the 5′ ends of the “double-stranded DEL”, the “DNA”, and the “primer”, respectively.

At that time, the cross linker is preferably a structure of any of the following formulae (AA) to (AE), or (BA) or (BB):

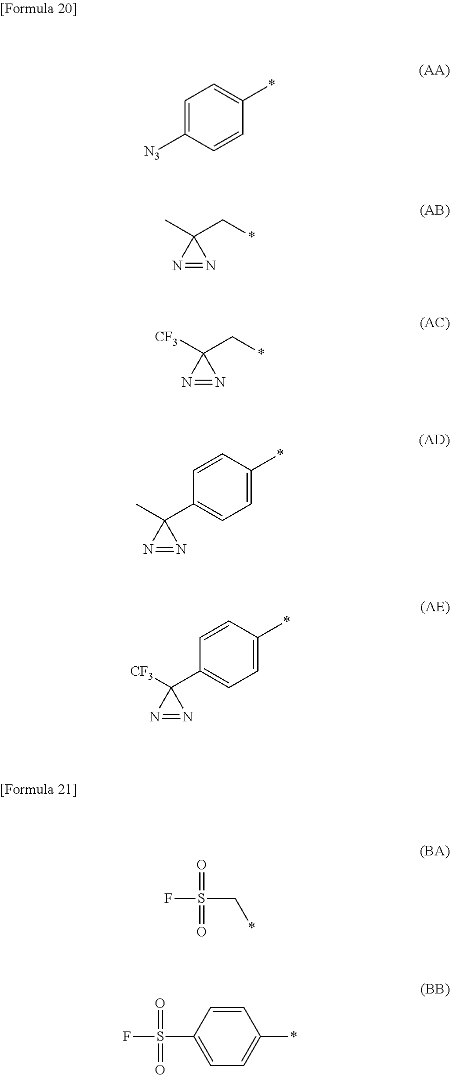

wherein * means a binding site with the 5′ end of the “double-stranded DEL”, “DNA”, or “primer”, or the bifunctional spacer side binding to the 5′ end.

As one preferred embodiment, the cross linker used in the present invention is a photoreactive cross linker. In the present invention, the photoreactive cross linker means a reactive group that is changed to a reactive group having a high reaction activity (for example, nitrene and carbene) by light irradiation and forms a covalent bond with a nearby biological target. For example, an azide group and a diazirine group are known, and the structures of the formulae (AA) to (AE) are known.

In addition, as one preferred embodiment, the cross linker used in the present invention is a reactive group comprising at least one sulfonyl fluoride group. For example, the sulfonyl fluoride group reacts with residues such as serine, threonine, tyrosine, lysine, cysteine, and histidine in a biological target protein to form a covalent bond. For example, the structures of the formulae (BA) and (BB) are known.

In the present invention, the crosslinking reaction between the cross linker and the biological target is preferably carried out in a temperature range in which the desired high-dimensional structure of the biological target does not change significantly. A preferred temperature is, for example, in a range of 4 to 40° C.

### (Bifunctional Spacer of Cross Linker)

As described above, the bifunctional spacer is a spacer portion having at least two reactive groups that enables binding between the partial structure An of the compound library and the head piece.

Furthermore, in the present invention, the bifunctional spacer is a spacer moiety having two reactive groups that enable the bonding between the cross linker and the “double-stranded DEL,” “DNA”, or “primer”. The bifunctional spacer of the embodiment may be referred to as a “bifunctional spacer of a cross linker”. In contrast, the bifunctional spacer bonded to the compound library described above may be referred to as a “bifunctional spacer of a compound library”.

As one embodiment, the preferred embodiment of the “bifunctional spacer of the cross linker” is the same as the preferred embodiment of the “bifunctional spacer of the compound library” described above.

In addition, as one embodiment, the “bifunctional spacer of the cross linker” preferably has a molecular chain length suitable for reacting the cross linker with a biological target when the compound library binds to the biological target at the time of screening, and preferably has a molecular chain length equivalent to the “bifunctional spacer of the compound library”.

### (Coding Sequence)

In the present invention, the “coding sequence” is a sequence moiety of an oligonucleotide having a sequence capable of identifying a structure of a library molecule among sequences of oligonucleotides comprised in DEL.

The “reactive group for cross linker modification” is not particularly limited as long as it is a reactive group capable of reacting with a cross linker unit described below.

As one embodiment, the “reactive group for cross linker modification” is a reactive group having reaction selectivity with a cross linker. By having reaction selectivity with the cross linker, reaction conditions under which the cross linker cannot be applied, such as the cross linker reacting first to undergoing structural transformation, can be applied to the present invention. That is, a unit having a reactive group for cross linker modification is introduced into the process according to the present invention, reaction conditions under which the cross linker cannot be applied are used in the process according to the present invention, and then the unit is reacted with the cross linker, whereby the cross linker required for the present invention can be introduced into the cross linker-modified DEL according to the present invention.

As one embodiment, the “reactive group for cross linker modification” and the “reactive group paired with the reactive group for cross linker modification” are a pair of reactive groups having high affinity in a binding reaction. When two compounds each having the pair are bonded, even if there are various other functional groups in the compound, the pair preferentially reacts with high selectivity to form a bond.

Examples of the above pairs include pairs of functional groups in the click reaction.

The “click reaction” is a concept well known to those skilled in the art. (See H. C. Kolb, M. G. Finn & K. B. Sharpless: Angew. Chem. Int. Ed., 40, 2004 (2001), etc.)

The “click reaction” is understood as one embodiment as follows.

The “click reaction” refers to a reaction that has at least the following characteristics: (1) exhibiting orthogonality of a functional group (that is, a functional moiety only reacts with a reactive site complementary to the functional moiety without reacting with other reactive sites); and (2) irreversibility of the obtained bond (that is, when the reactant reacts to form a product, decomposition of the product into the reactant becomes difficult), or in some cases, possible reversibility of the obtained bond (that is, under appropriate conditions, returns to the reactant). Optionally, the “click” chemistry can further have one or more of the following characteristics: (1) stereospecificity; (2) reaction conditions without strict purification, atmosphere control, etc.; (3) readily available starting materials and reagents; (4) harmless solvents available or no solvents available at all; (5) isolation of products by crystallization or distillation; (6) physiological stability; (7) high thermodynamic driving power (for example, 10 to 20 kcal/mol); (8) single reaction products; and (9) high chemical yields (for example, more than 50%).

As one embodiment, the “reactive group for cross linker modification” and the “reactive group paired with the reactive group for cross linker modification” are preferably reactive groups for the click reaction, more preferably an alkynyl group, an alkenyl group, an azide group, or a tetrazinyl group, and still more preferably any of the formulae (CA) to (CL).

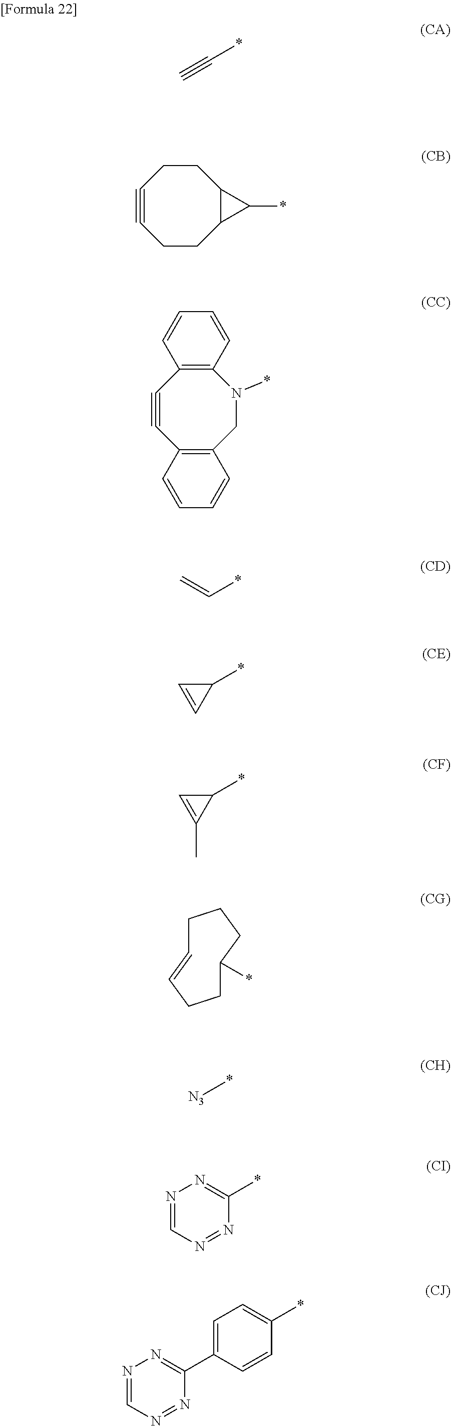

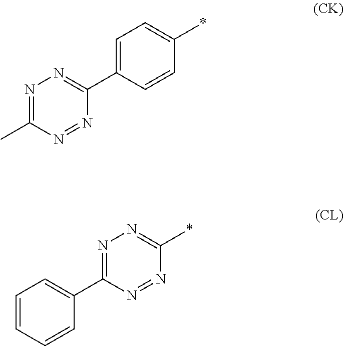 [SMILES: *c1ccc(-c2nnc(C)nn2)cc1 ; *c1nnc(-c2ccccc2)nn1]

Here, examples of the pair of “a reactive group for cross linker modification” and “a reactive group that is paired with a reactive group for cross linker modification” preferably include an azide group for an alkynyl group and a tetrazinyl group for an alkenyl group. These pairs are so-called bolt and nut relationships and are interchangeable. For example, when an alkynyl group is used as the “reactive group for cross linker modification”, an azide group can be used as the “reactive group paired with the reactive group for cross linker modification”. Their selection is well known to those skilled in the art.

Preferred examples of the “reactive group for cross linker modification” and the “reactive group paired with a reactive group for cross linker modification” include (CA) and (CH), (CB) and (CH), (CC) and (CH), (CE) and (CI), (CE) and (CJ), (CE) and (CK), (CE) and (CL), (CF) and (CI), (CF) and (CJ), (CF) and (CK), (CF) and (CL), (CG) and (CI), (CG) and (CJ), (CG) and (CK), (CG) and (CL), etc.

The “cross linker unit” is not particularly limited as long as it is a unit having the “reactive group paired with the reactive group for cross linker modification” as described above and a cross linker.

As one embodiment, the “cross linker unit” is composed of a “reactive group paired with a reactive group for cross linker modification”, a “bifunctional spacer”, and a “cross linker”. The embodiment of the “bifunctional spacer” is as described above.

The “DNA having a reactive group for cross linker modification” is not particularly limited as long as it is a compound having the “reactive group for cross linker modification” described above described above.

As one embodiment, the “DNA having a reactive group for cross linker modification” is composed of a “reactive group for cross linker modification,” a “bifunctional spacer,” and “DNA.” The embodiment of the “bifunctional spacer” is as described above.

For an embodiment of a “modified primer having a reactive group for cross linker modification”, the “cross linker-modified primer” described above is referred to. However, the term “cross linker” is replaced to refer to as a “reactive group for cross linker modification”.

Hereinafter, Examples are shown and the present invention will be described in more detail, but the present invention is not limited to these Examples. Various kinds of nucleic acids having sequences in Examples can be prepared, for example, according to a conventional method by an automated polynucleotide synthesizer. Examples of the automated polynucleotide synthesizer include nS-811 (manufactured by GeneDesign, Inc.), etc. In addition, for the preparation of the nucleic acid, consignment synthesis, contract labs, etc., can also be used. The contract labs well known to those skilled in the art include GeneDesign, Inc., LGC Biosearch Technologies, etc. In general, these contract labs prepare nucleic acids having the sequence specified by the consignor and deliver them to the consignor under a confidentiality agreement.

### Example 1

### [Verification of Cleavage Reaction of Partial Structure of Hairpin Type DEL Containing Deoxyuridine by USER® Enzyme]

The compound having the sequence shown in Table 1 was prepared using the automated polynucleotide synthesizer nS-811 (manufactured by GeneDesign, Inc.). In the sequence notations in Table 1, as is obvious to those skilled in the art, each sequence unit is bound by a phosphodiester bond, and “A” means deoxyadenosine, “T” means thymidine, “G” means deoxyguanosine, “C” means deoxycytidine, “(dU)” means deoxyuridine, “(p)” means phosphoric acid, and “(amino-C6-dT)” means the modified nucleic acid represented by the following formula (1)

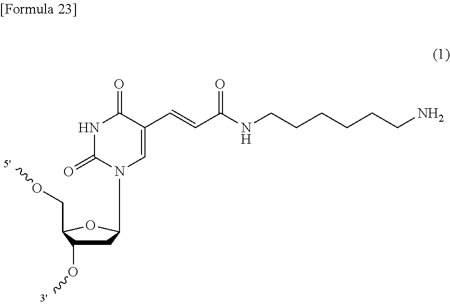

“(amino-NC6-dT)” means the modified nucleic acid represented by the following formula (2)

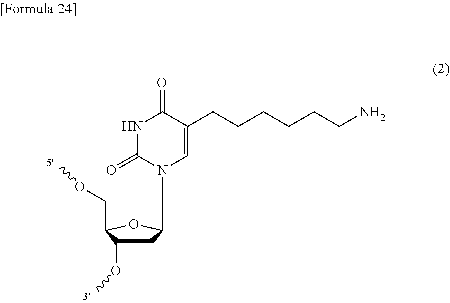

“(dSpacer)” means the group represented by the following formula (3)

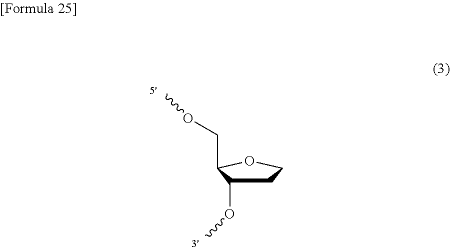

and “(aminoC7)” means the group represented by the following formula (4)

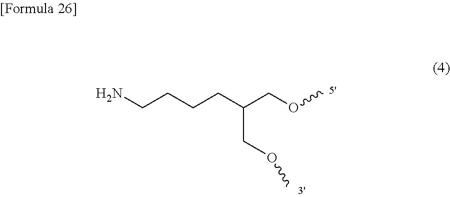

In addition, amino-NC6-dT was introduced using the nucleic acid synthetic reagent of the following formula (5)

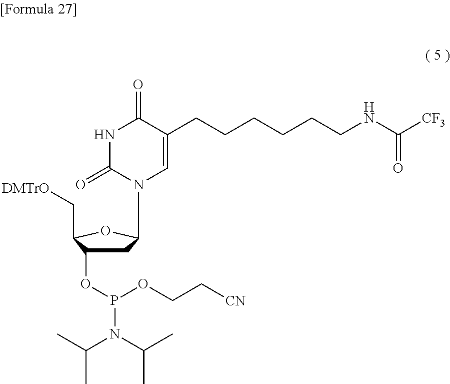

synthesized according to the method described in Journal of the American Chemical Society, 1993, vol. 115, pp. 7128-7134.

In Table 1, “No.” in the left column represents SEQ ID NO and “Seq.” in the right column represents a sequence. The left side of the sequence represents the 5′ side and the right side represents 3′ side. Also, the names of the compounds corresponding to each SEQ ID NO (No.) are as follows.

| | |
|---|---|
| No. 1: U-DEL1-sh | No. 2: U-DEL2-sh |
| No. 3: U-DEL3-sh | No. 4: U-DEL4-sh |
| No. 5: U-DEL5-HP | No. 6: U-DEL6-HP |
| No. 7: U-DEL7-HP | No. 8: U-DEL8-HP |
| No. 9: U-DEL9-HP | No. 10: U-DEL10-HP |

TABLE 1

| No. | Seq. |
|---|---|
| 1 | CATCGATTTGGGAGTCA(dU)T(amino-C6-dT)TTTGACTCCCAAATCGATGTG |
| 2 | CATCGATTTGGGAGTCATT(amino-C6-dT)T(dU)TGACTCCCAAATCGATGTG |
| 3 | CATCGATTTGGGAGTCATT(amino-C6-dT)TTTGACTCCCAAATCGA(dU)GTG |
| 4 | CATCGATTTGGGAGTCA(dU)T(amino-C6-dT)T(dU)TGACTCCCAAATCGATGTG |
| 5 | (p)GAGTCATT(amino-NC6-dT)T(dU)TGACTCCC |
| 6 | (p)GAGTCA(dU)T(amino-NC6-dT)T(dU)TGACTCCC |
| 7 | (p)GAGTCA(dU)T(dSpacer)(dSpacer)(AminoC7)(dSpacer)(dSpacer)TTTGACTCCC |
| 8 | (p)GAGTCATT(dSpacer)(dSpacer)(AminoC7)(dSpacer)(dSpacer)T(dU)TGACTCCC |
| 9 | (p)GAGTCAA(dSpacer)(dSpacer)(AminoC7)(dSpacer)(dSpacer)(dU)TGACTCCC |
| 10 | (p)GAGTCAT(dSpacer)(AminoC7)(dSpacer)(dU)TGACTCCC |

A 0.1 mM aqueous solution of each of the compounds having the sequences shown in Table 1 was prepared and investigation of the cleavage reaction by USER® enzyme was carried out by the following procedures.

To a PCR tube were added 1 μL of 0.1 mM aqueous solution of the compound having the sequence shown in Table 1; 10 μL of CutSmart® Buffer (available from New England BioLabs, Catalog number: B7204S); and 79 μL of deionized water. To the solution was added 10 μL of USER® enzyme (available from New England BioLabs, Catalog number: M5505S) and incubation of the obtained solution was started at 37° C.

Each reaction solution was sampled with each 20 μL after starting the incubation, 1 hour and 3 hours lapsed, respectively. U-DEL1-sh, U-DEL5-HP, U-DEL6-HP, U-DEL7-HP, U-DEL8-HP, U-DEL9-HP, and U-DEL10-HP were sampled with each 20 μL after 20 hours lapsed. U-DEL8-HP and U-DEL9-HP were further incubated at 90° C. for 1 hour and then sampled with each 20 μL, respectively.

Among the sampled solutions, U-DEL1-sh, U-DEL2-sh, U-DEL3-sh, and U-DEL4-sh were analyzed by Analytical condition 1 shown below, and U-DEL5-HP, U-DEL6-HP, U-DEL7-HP, U-DEL8-HP, U-DEL9-HP, and U-DEL10-HP were analyzed by Analytical condition 2 shown below.

### Analytical Condition 1:

Device: maXis (manufactured by Bruker), UltiMate 3000 (manufactured by Dionex)
Column: ACQUITY UPLC Oligonucleotide BEH C18 Column (130 Å, 1.7 μm, 2.1×50 mm)
Column temperature: 50° C.

### Solvent:

Solution A: Water (0.75% v/v hexafluoroisopropanol; 0.038% v/v triethylamine; 5 μM ethylenediamine tetraacetic acid)
Solution B: 90% v/v methanol aqueous solution (0.75% v/v hexafluoroisopropanol; 0.038% v/v triethylamine; 5 μM ethylenediamine tetraacetic acid)

### Gradient Conditions:

Flow rate of 0.36 mL/min; the measurement was started by fixing the mixing ratio of Solution A and Solution B to 95/5 (v/v), and after 0.56 minute, the mixing ratio of Solution A and Solution B was linearly changed to 40/60 (v/v) in 5.5 minutes.
Detection wavelength: 260 nm

### Analytical Condition 2:

Device: Waters ACQUITY UPLC/SQ Detector
Column: ACQUITY UPLC Oligonucleotide BEH C18 Column (130 Å, 1.7 μm, 2.1×50 mm)
Column temperature: 50° C.

### Solvent:

Solution A: Water (0.75% v/v hexafluoroisopropanol; 0.038% v/v triethylamine; 5 μM ethylenediamine tetraacetic acid)
Solution B: 90% v/v methanol aqueous solution (0.75% v/v hexafluoroisopropanol; 0.038% v/v triethylamine; 5 μM ethylenediamine tetraacetic acid)

### Gradient Conditions:

Flow rate of 0.36 mL/min; the measurement was started by fixing the mixing ratio of Solution A and Solution B to 95/5 (v/v), and after 0.56 minute, the mixing ratio of Solution A and Solution B was linearly changed to 40/60 (v/v) in 5.5 minutes.
Detection wavelength: 260 nm

The sequences and the expected molecular weights of the products (abasic product of deoxyuridine portion and cleaved fragments) assumed in each reaction solution and the molecular weight observed in each reaction solution are shown in Tables 2 and 3. In Tables 2 and 3, the notation of each column is as follows.

### “Entry” (Leftmost):

It indicates the experimental number and the substrates corresponding to each experimental number (Entry) are as follows.

| | |
|---|---|
| Entry. 1: U-DEL1-sh | Entry. 2: U-DEL2-sh |
| Entry. 3: U-DEL3-sh | Entry. 4: U-DEL4-sh |
| Entry. 5: U-DEL5-HP | Entry. 6: U-DEL6-HP |
| Entry. 7: U-DEL7-HP | Entry. 8: U-DEL8-HP |
| Entry. 9: U-DEL9-HP | Entry. 10: U-DEL10-HP |

“No.” (Second from the Left):

It indicates the sequence number. Among the respective SEQ ID NOs (Nos.), Nos. 1, 2, 3, 4, 5, 6, 7, 8, 9, and 10 are substrates of each reaction solution, Nos. 11, 14, 17, 20, 22, 25, 29, 31, 33, and 35 are dibasic products of the deoxyuridine portion of each substrate, and the remaining SEQ ID NOs are fragments of each substrate cleaved.

“Seq.” (Third from Left):

It represents the sequence, the left side represents the 5′ side, and the right side represents the 3′ side.

In the sequence notation, “(B)” means the group (debasic site) represented by the following formula (6)

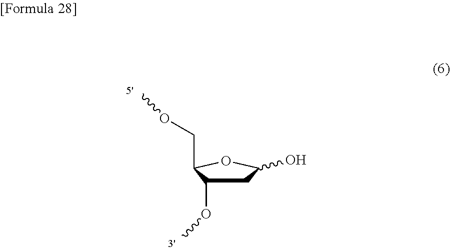

and other notations are the same as in Table 1.

“Expected MW.” (fourth from left):

It represents the numerical values of the expected molecular weight (Da) of each sequence.

### “Observed MW.” (Rightmost):

It represents the numerical value of the observed molecular weight (Da) identified as each sequence. “-” notation indicates that it has not been detected.

TABLE 2

| | | | Expected | Observed MW. |
|---|---|---|---|---|
| Entry | No. | Seq. | MW. | (deconvolution) |
| 1 | 1 | CATCGATTTGGGAGTCA(dU)T(amino-C6-dT)T | 12736.4 | 12736.3 |
| | | TTGACTCCCAAATCGATGTG | | |
| | 11 | CATCGATTTGGGAGTCA(B)T(amino-C6-dT)T | 12642.2 | 12643.3 |
| | | TTGACTCCCAAATCGATGTG | | |
| | 12 | CATCGATTTGGGAGTCA(P) | 5305.4 | 5303.8 |
| | 13 | (p)T(amino-C6-dT)TTTGACTCCCAAATCGATG | 7238.8 | 7239.3 |
| | | TG | | |
| 2 | 2 | CATCGATTTGGGAGTCATT(amino-C6-dT)T | 12736.4 | 12735.2 |
| | | (dU)TGACTCCCAAATCGATGTG | | |
| | 14 | CATCGATTTGGGAGTCATT(amino-C6-dT)T | 12642.2 | — |
| | | (B)TGACTCCCAAATCGATGTG | | |
| | 15 | CATCGATTTGGGAGTCATT(amino-C6-dT)T(p) | 6676.4 | 6676.1 |
| | 16 | (p)TGACTCCCAAATCGATGTG | 5867.8 | 5866.0 |
| 3 | 3 | CATCGATTTGGGAGTCATT(amino-C6-dT)TT | 12736.4 | 12736.2 |
| | | TGACTCCCAAATCGA(dU)GTG | | |
| | 17 | CATCGATTTGGGAGTCATT(amino-C6-dT)TT | 12642.2 | — |
| | | TGACTCCCAAATCGA(B)GTG | | |
| | 18 | CATCGATTTGGGAGTCATT(amino-C6-dT)TT | 11563.6 | 11562.1 |
| | | TGACTCCCAAATCGA(p) | | |
| | 19 | (p)GTG | 980.6 | — |
| 4 | 4 | CATCGATTTGGGAGTCA(dU)T(amino-C6-dT)T | 12722.4 | 12721.2 |
| | | (dU)TGACTCCCAAATCGATGTG | | |
| | 20 | CATCGATTTGGGAGTCA(B)T(amino-C6-dT)T | 12534.0 | — |
| | | (B)TGACTCCCAAATCGATGTG | | |
| | 12 | CATCGATTTGGGAGTCA(p) | 5305.4 | 5304.9 |
| | 16 | (p)TGACTCCCAAATCGATGTG | 5867.8 | 5867.0 |
| | 21 | (p)T(amino-C6-dT)T(p) | 1164.8 | — |
| 5 | 5 | (p)GAGTCATT(amino-NC6-dT)T(dU)TGACTCCC | 5895.9 | 5896.7 |
| | 22 | (p)GAGTCATT(amino-NC6-dT)T(B)TGACTCCC | 5801.7 | 5801.7 |
| | 23 | (p)GAGTCATT(amino-NC6-dT)T(p) | 3278.1 | 3278.7 |
| | 24 | (p)TGACTCCC | 2425.6 | 2425.9 |
| 6 | 6 | (p)GAGTCA(dU)T(amino-NC6-dT)T(dU)TGACT | 5881.8 | 5882.0 |
| | | CCC | | |
| | 25 | (p)GAGTCA(B)T(amino-NC6-dT)T(B)TGACTCCC | 5693.5 | 5693.5 |
| | 26 | (p)GAGTCA(B)T(amino-NC6-dT)T(p) | 3170.0 | 3171.0 |
| | 27 | (p)GAGTCA(p) | 1976.2 | 1977.1 |
| | 24 | (P)TGACTCCC | 2425.6 | 2425.3 |
| | 28 | (p)T(amino-NC6-dT)T(p) | 1095.7 | — |
| 7 | 7 | (p)GAGTCA(dU)T(dSpacer)(dSpacer)(AminoC7) | 6436.1 | 6437.7 |
| | | (dSpacer)(dSpacer)TTTGACTCCC | | |
| | 29 | (p)GAGTCA(B)T(dSpacer)(dSpacer)(AminoC7) | 6341.9 | — |
| | | (dSpacer)(dSpacer)TTTGACTCCC | | |
| | 27 | (p)GAGTCA(p) | 1976.2 | 1976.4 |
| | 30 | (p)T(dSpacer)(dSpacer)(AminoC7)(dSpacer) | 4267.7 | 4268.8 |
| | | (dSpacer)TTTGACTCCC | | |
| 8 | 8 | (p)GAGTCATT(dSpacer)(dSpacer)(AminoC7) | 6436.1 | 6437.9 |
| | | (dSpacer)(dSpacer)T(dU)TGACTCCC | | |
| | 31 | (p)GAGTCATT(dSpacer)(dSpacer)(AminoC7) | 6341.9 | 6344.2 |
| | | (dSpacer)(dSpacer)T(B)TGACTCCC | | |
| | 32 | (p)GAGTCATT(dSpacer)(dSpacer)(AminoC7) | 3818.4 | 3818.7 |
| | | (dSpacer)(dSpacer)T(p) | | |
| | 24 | (p)TGACTCCC | 2425.6 | 2425.8 |
| 9 | 9 | (p)GAGTCAA(dSpacer)(dSpacer)(AminoC7) | 5836.7 | 5838.5 |
| | | (dSpacer)(dSpacer)(dU)TGACTCCC | | |
| | 33 | (p)GAGTCAA(dSpacer)(dSpacer)(AminoC7) | 5742.6 | 5743.8 |
| | | (dSpacer)(dSpacer)(B)TGACTCCC | | |
| | 34 | (p)GAGTCAA(dSpacer)(dSpacer)(AminoC7) | 3219.0 | 3219.7 |
| | | (dSpacer)(dSpacer)(p) | | |
| | 24 | (p)TGACTCCC | 2425.6 | 2425.7 |
| 10 | 10 | (p)GAGTCAT(dSpacer)(AminoC7)(dSpacer)(du)T | 5467.5 | 5469.4 |
| | | GACTCCC | | |
| | 35 | (p)GAGTCAT(dSpacer)(AminoC7)(dSpacer)(B)TG | 5373.4 | 5375.9 |
| | | ACTCCC | | |
| | 36 | (p)GAGTCAT(dSpacer)(AminoC7)(dSpacer)(p) | 2849.8 | 2850.2 |
| | 24 | (p)TGACTCCC | 2425.6 | 2426.0 |

From the area ratio of the peak corresponding to each sequence detected, the conversion rate of the debasic reaction and of the cleavage reaction were calculated. In the debasic reaction, 9900 or more was converted in all the substrate at the stage of 1 hour at 37° C. (the peak of the substrate was less than 100 and the remaining peak was of the debasic product and the cleaved fragment only).

A graph showing the conversion rate of the cleavage reaction is also shown in FIG. 10. As shown in the graph, in all the substrates except for U-DEL8-HIP and U-DEL9-HP, the cleavage reaction proceeded 95% or more by 20 hours at 37° C., and in U-DEL8-1IP and U-DEL9-HIP, 10000 of the cleavage reaction was completed by adding the incubation of 1 hour at 90° C.

From the above results, at the partial structure of the hairpin type DEL containing various kinds of deoxyuridines, it was shown that, at deoxyuridine site, a debasic reaction by USER® enzyme and subsequently a cleavage reaction proceeded.

### Example 2

### [Comparison of PCR Efficiency Between Conventional Type Hairpin DEL and Cleavable Hairpin DEL (Hairpin Type DEL Containing Deoxyuridine)]

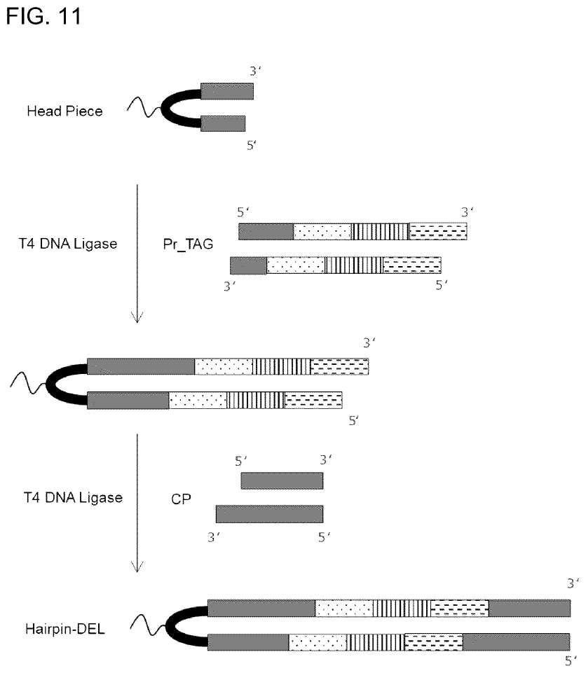

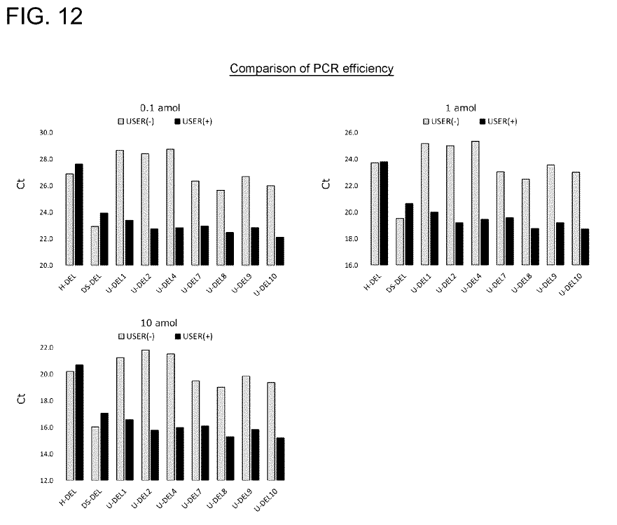

As in the schematic diagram shown in FIG. 11, the compounds having the sequence shown in Table 4 (hairpin DEL) were synthesized by the following procedures. In the sequence notation in Table 4, “(S)” means the group represented by the following formula (7)

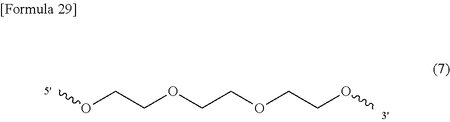

and other notations are the same as in Table 1.

The names of the compounds corresponding to each SEQ ID NO (No.) are as follows.

| | |
|---|---|
| No. 37: U-DEL1 | No. 38: U-DEL2 |
| No. 39: U-DEL4 | No. 40: U-DEL7 |
| No. 41: U-DEL8 | No. 42: U-DEL9 |
| No. 43: U-DEL10 | No. 44: H-DEL |

TABLE 4

| | | Expected | Observed MW. |
|---|---|---|---|
| No. | Seq. | MW. | (deconvolution) |
| 37 | GCAGGTGAAGCTTGTCTGAAGACTGCTTCTGGAAC | 46707.3 | 46702.9 |
| | CACCATCGCACTTCCTGACCACATCGATTTGGGAGT | | |
| | CA(dU)T(amino-C6-dT)TTTGACTCCCAAATCGATGTG | | |
| | GTCAGGAAGTGCGATGGTGGTTCCAGAAGCAGTCT | | |
| | TCAGACAAGCTTCACCTGC | | |
| 38 | GCAGGTGAAGCTTGTCTGAAGACTGCTTCTGGAAC | 46707.3 | 46711.8 |
| | CACCATCGCACTTCCTGACCACATCGATTTGGGAGT | | |
| | CATT(amino-C6-dT)T(dU)TGACTCCCAAATCGATGTG | | |
| | GTCAGGAAGTGCGATGGTGGTTCCAGAAGCAGTCT | | |
| | TCAGACAAGCTTCACCTGC | | |
| 39 | GCAGGTGAAGCTTGTCTGAAGACTGCTTCTGGAAC | 46693.2 | 46695.5 |
| | CACCATCGCACTTCCTGACCACATCGATTTGGGAGT | | |
| | CA(dU)T(amino-C6-dT)T(dU)TGACTCCCAAATCGATGT | | |
| | GGTCAGGAAGTGCGATGGTGGTTCCAGAAGCAGTC | | |
| | TTCAGAGAAGCTTCACCTGC | | |
| 40 | GCAGGTGAAGCTTGTCTGAAGACTGCTTCTGGAAC | 47178.4 | 47184.8 |
| | CACCATCGCACTTCCTGACCACATCGATTTGGGAGT | | |
| | CA(dU)T(dSpacer)(dSpacer)(AminoC7)(dSpacer) | | |
| | (dSpacer)TTTGACTCCCAAATCGATGTGGTCAGGAAG | | |
| | TGCGATGGTGGTTCCAGAAGCAGTCTTCAGACAAG | | |
| | CTTCACCTGC | | |
| 41 | GCAGGTGAAGCTTGTCTGAAGACTGCTTCTGGAAC | 47178.4 | 47189.7 |
| | CACCATCGCACTTCCTGACCACATCGATTTGGGAGT | | |
| | CATT(dSpacer)(dSpacer)(AminoC7)(dSpacer)(dSpacer)T | | |
| | (dU)TGAGTCCCAAATCGATGTGGTCAGGAAGTGCGA | | |
| | TGGTGGTTCCAGAAGCAGTCTTGAGACAAGCTTCAC | | |
| | CTGC | | |
| 42 | GCAGGTGAAGCTTGTCTGAAGACTGCTTCTGGAAC | 46579.1 | 46592.0 |
| | CACCATCGCACTTCCTGACCACATCGATTTGGGAGT | | |
| | CAA(dSpacer)(dSpacer)(AmnioC7)(dSpacer) | | |
| | (dSpacer)d(U)TGACTCCCAAATCGATGTGGTCAGGAA | | |
| | GTGCGATGGTGGTTCCAGAAGCAGTCTTCAGACAA | | |
| | GCTTCACCTGC | | |
| 43 | GCAGGTGAAGCTTGTCTGAAGACTGCTTCTGGAAC | 46209.9 | 46220.0 |
| | CACCATCGCACTTCCTGACCACATCGATTTGGGAGT | | |
| | CAT(dSpacer)(AminoC7)(dSpacer)(dU)TGACTCCCAAA | | |
| | TCGATGTGGTCAGGAAGTGCGATGGTGGTTCCAGA | | |
| | AGCAGTCTTCAGACAAGCTTCACCTGC | | |
| 44 | GCAGGTGAAGCTTGTCTGAAGACTGCTTCTGGAAG | 45679.6 | 45666.6 |
| | CACCATCGCACTTCCTGACCACATCGATTTGGGAGT | | |
| | CAS(AminoC7)STGACTCCCAAATCGATGTGGTCAGG | | |
| | AAGTGCGATGGTGGTTCCAGAAGCAGTCTTCAGAC | | |
| | AAGCTTCACCTGC | | |

The names of the compounds of the starting material head piece for synthesizing each hairpin DEL are each as follows.

| Hairpin | Starting material |
|---|---|
| DEL: | head piece |
| U-DEL1: | U-DEL1-HP |
| U-DEL2: | U-DEL2-HP |
| U-DEL4: | U-DEL4-HP |
| U-DEL7: | U-DEL7-HP |
| U-DEL8: | U-DEL8-HP |
| U-DEL9: | U-DEL9-HP |
| U-DEL10: | U-DEL10-HP |
| H-DEL: | H-DEL-HP |

Further, SEQ ID NO “No.” and the sequence “Seq” of U-DEL1-HP, U-DEL2-HP, U-DEL4-HP, and H-DEL-HP are as shown in Table 5.

TABLE 5

| | No. | Seq. |
|---|---|---|
| U-DEL1-HP | 45 | (p)GAGTCA(dU)T(amino-C6-dT)TTTGACTCCC |
| U-DEL2-HP | 46 | (p)GAGTCATT(amino-C6-dT)T(dU)TGACTCCC |
| U-DEL4-HP | 47 | (p)GAGTCA(dU)T(amino-C6-dT)T(dU)TGACTCCC |
| H-DEL-HP | 48 | (p)GAGTCAS(AminoC7)STGACTCCC |

The starting material head piece shown in Table 5 were prepared using the automated polynucleotide synthesizer nS-811 (manufactured by GeneDesign, Inc.) in the same manner as in Example 1.

To a PCR tube were added 2.0 μL of 1 mM aqueous solution of various kinds of the starting material head piece; 2.4 μL of 1 mM aqueous solution of Pr_TAG (it was prepared by annealing Pr_TAG_a and Pr_TAG_b synthesized in the same manner as in Example 1; the sequences are shown in Table 6); 0.8 μL of 10× ligase buffer (500 mM Tris hydrochloride, pH 7.5; 500 mM sodium chloride; 100 mM magnesium chloride; 100 mM dithiothreitol; and 20 mM adenosine triphosphate); and 2.0 μL of deionized water. To the solution was added 0.8 μL of a 10-fold diluted aqueous solution of T4DNA ligase (available from Thermo Fisher, Catalog number: EL0013) and the obtained solution was incubated at 16° C. for 24 hours. The sequence notation in Table 6 is the same as in Table 1. Also, the names of the compounds corresponding to each SEQ ID NO (No.) are as follows.

| | |
|---|---|
| No. 49: Pr_TAG_a | No. 50: Pr_TAG_b |

TABLE 6

| No. | Seq. |
|---|---|
| 49 | (p)GACTGCTTCTGGAACCACCATCGCACTTCCTGACCACATCGATTTGG |
| 50 | (p)AAATCGATGTGGTCAGGAAGTGCGATGGTGGTTCCAGAAGCAGTCTT |

The reaction solution was treated with 0.8 μL of 5 M aqueous sodium chloride solution and 17.6 μL of cooled (−20° C.) ethanol and allowed to stand at −78° C. for 2 hours. After centrifugation, the supernatant was removed and the obtained pellets were air-dried. To each pellet was added 2.0 μL of deionized water to prepare a solution.

To the obtained each solution were added 2.4 μL of 1 mM aqueous solution of CP (it was prepared by annealing CP_a and CP_b synthesized in the same manner as in Example 1; the sequences are shown in Table 7); 0.8 μL of 10× ligase buffer (500 mM Tris hydrochloride, pH 7.5; 500 mM sodium chloride; 100 mM magnesium chloride; 100 mM dithiothreitol; and 20 mM adenosine triphosphate), and 2.0 μL of deionized water. To the solution was added 0.8 μL of a 10-fold diluted aqueous solution of T4DNA ligase (available from Thermo Fisher, Catalog number: EL0013) and the obtained solution was incubated at 16° C. for 24 hours. The sequence notations in Table 7 are the same as in Table 1. Also, the names of the compounds corresponding to each SEQ ID NO (No.) are as follows.

| | |
|---|---|
| No. 51: CP_a | No. 52: CP_b |

TABLE 7

| No. | Seq. |
|---|---|
| 51 | GCAGGTGAAGCTTGTCTGAA |
| 52 | (p)CAGACAAGCTTCACCTGC |

The reaction solution was treated with 0.8 μL of 5 M aqueous sodium chloride solution and 17.6 μL of cooled (−20° C.) ethanol and allowed to stand at −78° C. for 2 hours. After centrifugation, the supernatant was removed and the obtained pellets were air-dried. To the pellets was added 10 μL of deionized water to prepare a solution.

From the obtained solution, 1.0 μL was sampled, and after diluting with deionized water, mass spectrometry by ESI-MS was carried out under the conditions of Analytical condition 2 in Example 1 to identify the target product (the expected molecular weight and the observed molecular weight of each sequence are shown in Table 4). After lyophilizing the rest of the solution, deionized water was added to prepare a 20 M solution.

Among the eight kinds of the hairpin type DEL obtained as mentioned above, H-DEL is a conventional type hairpin DEL and the remaining seven kinds are cleavable hairpin DELs containing deoxyuridine. Real-time PCR analysis was carried out to compare the PCR efficiency of various kinds of hairpin type DELs before treatment with USER® enzyme and the PCR efficiency after the treatment. Also, as the double-stranded DEL to be compared, DS-DEL (it was prepared by annealing the compounds having the sequences No. 47 and No. 48) shown in Table 7 was used. In the sequence notations in Table 8, “(amino-C6-L)” means the group represented by the following formula (8)

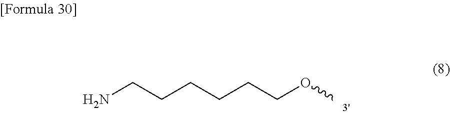

and other notations are the same as in Table 1.

TABLE 8

| | No. | Seq. |
|---|---|---|
| DS-DEL | 53 | (amino-C6-L)TGACTCCCAAATCGATGTGGTCAGGAAGTGCGATGGTGGTTCCAGAAGCAGTC |
| | | TTCAGACAAGCTTCACCTGC |
| | 54 | GCAGGTGAAGCTTGTCTGAAGACTGCTTCTGGAACCACCATCGCACTTCCTGACCACATCGA |
| | | TTTGGGAGTCA |

<Treatment Step with USER® Enzyme>

Treatment of eight kinds of hairpin DELs and double-stranded DEL (DS-DEL) with USER® enzyme was carried out by the following procedures.

To a PCR tube were added 1 μL of various kinds of 20 M DEL aqueous solution; 1 μL of CutSmart® Buffer (available from New England BioLabs, Catalog number: B7204S); and 7 μL of deionized water. To the solution was added 1 μL of USER® enzyme (available from New England BioLabs, Catalog number: M5505S) and the obtained solution was incubated at 37° C. for 1 hour.

### <Preparation of DEL Samples>

Samples of various kinds of DELs before the treatment with USER® enzyme and the reaction solutions after the treatment were each diluted with deionized water to prepare DEL samples with 0.05 pM, 0.5 pM, and 5 pM.

### <Measurement of Ct Value by Real-Time PCR>

The Ct value of various kinds of the DEL samples obtained as mentioned above was measured by real-time PCR and the PCR efficiencies were compared. The conditions are as mentioned below and the results are shown in FIG. 12. The Ct value is the number of cycles in which the fluorescent signal generated by amplification of DNA reaches an arbitrary threshold value in real-time PCR. That is, when the initial number of DNA molecules is the same, the higher the PCR efficiency, the lower the Ct value.
Device: 7500 real-time PCR system (manufactured by Applied Biosystems)
Plate: MicroAmp 96-Well plate (manufactured by Applied Biosystems,
Catalog number: N8010560)
PCR the reaction solution:
   TB Green Premix Ex taqII (available from Takara Bio Inc., Catalog number: RR820): 10 μL
   Forward primer (Table 9, SEQ ID NO: 55): 0.80 μL
   Reverse primer (Table 9, SEQ ID NO: 56): 0.80 μL
   ROX Refference DyeII (available from Takara Bio Inc., Catalog number: RR39LR): 0.40 μL
   Various kinds of aqueous solutions of DEL samples (0.05 pM, 0.5 pM, 5 pM)*1: 2.0 μL
   Deionized water: 6.0 μL
      1: The moles of the DEL samples are 0.1 amol, 1 amol, and 10 amol.
Temperature conditions:
   After holding at 95° C. for 2 minutes, the following cycle was repeated for 35 cycles.
   95° C., 5 seconds
   52° C., 30 seconds
   72° C., 30 seconds

TABLE 9

| No. | Seq. |
|---|---|
| 55 | TGACTCCCAAATCGA |
| 56 | GCAGGTGAAGCTTGTC |

The sequence notations in Table 9 are the same as in Table 1.

As shown in FIG. 12, in the conventional type hairpin DEL (H-DEL), the Ct value does not change before and after the USER® enzyme treatment, but in the cleavable hairpin DEL (U-DEL1, U-DEL2, U-DEL4, U-DEL7, U-DEL8, U-DEL9, and U-DEL10) containing deoxyuridine, the Ct value was lowered to the same level as that of DS-DEL, which is a double-stranded DEL, after the USER® enzyme treatment.

This result shows that the DEL cleaved by the USER® enzyme has improved PCR efficiency than that before the cleavage and that the cleavable hairpin DEL containing deoxyuridine was cleaved by USER® enzyme with high efficiency and high selectively.

### Example 3

### [Verification of Cleavage Reaction of Hairpin DEL Containing Deoxyuridine by USER® Enzyme]<Synthesis of Four Kinds of Hairpin DELs (U-DEL5, U-DEL11, U-DEL12, and U-DEL13)>

The compounds having the sequences shown in Table 10 (hairpin DEL) were synthesized by the following procedures. In the sequence notations in Table 10, “[mdC (TEG-Amino)]” means the group represented by the following formula (9)

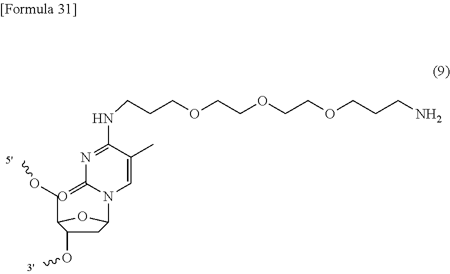

and other notations are the same as in Table 4.

The names of the compounds corresponding to each SEQ ID NO (No.) are as follows.

| | |
|---|---|
| No. 57: U-DEL5 | No. 58: U-DEL11 |
| No. 59: U-DEL12 | No. 60: U-DEL13 |

TABLE 10

| | | Expected | Observed MW. |
|---|---|---|---|
| No. | Seq. | MW. | (deconvoluiton) |
| 57 | GCAGGTGAAGCTTGTCTGAAGACTGCTTCTGGAACCACCATCGCACTTCCTGACC | 46638.2 | 46674.1 |
| | ACATCGATTTGGGAGTCATT(amino-NC6-dT)T(dU)TGACTCCCAAATCGATGTGGTC | | |
| | AGGAAGTGCGATGGTGGTTCCAGAAGCAGTCTTCAGACAAGCTTCACCTGC | | |
| 58 | GCAGGTGAAGCTTGTCTGAAGACTGCTTCTGGAACCACCATCGCACTTCCTGACC | 46283.0 | 46311.0 |
| | ACATCGATTTGGGAGTCAAS(aminoC7)S(dU)TGACTCCCAAATCGATGTGGTCAGG | | |
| | AAGTGCGATGGTGGTTCCAGAAGCAGTCTTCAGACAAGCTTCACCTGC | | |
| 59 | GCAGGTGAAGCTTGTCTGAAGACTGCTTCTGGAACCACCATCGCACTTCCTGACC | 46156.0 | 46174.6 |
| | ACATCGATTTGGGAGTCAA[mdC(TEG-amino)](dU)TGACTCCCAAATCGATGTGGT | | |
| | CAGGAAGTGCGATGGTGGTTCCAGAAGCAGTCTTCAGACAAGCTTCACCTGC | | |
| 60 | GCAGGTGAAGCTTGTCTGAAGACTGCTTCTGGAACCACCATCGCACTTCCTGACC | 46755.4 | 46769.6 |
| | ACATCGATTTGGGAGTCATT[mdC(TEG-amino)](dU)TTGACTCCCAAATCGATGTGG | | |
| | TCAGGAAGTGCGATGGTGGTTCCAGAAGCAGTCTTCAGACAAGCTTCACCTGC | | |

The names of the compounds of the starting material head piece for synthesizing each hairpin DEL are each as follows.

| Hairpin | Starting material |
|---|---|
| DEL: | head piece |
| U-DEL5: | U-DEL5-HP |
| U-DEL11: | U-DEL11-HP |
| U-DEL12: | U-DEL12-HP |
| U-DEL13: | U-DEL13-HP |

Further, SEQ ID NO “No.” and the sequences “Seq” of U-DEL11-HP, U-DEL12-HP, and U-DEL13-HP are as mentioned in the following Table 11. The notations in Table 11 are the same as in Table 10.

TABLE 11

| | No. | Seq. |
|---|---|---|
| U-DEL11-HP | 61 | (p)GAGTCAAS (aminoC7)S (dU)TGACTCCC |
| U-DEL12-HP | 62 | (p)GAGTCAA [m dC(TEG-amino)](du) |
| | | TGACTCCC |
| U-DEL13-HP | 63 | (p)GAGTCATT [m dC(TEG-amino)](dU) |
| | | TTGACTCCC |

Among the starting material head piece shown in Table 11, U-DEL12-HP and U-DEL13-HP were prepared using the automated polynucleotide synthesizer nS-811 (manufactured by GeneDesign, Inc.) in the same manner as in Example 1. U-DEL11-HP was also prepared according to a conventional method.

Similar to Example 2, using various kinds of starting material head pieces, two-step double-stranded ligation with the double-stranded oligonucleotide Pr_TAG and CP was carried out.

A part of the obtained solution was sampled, and after diluting with deionized water, mass spectrometry by ESI-MS was carried out under Analytical condition 3 shown below to identify the target product (the expected molecular weight and the observed molecular weight of each sequence are shown in Table 10). After lyophilizing the rest of the solution, deionized water was added to prepare a 20 M solution.

### Analytical Condition 3:

Device: Waters ACQUITY UPLC/SQ Detector
Column: ACQUITY UPLC Oligonucleotide BEH C18 Column (130 Å, 1.7 m, 2.1×50 mm)
Column temperature: 60° C.
Solvent:
   Solution A: Water (0.75% v/v hexafluoroisopropanol; 0.038% v/v triethylamine; and 5 M ethylenediamine tetraacetic acid)
   Solution B: 90% v/v methanol aqueous solution (0.75% v/v hexafluoroisopropanol; 0.038% v/v triethylamine; and 5 μM ethylenediamine tetraacetic acid)
Gradient conditions:
The flow rate of 0.36 mL/min; The measurement was started by fixing the mixing ratio of Solution A and Solution B to 95/5 (v/v), and after 0.56 minute, the mixing ratio of Solution A and Solution B was linearly changed to 40/60 (v/v) in 5.5 minutes.
Detection wavelength: 260 nm
Deconvolution:
Ion signals were analyzed using ProMass for MassLynx Software (manufactured by Waters).

### <Cleavage Reaction by USER® Enzyme>

Verification of the cleavage reaction of the hairpin DEL (U-DEL5, U-DEL7, U-DEL9, U-DEL11, U-DEL12, and U-DEL13) containing 6 kinds of deoxyuridines by USER® enzyme was carried out by the following procedures.

To a PCR tube were added 2 μL of various kinds of 20 M hairpin DEL aqueous solution; 2 μL of CutSmart® Buffer (available from New England BioLabs, Catalog number: B7204S); and 14 μL of deionized water. To the solution was added 2 μL of USER® enzyme (available from New England BioLabs, Catalog number: M5505S), and the obtained solution was incubated at 37° C. for 16 hours and further incubated at 90° C. for 1 hour.

<Confirmation of Product after Cleavage by LC-MS Measurement>

From the obtained reaction solutions, 5.0 μL was sampled, and after diluting with deionized water, mass spectrometry by ESI-MS was carried out under Analytical condition 3. The sequences and the expected molecular weights of the products after the cleavage assumed in each reaction solution and the molecular weight observed in each reaction solution are shown in Table 12. The substrates corresponding to each experimental numbers (Entry) are as follows and other notations are the same as in Table 10.

| | |
|---|---|
| Entry. 1: U-DEL5 | Entry. 2: U-DEL7 |
| Entry. 3: U-DEL9 | Entry. 4: U-DEL11 |
| Entry. 5: U-DEL12 | Entry. 6: U-DEL13 |

TABLE 12

| | | | Expect- | Observed MW. |
|---|---|---|---|---|
| | | | ed | (deconvo- |
| Entry | No. | Seq. | MW. | lution) |
| 1 | 64 | GCAGGTGAAGCTTGTCTGAAGACTGCTTCTGGAACCACCATCGCACTTCCTGACCA | 23829.5 | 23831.6 |
| | | CATCGATTTGGGAGTCATT (amino-N C 6-dT)T(p) | | |
| | 65 | (p)TGACTCCCAAATCGATGTGGTCAGGAAGTGCGATGGTGGTTCCAGAAGCAGTCT | 22616.6 | 22617.4 |
| | | TCAGACAAGCTTCACCTGC | | |
| 2 | 66 | GCAGGTGAAGCTTGTCTGAAGACTGCTTCTGGAACCACCATCGCACTTCCTGACCA | 22527.5 | 22527.6 |
| | | CATCGATTTGGGAGTCA(p) | | |
| | 67 | (p)T(dSpacer)(dSpacer)(aminoC7)(dSpacer)(dSpacer) | 24458.7 | 24459.9 |
| | | TTTGACTCCCAAATCGATGTGGTCAGGAAGTGCGATGGTGGTTCCAGAAGCAGTCT | | |
| | | TCAGACAAGCTTCACCTGC | | |
| 3 | 68 | GCAGGTGAAGCTTGTCTGAAGACTGCTTCTGGAACCACCATCGCACTTCCTGACCA | 23770.3 | 23780.4 |
| | | CATCGATTTGGGAGTCAA (dSpacer)(dSpacer(aminoC7)(dSpacer) | | |
| | | (dSpacer)(p) | | |
| | 65 | (p)TGACTCCCAAATCGATGTGGTCAGGAAGTGCGATGGTGGTTCCAGAAGCAGTCT | 22616.6 | 22615.8 |
| | | TCAGACAAGCTTCACCTGC | | |
| 4 | 69 | GCAGGTGAAGCTTGTCTGAAGACTGCTTCTGGAACCACCATCGCACTTCCTGACCA | 23474.2 | 23475.7 |
| | | CATCGATTTGGGAGTCAAS (aminoC7)S(p) | | |
| | 65 | (p)TGACTCCCAAATCGATGTGGTCAGGAAGTGCGATGGTGGTTCCAGAAGCAGTCT | 22616.6 | 22617.7 |
| | | TCAGACAAGCTTCACCTGC | | |
| 5 | 70 | GCAGGTGAAGCTTGTCTGAAGACTGCTTCTGGAACCACCATCGCACTTCCTGACCA | 23347.2 | 23327.2 |
| | | CATCGATTTGGGAGTCAA [m dC (TEG-amino)](p) | | |
| | 65 | (p)TGACTCCCAAATGGATGTGGTCAGGAAGTGCGATGGTGGTTCCAGAAGCAGTCT | 22616.6 | 22614.6 |
| | | TCAGACAAGCTTCACCTGC | | |
| 6 | 71 | GCAGGTGAAGCTTGTCTGAAGACTGCTTCTGGAACCACCATCGCACTTCCTGACCA | 23642.4 | 23646.3 |
| | | CATCGATTTGGGAGTCATT [m dC (TEG-amino)](p) | | |
| | 72 | (p)TTGACTCCCAAATCGATGTGGTCAGGAAGTGCGATGGTGGTTCCAGAAGCAGTC | 22920.8 | 22929.0 |
| | | TTCAGACAAGCTTCACCTGC | | |

In any of the samples, no MS of the substrate was detected, and the MS of the product after the cleavage was observed as the main peak.

### <Confirmation of Cleavage Reaction Using Gel Electrophoresis>

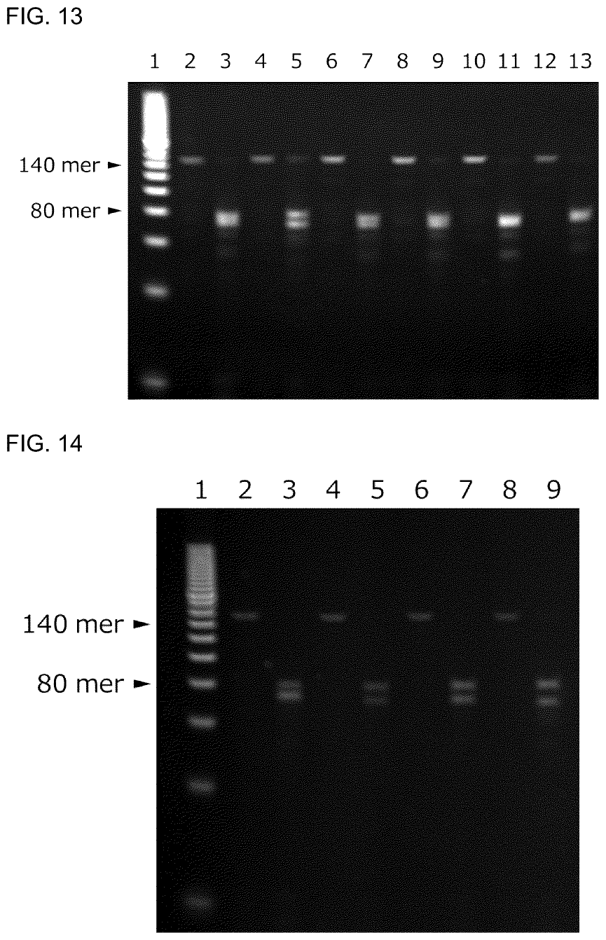

Also, among the obtained reaction solutions, a part thereof was sampled and analyzed by modified polyacrylamide gel electrophoresis under the conditions shown below. From the results shown in FIG. 13, it was confirmed that the cleavage reaction proceeded in high yield for all the substrates. The samples of each Lane in FIG. 13 are as follows.
Lane 1: 20 bp DNA Ladder (manufactured by Lonza, Lonza 20 bp DNA Ladder, Catalog number: 50330)
Lane 2: U-DEL5
Lane 3: Sample after subjecting to cleavage reaction of U-DEL5
Lane 4: U-DEL7
Lane 5: Sample after subjecting to cleavage reaction of U-DEL7
Lane 6: U-DEL9
Lane 7: Sample after subjecting to cleavage reaction of U-DEL9
Lane 8: U-DEL11
Lane 9: Sample after subjecting to cleavage reaction of U-DEL11
Lane 10: U-DEL12
Lane 11: Sample after subjecting to cleavage reaction of U-DEL12
Lane 12: U-DEL13
Lane 13: Sample after subjecting to cleavage reaction of U-DEL13

### Modified Polyacrylamide Gel Electrophoresis:

Gel: Novex (trademark) 10% TBE-urea gel (available from Invitrogen by Thermo Fisher SCIENTIFIC, Catalog number: EC68755BOX)
Loading Buffer: Novex (trademark) 10% TBE-Urea Sample Buffer (2×) (available from Invitrogen by Thermo Fisher SCIENTIFIC, Catalog number: LC6876)
Temperature: 60° C.
Voltage: 180V
Electrophoresis time: 30 min
Dyeing reagent: SYBER (trademark) GreenII Nucleic Acid Gel Stain (available from Takara Bio Inc., Catalog number: 5770A)

From the above results, in the hairpin type DEL containing various kinds of deoxyuridines, it was shown that the cleavage reaction by USER® enzyme proceeded at the deoxyuridine site.

### Example 4

### [Verification of Cleavage Reaction of Hairpin DEL Containing Deoxyinosine by Endonuclease V]

### <Syntheses of Hairpin DELs (I-DEL1, I-DEL2, I-DEL3, and I-DEL4) Containing 4 Kinds of Deoxyinosines>

The compounds having the sequence shown in Table 13 (hairpin DEL) were synthesized by the following procedures. In the sequence notations in Table 13, “I” means deoxyinosine and other notations are the same as in Table 2.

The names of the compounds corresponding to each SEQ ID NO (No.) are as follows.

| | |
|---|---|
| No. 73: I-DEL1 | No. 74: I-DEL2 |
| No. 75: I-DEL3 | No. 76: I-DEL4 |

TABLE 13

| | | | Observed |
|---|---|---|---|
| | | | MW. |
| | | Expected | (deconvo- |
| No. | Seq. | MW. | lution) |
| 73 | GCAGGTGAAGCTTGTCTGAAGACTGCTTCTGGAACCACCATCGCACTTCCTGACC | 47211.5 | 47241.5 |
| | ACATCGATTTGGGAGTCTAA (dSpacer)(dSpacer)(aminoC7)(dSpacer)(dSpacer) | | |
| | TTTGACTCCCAAATCGATGTGGTCAGGAAGTGCGATGGTGGTTCCAGAAGCAGTCTTCA | | |
| | GACAAGCTTCACCTGC | | |
| 74 | GCAGGTGAAGCTTGTCTGAAGACTGCTTCTGGAACCACCATCGCACTTCCTGACC | 46594.1 | 46621.2 |
| | ACATCGATTTGGGAGTCAT (dSpacer)(dSpacer)(aminoC7)(dSpacer)(dSpacer) | | |
| | TTGACTCCCAAATCGATGTGGTCAGGAAGTGCGATGGTGGTTCCAGAAGCAGTCTTCAGA | | |
| | CAAGCTTCACCTGC | | |
| 75 | GGAGGTGAAGCTTGTCTGAAGACTGCTTCTGGAACCACCATCGCACTTCCTGACC | 47211.5 | 47249.6 |
| | ACATCGATTTGGGAGTCAAT (dSpacer)(dSpacer)(aminoC7)(dSpacer)(dSpacer) | | |
| | TTTGACTCCCAAATCGATGTGGTCAGGAAGTGCGATGGTGGTTCCAGAAGCAGTCTTCA | | |
| | GACAAGCTTCACCTGC | | |
| 76 | GCAGGTGAAGCTTGTCTGAAGACTGCTTCTGGAACCACCATCGCACTTCCTGACC | 47211.5 | 47356.8 |
| | ACATCGATTTGGGAGTCATA (dSpacer)(dSpacer)(aminoC7)(dSpacer)(dSpacer) | | |
| | TTTGACTCCCAAATCGATGTGGTCAGGAAGTGCGATGGTGGTTCCAGAAGCAGTCTTCA | | |
| | GACAAGCTTCACCTGC | | |

The names of the compounds of the starting material head piece for synthesizing each hairpin DEL are each as follows.

| Hairpin | Starting material |
|---|---|
| DEL: | head piece |
| I-DEL1: | I-DEL1-HP |
| I-DEL2: | I-DEL2-HP |
| I-DEL3: | I-DEL3-HP |
| I-DEL4: | I-DEL4-HP |

Further, SEQ ID NO “No.” and the sequence “Seq” of I-DEL1-HIP, I-DEL2-HP, I-DEL3-HP, and I-DEL4-HP are as shown in Table 14. The notations in Table 14 are the same as in Table 13.

TABLE 14

| | No. | Seq. |
|---|---|---|
| I-DEL1-HP | 77 | (p)GAGTCTAA (dSpacer)(dSpacer)(aminoC7)(dSpacer)(dSpacer)TTTGACTCCC |
| I-DEL2-HP | 78 | (p)GAGTCAT (dSpacer)(dSpacer)(aminoC7)(dSpacer)(dSpacer)TTGACTCCC |
| I-DEL3-HP | 79 | (p)GAGTCAAT (dSpacer)(dSpacer)(aminoC7)(dSpacer)(dSpacer)TTTGACTCCC |
| I-DEL4-HP | 80 | (p)GAGTCATA (dSpacer)(dSpacer)(aminoC7)(dSpacer)(dSpacer)TTTGACTCCC |

The starting material head pieces shown in Table 14 were prepared according to a conventional method.

Similar to Example 2, using various kinds of starting material head pieces, two-step double-stranded ligation with the double-stranded oligonucleotide Pr_TAG and CP was carried out.

A part of the obtained solution was sampled, and after diluting with deionized water, mass spectrometry by ESI-MS was carried out under Analytical condition 3 to identify the target product (the expected molecular weight and the observed molecular weight of each sequence are shown in Table 13). After lyophilizing the rest of the solution, deionized water was added to prepare a 20 M solution.

### <Cleavage Reaction by Endonuclease V>

Verification of the cleavage reaction of the 4 kinds of hairpin DELs (I-DEL1, I-DEL2, I-DEL3, and I-DEL4) containing deoxyinosines by endonuclease V was carried out by the following procedures.

To a PCR tube were added 1 μL of various kinds of 20 M hairpin DEL aqueous solution; 2 μL of NEBuffer® 4 (available from New England BioLabs, Catalog number: B7004); and 15 μL of deionized water. To the solution was added 2 μL of Endonuclease V (available from New England BioLabs, Catalog number: M0305S), and the obtained solution was incubated at 37° C. for 24 hours.

<Confirmation of Product after Cleavage Using LC-MS Measurement>

Among the obtained reaction solutions, 8.0 μL was sampled, and after diluting with deionized water, mass spectrometry by ESI-MS was carried out under Analytical condition 3. The sequences and the expected molecular weights of the products after the cleavage assumed in each reaction solution and the molecular weight observed in each reaction solution are shown in Table 15. The substrates corresponding to each experimental numbers (Entry) are as follows and other notations are the same as in Table 13.

| | |
|---|---|
| Entry. 1: I-DEL1 | Entry. 2: I-DEL2 |
| Entry. 3: I-DEL3 | Entry. 4: I-DEL4 |

TABLE 15

| | | | | Observed |
|---|---|---|---|---|
| En- | | | Expected | MW.(decon- |
| try | No. | Seq. | MW. | volution) |
| 1 | 81 | GCAGGTGAAGCTTGTCTGAAGACTGCTTCTGGAACCACCATCGCACTTCCTGACCA | 22761.7 | 22758.0 |
| | | CATCGATTTGGGAGTCTA | | |
| | 82 | (p)A(dSpacer)(dSpacer)(aminoC7)(dSpacer)(dSpacer)TTTGA | 24467.8 | 24464.7 |
| | | CTCCCAAATCGATGTGGTCAGGAAGTGCGATGGTGGTTCCAGAAGCAGTCTTCAGA | | |
| | | CAAGCTTCACCTGC | | |
| 2 | 83 | GCAGGTGAAGCTTGTCTGAAGACTGCTTCTGGAACCACCATCGCACTTCCTGACCA | 24299.7 | 24297.5 |
| | | CATCGATTTGGGAGTCAT(dSpacer)(dSpacer)(aminoC7) (dSpacer) | | |
| | | (dSpacer)TT | | |
| | 84 | (p)GACTCCCAAATCGATGTGGTCAGGAAGTGCGATGGTGGTTCCAGAAGCAGTCTT | 22312.4 | 22316.7 |
| | | CAGACAAGCTTCACCTGC | | |
| 3 | 85 | GCAGGTGAAGCTTGTCTGAAGACTGCTTCTGGAACCACCATCGCACTTCCTGACCA | 24612.9 | 24614.4 |
| | | CATCGATTTGGGAGTCAAT (dSpacer)(dSpacer)(aminoC7) | | |
| | | (dSpacer)(dSpacer)TT | | |
| | 65 | (p)TGACTCCCAAATCGATGTGGTCAGGAAGTGCGATGGTGG | 22616.6 | 22615.0 |
| | | TTCCAGAAGCAGTCTTCAGACAAGCTTCACCTGC | | |
| 4 | 86 | GCAGGTGAAGCTTGTCTGAAGACTGCTTCTGGAACCACCATCGCACTTCCTGACCA | 24917.1 | 24920.5 |
| | | CATCGATTTGGGAGTCATA (dSpacer)(dSpacer)(aminoC7) (dSpacer) | | |
| | | (dSpacer)TTT | | |
| | 84 | (p)GACTCCCAAATCGATGTGGTCAGGAAGTGCGATGGTGGTTC | 22312.4 | 22310.3 |
| | | CAGAAGCAGTCTTCAGACAAGCTTCACCTGC | | |

In any of the samples, no MS of the substrate was detected, and the MS of the product after the cleavage was observed as the main peak.

### <Confirmation of Cleavage Reaction Using Gel Electrophoresis>

Also, among the obtained reaction solutions, a part was sampled and analyzed by modified polyacrylamide gel electrophoresis under the same conditions as in Example 3. From the results shown in FIG. 14, it was confirmed that the cleavage reaction proceeded in high yield for all the substrates. The samples of each Lane in FIG. 14 are as follows.
Lane 1: 20 bp DNA Ladder (manufactured by Lonza, Lonza 20 bp DNA Ladder, Catalog number: 50330)
Lane 2: I-DEL1
Lane 3: Sample after subjecting to cleavage reaction of I-DEL1
Lane 4: I-DEL2
Lane 5: Sample after subjecting to cleavage reaction of I-DEL2
Lane 6: I-DEL3
Lane 7: Sample after subjecting to cleavage reaction of I-DEL3
Lane 8: I-DEL4
Lane 9: Sample after subjecting to cleavage reaction of I-DEL4

From the above results, in the various kinds of hairpin type DEL containing deoxyinosines, it was shown that the second phosphodiester bond in the 3′ direction from the deoxyinosine was cleaved by endonuclease V.

### Example 5

### [Verification of Cleavage Reaction of Hairpin DEL Containing Ribonucleoside by RNaseHII]

### <Synthesis of Hairpin DEL (R-DEL1) Containing Ribonucleoside>

The compound having the sequence shown in Table 16 (hairpin DEL) was synthesized by the following procedures. In the sequence notations in Table 16, “u” means uridine and other notations are the same as in Table 2.

The name of the compound corresponding to SEQ ID NO (No.) is as follows.
No. 87: R-DEL1

TABLE 16

| | | | Observed |
|---|---|---|---|
| | | | MW. |
| | | Expected | (decon- |
| No. | Seq. | MW. | volution) |
| 87 | GCAGGTGAAGCTTGTCTGAAGACTGCTTCTGGAACCACCATCGCACTTCCTGACC | 47212.5 | 47237.7 |
| | ACATCGATTTGGGAGTCAAA (dSpacer)(dSpacer)(aminoC7) (dSpacer) | | |
| | (dSpacer) TuTGACTCCCAAATCGATGTGGTCAGGAAGTGCGATGGTGGTTCCAGAAGC | | |
| | AGTCTTCAGACAAGCTTCACCTGC | | |

The name of the compound of the starting material head piece for synthesizing each hairpin DEL is as follows.

| Hairpin | Starting material |
|---|---|
| DEL: | head piece |
| R-DEL1: | R-DEL1-HP |

Further, SEQ ID NO “No.” of R-DEL1-HP and the sequence “Seq” are as shown in Table 17. The notations in Table 17 are the same as in Table 16.

TABLE 17

| | No. | Seq. |
|---|---|---|
| R-DEL1-HP | 88 | (p)GAGTCAAA (dSpacer)(dSpacer)(aminoC7)(dSpacer)(dSpacer) |
| | | TuTGACTCCC |

The starting material head pieces shown in Table 17 were prepared according to a conventional method.

Similar to Example 2, using starting material head pieces, two-step double-stranded ligation with the double-stranded oligonucleotide Pr_TAG and CP was carried out.

A part of the obtained solutions was sampled, and after diluting with deionized water, mass spectrometry by ESI-MS was carried out under Analytical condition 3 to identify the target product (the expected molecular weight and the observed molecular weight of each sequence are shown in Table 16). After lyophilizing the rest of the solution, deionized water was each added to prepare a 200 pM solution.

### <Cleavage Reaction by RNaseHII>

Verification of the cleavage reaction of the hairpin DEL (R-DEL1) containing ribonucleoside by RNaseHII was carried out by the following procedures.

To a PCR tube were added 0.5 μL of 200 M hairpin DEL aqueous solution; 4.9 μL of ThermoPol® Reaction Buffer Pack (available from New England BioLabs, Catalog number: B9004); and 43.6 μL of deionized water. To the solution was added 1 μL of RNase HII (available from New England BioLabs, Catalog number: M0288S), and the obtained solution was incubated at 37° C. for 8 hours.

<Confirmation of Product after Cleavage Using LC-MS Measurement>

Among the obtained reaction solutions, 10 μL was sampled and mass spectrometry by ESI-MS was carried out under Analytical condition 3. The sequences and the expected molecular weights of the products after the cleavage assumed in each reaction solution and the molecular weight observed in each reaction solution are shown in Table 18. The substrates corresponding to each experimental numbers (Entry) are as follows and other notations are the same as in Table 16.
Entry. 1: R-DEL1

TABLE 18

| | | | | Observed |
|---|---|---|---|---|
| | | | Expected | MW.(decon- |
| Entry | No. | Seq. | MW. | volution) |
| 1 | 89 | GCAGGTGAAGCTTGTCTGAAGACTGCTTCTGGAACCACCATCGCACTTCCTGACCA | 24307.7 | 24303.6 |
| | | CATCGATTTGGGAGTCAAA (dSpacer)(dSpacer)(aminoC7) | | |
| | | (dSpacer)(dSpacer)T | | |
| | 90 | (p)uTGACTCCCAAATCGATGTGGTCAGGAAGTGCGATGGTGGTTCCAGAAGCAGTC | 22922.8 | 22918.3 |
| | | TTCAGACAAGCTTCACCTGC | | |

In any of the samples, no MS of the substrate was detected, and the MS of the product after the cleavage was observed as the main peak.

### <Confirmation of Cleavage Reaction Using Gel Electrophoresis>

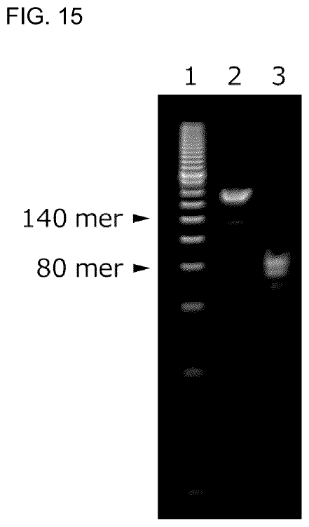

Also, among the obtained reaction solutions, a part was sampled and analyzed by modified polyacrylamide gel electrophoresis under the same conditions as in Example 3. From the results shown in FIG. 15, it was confirmed that the cleavage reaction proceeded in high yield for all the substrates. The samples of each Lane in FIG. 15 are as follows.
Lane 1: 20 bp DNA Ladder (manufactured by Lonza, Lonza 20 bp DNA Ladder, Catalog number: 50330)
Lane 2: R-DEL1
Lane 3: Sample after subjecting to cleavage reaction of R-DEL1

From the above results, in the hairpin type DEL containing ribonucleoside, it was shown that the phosphodiester bond at the 5′ side of the ribonucleotide was cleaved by RNaseHII.

### Example 6

### [Creation of Model Library Using U-DEL9-HP as Starting Material]

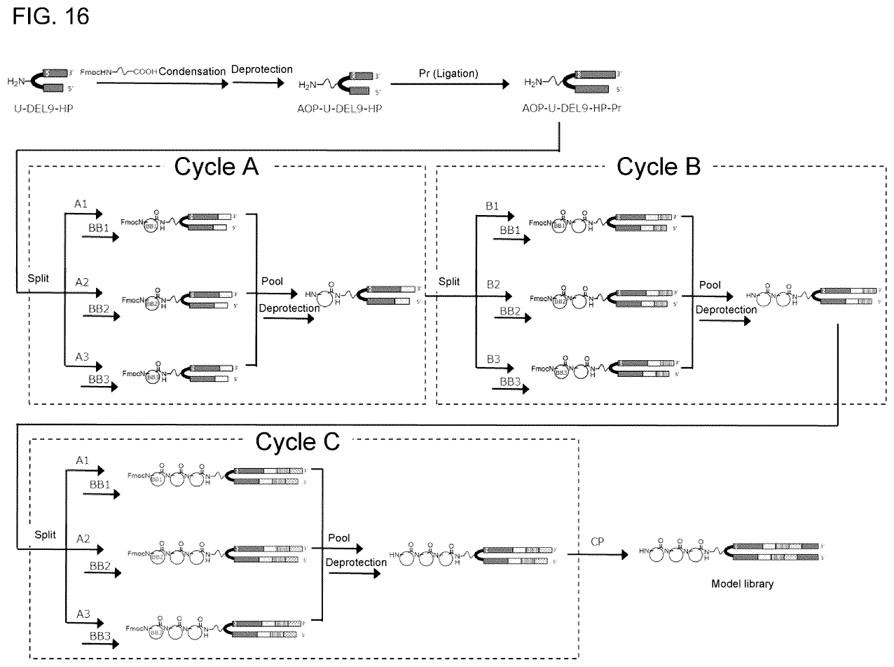

As in the schematic diagram shown in FIG. 16, using U-DEL9-HP as a starting material, synthesis of the model library containing 3×3×3 (27) compound species was carried out by the split and pool synthesis using the following reagents.
U-DEL9-HP
3 kinds of building blocks (BB1, BB2, and BB3):

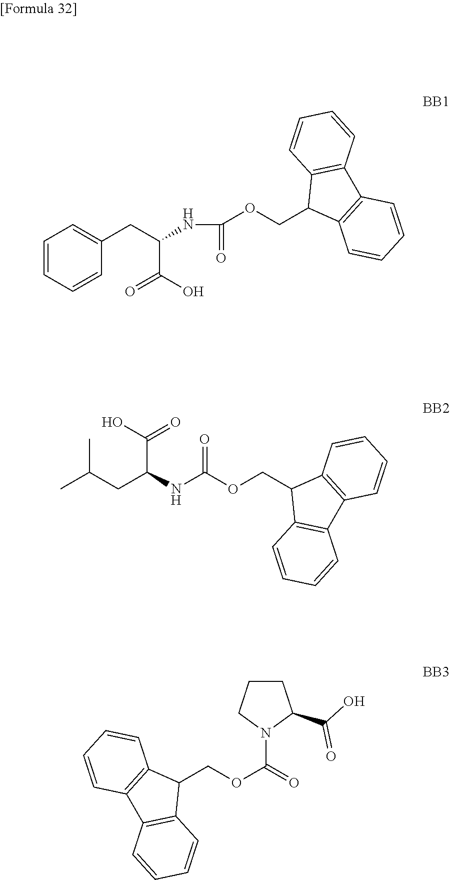 [SMILES: O=C(N[C@@H](Cc1ccccc1)C(=O)O)OCC1c2ccccc2-c2ccccc21 ; CC(C)C[C@H](NC(=O)OCC1c2ccccc2-c2ccccc21)C(=O)O ; O=C(O)[C@@H]1CCCN1C(=O)OCC1c2ccccc2-c2ccccc21]

10 kinds of double-stranded oligonucleotide tags (tag number: Pr, A1, A2, A3, B1, B2, B3, C1, C2, and C3 in Table 19)

In Table 19, “Tag No.” (leftmost) represents a tag number, “No.” (second from the left) represents SEQ ID NO, and “Seq.” (third from the left) represents a sequence. The sequence notations are the same as in Table 1.

Each double-stranded oligonucleotide tag was prepared by annealing 2 kinds of oligonucleotides having a SEQ ID NO corresponding to each tag number, as shown in Table 19.

TABLE 19

| Tag No. | No. | Seq. |
|---|---|---|
| Pr | 91 | (p)TCCTGACCACATCGATTTGG |
| | 92 | (p)AAATCGATGTGGTCAGGAAG |
| A1 | 93 | (p)GCAACCACT |
| | 94 | (p)TGGTTGCGT |
| A2 | 95 | (p)GGAGACACT |
| | 96 | (p)TGTCTCCGT |
| A3 | 97 | (p)TAGGCGACT |
| | 98 | (p)TCGCCTAGT |
| B1 | 99 | (p)CACGCATAC |
| | 100 | (p)ATGCGTGGA |
| B2 | 101 | (p)CGTCAACAC |
| | 102 | (p)GTTGACGGA |
| B3 | 103 | (p)GCATGTCAC |
| | 104 | (p)GACATGCGA |
| C1 | 105 | (p)CTCTCCTTC |
| | 106 | (p)AGGAGAGTT |
| C2 | 107 | (p)GGATCGTTC |
| | 108 | (p)ACGATCCTT |
| C3 | 109 | (p)TGAACGCTC |
| | 110 | (p)GCGTTCATT |

### <Synthesis of Compound “AOP-U-DEL9-HP”>

The compound “AOP-U-DEL9-HP” having the sequence shown in Table 20 was synthesized by the following procedures. In the sequence notations in Table 20, “(AOP-AminoC7)” means the group represented by the following formula (10)

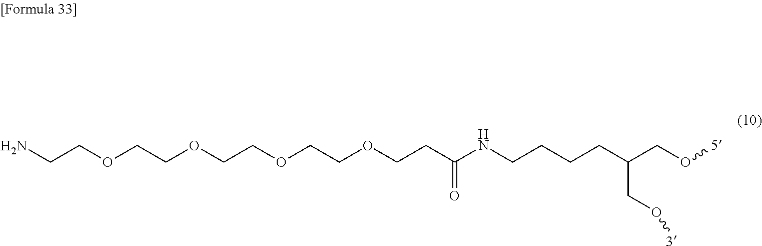

and other notations are the same as in Table 2.

TABLE 20

| | | | Observed |
|---|---|---|---|
| | | Expected | MW. (decon- |
| No. | Seq. | MW. | volution) |
| 111 | (p)GAGTCAA (dSpacer)(dSpacer)(AOP- | 6084.0 | 6082.9 |
| | AminoC7)(dSpacer)(dSpacer)(dU)TGACTCCC | | |

To four violamo centrifuge tubes was added a solution (2.5 mL, 1 mM) of U-DEL9-HP in a sodium borate buffer (150 mM, pH 9.4) cooled to 10° C. To the respective tubes were added 40 equivalent of N-Fmoc-15-amino-4,7,10,13-tetraoxaoctadecanoic acid (250 μL, 0.4M N,N-dimethylacetamide solution), subsequently 40 equivalents of 4-(4,6-dimethoxy[1.3.5]triazin-2-yl)-4-methylmorpholinium chloride hydrate (DMTMM) (200 μL, 0.5 M aqueous solution), and the obtained solution was shaken at 10° C. for 5 hours.

The above-mentioned solutions were each treated by 295 μL of a 5 M aqueous sodium chloride solution and 9.7 mL of cooled (−20° C.) ethanol, and allowed to stand at −78° C. overnight. After centrifugation, the supernatant was removed and the obtained pellets were air-dried. To the pellets were each added 2.75 mL of deionized water to dissolve them, 306 μL of piperidine was added at 0° C., and the mixture was shaken at 10° C. for 3 hours. After the mixture was centrifuged, the precipitates were removed by filtration and washed with 1.47 mL of deionized water twice. The obtained filtrates were each treated with 600 μL of a 5 M aqueous sodium chloride solution and 19.8 mL of cooled (−20° C.) ethanol and allowed to stand at −78° C. overnight. After centrifugation, the supernatant was removed and the obtained pellets were air-dried.

To the obtained pellets was added 10 mL of deionized water to make it a solution. Among the obtained solutions, a part was sampled and diluted with deionized water. After then, mass spectrometry by ESI-MS was carried out under the conditions of Analytical condition 2 in Example 1 to identify the target product (the expected molecular weight and the observed molecular weight of the compound are shown in Table 20). After lyophilizing the rest of the solution, deionized water was each added to prepare a 5 mM solution.

### <Introduction of Double-Stranded Oligonucleotide Tag “Pr”>

The compound “AOP-U-DEL9-HP-Pr” having the sequence shown in Table 21 was synthesized by ligating the compound “AOP-U-DEL9-HP” and the double-stranded oligonucleotide tag “Pr” according to the following procedures. The sequence notations in Table 21 are the same as in Table 20.

TABLE 21

| | | | Observed |
|---|---|---|---|
| | | Expected | MW.(decon- |
| No. | Seq. | MW. | volution) |
| 112 | (p)TCCTGACCACATCGATTTGGGAGTCAA(dSpacer)(dSpacer)((AOP- | 18506.0 | 18501.4 |
| | AminoC7)(dSpacer)(dSpacer)(dU)TGACTCCCAAATCGATGTGGTCAGGAAG | | |

To a violamo centrifuge tube were added 40 μL of 5 mM aqueous solution of the compound “AOP-U-DEL9-HP”; 160 μL of 100 mM aqueous sodium hydrogen carbonate solution water; 240 μL of 1 mM aqueous solution of the double-stranded oligonucleotide tag “Pr”; 80 μL of 10× ligase buffer (500 mM Tris hydrochloride, pH 7.5; 500 mM sodium chloride; 100 mM magnesium chloride; 100 mM dithiothreitol; and 20 mM adenosine triphosphate); and 272 μL of deionized water. To the solution was added 8.0 μL of T4DNA ligase (available from Thermo Fisher, Catalog number: EL0013), and the obtained solution was incubated at 16° C. for 24 hours.

The reaction solution was treated with 80 μL of 5 M aqueous sodium chloride solution and 2640 μL of cooled (−20° C.) ethanol and allowed to stand at −78° C. for 2 hours. After centrifugation, the supernatant was removed, and 400 μL of deionized water was added to the obtained pellets. The obtained solution was concentrated by Amicon® Ultra Centrifugal filter (30 kD cutoff). A part of the obtained solution was sampled, and mass spectrometry by ESI-MS was carried out under the conditions of Analytical condition 2 to identify the target product (the expected molecular weight and the observed molecular weight of the compound are shown in Table 21). According to the above procedures, 133 nmol of the compound “AOP-U-DEL9-HP-Pr” with a purity of 84.5% was obtained. To the obtained compound “AOP-U-DEL9-HP-Pr” was added a 100 mM aqueous sodium hydrogen carbonate solution to prepare a 1 mM solution.

### <Cycle A>

To each of three PCR tubes were added 20 μL of 1 mM solution of the compound “AOP-U-DEL9-HP-Pr” obtained as mentioned above; 30 μL of a 1 mM aqueous solution of one of the double-stranded oligonucleotide tags A1 to A3; 8.0 μL of 10× ligase buffer (500 mM Tris hydrochloride, pH 7.5; 500 mM sodium chloride; 100 mM magnesium chloride; 100 mM dithiothreitol; and 20 mM adenosine triphosphate); and 21.6 μL of deionized water. To the solution was added 0.4 μL of T4DNA ligase (available from Thermo Fisher, Catalog number: EL0013), and the obtained solution was incubated at 16° C. for 18 hours.

The reaction solutions were each treated with 8.0 μL of a 5 M aqueous sodium chloride solution and 264 μL of cooled (−20° C.) ethanol, and allowed to stand at −78° C. for 30 minutes. After centrifugation, the supernatant was removed and the obtained pellets were each dissolved in 20 μL of 150 mM sodium borate buffer (pH 9.4).

To each tube were added 40 equivalents of one of the building blocks BB1 to BB3 (4.0 μL, 200 mM N,N-dimethylacetamide solution), subsequently 40 equivalents of 4-(4,6-dimethoxy[1.3.5]triazin-2-yl)-4-methylmorpholinium chloride hydrate (DMTMM) (4.0 μL, 200 mM aqueous solution), and the mixture was shaken at 10° C. for 2 hours. Further, to each tube were added 20 equivalents of building blocks (2.0 μL, 200 mM N,N-dimethylacetamide solution), subsequently 20 equivalents of DMTMM (2.0 μL, 200 mM aqueous solution), and the mixture was shaken at 10° C. for 30 minutes.

The reaction solutions were each treated with 3.2 μL of a 5 M aqueous sodium chloride solution and 106 μL of cooled (−20° C.) ethanol, and allowed to stand at −78° C. for 30 minutes. After centrifugation, the supernatant was removed, and to the obtained pellets was added each 18 μL of deionized water, and 3 kinds of the solutions were mixed in one PCR tube.

To the mixed solution was added 6.0 μL of piperidine at 0° C., and the mixture was shaken at room temperature for 1 hour. The reaction solution was treated with 6.0 μL of a 5 M aqueous sodium chloride solution and 198 μL of cooled (−20° C.) ethanol, and allowed to stand at −78° C. for 18 hours. After centrifugation, the supernatant was removed, and 400 μL of deionized water was added to the obtained pellets. The obtained solution was concentrated by Amicon® Ultra Centrifugal filter (30 kD cutoff), a 100 mM aqueous sodium hydrogen carbonate solution was added to prepare a 1 mM solution, and used in the next step as a starting material.

### <Cycle B>

To each of three PCR tubes were added 13.7 μL of 1 mM solution obtained in Cycle A as a starting material; 20.6 μL of 1 mM aqueous solution of one of the three double-stranded oligonucleotide tags B1 to B3; 5.5 μL of 10× ligase buffer (500 mM Tris hydrochloride, pH 7.5; 500 mM sodium chloride; 100 mM magnesium chloride; 100 mM dithiothreitol; and 20 mM adenosine triphosphate); and 14.8 μL of deionized water. To the solution was added 0.3 μL of T4DNA ligase (available from Thermo Fisher, Catalog number: EL0013), and the obtained solution was incubated at 16° C. for 16 hours.

The reaction solutions were each treated with 5.5 μL of a 5 M aqueous sodium chloride solution and 181 μL of cooled (−20° C.) ethanol, and allowed to stand at −78° C. for 30 minutes. After centrifugation, the supernatant was removed and the obtained pellets were each dissolved in 13.7 μL of 150 mM sodium borate buffer (pH 9.4).

To each tube were added 80 equivalents of one of the building blocks BB1 to BB3 (5.5 μL, 200 mM N,N-dimethylacetamide solution), subsequently 80 equivalents of DMTMM (5.5 μL, 200 mM aqueous solution), and the mixture was shaken at 10° C. for 1 hour. Further, to each tube were added 40 equivalents of the building block (2.3 μL, 200 mM N,N-dimethylacetamide solution), subsequently 40 equivalents of DMTMM (2.3 μL, 200 mM aqueous solution), and the mixture was shaken at 10° C. for 2 hours.

The reaction solutions were each treated with 2.5 μL of a 5 M aqueous sodium chloride solution and 81.4 μL of cooled (−20° C.) ethanol, and allowed to stand at −78° C. for 30 minutes. After centrifugation, the supernatant was removed, and to the obtained pellets was added each 12.3 μL of deionized water, and 3 kinds of the solutions were mixed in one PCR tube.

To the mixed solution was added 4.1 μL of piperidine at 0° C., and the mixture was shaken at room temperature for 3 hours. The reaction solution was treated with 4.1 μL of a 5 M aqueous sodium chloride solution and 136 μL of cooled (−20° C.) ethanol, and allowed to stand at −78° C. for 3 hours. After centrifugation, the supernatant was removed and 400 μL of deionized water was added to the obtained pellets. The obtained solution was concentrated by Amicon® Ultra Centrifugal filter (30 kD cutoff), a 100 mM aqueous sodium hydrogen carbonate solution was added to prepare a 0.48 mM solution, and used in the next step as a starting material.

### <Cycle C>

To each of three PCR tubes were added 14.5 μL of 0.48 mM solution of the starting material obtained in Cycle B; 10.5 μL of 1 mM aqueous solution of one of the three double-stranded oligonucleotide tags C1 to C3; and 2.8 μL of 10× ligase buffer (500 mM Tris hydrochloride, pH 7.5; 500 mM sodium chloride; 100 mM magnesium chloride; 100 mM dithiothreitol; and 20 mM adenosine triphosphate). To the solution was added 0.14 μL of T4DNA ligase (available from Thermo Fisher, Catalog number: EL0013), and the obtained solution was incubated at 16° C. for 16 hours.

The reaction solutions were each treated with 2.8 μL of a 5 M aqueous sodium chloride solution and 92 μL of cooled (−20° C.) ethanol, and allowed to stand at −78° C. for 30 minutes. After centrifugation, the supernatant was removed, and the obtained pellets were each dissolved in 7.0 μL of 150 mM sodium borate buffer (pH 9.4).

To each tube were added 80 equivalents of one of the building blocks BB1 to BB3 (2.8 μL, 200 mM N,N-dimethylacetamide solution), subsequently 80 equivalents of DMTMM (2.8 μL, 200 mM aqueous solution), and the mixture was shaken at 10° C. for 1 hour. Further, to each tube were added 40 equivalents of the building block (1.4 μL, 200 mM N,N-dimethylacetamide solution), subsequently 40 equivalents of DMTMM (1.4 μL, 200 mM aqueous solution), and the mixture was shaken at 10° C. for 2 hours.

The reaction solutions were each treated with 1.3 μL of a 5 M aqueous sodium chloride solution and 41.4 μL of cooled (−20° C.) ethanol, and allowed to stand at −78° C. for 30 minutes. After centrifugation, the supernatant was removed, and each 6.3 μL of deionized water was added to the obtained pellets, and then 3 kinds of the solutions were mixed in one PCR tube.

To the mixed solution was added 2.1 μL of piperidine at 0° C., and the mixture was shaken at room temperature for 2 hours. The reaction solution was treated with 2.1 μL of a 5 M aqueous sodium chloride solution and 69 μL of cooled (−20° C.) ethanol, and allowed to stand at −78° C. for 3 hours. After centrifugation, the supernatant was removed, and 400 μL of deionized water was added to the obtained pellets. The obtained solution was concentrated by Amicon® Ultra Centrifugal filter (30 kD cutoff), a 100 mM aqueous sodium hydrogen carbonate solution was added to prepare a 0.41 mM solution, and used in the next step as a starting material.

### <Ligation of CP>

To a PCR tube were added 12.2 μL of 0.41 mM solution of the starting material obtained in Cycle C; 6.0 μL of 1 mM aqueous solution of CP (the same as used in Example 2); 2.1 μL of 10× ligase buffer (500 mM Tris hydrochloride, pH 7.5; 500 mM sodium chloride; 100 mM magnesium chloride; 100 mM dithiothreitol; and 20 mM adenosine triphosphate), and 0.7 μL of deionized water. To the solution was added 0.1 μL of T4DNA ligase (available from Thermo Fisher, Catalog number: EL0013), and the obtained solution was incubated at 16° C. for 16 hours.

The reaction solution was treated with 2.1 μL of a 5 M aqueous sodium chloride solution and 69.6 μL of cooled (−20° C.) ethanol, and allowed to stand at −78° C. for 30 minutes. After centrifugation, the supernatant was removed, and 400 μL of deionized water was added to the obtained pellets. The obtained solution was concentrated by Amicon® Ultra Centrifugal filter (30 kD cutoff), and deionized water was added to prepare a 20 pM solution.

### <Results>

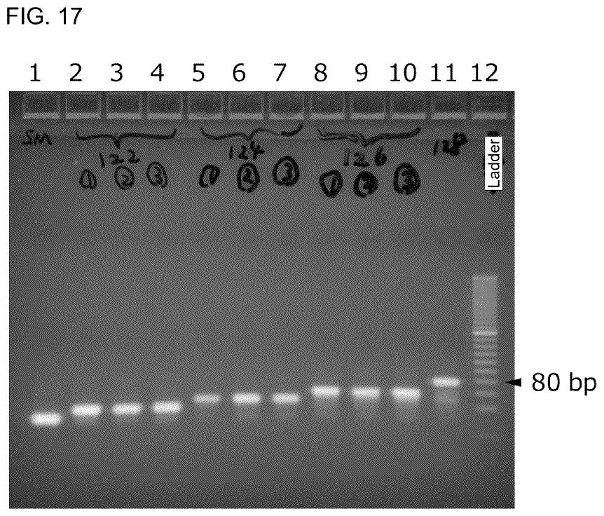

The samples after ligation of the double-stranded oligonucleotide tag in each cycle were analyzed by electrophoresis using a 2.2% agarose gel (manufactured by Lonza, FlashGel® cassette, Catalog number: 57031). From the results shown in FIG. 17, in each cycle, it was confirmed that coding with the double-stranded oligonucleotide tag was achieved with high efficiency. The samples in each lane in FIG. 17 are as follows.
Lane 1: AOP-U-DEL9-HP-Pr
Lane 2: Sample after ligation of the double-stranded oligonucleotide tag A1 in Cycle A
Lane 3: Sample after ligation of the double-stranded oligonucleotide tag A2 in Cycle A
Lane 4: Sample after ligation of the double-stranded oligonucleotide tag A3 in Cycle A
Lane 5: Sample after ligation of the double-stranded oligonucleotide tag B1 in Cycle B
Lane 6: Sample after ligation of the double-stranded oligonucleotide tag B2 in Cycle B
Lane 7: Sample after ligation of the double-stranded oligonucleotide tag B3 in Cycle B
Lane 8: Sample after ligation of the double-stranded oligonucleotide tag C1 in Cycle C
Lane 9: Sample after ligation of the double-stranded oligonucleotide tag C2 in Cycle C
Lane 10: Sample after ligation of the double-stranded oligonucleotide tag C3 in Cycle C
Lane 11: Sample after CP ligation
Lane 12: 20 bp DNA ladder (manufactured by Lonza, Lonza 20 bp DNA Ladder, Catalog number: 50330)

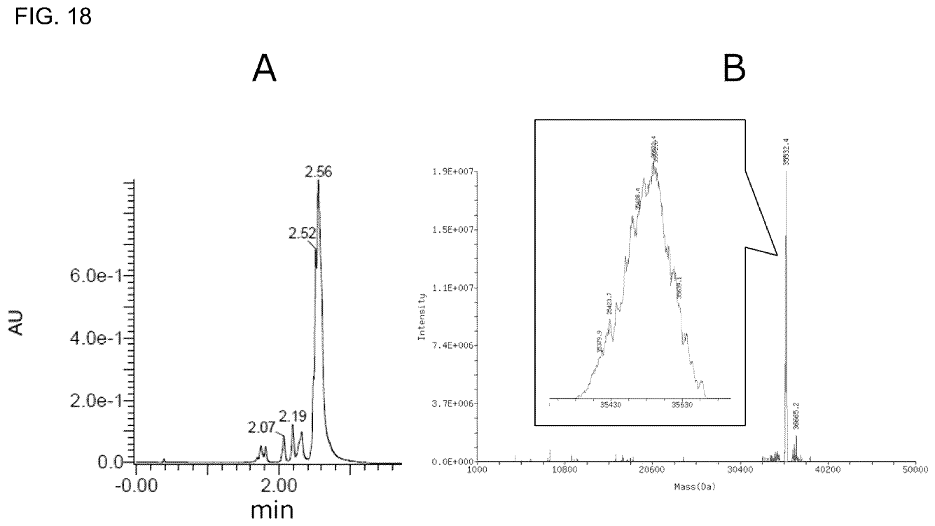

The samples after the completion of Cycle C were analyzed under Analytical condition 3. FIG. 18 shows the results of chromatograph and mass spectrum. By deconvolution of the obtained mass spectrum, 35532.4 was observed as an average molecular weight. This result is consistent with the average molecular weight (35514.2) expected after the completion of Cycle C, and it was shown that the reaction for the synthesis of library (ligation of the double-stranded oligonucleotide tag and introduction of the building blocks) was achieved with high efficiency.

According to the above, by the above-mentioned synthesis procedures, the synthesis of the model library containing the 3×3×3 (27) compound species using U-DEL9-HP as a starting material was achieved.

### <Cleavage of the Obtained Model Library by USER® Enzyme>

The cleavage reaction of the model library obtained above by USER® enzyme was carried out by the following procedures.

To a PCR tube were added 2.0 μL of 20 M aqueous solution of the model library; 2 μL of CutSmart® Buffer (available from New England BioLabs, Catalog number: B7204S), and 14 μL of deionized water. To the solution was added 2 μL of USER® enzyme (available from New England BioLabs, Catalog number: M5505S), and the obtained solution was incubated at 37° C. for 16 hours and further incubated at 90° C. for 1 hour.

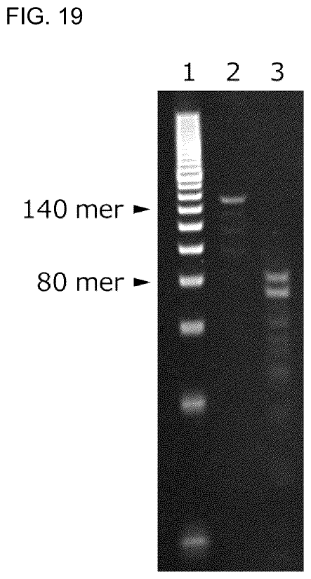

Among the obtained reaction solutions, a part thereof was sampled, and analysis was carried out by modified polyacrylamide gel electrophoresis under the same conditions as in Example 3. From the results shown in FIG. 19, it was confirmed that the model library using U-DEL9-HP as a starting material was able to proceed with the cleavage reaction by USER® enzyme with high efficiency. The samples of each lane in FIG. 19 are as follows.
Lane 1: 20 bp DNA Ladder (manufactured by Lonza, Lonza 20 bp DNA Ladder, Catalog number: 50330)
Lane 2: Model library
Lane 3: Sample after subjecting to cleavage reaction by USER® enzyme of the model library

### Example 7

[Conversion of DEL Compound from Hairpin DNA to Single-Stranded DNA and Addition of New Function]

### <Synthesis of the Starting Material Head Piece (AAZ-DEL-HP) of DEL Compound>

AAZ-DEL-HP having a sequence shown in Table 22 was synthesized by the following procedures. In the sequence notation in Table 22, “(AAZ-AOP-AminoC7)” means the group represented by the following formula (11)

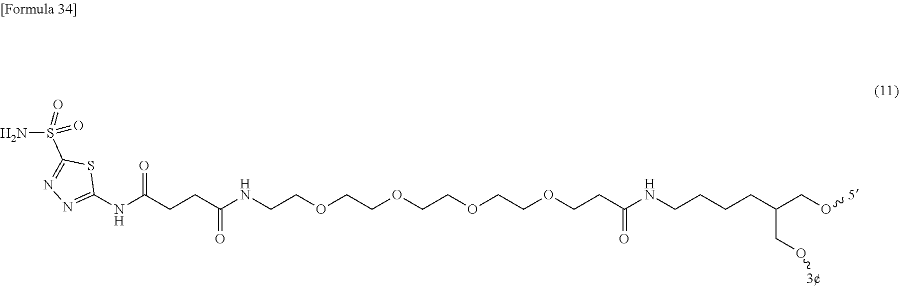

and other notations are the same as in Table 2.

TABLE 22

| | | | Observed |
|---|---|---|---|
| | | Expected | MW. (decon- |
| No. | Seq. | MW. | volution) |
| 113 | (p)GAGTCAA (dSpacer)(dSpacer)(AAZ- | 6346.3 | 6348.2 |
| | AOP-AminoC7)(dSpacer)(dSpacer)UTGACTCCC | | |

To each of eight PCR tubes were added dimethyl sulfoxide (599 L), 4-oxo-4-[(5-sulfamoyl-1,3,4-thiadiazol-2-yl)amino)butanoic acid (37.5 μL, 0.2 M dimethyl sulfoxide solution), sodium 1-hydroxy-2,5-dioxopyrrolidine-3-sulfonate (60 μL, 0.33 M dimethyl sulfoxide/deionized water (2:1, v/v) solution), subsequently 1-(3-dimethylaminopropyl)-3-ethylcarbodiimide hydrochloride (72 μL, 0.1 M dimethyl sulfoxide solution), and the obtained solution was shaken at 30° C. for 30 minutes. Then, to each solution were added 150 μL of triethylamine hydrochloride buffer (500 mM, pH 10), subsequently an aqueous solution of AOP-U-DEL9-HP (synthesized in Example 6) (75 μL, 0.67 mM), and the mixture was shaken at 37° C. for 6 hours.

The reaction solutions was combined into one violamo centrifuge tube, treated with 800 μL of 5 M aqueous sodium chloride solution and 26.3 mL of cooled (−20° C.) ethanol, and allowed to stand at −78° C. for 30 minutes. After centrifugation, the supernatant was removed and the obtained pellets were air-dried. The pellets were dissolved in deionized water, and purified by reverse phase HPLC using Phenomenex Gemini C18 column. Using a dual mobile phase gradient profile, the target product was eluted using 50 mM triethyl ammonium acetate buffer (pH7.5) and acetonitrile/water (100:1, v/v). Fractions containing the target product were collected, mixed and concentrated, respectively. The obtained solution was desalted by Amicon® Ultra Centrifugal filter (3 kD cutoff), and ethanol precipitation was carried out, and then deionized water was added to the pellets to prepare a 1 mM solution.

A part of the obtained solution was sampled and diluted with deionized water, and then mass spectrometry by ESI-MS was carried out under the conditions of Analytical condition 2 in Example 2 to identify the target product AAZ-DEL-HP (the expected molecular weight and the observed molecular weight of the compound are shown in Table 22).

<Synthesis of the Starting Material Head Pieces for 3 Kinds of DEL Compounds (SABA-DEL-HP, ClSABA-DEL-HP and mSABA-DEL-HP)>

The compound having the sequence shown in Table 23 was synthesized by the following procedures. In the sequence notation in Table 23, “(SABA-AOP-AminoC7)” means the group represented by the following formula (12)

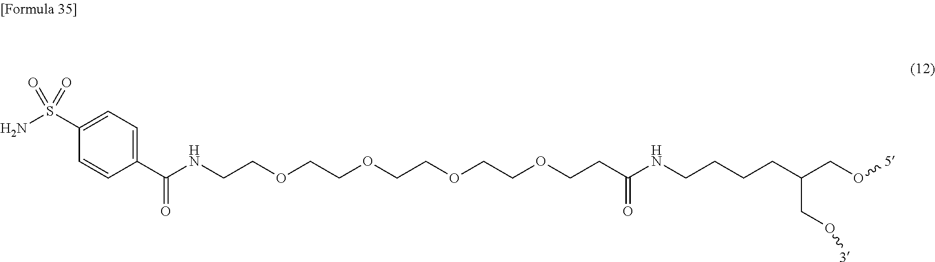

“(ClSABA-AOP-AminoC7)” means the group represented by the following formula

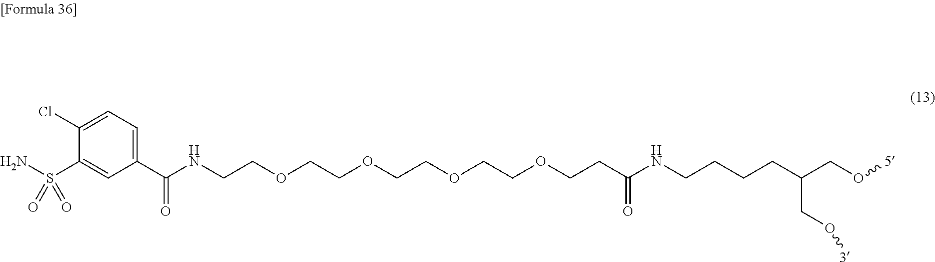

“(mSABA-AOP-AminoC7)” means the group represented by the following formula (14)

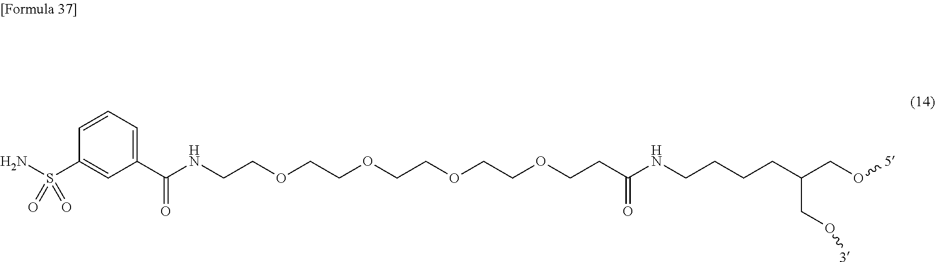

and other notations are the same as in Table 2.

The names of the compounds corresponding to each SEQ ID NO: (No.) are as follows.
No. 114: SABA-DEL-HP
No. 115: ClSABA-DEL-HP
No. 116: mSABA-DEL-HP

TABLE 23

| | | | Observed |
|---|---|---|---|
| | | Expected | MW. (decon- |
| No. | Seq. | MW. | volution) |
| 114 | (p)GAGTCAA(dSpacer)(dSpacer)(SABA-AOP- | 6267.2 | 6268.3 |
| | AminoC7)(dSpacer)(dSpacer)UTGACTCCC | | |
| 115 | (p)GAGTCAA(dSpacer)(dSpacer)(CISABA-AOP- | 6301.6 | 6303.6 |
| | AminoC7)(dSpacer)(dSpacer)UTGACTCCC | | |
| 116 | (p)GAGTCAA(dSpacer)(dSpacer)(mSABA-AOP- | 6267.2 | 6268.4 |
| | AminoC7)(dSpacer)(dSpacer)UTGACTCCC | | |

The starting material carboxylic acids for synthesizing each compound are as follows.

| Compound: | Starting material carboxylic acid |
|---|---|
| SABA-DEL-HP: | 4-sulfamoylbenzoic acid |
| ClSABA-DEL-HP: | 4-chloro-3-sulfamoylbenzoic acid |
| mSABA-DEL-HP: | 3-sulfamoylbenzoic acid |

To each PCR tube was added a starting material carboxylic acid (50 μL, 0.2 M N,N-dimethylacetamide solution). To each tube were added 3-hydroxytriazolo[4,5-b]pyridine (16.7 μL, 0.6 M N,N-dimethylacetamide solution), N,N′-diisopropylcarbodiimide (16.7 μL, 0.6 M N,N-dimethylacetamide solution), subsequently N,N-diisopropylethylamine (16.7 μL, 0.6 M N,N-dimethylacetamide solution), and the obtained solution was shaken at 25° C. for 30 minutes. Then, to each solution was added a solution (100 μL, 1 mM) of AOP-U-DEL9-HP (synthesized in Example 6) in a sodium borate buffer (250 mM, pH 9.4), and the mixture was shaken at 25° C. for 90 minutes.

The respective solutions were treated with 20 μL of 5 M aqueous sodium chloride solution and 660 μL of cooled (−20° C.) ethanol, and allowed to stand at −78° C. for 30 minutes. After centrifugation, the supernatant was removed and the obtained pellets were air-dried. The respective pellets were dissolved in 50 mM triethyl ammonium acetate buffer (pH 7.5), and purified by reverse phase HPLC using Phenomenex Gemini C18 column. Using a dual mobile phase gradient profile, the target product was eluted using 50 mM triethyl ammonium acetate buffer (pH7.5) and acetonitrile/water (100:1, v/v). Fractions containing the target product were collected, mixed, and concentrated, respectively. Each of the obtained solutions was desalted by the Amicon® Ultra Centrifugal filter (3 kD cutoff), and ethanol precipitation was carried out, and then 25 μL of deionized water was added to the pellets to prepare a 1 mM aqueous solution.

A part of the obtained solution was sampled and diluted with deionized water, and then mass spectrometry by ESI-MS was carried out under the conditions of Analytical condition 2 in Example 2 to identify each target product (the expected molecular weight and the observed molecular weight of each compound are shown in Table 23).

<Synthesis of 5 Kinds of DEL Compounds Having Biotin at 3′ End (“AAZ-BIO-DEL”, “SABA-BIO-DEL”, “ClSABA-BIO-DEL”, “mSABA-BIO-DEL”, and “Amino-BIO-DEL”)>

The DEL compounds having the sequences shown in Table 24 were synthesized by the following procedures. In the sequence notation in Table 24, “(BIO)” means the group represented by the following formula (15)

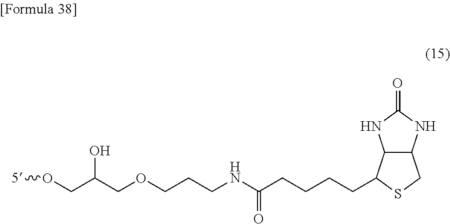

and other notations are the same as in Tables 2, 20, 22, and 23.

The names of the compounds corresponding to each SEQ ID NO: (No.) are as follows.
No. 117: AAZ-BIO-DEL
No. 118: SABA-BIO-DEL
No. 119: ClSABA-BIO-DEL
No. 120: mSABA-BIO-DEL
No. 121: Amino-BIO-DEL

TABLE 24

| | | | Observed |
|---|---|---|---|
| | | Expected | MW. (decon- |
| No. | Seq. | MW. | volution) |
| 117 | GCAGGTGAAGCTTGTCTGAATACTCGGTCACTTGCCACTGCCTTGCTTCCTG | 47526.1 | 47579.6 |
| | ACCACATCGATTTGGGAGTCAA(dSpacer)(dSpacer)(AAZ-AOP-AminoC7) | | |
| | (dSpacer)(dSpacer)(dU)TGACTCCCAAATCGATGTGGTCAGGAAGCAAGGCAG | | |
| | TGGCAAGTGACCGAGTATTCAGACAAGCTTCACCTGC(BIO) | | |
| 118 | GCAGGTGAAGCTTGTCTGAATACTCGGTCACTTGCCACTGCCTTGCTTCCTG | 47447.0 | 47455.9 |
| | ACCACATCGATTTGGGAGTCAA (dSpacer)(dSpacer)(SABA-AOP-AminoC7) | | |
| | (dSpacer)(dSpacer)(dU)TGACTCCCAAATCGATGTGGTCAGGAAGCAAGGCAG | | |
| | TGGCAAGTGACCGAGTATTCAGACAAGCTTCACCTGC(BIO) | | |
| 119 | GCAGGTGAAGCTTGTCTGAATACTCGGTCACTTGCCACTGCCTTGCTTCCTG | 47481.4 | 47486.3 |
| | ACCACATCGATTTGGGAGTCAA(dSpacer)(dSpacer)(CISABA-AOP-AminoC7) | | |
| | (dSpacer)(dSpacer)(dU)TGACTCCCAAATCGATGTGGTCAGGAAGCAAGGCAG | | |
| | TGGCAAGTGACCGAGTATTCAGACAAGCTTCACCTGC(BIO) | | |
| 120 | GCAGGTGAAGCTTGTCTGAATACTCGGTCACTTGCCACTGCCTTGCTTCCTG | 47447.1 | 47520.3 |
| | ACCACATCGATTTGGGAGTCAA (dSpacer)(dSpacer)(mSABA-AOP-AminoC7) | | |
| | (dSpacer)(dSpacer)(dU)TGACTCCCAAATCGATGTGGTCAGGAAGCAAGGCAG | | |
| | TGGGAAGTGACCGAGTATTCAGACAAGCTTCACCTGC(BIO) | | |
| 121 | GCAGGTGAAGCTTGTCTGAATACTCGGTCACTTGCCACTGCCTTGCTTCCTG | 47263.8 | 47288.8 |
| | ACCACATCGATTTGGGAGTCAA (dSpacer)(dSpacer)(AOP-AminoC7) | | |
| | (dSpacer)(dSpacer)(dU)TGACTCCCAAATCGATGTGGTCAGGAAGCAAGGCAG | | |
| | TGGCAAGTGACCGAGTATTCAGACAAGCTTCACCTGC(BIO) | | |

The names of the compounds of the starting material head pieces for synthesizing each DEL compound are each as follows.

Starting material head piece

| | Starting material |
|---|---|
| DEL compound: | head piece |
| AAZ-BIO-DEL: | AAZ-DEL-HP |
| SABA-BIO-DEL: | SABA-DEL-HP |
| ClSABA-BIO-DEL: | ClSABA-DEL-HP |
| mSABA-BIO-DEL: | mSABA-DEL-HP |
| Amino-BIO-DEL: | AOP-U-DEL9-HP |

To a PCR tube were added 10 μL of 1 mM aqueous solution of various kinds of the starting material head pieces; 12 μL of 1 mM aqueous solution of Pr_TAG2_CP-BIO (it was prepared by annealing Pr_TAG2_CP_a and Pr_TAG2_CP-BIO_b synthesized in the same manner as in Example 1, the sequence is shown in Table 25); 4 μL of 10× ligase buffer (500 mM Tris hydrochloride, pH 7.5; 500 mM sodium chloride; 10 mM magnesium chloride; 100 mM dithiothreitol; and 20 mM adenosine triphosphate), and 10 μL of deionized water. To the solution, 4 μL of a 10-fold diluted aqueous solution of T4DNA ligase (available from Thermo Fisher, Catalog number: EL0013) was added, and the obtained solution was incubated at 16° C. overnight. The sequence notation in Table 25 is the same as in Table 24. Also, the names of the compounds corresponding to each SEQ ID NO: (No.) are as follows.
No. 122: Pr_TAG2_CP_a
No. 123: Pr_TAG2_CP-BIO_b

TABLE 25

| No. | Seq. |
|---|---|
| 122 | GCAGGTGAAGCTTGTCTGAATACTCGGTCACTTGCCACTGCC |
| | TTGCTTCCTGACCACATCGATTTGG |
| 123 | (p)AAATCGATGTGGTCAGGAAGCAAGGCAGTGGCAAGTGAC |
| | CGAGTATTCAGACAAGCTTCACCTGC(BIO) |

The reaction solution was treated with 4 μL of 5 M aqueous sodium chloride solution and 132 μL of cooled (−20° C.) ethanol, and allowed to stand at −78° C. for 30 minutes. After centrifugation, the supernatant was removed, the obtained pellets were air-dried, and the pellets were dissolved in deionized water. The obtained solution was desalted by the Amicon® Ultra Centrifugal filter (3 kD cutoff).

Of the obtained supernatant, a part thereof was sampled and diluted with deionized water, and then mass spectrometry by ESI-MS was carried out under the conditions of Analytical condition 3 in Example 3 to identify each target product (the expected molecular weight and the observed molecular weight of each compound are shown in Table 24).

<Cleavage by USER® Enzyme of 5 Kinds of DEL Compounds Having Biotin at 3′ End (AAZ-BIO-DEL, SABA-BIO-DEL, ClSABA-BIO-DEL, mSABA-BIO-DEL, and Amino-BIO-DEL)>

The 5 kinds of DEL compounds “AAZ-BIO-DEL”, “SABA-BIO-DEL”, “ClSABA-BIO-DEL”, “mSABA-BIO-DEL”, and “Amino-BIO-DEL” obtained above were cleaved by USER® enzyme in the following procedures to convert into DEL compounds “DS-AAZ-BIO-DEL”, “DS-SABA-BIO-DEL”, “DS-ClSABA-BIO-DEL”, “DS-mSABA-BIO-DEL”, and “DS-Amino-BIO-DEL”, each having the double-stranded nucleic acid having the sequence shown in Table 26. The sequence notation in Table 26 is the same as in Table 24, and it means that the 5 kinds of compounds are formed by double strands of the oligonucleotide chains of SEQ ID NOs: 124 and 125, SEQ ID NOs: 126 and 127, SEQ ID NOs: 128 and 129, SEQ ID NOs: 130 and 131, and SEQ ID NOs: 132 and 133, respectively.

TABLE 26

| | | | Expected | Observed MW. |
|---|---|---|---|---|
| | No. | Seq. | MW. | (deconvoluton) |
| DS-AAZ- | 124 | (p)TGACTCCCAAATCGATGTGGTCAGGAAGCAAGGCAGT | 23081.1 | 23092.6 |
| BIO-DEL | | GGCAAGTGACCGAGTATTCAGACAAGCTTCACCTGC(BIO) | | |
| | 125 | GCAGGTGAAGCTTGTCTGAATACTCGGTCACTTGCCACT | 24252.8 | 24253.8 |
| | | GCCTTGCTTCCTGACCACATCGATTTGGGAGTCAA | | |
| | | (dSpacer)(dSpacer)(AAZ-AOP-AminoC7) | | |
| | | (dSpacer)(dS pacer)(p) | | |
| DS-SABA- | 126 | (p) TGACTCCCAAATCGATGTGGTCAGGAAGCAAGGCAGT | 23081.1 | 23078.4 |
| BIO-DEL | | GGCAAGTGACCGAGTATTCAGACAAGCTTCACCTGC(BIO) | | |
| | 127 | GCAGGTGAAGCTTGTCTGAATACTCGGTCACTTGCCACT | 24173.5 | 24171.7 |
| | | GCCTTGCTTCCTGACCACATCGATTTGGGAGTCAA | | |
| | | (dSpacer)(dSpacer)(SABA-AOP-AminoC7) | | |
| | | (Spacer)(dSpacer)(p) | | |
| DS-CISAA- | 128 | (p)TGACTCCCAAATCGATGTGGTCAGGAAGCAAGGCAGT | 23081.1 | 23078.5 |
| BIO-DEL | | GGCAAGTGACCGAGTATTCAGACAAGCTTCACCTGC(BIO) | | |
| | 129 | GCAGGTGAAGCTTGTCTGAATACTCGGTCACTTGCCACT | 24208.2 | 24206.2 |
| | | GCCTTGCTTCCTGACCACATCGATTTGGGAGTCAA | | |
| | | (dSpacer)(dSpacer)(CISABA-AOP-AminoC7) | | |
| | | (dSpacer)(dSpacer)(p) | | |
| DS-mSABA- | 130 | (p)TGACTCCCAAATCGATGTGGTCAGGAAGCAAGGCAGT | 23081.1 | 23075.1 |
| BIO-DEL | | GGCAAGTGACCGAGTATTCAGACAAGCTTCACCTGC(BIO) | | |
| | 131 | GCAGGTGAAGCTTGTCTGAATACTCGGTCACTTGCCACT | 24173.5 | 24168.4 |
| | | GCCTTGCTTCCTGACCACATCGATTTGGGAGTCAA | | |
| | | (dSpacer)(dSpacer)(mSABA-AOP-AminoC7) | | |
| | | (Spacer)(dSpacer)(p) | | |
| DS-Amino- | 132 | (p)TGACTCCCAAATCGATGTGGTCAGGAAGCAAGGCAGT | 23081.1 | 23083.6 |
| BIO-DEL | | GGCAAGTGAGCGAGTATTCAGACAAGCTTCACCTGC(BIO) | | |
| | 133 | GCAGGTGAAGCTTGTCTGAATACTCGGTCACTTGCCACT | 23990.5 | 24002.0 |
| | | GCCTTGCTTCCTGACCACATCGATTTGGGAGTCAA | | |
| | | (dSpacer)(dSpacer)(AOP-AminoC7) | | |
| | | (dSpacer)(Spacer)(p) | | |

To a PCR tube were added 10 μL of 100 μM aqueous solution of various kinds of DEL compounds; 100 μL of CutSmart® Buffer (available from New England BioLabs, Catalog number: 72405), and 860 μL of deionized water. To the solutions was each added 30 μL of USER® enzyme (available from New England BioLabs, Catalog number: 5505S), and the obtained solution was incubated at 37° C. for 1 hour.

The obtained reaction solution was desalted and concentrated by the Amicon® Ultra Centrifugal filter (3 kD cutoff), and ethanol precipitation was carried out, and then deionized water was added to each obtained pellet to prepare an aqueous solution.

A part of the obtained solution was sampled and diluted with deionized water, and then mass spectrometry by ESI-MS was carried out under Analytical condition 3 in Example 3 to identify the DEL compounds “DS-AAZ-BIO-DEL”, “DS-SABA-BIO-DEL”, “DS-ClSABA-BIO-DEL”, “DS-mSABA-BIO-DEL”, and “DS-Amino-BIO-DEL” having the double-stranded nucleic acid of interest (the expected molecular weights and the observed molecular weights of the compounds are shown in Table 26).

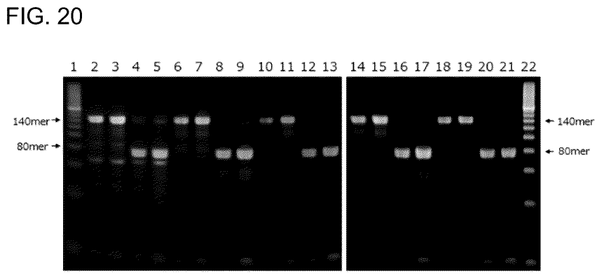

Also, a part of the obtained reaction solution was sampled, and analysis by modified polyacrylamide gel electrophoresis was carried out under the same conditions as in Example 3. From the results shown in FIG. 20, it was confirmed that “AAZ-BIO-DEL”, “SABA-BIO-DEL”, “ClSABA-BIO-DEL”, “mSABA-BIO-DEL”, and “Amino-BIO-DEL” were cleaved with high yield and converted into “DS-AAZ-BIO-DEL”, “DS-SABA-BIO-DEL”, “DS-ClSABA-BIO-DEL”, “DS-mSABA-BIO-DEL”, and “DS-Amino-BIO-DEL”, respectively. The samples of each Lane in FIG. 20 were as follows. In addition, concentration 1 and concentration 2 mean that the samples were prepared so that the loading amounts of the DEL compounds were about 40 ng and about 80 ng, respectively.
Lane 1: 20 bp DNA Ladder (manufactured by Lonza, Lonza 20 bp DNA Ladder, Catalog number: 50330)
Lane 2: AAZ-BIO-DEL (concentration 1)
Lane 3: AAZ-BIO-DEL (concentration 2)
Lane 4: Sample after subjecting to cleavage reaction of AAZ-BIO-DEL by USER® enzyme (concentration 1)
Lane 5: Sample after subjecting to cleavage reaction of AAZ-BIO-DEL by USER® enzyme (concentration 2)
Lane 6: SABA-BIO-DEL (concentration 1)
Lane 7: SABA-BIO-DEL (concentration 2)
Lane 8: Sample after subjecting to cleavage reaction of SABA-BIO-DEL by USER® enzyme (concentration 1)
Lane 9: Sample after subjecting to cleavage reaction of SABA-BIO-DEL by USER® enzyme (concentration 2)
Lane 10: ClSABA-BIO-DEL (concentration 1)
Lane 11: ClSABA-BIO-DEL (concentration 2)
Lane 12: Sample after subjecting to cleavage reaction of ClSABA-BIO-DEL by USER® enzyme (concentration 1)
Lane 13: Sample after subjecting to cleavage reaction of ClSABA-BIO-DEL by USER® enzyme (concentration 2)
Lane 14: mSABA-BIO-DEL (concentration 1)
Lane 15: mSABA-BIO-DEL (concentration 2)
Lane 16: Sample after subjecting to cleavage reaction of mSABA-BIO-DEL by USER® enzyme (concentration 1)
Lane 17: Sample after subjecting to cleavage reaction of mSABA-BIO-DEL by USER® enzyme (concentration 2)
Lane 18: Amino-BIO-DEL (concentration 1)
Lane 19: Amino-BIO-DEL (concentration 2)
Lane 20: Sample after subjecting to cleavage reaction of Amino-BIO-DEL by USER® enzyme (concentration 1)
Lane 21: Sample after subjecting to cleavage reaction of Amino-BIO-DEL by USER® enzyme (concentration 2)
Lane 22: 20 bp DNA ladder (manufactured by Lonza, Lonza 20 bp DNA Ladder, Catalog number: 50330)

### <Preparation of DEL Having a Single-Stranded DNA Using Streptavidin Beads>

The DEL compounds having the double-stranded nucleic acids obtained above “DS-AAZ-BIO-DEL”, “DS-SABA-BIO-DEL”, “DS-CISABA-BIO-DEL”, “DS-mSABA-BIO-DEL”, and “DS-Amino-BIO-DEL” were each treated with streptavidin beads, and the DEL compounds having a single-stranded DNA “SS-AAZ-DEL”, “SS-SABA-DEL”, “SS-ClSABA-DEL”, “SS-mSABA-DEL”, and “SS-Amino-DEL” were prepared by the following procedures. The 5 kinds of compounds are oligonucleotide chains of SEQ ID NOs: 125, 127, 129, 131, and 133 in Table 26, respectively.

To five PCR tubes was each added 450 μL of Magnosphere (trademark) MS160/Streptavidin (JSR Life Sciences, Catalog number: J-MS-S160S), the supernatant was removed by magnetic separation, and then 900 μL of 1× binding buffer (10 mM Tris hydrochloride, pH 7.5; 0.5 mM ethylenediamine tetraacetic acid; 1 M sodium chloride; and 0.05% v/v Tween 20) was added and the supernatant was removed by magnetic separation. An aqueous solution (700 μmol, 450 μL, each) of “DS-AAZ-BIO-DEL”, “DS-SABA-BIO-DEL”, “DS-ClSABA-BIO-DEL”, “DS-mSABA-BIO-DEL”, or “DS-Amino-BIO-DEL” and 450 μL of 2× binding buffer (20 mM Tris hydrochloric acid, pH 7.5; 1 mM ethylenediaminetetraacetic acid; 2 M sodium chloride; and 0.1% v/v Tween 20) were added to the obtained particles and mixed, and the mixture was shaken at room temperature for 20 minutes.

The supernatant was removed from each mixture by magnetic separation, and washing of particles using 900 μL of 1× binding buffer (10 mM Tris hydrochloride, pH 7.5; 0.5 mM ethylenediamine tetraacetic acid; 1 M sodium chloride; and 0.05% v/v Tween 20) and removal of the supernatant by magnetic separation were each repeated twice. Then, each 900 μL of a denaturing solution (0.1 M sodium hydroxide; and 0.1 M sodium chloride) was added, and the supernatant was recovered by magnetic separation.

900 μL of 3-(N-morpholino)propanesulfonic acid buffer (1.0 M, pH 7.0) was each added to the obtained supernatant and desalted by the Amicon® Ultra Centrifugal filter (3 kD cutoff). Each of the obtained supernatants was subjected to ethanol precipitation, and then deionized water was added to the pellets to prepare a solution.

A part of the obtained solution was sampled and diluted with deionized water, and then mass spectrometry by ESI-MS was carried out under the conditions of Analytical condition 3 in Example 3. The molecular weights of 24255.3, 24170.5, 24208.8, 24176.7, and 23984.8 were observed and the object DEL compounds having the single-stranded DNA of interest “SS-AAZ-DEL”, “SS-SABA-DEL”, “SS-ClSABA-DEL”, “SS-mSABA-DEL”, and “SS-Amino-DEL” were identified.

### <Synthesis of Photoreactive Cross Linker-Modified Primer>

The photoreactive cross linker-modified primer having the sequence shown in Table 27 “PXL-Pr” was synthesized by the following procedures. In the sequence notation in Table 27, “X” means the group represented by the following formula (16)

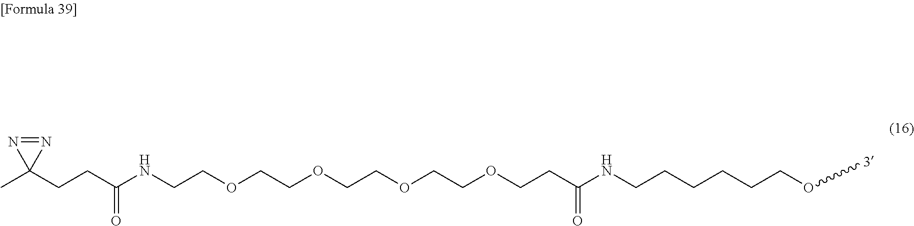

and other notations are the same as in Table 2.

TABLE 27

| | | | Expected | Observed MW. |
|---|---|---|---|---|
| | No. | Seq. | MW. | (deconvolution) |
| PXL-Pr | 134 | XTTGACTCCCAA | 6628.6 | 6627.6 |
| | | ATCGATGTG | | |

To a PCR tube was added a solution (200 TL, 1 mM) of L-Pr (synthesized in the same manner as in Example 1; the sequence is shown in Table 28) in a sodium borate buffer (150 mM, pH 9.4) cooled to 10° C. To the tube were added 40 equivalents of N-Fmoc-15-amino-4,7,10,13-tetraoxaoctadecanoic acid (20 μL, 0.4 M N,N-dimethylacetamide solution), subsequently 40 equivalents of 4-(4,6-dimethoxy[1.3.5]triazin-2-yl)-4-methylmorpholinium chloride hydrate (DMTMM) (16 μL, 0.5 M aqueous solution), and the resulting mixture was shaken at 10° C. for 4 hours. The sequence notation in Table 28 is the same as in Table 8.

TABLE 28

| | No. | Seq. |
|---|---|---|
| L-Pr | 135 | (amino-C6-L)TTGACTCCCAAATCGATGTG |

The reacted solution was treated with 23.6 μL of 5 M aqueous sodium chloride solution and 778.8 μL of cooled (−20° C.) ethanol, and allowed to stand at −78° C. overnight. After centrifugation, the supernatant was removed and the obtained pellets were air-dried. To the pellets was added 180 μL of deionized water to prepare a solution, and then 20 μL of piperidine was added and the mixture was shaken at 10° C. for 3 hours.

The resulting solution was treated with 20 μL of 5 M aqueous sodium chloride solution and 660 μL of cooled (−20° C.) ethanol, and allowed to stand at −78° C. for 30 minutes. After centrifugation, the supernatant was removed, and to the obtained pellets was added 200 μL of deionized water to prepare a 1 mM solution.

To 100 μL of the solution obtained above were added 75 μL of triethylamine hydrochloride buffer (500 mM, pH 10), subsequently 50 equivalents of sodium 1-((3-(3-methyl-3H-diazirin-3-yl)propanoyl)oxy)-2,5-dioxopyrrolidine-3-sulfonate (Sulfo-SDA) (25 μL, 200 mM aqueous solution), and the mixture was shaken at 37° C. for 2 hours.

The resulting solution was treated with 20 μL of 5 M aqueous sodium chloride solution and 660 μL of cooled (−20° C.) ethanol, and allowed to stand at −78° C. for 30 minutes. After centrifugation, the supernatant was removed, and to the obtained pellets were added 100 μL of deionized water, subsequently 75 μL of triethylamine hydrochloride buffer (500 mM, pH 10) and 50 equivalents of Sulfo-SDA (25 μL, 200 mM aqueous solution), and the mixture was shaken at 37° C. for 1 hour and 20 minutes. 50 equivalents of Sulfo-SDA (25 μL, 200 mM aqueous solution) was further added and the mixture was shaken at 37° C. for 40 minutes.

The resulting solution was treated with 22.5 μL of 5 M aqueous sodium chloride solution and 743 μL of cooled (−20° C.) ethanol, and allowed to stand at −78° C. overnight. After centrifugation, the supernatant was removed, and to the obtained pellets were added 100 μL of deionized water, subsequently 75 μL of triethylamine hydrochloride buffer (500 mM, pH 10), subsequently 50 equivalents of Sulfo-SDA (25 μL, 200 mM aqueous solution), and the mixture was shaken at 37° C. for 3 hours.

The resulting solution was treated with 20 μL of 5 M aqueous sodium chloride solution and 660 μL of cooled (−20° C.) ethanol, and allowed to stand at −78° C. overnight. After centrifugation, the supernatant was removed and the obtained pellets were air-dried. The pellets were dissolved in 50 mM triethyl ammonium acetate buffer (pH 7.5) and purified by reverse phase HPLC using Phenomenex Gemini C18 column. Using a dual mobile phase gradient profile, the target product was eluted using 50 mM triethylammonium acetate buffer (pH7.5) and acetonitrile/water (100:1, v/v). Fractions containing the target product were collected, mixed, and concentrated. The resulting solution was desalted by the Amicon® Ultra Centrifugal filter (3 kD cutoff), ethanol precipitation was carried out, and then 100 μL of deionized water was added to the pellets to prepare a solution.

A part of the obtained solution was sampled and diluted with deionized water, and then mass spectrometry by ESI-MS was carried out under the conditions of Analytical condition 3 in Example 3 to identify the target photoreactive cross linker-modified primer “PXL-Pr” (the expected molecular weight and the observed molecular weight of the compound are shown in Table 27).

### <Synthesis of Photoreactive Cross Linker-Modified Double-Stranded DEL>

Using the DEL compounds having the single-stranded DNA obtained above (“SS-AAZ-DEL”, “SS-SABA-DEL”, “SS-ClSABA-DEL”, “SS-mSABA-DEL”, and “SS-Amino-DEL”) as template DNAs, a primer elongation reaction using “PXL-Pr” was carried out in the following procedures to synthesize the photoreactive cross linker-modified double-stranded DEL compounds having the sequences shown in Table 29 (“PXL-DS-AAZ-DEL”, “PXL-DS-SABA-DEL”, “PXL-DS-CISABA-DEL”, “PXL-DS-mSABA-DEL”, and “PXL-DS-Amino-DEL”). The sequence notation in Table 29 is the same as in Tables 26 and 27, and it means that the 5 kinds of the compounds are formed by double strands of the oligonucleotide chains of SEQ ID NOs: 136 and 125, SEQ ID NOs: 136 and 127, SEQ ID NOs: 136 and 129, SEQ ID NOs: 136 and 131, and SEQ ID NOs: 136 and 133, respectively.

TABLE 29

| | | | Expected | Observed MW. |
|---|---|---|---|---|
| | No. | Seq. | MW. | (deconvolution) |
| PXLDS- | 136 | XTTGACTCCCAAATCGATGTGGTCAGGAAGCAAGGCAGT | 23404.4 | 23407.0 |
| AAZ-DEL | | GGCAAGTGACCGAGTATTCAGACAAGCTTCACCTGC | | |
| | 125 | GCAGGTGAAGCTTGTCTGAATACTCGGTCACTTGCCACT | 24252.8 | 24260.0 |
| | | GCCTTGCTTCCTGACCACATCGATTTGGGAGTC(dSpacer) | | |
| | | (dSpacer)(AAZ-AOP-AminoC7)(dSpacer)(dSpacer)(p) | | |
| PXL-DS- | 136 | XTTGACTCCCAAATCGATGTGGTCAGGAAGCAAGGGAGT | 23404.4 | 23398.4 |
| SABA-DEL | | GGCAAGTGACCGAGTATTCAGACAAGCTTCACCTGC | | |
| | 127 | GCAGGTGAAGCTTGTCTGAAT?CTCGGTCACTTGCCACT | 24173.5 | 24165.7 |
| | | GCCTTGCTTCCTGACCACATCGATTTGGGAGTCAA(dSpacer) | | |
| | | (dSpacer)(SABA-AOP-AminoC7)(dSpacer)(Spacer)(p) | | |
| PXL-DS- | 136 | XTTGACTCGCAAATCGATGTGGTCAGGAAGCAAGGCAGT | 23404.4 | 23406.6 |
| CISABA-DEL | | GGCAAGTGACCGAGTATTCAGACAAGCTTCACCTGC | | |
| | 129 | GCAGGTGAAGCTTGTCTGAATACTGGGTCACTTGCCACT | 24208.2 | 24203.7 |
| | | GCCTTGCTTCCTGACCACATCGATTTGGGAGTCAA(dSpacer) | | |
| | | (dSpacer)(CISABA-AOP-C7)(dS pacer)(dSpacer)(p) | | |
| PXL-DS- | 136 | XTTGACTCCCAAATCGATGTGGTCAGGAAGCAAGGCAGT | 23404.4 | 23401.4 |
| mSABA-DEL | | GGCAAGTGACCGAGTATTCAGACAAGCTTCACCTGC | | |
| | 131 | GCAGGTGAAGCTTGTCTGAATACTGGGTCACTTGCCACT | 24173.5 | 24173.2 |
| | | GCCTTGCTTCCTGACCACATCGATTTGGGAGTCAA(dSpacer) | | |
| | | (dSpacer)(SABA-AOP-AminoC7)(Spacer)(dSpacer)(p) | | |
| PXL-DS- | 136 | XTTGACTCCCAAATCGATGTGGTCAGGAAGCAAGGCAGT | 23404.4 | 23400.4 |
| Amino-DEL | | GGCAAGTGACCGAGTATTCAGACAAGCTTCACCTGC | | |
| | 133 | GCAGGTGAAGCTTGTCTGAATACTCGGTCACTTGCCACT | 23990.5 | 23987.8 |
| | | GCCTTGCTTCCTGACCACATCGATTTGGGAGTCAA(dSpacer) | | |
| | | (dSpacer)(AOP-AminoC7)(dSpacer)(Spacer)(p) | | |

To a PCR tube were added 30 μL of 10 μM aqueous solution of DEL compounds having various single-stranded DNAs; 0.505 μL of 594 μM “PXL-Pr” aqueous solution; 60 μL of 10× NEBluffer® 2 (available from New England BioLabs, 5 Catalog number: B7002S), and 476 μL of deionized water. To the solution were added 6 μL of DNA Polymerase I, Large (Klenow) Fragment (available from New England BioLabs, Catalog number: M0210), and 12 μL of Deoxynucleotide (dNTP) Solution Mix (available from New England BioLabs, Catalog number: N0447), and the resulting solution was incubated at 25° C. for 90 minutes.

The resulting solution was desalted by the Amicon® Ultra Centrifugal filter (3 kD cutoff). To the obtained supernatant was added deionized water to obtain a 60 μL solution. Then, the solution was treated with 6 μL of 5 M aqueous sodium chloride solution and 198 μL of cooled (−20° C.) ethanol, and allowed to stand at −78° C. for 30 minutes. After centrifugation, the supernatant was removed and the obtained pellets were air-dried. To the pellets was added 30 μL of deionized water to prepare a solution.

A part of the obtained solution was sampled and diluted with deionized water, and then mass spectrometry by ESI-MS was carried out under the conditions of Analytical condition 3 in Example 3 to identify the target photoreactive cross linker-modified double-stranded DELs “PXL-DS-AAZ-DEL”, “PXL-DS-SABA-DEL”, “PXL-DS-ClSABA-DEL”, “PXL-DS-mSABA-DEL”, and “PXL-DS-Amino-DEL” (the expected molecular weights and the observed molecular weights of the compounds are shown in Table 29).

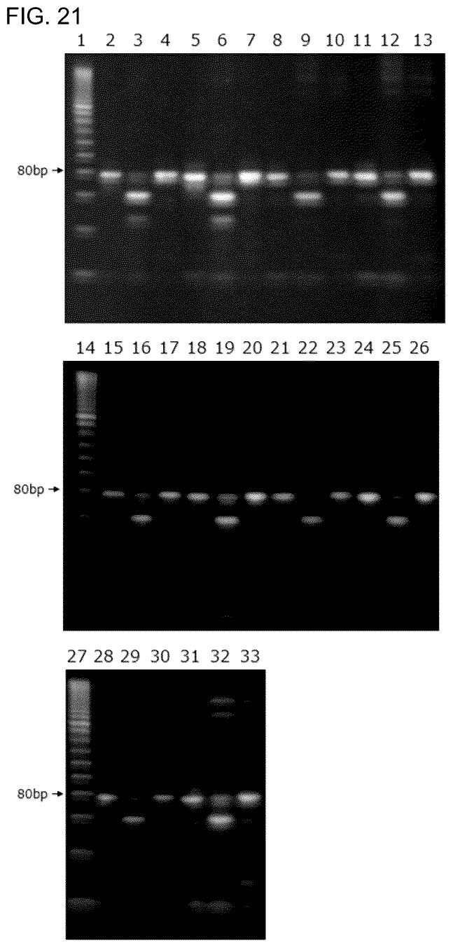

In addition, a part of the obtained reaction solution was sampled and analysis by polyacrylamide gel electrophoresis was carried out under the conditions mentioned below. From the results shown in FIG. 21, it was confirmed that conversion to each of “PXL-DS-AAZ-DEL”, “PXL-DS-SABA-DEL”, “PXL-DS-CISABA-DEL”, “PXL-DS-mSABA-DEL”, and “PXL-DS-Amino-DEL” with high yield by an elongation reaction of the primer. The samples of each Lane of FIG. 21 are as follows. In addition, concentration 1 and concentration 2 mean that the samples were prepared so that the loading amounts of the DEL compounds were about 40 ng and about 80 ng, respectively.
Lane 1: 20 bp DNA Ladder (manufactured by Lonza, Lonza 20 bp DNA Ladder, Catalog number: 50330)
Lane 2: DS-AAZ-BIO-DEL (concentration 1)
Lane 3: SS-AAZ-DEL (concentration 1)
Lane 4: Sample (PXL-DS-AAZ-DEL) after subjecting to primer elongation reaction of SS-AAZ-DEL (concentration 1)
Lane 5: DS-AAZ-BIO-DEL (concentration 2)
Lane 6: SS-AAZ-DEL (concentration 2)
Lane 7: Sample (PXL-DS-AAZ-DEL) after subjecting to primer elongation reaction of SS-AAZ-DEL (concentration 2)
Lane 8: DS-SABA-BIO-DEL (concentration 1)
Lane 9: SS-SABA-DEL (concentration 1)
Lane 10: Sample (PXL-DS-SABA-DEL) after subjecting to primer elongation reaction of SS-SABA-DEL (concentration 1)
Lane 11: DS-SABA-BIO-DEL (concentration 2)
Lane 12: SS-SABA-DEL (concentration 2)
Lane 13: Sample (PXL-DS-SABA-DEL) after subjecting to primer elongation reaction of SS-SABA-DEL (concentration 2)
Lane 14: 20 bp DNA ladder (manufactured by Lonza, Lonza 20 bp DNA Ladder, Catalog number: 50330)
Lane 15: DS-ClSABA-BIO-DEL (concentration 1)
Lane 16: SS-ClSABA-DEL (concentration 1)
Lane 17: Sample (PXL-DS-CISABA-DEL) after subjecting to primer elongation reaction of SS-ClSABA-DEL (concentration 1)
Lane 18: DS-ClSABA-BIO-DEL (concentration 2)
Lane 19: SS-ClSABA-DEL (concentration 2)
Lane 20: Sample (PXL-DS-CISABA-DEL) after subjecting to primer elongation reaction of SS-ClSABA-DEL (concentration 2)
Lane 21: DS-mSABA-BIO-DEL (concentration 1)
Lane 22: SS-mSABA-DEL (concentration 1)
Lane 23: Sample (PXL-DS-mSABA-DEL) after subjecting to primer elongation reaction of SS-mSABA-DEL (concentration 1)
Lane 24: DS-mSABA-BIO-DEL (concentration 2)
Lane 25: SS-mSABA-DEL (concentration 2)
Lane 26: Sample (PXL-DS-mSABA-DEL) after subjecting to primer elongation reaction of SS-mSABA-DEL (concentration 2)
Lane 27: 20 bp DNA ladder (manufactured by Lonza, Lonza 20 bp DNA Ladder, Catalog number: 50330)
Lane 28: DS-Amino-BIO-DEL (concentration 1)
Lane 29: SS-Amino-DEL (concentration 1)
Lane 30: Sample (PXL-DS-Amino-DEL) after subjecting to primer elongation reaction of SS-Amino-DEL (concentration 1)
Lane 31: DS-Amino-BIO-DEL (concentration 2)
Lane 32: SS-Amino-DEL (concentration 2)
Lane 33: Sample (PXL-DS-Amino-DEL) after subjecting to primer elongation reaction of SS-Amino-DEL (concentration 2)

### Polyacrylamide Gel Electrophoresis:

Gel: SuperSep (trademark) DNA 15% TBE gel (available from Fujifilm Wako Pure Chemical Corporation, Catalog number: 190-15481)
Loading Buffer: 6× Loading Buffer (available from Takara Bio Inc., Catalog number: 9156)
Temperature: room temperature
Voltage: 200V
Electrophoresis duration: 50 min
Dyeing reagent: SYBER (trademark) GreenII Nucleic Acid Gel Stain (available from Takara Bio Inc., Catalog number: 5770A)

### Example 8

### [Comparison of Binder Recovery Efficiencies in Presence or Absence of Photocrosslinking Reaction of Photoreactive Cross Linker-Modified Double-Stranded DEL]

### <Preparation of DEL Samples>

Each of the 5 kinds of the photoreactive cross linker-modified double-stranded DELs obtained above was diluted with deionized water to prepare a 50 nM DEL sample.

### <Photocrosslinking Reaction>

Device: CL-1000 Ultraviolet Crosslinker (UVP, manufactured by INC Corporation)
Reaction tube: bottom vial for 96 holes (manufactured by Technolabsc Co., Ltd., Catalog number 96-V050FB)
Photocrosslinking reaction solution:
   Salmon Sperm DNA, sheared (Invitrogen, Catalog number: AM9680): 1.6 μL
   1M NaCl aqueous solution: 5.0 μL
   D-PBS(−) (9, available from FUJIFILM Corporation, Catalog number: 045-29795): 32.4 μL
   Carbonic Anhydrase IX/CA9 (available from Sino Biological Inc., Catalog number: 10107-H08H): 10.0 μL
   50 nM aqueous solutions of various DEL samples: 1.0 μL
Reaction conditions:
   A mixture of the CA9 protein having the above composition and the DEL solution was incubated on ice for 1 hour. Then, a part of the solution was collected (solution S). The remaining solution was held on ice, and UV radiation at 365 nm was carried out for 20 minutes.
      <Recovery of DEL Crosslinked with Protein>
Dynabeads Histag isolation & pulldown (manufactured by Invitrogen Corporation, Catalog number 10104D): 10.0 μL
Tween 20 (available from Sigma Corporation, Catalog number: P7949-100ML)
10% SDS (available from NIPPON GENE CO., LTD., Catalog number: 311-90271)
Wash buffer (prepared by diluting D-PBS(−) with Tween 20 and 10% SDS to 0.2%)

The reaction solution after the UV radiation was mixed with Dyanebeads his-tag pulldown and incubated at room temperature for 30 minutes. The mixture was fixed to a magnet stand and left to stand for 2 minutes, and then the supernatant was removed and 200 μL of Wash buffer was added to suspend Dynabeads. This washing operation was further repeated five times. 80 μL of D-PBS(−) was added to the Dybabeads after the washing and the mixture was reacted at 95° C. for 10 minutes. After the reaction, Dynabeads were placed on a magnet stand and the supernatant was collected after 2 minutes (solution E).

<Preparation of Sample without Photocrosslinking Reaction>

The above series of operations were carried out without UV radiation, and the solution S and the solution E were prepared as samples, respectively.

### <Measurement of Ct Value by Real-Time PCR>

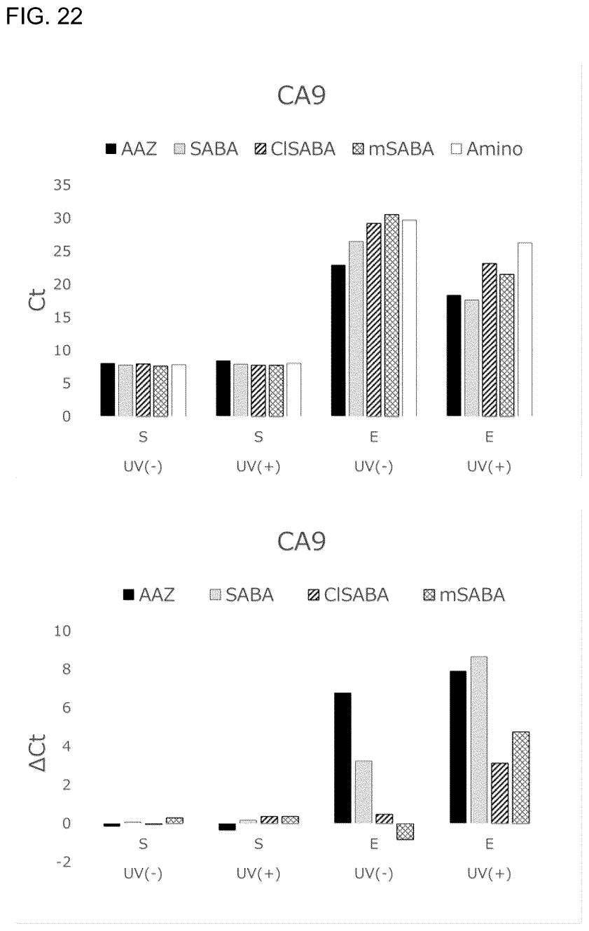

The Ct value of various kinds of DEL samples obtained above was measured by real-time PCR and the PCR efficiencies were compared. The conditions are as mentioned below and the results are shown in FIG. 22.
Device: 7500 real-time PCR system (manufactured by Applied Biosystems)
Plate: MicroAmp 96-Well plate (manufactured by Applied Biosystems, Catalog number: N8010560)

### PCR the Reaction Solution:

TaqMan Gene Expression Master Mix (manufactured by Applied Biosystems, Catalog number: 4369016): 10.0 μL
Forward primer 1 (Table 30, SEQ ID NO: 137): 1.0 μL
Reverse primer 1 (Table 30, SEQ ID NO: 138): 1.0 μL
Taqman MGB probe (manufactured by Thermo Fisher Scientific, product number: 4316034, Taqman® probe labeled with FAM® fluorescent dye at 5′ end and NFQ and MGB at 3′ end of the base sequence of SEQ ID NO: 139 in Table 30): 0.50 μL
Aqueous solutions (S and E samples) of various kinds of DEL samples: 2.0 μL
Deionized water: 5.5 μL

### Temperature Conditions:

After holding at 50° C. for 2 hours, holding at 95° C. for 10 minutes, and the following cycle was repeated for 40 cycles.
95° C., 15 seconds
59° C., 1 minute

TABLE 30

| No. | Seq. |
|---|---|
| 137 | AATCGATGTGGTCAGGAAGCA |
| 138 | GGCAGGTGAAGCTTGTCTGAA |
| 139 | CAGTGGCAAGTGACCGA |

The strength of the affinity between the CA9 protein and each compound as a binder is expected to be in the following order (Non-Patent Documents 6 and 7). “PXL-DS-AAZ-DEL”>“PXL-DS-SABA-DEL”>“PXL-DS-CISABA-DEL”>“PXL-DS-mSABA-DEL”>“PXL-DS-Amino-DEL (negative control)”

As shown in the graph of FIG. 22, in the solution E without UV radiation, the Ct values of “PXL-DS-AAZ-DEL” and “PXL-DS-SABA-DEL” having a high affinity binder were significantly reduced as compared to the negative control. However, “PXL-DS-ClSABA-DEL” and “PXL-DS-mSABA-DEL” having a medium affinity binder did not show any significant Ct value change as compared to the negative control. The result suggests that in the case of not carrying out the photocrosslinking reaction in the DEL screening, acquisition of a binder having high affinity can be expected but acquisition of a binder having medium affinity is difficult.

On the other hand, in the solution E with UV radiation, the Ct values of any compounds were significantly reduced as compared to the negative control. That is, the result suggests that in the case of carrying out the photocrosslinking reaction in the DEL screening, acquisition of a binder having medium affinity can be expected as well.

The result means that a photoreactive cross linker-modified double-stranded DEL derived from a hairpin type DEL having “selectively cleavable site(s)” is useful in DEL screening utilizing a photocrosslinking reaction.

### Example 9

### [Preparation of DEL Having a Single-Stranded DNA Using Lambda Exonuclease]

Conversion of the DEL compound having a double-stranded nucleic acid “DS-Amino-BIO-DEL” to the DEL compound having a single-stranded DNA “SS-Amino-DEL” by Lambda Exonuclease treatment was carried out by the following procedures.

To a PCR tube were added an aqueous solution of DS-Amino-BIO-DEL (500 μmol); 5 μL of 10× Lambda Exonuclease Reaction Buffer (available from New England BioLabs, Catalog number: B0262); and 1 μL of Lambda Exonuclease (available from New England BioLabs, Catalog number: M0262), and then a solution was prepared with deionized water so that a total solution amount was 50 μL. The resulting solution was incubated at 37° C. for 30 minutes.

10 μL of the obtained reaction solution was sampled and mass spectrometry by ESI-MS was carried out under the conditions of Analytical condition 3 in Example 3, whereby the molecular weight of 24024.9 was observed and the DEL compound having the single-stranded DNA of interest “SS-Amino-DEL” was identified.

MS of one oligonucleotide chain (SEQ ID NO: 132) comprised in DS-Amino-BIO-DEL was not detected and MS of the product after becoming the single-strand was observed as a main peak, whereby it was confirmed that becoming the single-strand was carried out with high yield.

### Example 10

### [Synthesis of Photoreactive Cross Linker-Modified Double-Stranded DEL Used in Example 8 and Optical Cross Linker-Modified Double-Stranded DEL Having a Different Linker Structure]

### <Preparation of 5 Kinds of DELs Having a Single-Stranded DNA>

In the same manner as in Example 7, 5 kinds of DEL compounds having a single-stranded DNA (“SS-AAZ-DEL”, “SS-SABA-DEL”, “SS-ClSABA-DEL”, “SS-mSABA-DEL”, and “SS-Amino-DEL”) were prepared. However, in the step of <Synthesis of 5 kinds of DEL compounds having biotin at 3′ end> described in Example 7, Pr_TAG2_CP (Pr_TAG2_CP_a and Pr_TAG2_CP_b synthesized in the same manner as in Example 1 were annealed, and the sequences are shown in Table 31) was used instead of Pr_TAG2_CP-BIO, and the preparation of DEL having a single-stranded DNA was carried out in the same manner as in Example 9 instead of the step of <Preparation of DEL having a single-stranded DNA using streptavidin beads>. The sequence notation in Table 31 is the same as in Table 25. Also, the names of the compounds corresponding to each SEQ ID NO: (No.) are as follows.
No. 122: Pr_TAG2_CP_a
No. 140: Pr_TAG2_CP_b

TABLE 31

| No. | Seq. |
|---|---|
| 122 | GCAGGTGAAGCTTGTCTGAATACTCGGTCACTTGCCACTGCCT |
| | TGCTTCCTGACCACATCGATTTGG |
| 140 | (p)AAATCGATGTGGTCAGGAAGCAAGGCAGTGGCAAGTGACC |
| | GAGTATTCAGACAAGCTTCACCTGC |

### <Synthesis of Photoreactive Cross Linker-Modified Primer “PXL-Pr2”>

The photoreactive cross linker-modified primer “PXL-Pr2” having the sequence shown in 32 was synthesized by the following procedures. In the sequence notation in Table 32, “(X2)” means the group represented by the following formula (17)

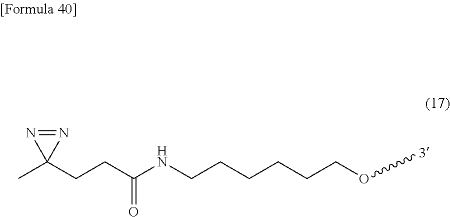

and other notations are the same as in Table 2.

TABLE 32

| | | | Expected | Observed MW. |
|---|---|---|---|---|
| | No. | Seq. | MW. | (deconvoluton) |
| PXL- | 141 | (2)TTGACTCCCAA | 6381.3 | 6380.2 |
| Pr2 | | ATCGATGTG | | |

To a PCR tube was added 3-(3-methyl-3H-diazilin-3-yl)propanoic acid (5 μL, 0.2 M N,N-dimethylacetamide solution). To the tube were added 1-[bis(dimethylamino)methylene]-1H-1,2,3-triazolo[4,5-b]pyridinium 3-oxide hexafluorophosphate (2.5 μL, 0.4 M N,N-dimethylacetamide solution), subsequently N,N-diisopropylethylamine (2.5 μL, 0.4 M N,N-dimethylacetamide solution), and the resulting solution was shaken at 4° C. for 10 minutes. To the resulting solution was added a solution (100 μL, 1 mM) of L-Pr (the sequence is shown in Table 28) in a sodium borate buffer (250 mM, pH 9.5) and the mixture was shaken at 10° C. for 30 minutes.

The solution was treated with 11 μL of 5 M aqueous sodium chloride solution and 363 μL of cooled (−20° C.) ethanol, and allowed to stand at −78° C. overnight. After centrifugation, the supernatant was removed and the obtained pellets were air-dried. The obtained pellets were dissolved in deionized water and the solution was desalted by the Amicon® Ultra Centrifugal filter (3 kD cutoff)

A part of the obtained supernatant was sampled and diluted with deionized water, and then mass spectrometry by ESI-MS was carried out under the conditions of Analytical condition 3 in Example 3 to identify the photoreactive cross linker-modified primer of interest “PXL-Pr2” (the expected molecular weight and the observed molecular weight of each compound are shown in Table 32).

### <Synthesis of Photoreactive Cross Linker-Modified Double-Stranded DEL>

Using the DEL compounds having the single-stranded DNA (“SS-AAZ-DEL”, “SS-SABA-DEL”, “SS-ClSABA-DEL”, “SS-mSABA-DEL”, and “SS-Amino-DEL”) obtained above as template DNAs, a primer elongation reaction using “PXL-Pr2” was carried out in the same procedures as in Example 7 to synthesize photoreactive cross linker-modified double-stranded DEL compounds having the sequences shown in Table 33 (“PXL-DS-AAZ-DEL2”, “PXL-DS-SABA-DEL2”, “PXL-DS-CISABA-DEL2”, “PXL-DS-mSABA-DEL2”, and “PXL-DS-Amino-DEL2”). The sequence notation in Table 33 is the same as in Tables 26 and 32, and it means that the 5 kinds of the compounds are formed by double strands of the oligonucleotide chains of SEQ ID NOs: 142 and 125, SEQ ID NOs: 142 and 127, SEQ ID NOs: 142 and 129, SEQ ID NOs: 142 and 131, and SEQ ID NOs: 142 and 133, respectively.

TABLE 33

| | | | Expected | Observed MW. |
|---|---|---|---|---|
| | No. | Seq. | MW. | (deconvoluton) |
| PXL-DS- | 142 | (X2)TTGACTCCCAAATCGATGTGGTCAGGAAGCAAGGCA | 23157.1 | 23176.8 |
| AAZ-DEL2 | | GTGGCAAGTGACCGAGTATTCAGACAAGCTTCACCTGC | | |
| | 125 | GCAGGTGAAGCTTGTCTGAATACTCGGTCACTTGCCACT | 24252.8 | 24263.0 |
| | | GCCTTGCTTCCTGACCACATCGATTTGGGAGTCAA(dSpacer) | | |
| | | (dSpacer)(AAZ-AOP-AminoC7)(dSpacer)(dSpacer)(p) | | |
| PXL-DS- | 142 | (X2)TTGACTCCCAAATCGATGTGGTCAGGAAGCAAGGCA | 23157.1 | 23176.1 |
| SABA-DEL2 | | GTGGCAAGTGACCGAGTATTCAGACAAGCTTCACCTGC | | |
| | 127 | GCAGGTGAAGCTTGTCTGAATACTCGGTCACTTGCCACT | 24173.5 | 24190.1 |
| | | GCCTTGCTTCCTGACCACATCGATTTGGGAGTCAA(dSpacer) | | |
| | | (dSpacer)(SABA-AOP-AminoC7)(dSpacer)(dSpacer)(p) | | |
| PXL-DS- | 142 | (X2)TTGACTCCCAAATCGATGTGGTCAGGAAGCAAGGCA | 23157.1 | 23201.3 |
| CISABA-DEL2 | | GTGGCAAGTGACCGAGTATTCAGACAAGCTTCACCTGC | | |
| | 129 | GCAGGTGAAGCTTGTCTGAATACTCGGTCACTTGCCACT | 24208.2 | 24216.6 |
| | | GCCTTGCTTCCTGACCACATCGATTTGGGAGTCAA(dSpacer) | | |
| | | (dSpacer)(CISABA-AOP-AminoC7)(Spacer)(dSpacer)(p) | | |
| PXL-DS- | 142 | (X2)TTGACTCCCAAATCGATGTGGTCAGGAAGCAAGGCA | 23157.1 | 23175.3 |
| mSABA-DEL2 | | GTGGCAAGTGACCGAGTATTCAGACAAGCTTCACCTGC | | |
| | 131 | GCAGGTGAAGCTTGTCTGAATACTCGGTCACTTGCCACT | 24173.5 | 24197.1 |
| | | GCCTTGCTTCCTGACCACATCGATTTGGGAGTCAA(dSpacer) | | |
| | | (dSpacer)(mSABA-AOP-AminoC7)(Spacer)(dSpacer)(p) | | |
| PXL-DS- | 142 | (X2)TTGACTCCCAAATCGATGTGGTCAGGAAGCAAGGCA | 23157.1 | 23162.3 |
| Amino-DEL2 | | GTGGCAAGTGACCGAGTATTCAGACAAGCTTCACCTGC | | |
| | 133 | GCAGGTGAAGCTTGTCTGAATACTCGGTCACTTGCCACT | 23990.5 | 24002.9 |
| | | GCCTTGCTTCCTGACCACATCGATTTGGGAGTCAA(dSpacer) | | |
| | | (dSpacer)(AOP-AminoC7)(Spacer)(Spacer)(p) | | |

A part of the obtained solution was sampled and diluted with deionized water, and then mass spectrometry by ESI-MS was carried out under the conditions of Analytical condition 3 in Example 3 to identify the photoreactive cross linker-modified double-stranded DELs of interest “PXL-DS-AAZ-DEL2”, “PXL-DS-SABA-DEL2”, “PXL-DS-ClSABA-DEL2”, “PXL-DS-mSABA-DEL2”, and “PXL-DS-Amino-DEL2” (the expected molecular weights and the observed molecular weights of the compounds are shown in Table 33).

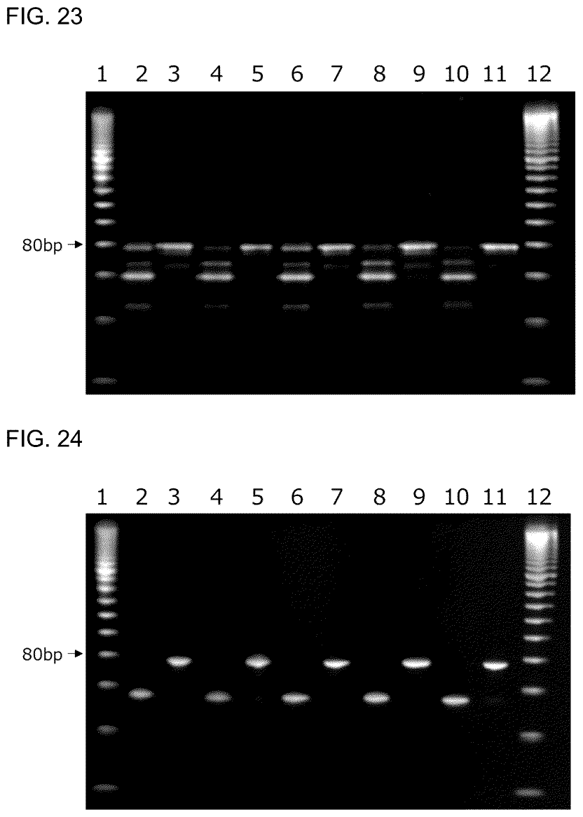

Also, a part of the obtained reaction solution was sampled and analysis by polyacrylamide gel electrophoresis was carried out under the conditions shown in Example 7. From the results shown in FIG. 23, it was confirmed that conversion to each of “PXL-DS-AAZ-DEL2”, “PXL-DS-SABA-DEL2”, “PXL-DS-ClSABA-DEL2”, “PXL-DS-mSABA-DEL2”, and “PXL-DS-Amino-DEL2” was accomplished with high yield by an elongation reaction of the primer. The samples of each Lane in FIG. 23 are as follows. The samples were prepared so that the loading amount of each DEL compound was about 40 ng.
Lane 1: 20 bp DNA Ladder (manufactured by Lonza, Lonza 20 bp DNA Ladder, Catalog number: 50330)
Lane 2: SS-AAZ-DEL
Lane 3: Sample (PXL-DS-AAZ-DEL2) after subjecting to primer elongation reaction of SS-AAZ-DEL
Lane 4: SS-SABA-DEL
Lane 5: Sample (PXL-DS-SABA-DEL2) after subjecting to primer elongation reaction of SS-SABA-DEL
Lane 6: SS-ClSABA-DEL
Lane 7: Sample (PXL-DS-CISABA-DEL2) after subjecting to primer elongation reaction of SS-ClSABA-DEL
Lane 8: SS-mSABA-DEL
Lane 9: Sample (PXL-DS-mSABA-DEL2) after subjecting to primer elongation reaction of SS-mSABA-DEL
Lane 10: SS-Amino-DEL
Lane 11: Sample (PXL-DS-Amino-DEL2) after subjecting to primer elongation reaction of SS-Amino-DEL
Lane 12: 20 bp DNA ladder (manufactured by Lonza, Lonza 20 bp DNA Ladder, Catalog number: 50330)

### <Preparation of DEL Compound Having a Single-Stranded DNA “SS-Amino-DEL3”>

A DEL compound having a single-stranded DNA “SS-Amino-DEL3” (the sequence is shown in Table 34) was prepared using an automated polynucleotide synthesizer nS-811 (manufactured by GeneDesign, Inc.) in the same manner as in Example 1. “SS-Amino-DEL3” has the same structure as the oligonucleotide derived by the same procedures as <Preparation of 5 kinds of DELs having a single-stranded DNA> using “U-DEL12-HP” (the sequence is shown in Table 11) as a starting material head piece. In addition, the sequence notation in Table 34 is the same as in Table 10.

TABLE 34

| No. | Seq. |
|---|---|
| 143 | GCAGGTGAAGCTTGTCTGAATACTCGGTCACTTGCCACTGCCTT |
| | GCTTCCTGACCACATCGATTTGGGAGTCAA |
| | [mdC(TEG-amino)](p) |

### <Synthesis of DEL Compound Having a Single-Stranded DNA “SS-AAZ-DEL3”>

A DEL compound having a single-stranded DNA having the sequence shown in Table 35 “SS-AAZ-DEL3” was synthesized by the following procedures. In the sequence notation in Table 35, “[AAZ-mdC (TEG-Amino)]” means the group represented by the following formula (18)

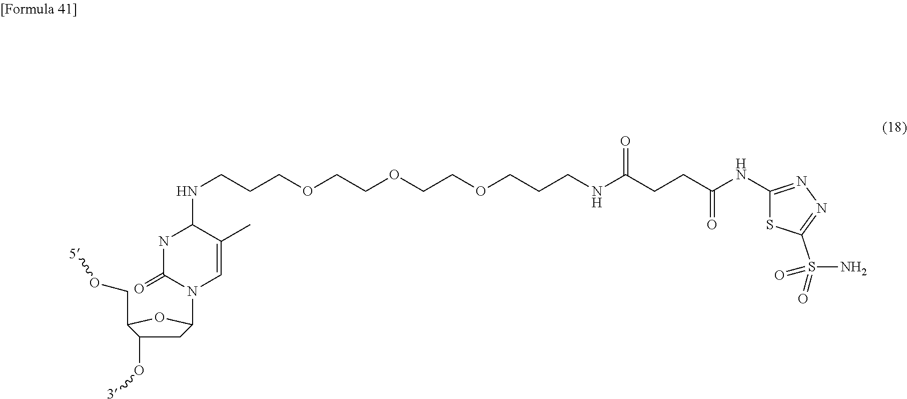

and other notations are the same as in Table 10.

TABLE 35

| | | Expected | Observed MW. |
|---|---|---|---|
| No. | Seq. | MW. | (deconvoluton) |
| 144 | GCAGGTGAAGCTTGTCTGAATACTCGGTCAC | 23582.4 | 23599.4 |
| | TTGCCACTGCCTTGCTTCCTGACCACATCGA | | |
| | TTTGGGAGTCAA[AAZ-mdC(TEG-amino)](p) | | |

In the same manner as in Example 7<Synthesis of starting material head piece (AAZ-DEL-HIP) of DEL compound>, a condensation reaction with 4-oxo-4-[(5-sulfamoyl-1,3,4-thiadiazol-2-yl)amino)butanoic acid was carried out using “SS-Amino-DEL3” as a starting material.

A part of the obtained solution was sampled and diluted with deionized water, and then mass spectrometry by ESI-MS was carried out under the conditions of Analytical condition 3 in Example 3 to identify the DEL compound having the single-stranded DNA of interest “SS-AAZ-DEL3” (the expected molecular weight and the observed molecular weight of the compound are shown in Table 35)

### <Synthesis of 3 Kinds of DEL Compounds Having a Single-Stranded DNA (“SS-SABA-DEL3”, “SS-ClSABA-DEL3”, and “SS-mSABA-DEL3”)>

3 kinds of DEL compounds having a single-stranded DNA having the sequences shown in Table 36 (“SS-SABA-DEL3”, “SS-ClSABA-DEL3”, and “SS-mSABA-DEL3”) were synthesized using the following procedures. In the sequence notation in Table 36, “[SABA-mdC (TEG-Amino)]” means the group represented by the following formula (19)

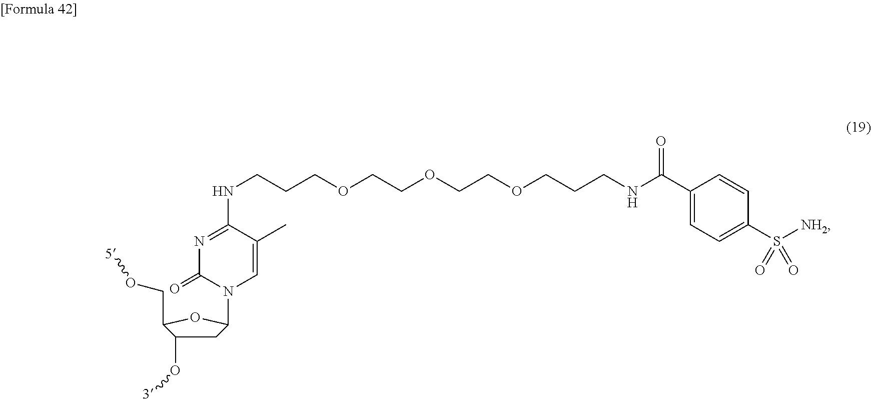

“[ClSABA-mdC (TEG-Amino)]” means the group represented by the following formula (20)

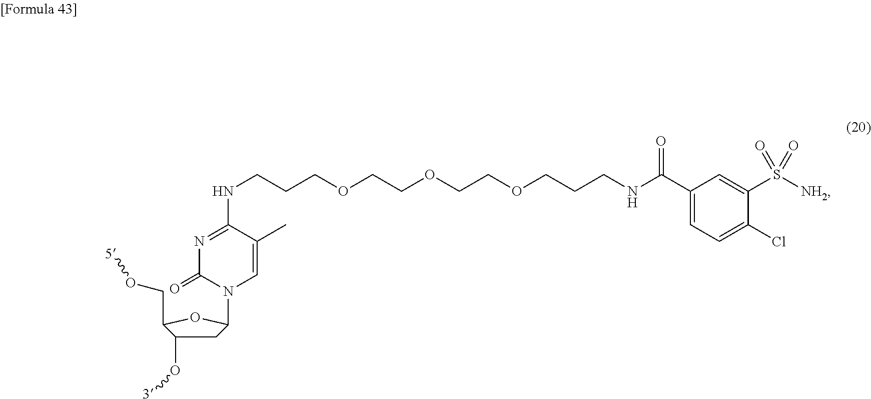

“[mSABA-mdC (TEG-Amino)]” means the group represented by the following formula (21)

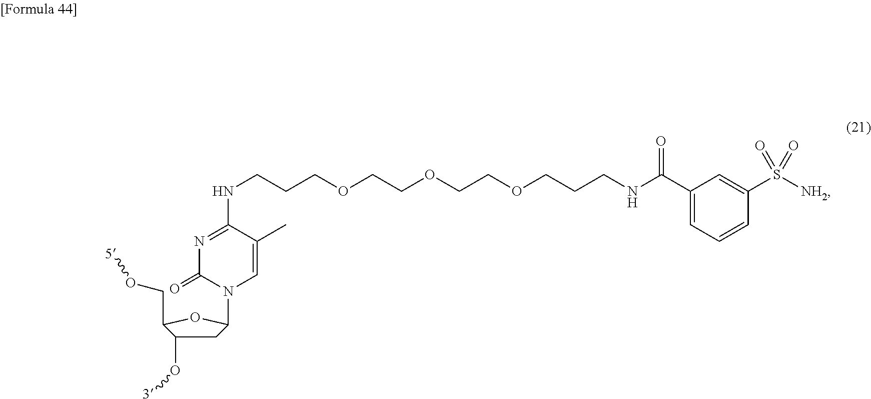

and other notations are the same as in Table 10. The names of the compounds corresponding to each SEQ ID NO: (No.) are as follows.
No. 145: SS-SABA-DEL3
No. 146: SS-ClSABA-DEL3
No. 147: SS-mSABA-DEL3

TABLE 36

| | | Expected | Observed MW. |
|---|---|---|---|
| No. | Seq. | MW. | (deconvoluton) |
| 145 | GCAGGTGAAGCTTGTCTGAATACTCGGTCAC | 23503.4 | 23523.3 |
| | TTGCCACTGCCTTGCTTCCTGACCACATCGA | | |
| | TTTGGGAGTCAA[SABA-mdC(TEG-amino)](p) | | |
| 146 | GCAGGTGAAGCTTGTCTGAATACTCGGTCAC | 23537.8 | 23557.7 |
| | TTGCCACTGCCTTGCTTCCTGACCACATCGA | | |
| | TTTGGGAGTCAA[CISABA-mdC(TEG-amino)](p) | | |
| 147 | GCAGGTGAAGCTTGTCTGAATACTCGGTCAC | 23503.4 | 23511.8 |
| | TTGCCACTGCCTTGCTTCCTGACCACATCGA | | |
| | TTTGGGAGTCAA[mSABA-mdC(TEG-amino)](p) | | |

The starting material carboxylic acids for synthesizing each compound are as follows.

| Compound: | Starting material carboxylic acid |
|---|---|
| SABA-DEL-HP: | 4-sulfamoylbenzoic acid |
| ClSABA-DEL-HP: | 4-chloro-3-sulfamoylbenzoic acid |
| mSABA-DEL-HP: | 3-sulfamoylbenzoic acid |

A starting material carboxylic acid (4 μL, 0.2M N,N-dimethylacetamide solution) was added to a PCR tube. To the tube were added 1-[bis(dimethylamino)methylene]-1H-1,2,3-triazolo[4,5-b]pyridinium 3-oxide hexafluorophosphate (2 μL, 0.4 M N,N-dimethylacetamide solution), subsequently N,N-diisopropylethylamine (2 μL, 0.4 M N,N-dimethylacetamide solution), and the resulting solution was shaken at 10° C. for 30 minutes. To the resulting solution was added a solution (50 μL, 1 mM) of SS-Amino-DEL3 in a sodium borate buffer (250 mM, pH 9.5) and the mixture was shaken at 10° C. for 2 hours.

The above solution was treated with 5.8 μL of 5 M aqueous sodium chloride solution and 192 μL of cooled (−20° C.) ethanol, and allowed to stand at −78° C. for 30 minutes. After centrifugation, the supernatant was removed and the obtained pellets were air-dried.

The obtained pellets were dissolved in 50 mM triethylammonium acetate buffer (pH 7.5) and purified by reverse phase HPLC using Phenomenex Gemini C18 column. Using a dual mobile phase gradient profile, the target product was eluted using 50 mM triethylammonium acetate buffer (pH 7.5) and acetonitrile/500 mM triethylammonium acetate buffer (9:1, v/v). Fractions containing the target product were collected, mixed, and concentrated. The resulting solution was desalted by the Amicon® Ultra Centrifugal filter (3 kD cutoff), ethanol precipitation was carried out, and then deionized water was added to the pellets to prepare a solution.

A part of the obtained solution was sampled and diluted with deionized water, and then mass spectrometry by ESI-MS was carried out under the conditions of Analytical condition 3 in Example 3 to identify “SS-SABA-DEL3”, “SS-ClSABA-DEL3”, and “SS-mSABA-DEL3” of interest (the expected molecular weight and the observed molecular weight of each compound are shown in Table 36).

<Synthesis of photoreactive cross linker-modified primer “PXL-Pr3”>

A photoreactive cross linker-modified primer having the sequence shown in Table 37 “PXL-Pr3” was synthesized by the same procedures as in the above <Synthesis of photoreactive cross linker-modified primer “PXL-Pr2”>. However, as a starting material, “L-Pr3” (synthesized in the same manner as in Example 1, and the sequence is shown in Table 38) was used instead of “L-Pr”. The sequence notation in Table 37 is the same as in Table 32. In addition, the sequence notation in Table 38 is the same as in Table 8.

TABLE 37

| | | | Expected | Observed MW. |
|---|---|---|---|---|
| | No. | Seq. | MW. | (deconvoluton) |
| PXL-Pr3 | 148 | (2)GTTGACTCCCAAA | 6710.5 | 6712.0 |
| | | TCGATGTG | | |

TABLE 38

| | No. | Seq. |
|---|---|---|
| L-Pr3 | 149 | (amino-C6-L)GTTGACTCCCAAATCGATGTG |

A part of the obtained solution was sampled and diluted with deionized water, and then mass spectrometry by ESI-MS was carried out under the conditions of Analytical condition 3 in Example 3 to identify the photoreactive cross linker-modified primer “PXL-Pr3” of interest (the expected molecular weight and the observed molecular weight of each compound are shown in Table 37).

### <Synthesis of 5 Kinds of Photoreactive Cross Linker-Modified Double-Stranded DELs “PXL-DS-DEL3”>

Using the DEL compounds having the single-stranded DNA obtained above (“SS-AAZ-DEL3”, “SS-SABA-DEL3”, “SS-ClSABA-DEL3”, “SS-mSABA-DEL3”, and “SS-Amino-DEL3”) as template DNAs, a primer elongation reaction using “PXL-Pr3” was carried out by the same procedures as in Example 7 to synthesize 5 kinds of the photoreactive cross linker-modified double-stranded DEL compounds having the sequences shown in Table 39 (“PXL-DS-AAZ-DEL3”, “PXL-DS-SABA-DEL3”, “PXL-DS-ClSABA-DEL3”, “PXL-DS-mSABA-DEL3”, and “PXL-DS-Amino-DEL3”). The sequence notation in Table 39 is the same as in Tables 34 to 37, and it means that the 5 kinds of the compounds are formed by double strands of the oligonucleotide chains of SEQ ID NOs: 150 and 144, SEQ ID NOs: 150 and 145, SEQ ID NOs: 150 and 146, SEQ ID NOs: 150 and 147, and SEQ ID NOs: 150 and 143, respectively.

TABLE 39

| | | | Expected | Observed MW. |
|---|---|---|---|---|
| | No. | Seq. | MW. | (deconvoluton) |
| PXL-DS- | 150 | (X2)GTTGACTCCCAAATCGATGTGGTCAGGAAGCAAGGC | 23486.3 | 23494.4 |
| AAZ-DEL3 | | AGTGGCAAGTGACCGAGTATTCAGACAAGCTTCACCTGC | | |
| | 144 | GCAGGTGAAGCTTGTCTGAATACTCGGTCACTTGCCACT | 23582.4 | 23591.4 |
| | | GCCTTGCTTCCTGACCACATCGATTTGGGAGTCAA | | |
| | | [AAZ-mdC(TEG-amino)](p) | | |
| PXL-DS- | 150 | (X2)GTTGACTCCCAAATCGATGTGGTCAGGAAGCAAGGC | 23486.3 | 23505.7 |
| SABA-DEL3 | | AGTGGCAAGTGAGCGAGTATTCAGACAAGCTTCACCTGC | | |
| | 145 | GCAGGTGAAGCTTGTCTGAATACTCGGTCACTTGCCACT | 23503.4 | 23513.1 |
| | | GCCTTGCTTCCTGACCACATCGATTTGGGAGTCAA | | |
| | | [SABA-mdC(TEG-amino)](p) | | |
| PXL-DS- | 150 | (X2)GTTGACTCCCAAATCGATGTGGTCAGGAAGCAAGGC | 23486.3 | 23505.3 |
| CISABA- | | AGTGGGAAGTGAGCGAGTATTCAGACAAGCTICACCTGC | | |
| DEL3 | 146 | GGAGGTGAAGCTTGTCTGAATACTCGGTCACTTGCCACT | 23537.8 | 23543.1 |
| | | GCCTTGCTTCCTGACCACATCGATTTGGGAGTCAA | | |
| | | [CISABA-mdC(TEG amino)](p) | | |
| PXL-DS- | 150 | (X2)GTTGACTCCCAAATCGATGTGGTCAGGAAGCAAGGC | 23486.3 | 23497.6 |
| mSABA- | | AGTGGCAAGTGACCGAGTATTCAGACAAGCTTCACCTGC | | |
| DEL3 | 147 | GCAGGTGAAGCTTGTCTGAATACTCGGTCACTTGCCACT | 23503.4 | 23508.0 |
| | | GCCTTGCTTCCTGACCACATCGATTTGGGAGTCAAM | | |
| | | [mSABA-mdC(TEG-amino)](p) | | |
| PXL-DS- | 150 | (X2)GTTGACTCCCAAATCGATGTGGTCAGGAAGCAAGGC | 23486.3 | 23497.5 |
| Amino- | | AGTGGCAAGTGACCGAGTATTCAGACAAGCTTCACCTGC | | |
| DEL3 | 143 | GCAGGTGAAGCTTGTCTGAATACTCGGTCACTTGCCACT | 23320.2 | 23327.3 |
| | | GCCTTGCTTCCTGACCACATCGATTTGGGAGTCAA | | |
| | | [mdC(TEG-amino)](p) | | |

A part of the obtained solution was sampled and diluted with deionized water, and then mass spectrometry by ESI-MS was carried out under the conditions of Analytical condition 3 in Example 3 to identify each photoreactive cross linker-modified double-stranded DEL compound “PXL-DS-AAZ-DEL3”, “PXL-DS-SABA-DEL3”, “PXL-DS-CISABA-DEL3”, “PXL-DS-mSABA-DEL3”, and “PXL-DS-Amino-DEL3” of interest (the expected molecular weights and the observed molecular weights of the compounds are shown in Table 39).

Also, a part of the obtained reaction solution was sampled and analysis by polyacrylamide gel electrophoresis was carried out under the conditions shown in Example 7. From the results shown in FIG. 24, it was confirmed that conversion to each of “PXL-DS-AAZ-DEL3”, “PXL-DS-SABA-DEL3”, “PXL-DS-ClSABA-DEL3”, “PXL-DS-mSABA-DEL3”, and “PXL-DS-Amino-DEL3” was accomplished with high yield by an elongation reaction of the primer. The samples of each Lane in FIG. 24 are as follows. The samples were prepared so that the loading amount of each DEL compound was about 40 ng.

Lane 1: 20 bp DNA Ladder (manufactured by Lonza, Lonza 20 bp DNA Ladder, Catalog number: 50330)
Lane 2: SS-AAZ-DEL3
Lane 3: Sample (PXL-DS-AAZ-DEL3) after subjecting to primer elongation reaction of SS-AAZ-DEL3
Lane 4: SS-SABA-DEL3
Lane 5: Sample (PXL-DS-SABA-DEL3) after subjecting to primer elongation reaction of SS-SABA-DEL3
Lane 6: SS-ClSABA-DEL3
Lane 7: Sample (PXL-DS-CISABA-DEL3) after subjecting to primer elongation reaction of SS-ClSABA-DEL3
Lane 8: SS-mSABA-DEL3
Lane 9: Sample (PXL-DS-mSABA-DEL3) after subjecting to primer elongation reaction of SS-mSABA-DEL3
Lane 10: SS-Amino-DEL3
Lane 11: Sample (PXL-DS-Amino-DEL3) after subjecting to primer elongation reaction of SS-Amino-DEL3
Lane 12: 20 bp DNA ladder (manufactured by Lonza, Lonza 20 bp DNA Ladder, Catalog number: 50330)

### Example 11

### [Comparison of Binder Recovery Efficiencies in Presence or Absence of Photocrosslinking Reaction of Photoreactive Cross Linker-Modified Double-Stranded DEL Synthesized in Example 10]

### <Preparation of DEL Samples>

Each of the 4 kinds of the photoreactive cross linker-modified double-stranded DELs obtained in Example 10 (PXL-DS-mSABA-DEL2, PXL-DS-Amino-DEL2, PXL-DS-mSABA-DEL3, and PXL-DS-Amino-DEL3) was diluted with deionized water to prepare a 50 nM DEL sample.

### <Photocrosslinking Reaction>

Device: CL-1000 Ultraviolet Crosslinker (UVP, manufactured by INC Corporation) Microtube: 1.5 mL siliconized microtube round bottom (manufactured by WATSON Co., Ltd., Catalog number: 131-615CH)
Reaction tube: bottom vial for 96 holes (manufactured by Technolabsc Co., Ltd., Catalog number 96-V050FB)
Photocrosslinking reaction solution:
   Salmon Sperm DNA, sheared (Invitrogen, Catalog number: AM9680): 1.6 μL
   1 M NaCl (available from FUJIFILM Corporation, Catalog number: 191-01665): 5.0 μL
   D-PBS(−) (available from FUJIFILM Corporation, Catalog number: 045-29795): 32.4 μL
   Carbonic Anhydrase IX/CA9 (available from Sino Biologocal Inc., Catalog number: 10107-H08H): 10.0 μL
   Various kinds of aqueous solutions (50 nM) of DEL samples: 1.0 μL
Reaction conditions:
   The CA9 protein having the above composition and the DEL solution were mixed in a microtube and reacted on ice for 1 hour. The entire amount of the mixture was dispensed into a reaction tube and held on ice, and UV radiation at 365 nm was carried out for 20 minutes.
      <Recovery of DEL Crosslinked with Protein>
Dynabeads Histag isolation & pulldown (manufactured by Invitrogen Corporation, Catalog number 10104D): 10.0 μL
Tween 20 (available from Sigma Corporation, Catalog number: P7949-100ML)
10% SDS (available from NIPPON GENE CO., LTD., Catalog number: 311-90271)
Wash buffer (prepared by diluting D-PBS(−) with Tween 20 and 10% SDS to 0.2%)
Imidazole (available from FUJIFILM Corporation, Catalog number: 097-05391)
Elution buffer (imidazole was dissolved in D-PBS(−) to 200 mM to prepare it at pH 7.4)

D-PBS(−) was added to the reaction solution after the UV radiation. Thereafter, the remaining solution was mixed with Dyanebeads his-tag pulldown and incubated at room temperature for 30 minutes. The mixture was fixed to a magnet stand and left to stand for 2 minutes, and then the supernatant was removed, 200 μL of Wash buffer was added to suspend Dynabeads. This operation was further repeated five times. 100 μL of Elution buffer was added to Dynabeads after the washing and the mixture was allowed to stand at room temperature for 10 minutes. After the reaction, Dynabeads were placed on a magnetic stand, and after 2 minutes, the supernatant was collected as a sample.

<Preparation of Sample without Photocrosslinking Reaction>

The above series of the operations was carried out without UV radiation, and each sample was collected.

### <Measurement of Ct Value by Real-Time PCR>

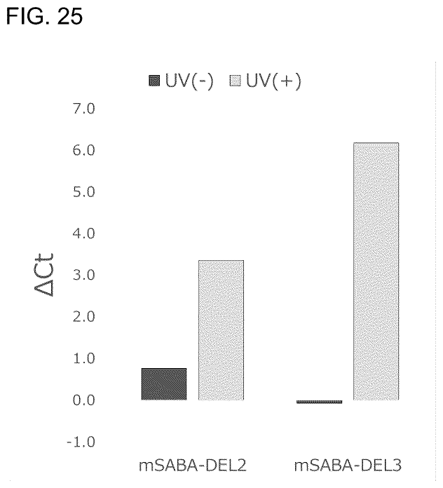

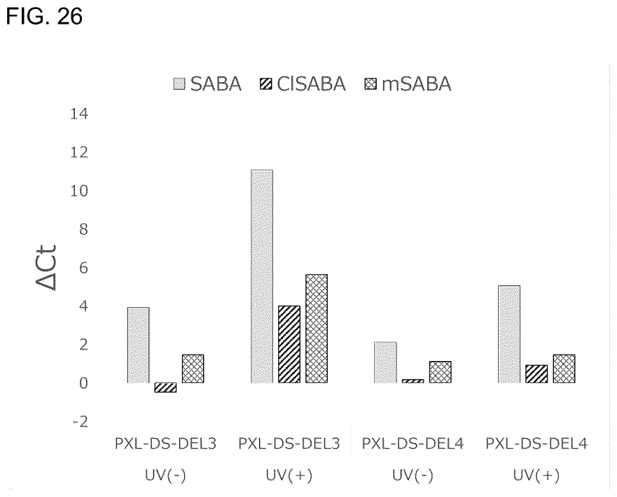

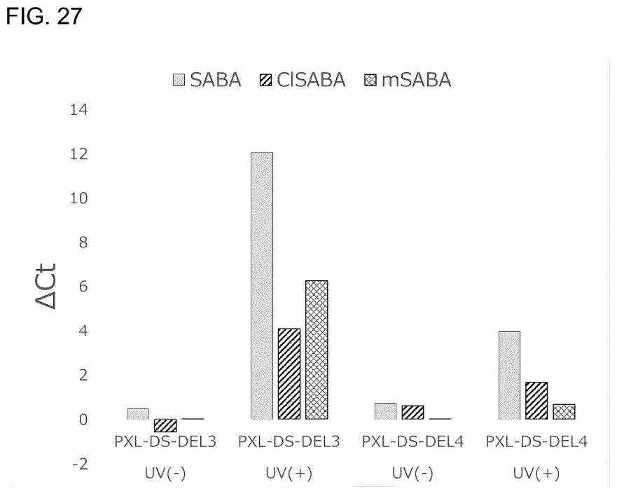

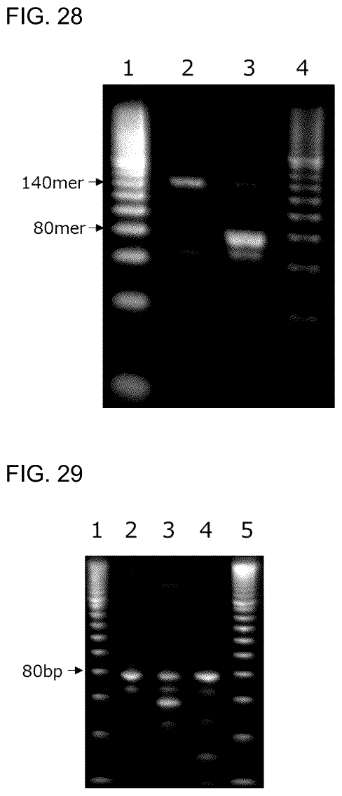

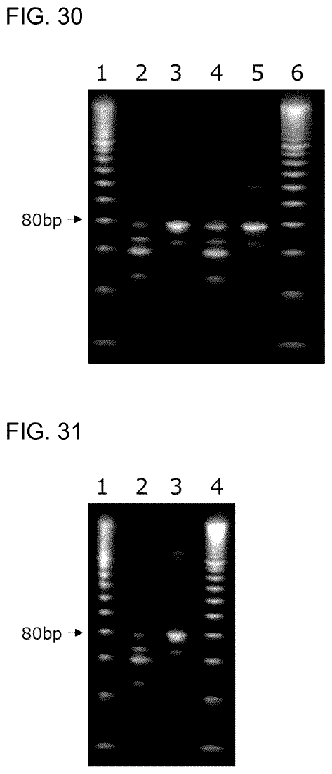

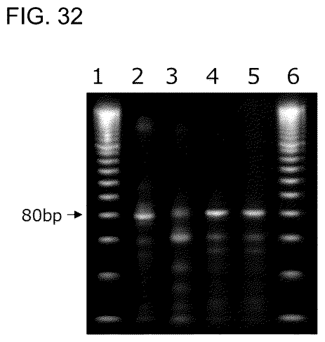

The Ct values of the various DEL samples obtained above were measured by real-time PCR in the same manner as in Example 8. The results of comparing the ΔCt values (difference from the Ct value of the negative control) are shown in FIG. 25.

As described in Example 8, the intensity of the affinities between the CA9 protein and “PXL-DS-mSABA-DEL2” and “PXL-DS-mSABA-DEL3” is expected to be medium (Non-Patent Documents 6 and 7).

As shown in the graph of FIG. 25, in the samples without UV radiation, every ΔCt values are small, which suggests that it is difficult to obtain a binder having medium affinity in the case of not carrying out the photocrosslinking reaction in the DEL screening as similar to the result of Example 8.

On the other hand, in the samples with UV radiation, every ΔCt values increased as compared to the samples without UV radiation, which suggests that acquisition of a binder having medium affinity can be expected also in the photoreactive cross linker-modified double-stranded DELs (all of them are different in the linker structure from the photoreactive cross linker-modified double-stranded DELs used in Example 8) used in this example.

The result means that a photoreactive cross linker-modified double-stranded DEL derived from a hairpin type DEL having “selectively cleavable site(s)” and having various linker structures is useful in DEL screening utilizing a photocrosslinking reaction.

### Example 12

[Comparison of Binder Recovery Efficiencies (DNA Detection Sensitivity) Between “Photoreactive Cross Linker-Modified Double-Stranded DEL Having Covalent Bond Between Cross Linker and Coding Sequence” and “Photoreactive Cross Linker-Modified Double-Stranded DEL without Covalent Bond Between Cross-Linker and Coding Sequence” ]
<Synthesis of Photoreactive Cross Linker-Modified Double-Stranded DEL without Covalent Bond Between Cross Linker and Coding Sequence>

Using the DEL compounds having the single-stranded DNA obtained in Example 10 (“SS-SABA-DEL3”, “SS-ClSABA-DEL3”, “SS-mSABA-DEL3”, and “SS-Amino-DEL3”), annealing using “PXL-Pr3” was carried out by the following procedures to synthesize 4 kinds of the photoreactive cross linker-modified double-stranded DEL compounds having the sequences shown in Table 40 (“PXL-DS-SABA-DEL4”, “PXL-DS-ClSABA-DEL4”, “PXL-DS-mSABA-DEL4”, and “PXL-DS-Amino-DEL4”). The sequence notation in Table 40 is the same as in Tables 34, 36, and 37, and it means that the 4 kinds of the compounds are formed by double strands of the oligonucleotide chains of SEQ ID NOs: 148 and 145, SEQ ID NOs: 148 and 146, SEQ ID NOs: 148 and 147, and SEQ ID NOs: 148 and 143, respectively.

TABLE 40

| | No. | Seq. |
|---|---|---|
| PXL-DS- | 148 | (X2)GTTGACTCCCAAATCGATGTG |
| SABA- | 145 | GCAGGTGAAGCTTGTCTGAATACTCGGTCACTTGCCA |
| DEL4 | | CTGCCTTGCTTCCTGACCACATCGATTTGGGAGTCAA |
| | | [SABA-mdC(TEG-amino)](p) |
| PXL-DS- | 148 | (X2)GTTGACTCCCAAATCGATGTG |
| CISABA- | 146 | GCAGGTGAAGCTTGTCTGAATACTCGGTCACTTGCCA |
| DEL4 | | CTGCCTTGCTTCCTGACCACATCGATTTGGGAGTCAA |
| | | [CISABA-mdC(TEG-amino)](p) |
| PXL-DS- | 148 | (X2)GTTGACTCCCAAATCGATGTG |
| mSABA- | 147 | GCAGGTGAAGCTTGTCTGAATACTCGGTCACTTGCCA |
| DEL4 | | CTGCCTTGCTTCCTGACCACATCGATTTGGGAGTCAA |
| | | [mSABA-mdC(TEG-amino)](p) |
| PXL-DS- | 148 | (X2)GTTGACTCCCAAATCGATGTG |
| Amino- | 143 | GCAGGTGAAGCTTGTCTGAATACTCGGTCACTTGCCA |
| DEL4 | | CTGCCTTGCTTCCTGACCACATCGATTTGGGAGTCAA |
| | | [mdC(TEG-amino)](p) |

To a PCR tube were added 30 μL of 10 μM aqueous solution of a DEL compound having various single-stranded DNAs; and 3.77 μL of 159 μM aqueous solution of “PXL-Pr3”. Deionized water was added to the resulting aqueous solution to have a total solution amount 60 μL. Thereafter, the mixture was incubated at 90° C. for 2 minutes and then cooled to room temperature over 30 minutes.

### <Preparation of DEL Samples>

4 kinds of the “photoreactive cross linker-modified double-stranded DELs without a covalent bond between a cross linker and a coding sequence” obtained above (“PXL-DS-SABA-DEL4”, “PXL-DS-ClSABA-DEL4”, “PXL-DS-mSABA-DEL4”, and “PXL-DS-Amino-DEL4”) and 4 kinds of the “photoreactive cross linker-modified double-stranded DELs with a covalent bond between a cross linker and a coding sequence” obtained in Example 10 (“PXL-DS-SABA-DEL3”, “PXL-DS-ClSABA-DEL3”, “PXL-DS-mSABA-DEL3”, and “PXL-DS-Amino-DEL3”) were each diluted with deionized water to prepare 50 nM DEL samples.

### <Photocrosslinking Reaction>

Device: CL-1000 Ultraviolet Crosslinker (UVP, manufactured by INC Corporation)
Microtube: 1.5 mL siliconized microtube round bottom (manufactured by WATSON Co., Ltd., Catalog number: 131-615CH)
Reaction tube: bottom vial for 96 holes (manufactured by Technolabsc Co., Ltd., Catalog number 96-V050FB)
Photocrosslinking reaction solution:
   Salmon Sperm DNA, sheared (Invitrogen, Catalog number: AM9680): 1.6 μL
   1 M NaCl (available from FUJIFILM Corporation, Catalog number: 191-01665): 5.0 μL
   D-PBS(−) (9, available from FUJIFILM Corporation, Catalog number: 045-29795): 32.4 μL
   Carbonic Anhydrase IX/CA9 (available from Sino Biologocal Inc., Catalog number:10107-H08H): 10.0 μL
   Aqueous solutions (50 nM) of various kinds of DEL samples: 1.0 μL
Reaction conditions:
   A mixture of the CA9 protein having the above composition and the DEL solution was incubated on ice for 2 hours. Thereafter, the mixture was held on ice, and UV radiation at 365 nm was carried out for 20 minutes.
      <Recovery of DEL Crosslinked with Protein>
Dynabeads Histag isolation & pulldown (manufactured by Invitrogen Corporation, Catalog number 10104D): 10.0 μL
   Tween 20 (available from Sigma Corporation, Catalog number: P7949-100ML)
   10% SDS (available from NIPPON GENE CO., LTD., Catalog number: 311-90271)
   Wash buffer (prepared by diluting D-PBS(−) with Tween 20 and 10% SDS to 0.2%)
   Imidazole (available from FUJIFILM Corporation, Catalog number: 097-05391)
   Elution buffer (imidazole was dissolved in D-PBS(−) to 200 mM to prepare it at pH 7.4)

The reaction solution after the UV radiation was mixed with Dynabeads his-tag pulldown and incubated at room temperature for 30 minutes. The mixture was fixed to a magnet stand and left to stand for 2 minutes, and then the supernatant was removed, 200 μL of Wash buffer was added to suspend Dynabeads. This operation was further repeated five times. 100 μL of Elution buffer was added to t Dynabeads after the washing and the mixture was reacted at room temperature for 10 minutes. After the reaction, Dynabeads were placed on a magnetic stand, and after 2 minutes, the supernatant was collected as a sample.

<Preparation of Sample without Photocrosslinking Reaction>

The above series of the operations were carried out without UV radiation, and each sample was collected.

### <Measurement of Ct Value by Real-Time PCR>

The Ct values of the various DEL samples obtained above were measured by real-time PCR in the same manner as in Example 8. The results of comparing the ΔCt values (difference from the Ct value of the negative control) are shown in FIG. 26.

Similar to Example 8, the strength of affinity between the CA9 protein and each compound as a binder is expected to be in the following order (Non-Patent Documents 6 and 7).

“PXL-DS-SABA-DEL3”, “PXL-DS-SABA-DEL4”>“PXL-DS-C1SABA-DEL3”, “PXL-DS-ClSABA-DEL4”>“PXL-DS-mSABA-DEL3”, “PXL-DS-mSABA-DEL4”>“PXL-DS-Amino-DEL3 (negative control)”, “PXL-DS-Amino-DEL4 (negative control)”

As shown in the graph of FIG. 26, in the samples with the UV radiation, the ΔCt value of the “photoreactive cross linker-modified double-stranded DEL with a covalent bond between a cross linker and a coding sequence (PXL-DS-DEL3)” was significantly higher than that of the “photoreactive cross linker-modified double-stranded DEL without a covalent bond between a cross linker and a coding sequence (PXL-DS-DEL4)” in every binders. Even if the kind of the binder is the same, the ΔCt value differs depending on the structure of the photoreactive cross linker-modified double-stranded DEL, which suggests that the “photoreactive cross linker-modified double-stranded DEL with a covalent bond between a cross linker and a coding sequence” has a higher binder recovery efficiency (DNA detection sensitivity).

The result means that the “photoreactive cross linker-modified double-stranded DEL with a covalent bond between a cross linker and a coding sequence” derived from hairpin type DEL having “selectively cleavable site(s)” is more useful than the “photoreactive cross linker-modified double-stranded DEL without a covalent bond between a cross linker and a coding sequence” in DEL screening using a photocrosslinking reaction.

### Example 13

[Verification of Binder Recovery Efficiency in Photocrosslinking Reaction of Photoreactive Cross Linker-Modified Double-Stranded DEL to which Strong Separation and Elution Conditions are Applied]

### <Preparation of DEL Samples>

Similar to Example 12, 4 kinds of the “photoreactive cross linker-modified double-stranded DEL without a covalent bond between a cross linker and a coding sequence” (“PXL-DS-SABA-DEL4”, “PXL-DS-ClSABA-DEL4”, “PXL-DS-mSABA-DEL4”, and “PXL-DS-Amino-DEL4”) and 4 kinds of the “photoreactive cross linker-modified double-stranded DEL with a covalent bond between a cross linker and a coding sequence” (“PXL-DS-SABA-DEL3”, “PXL-DS-ClSABA-DEL3”, “PXL-DS-mSABA-DEL3”, and “PXL-DS-Amino-DEL3”) were each diluted with deionized water to prepare 50 nM DEL samples.

### <Photocrosslinking Reaction>

Device: CL-1000 Ultraviolet Crosslinker (UVP, manufactured by INC Corporation)
Reaction tube: bottom vial for 96 holes (manufactured by Technolabsc Co., Ltd., Catalog number 96-V050FB)
Photocrosslinking reaction solution:
   Salmon Sperm DNA, sheared (Invitrogen, Catalog number: AM9680): 1.6 μL
   1 M NaCl (available from FUJIFILM Corporation, Catalog number: 191-01665): 5.0 μL
   D-PBS(−) (9, available from FUJIFILM Corporation, Catalog No. 045-29795): 39.4 μL
   Carbonic Anhydrase IX/CA9 (available from Sino Biologocal Inc., Catalog number:10107-H08H): 3.0 μL
   Aqueous solutions (50 nM) of various kinds of DEL samples: 1.0 μL
Reaction conditions:
   A mixture of the CA9 protein having the above composition and the DEL solution was incubated on ice for 2 hours. Thereafter, the mixture was held on ice and UV radiation at 365 nm was carried out for 20 minutes.
      <Recovery of DEL Crosslinked with Protein>
Dynabeads Histag isolation & pulldown (manufactured by Invitrogen Corporation, Catalog number 10104D): 10.0 μL
Tween 20 (available from Sigma Corporation, Catalog number: P7949-100ML)
Wash buffer (prepared by diluting D-PBS(−) with Tween 20 to 0.2%)
200 mM imidazole solution (manufactured by FUJIFILM Corporation, Catalog number: 097-05391)

The reaction solution after the UV radiation was mixed with Dynabeads his-tag pulldown and incubated at room temperature for 30 minutes. The mixture was fixed to a magnet stand and left to stand for 2 minutes, and then the supernatant was removed and 200 μL of Wash buffer was added to suspend Dynabeads and the mixture was reacted at 90° C. for 10 minutes. This operation was further repeated three times. 30 μL of 200 mM imidazole solution was added to Dynabeads after the washing and the mixture was reacted at room temperature for 10 minutes. After the reaction, Dynabeads were placed on a magnetic stand, and after 2 minutes, the supernatant was collected as a sample.

<Preparation of Sample without Photocrosslinking Reaction>

The above series of the operations were carried out without UV radiation, and each sample was collected.

As described in the above <Recovery of DEL crosslinked with protein>, the heating conditions were applied when the DEL crosslinked with protein was recovered, and therefore strong separation and elution conditions were applied.

### <Measurement of Ct Value by Real-Time PCR>

The Ct values of the various DEL samples obtained above were measured by real-time PCR in the same manner as in Example 8. The results of comparing the ΔCt values (difference from the Ct value of the negative control) are shown in FIG. 27.

As shown in the graph of FIG. 27, in the samples with the UV radiation, the ΔCt value of the “photoreactive cross linker-modified double-stranded DEL with a covalent bond between a cross linker and a coding sequence (PXL-DS-DEL3)” was significantly higher than that of the “photoreactive cross linker-modified double-stranded DEL without a covalent bond between a cross linker and a coding sequence (PXL-DS-DEL4)” in every binders. Even if the type of the binder is the same, the ΔCt value differs depending on the structure of the photoreactive cross linker-modified double-stranded DEL, which suggests that the “photoreactive cross linker-modified double-stranded DEL with a covalent bond between a cross linker and a coding sequence” has a higher binder recovery efficiency (DNA detection sensitivity).

The result means that the “photoreactive cross linker-modified double-stranded DEL with a covalent bond between a cross linker and a coding sequence” derived from a hairpin type DEL having “selectively cleavable site(s)” is more useful than the “photoreactive cross linker-modified double-stranded DEL without a covalent bond between a cross linker and a coding sequence” in DEL screening using a photocrosslinking reaction.

In addition, the present results suggest that the “photoreactive cross linker-modified double-stranded DEL with a covalent bond between a cross linker and a coding sequence” derived from a hairpin type DEL having “selectively cleavable site(s)” can also be adapted to DEL screening under strong separation conditions or elution conditions for the purpose of removing non-specific binders, etc.

### Example 14

### [Conversion of Hairpin DNA to Single-Stranded DNA Using U-DEL-13-HP as Starting Material and Addition of New Function]

<Synthesis of Starting Material Head Piece (“mSABA-DEL-HP5”) of DEL Compound>

“mSABA-DEL-HP5” having the sequence shown in Table 41 was synthesized using “U-DEL13-HP” as a starting material and by the same procedures as in <Synthesis of 3 kinds of DEL compounds having single-stranded DNA (“SS-SABA-DEL3”, “SS-ClSABA-DEL3”, and “SS-mSABA-DEL3”)> of Example 10. The notations in Table 41 are the same as in Table 36.

TABLE 41

| | | Expected | Observed MW. |
|---|---|---|---|
| No. | Seq. | MW. | (deconvoluton) |
| 151 | (p)GAGTCATT[mSABA-mdC | 6196.0 | 6196.2 |
| | (TEG-amino)](dU) | | |
| | TTGACTCCC | | |

A part of the obtained solution was sampled and diluted with deionized water, and then mass spectrometry by ESI-MS was carried out under the conditions of Analytical condition 3 in Example 3 to identify the “mSABA-DEL-HP5” of interest (the expected molecular weight and the observed molecular weight of each compound are shown in Table 41).

<Synthesis of Hairpin DEL Compound (“mSABA-DEL5”)>

The hairpin DEL compound having the sequence shown in Table 42 (“mSABA-DEL5”) was synthesized in the same manner as in Example 10 by double strand ligation of the starting material head piece “mSABA-DEL-HP5” with Pr_TAG2_CP. The sequence notation in Table 42 is the same as in Table 36.

TABLE 42

| | | Expected | Observed MW. |
|---|---|---|---|
| No. | Seq. | MW. | (deconvoluton) |
| 152 | GCAGGTGAAGCTTGTCTGAATAC | 46938.4 | 47021.5 |
| | TCGGTCACTTGCCACTGCCTTGC | | |
| | TTCCTGACCACATCGATTTGGGA | | |
| | GTCATT[mSABA-mdC(TEG- | | |
| | amino)](dU)TTGACTCGCAAA | | |
| | TCGATGTGGTCAGGAAGCAAGGC | | |
| | AGTGGCAAGTGACCGAGTATTCA | | |
| | GACAAGCTTCACCTGC | | |

A part of the obtained solution was sampled and diluted with deionized water, and then mass spectrometry by ESI-MS was carried out under the conditions of Analytical condition 3 in Example 3 to identify the “mSABA-DEL5” of interest (the expected molecular weight and the observed molecular weight of each compound are shown in Table 42).

<Cleavage of Hairpin DEL Compound (“mSABA-DEL5”) by USER® Enzyme>

The hairpin DEL compound obtained above “mSABA-DEL5” was cleaved by USER® enzyme in the same procedures as in <Cleavage of 5 kinds of DEL compounds having biotin at 3′ end (AAZ-BIO-DEL, SABA-BIO-DEL, ClSABA-BIO-DEL, mSABA-BIO-DEL, and Amino-BIO-DEL) by USER® enzyme> of Example 7 to convert it into the DEL compound having the double-stranded nucleic acid having the sequence of shown in Table 43 “DS-mSABA-DEL5”. The sequence notation in Table 43 is the same as in Table 36, and it means that the compound is formed by a double strand of the oligonucleotide chains of SEQ ID NOs: 153 and 154.

TABLE 43

| | | | Expected | Observed MW. |
|---|---|---|---|---|
| | No. | Seq. | MW. | (deconvoluton) |
| DS- | 153 | (p)TTGACTGCCAAATC | 22947.8 | 22947.4 |
| mSABA- | | GATGTGGTCAGGAAGCA | | |
| DEL5 | | AGGCAGTGGCAAGTGAC | | |
| | | CGAGTATTCAGACAAGC | | |
| | | TTCACCTGC | | |
| | 154 | GCAGGTGAAGCTTGTCT | 23798.4 | 23798.0 |
| | | GAATACTCGGTCACTTG | | |
| | | CCACTGCCTTGCTTCCT | | |
| | | GACCACATCGATTTGGG | | |
| | | AGTCATT | | |
| | | [mSABA-mdC | | |
| | | (TEG-amino)](p) | | |

A part of the obtained solution was sampled and diluted with deionized water, and then mass spectrometry by ESI-MS was carried out under the conditions of Analytical condition 3 in Example 3 to identify the DEL compound having the double-stranded nucleic acid of interest “DS-mSABA-DEL5” (the expected molecular weight and the observed molecular weight of each compound are shown in Table 43).

Also, a part of the obtained reaction solution was sampled and analysis by modified polyacrylamide gel electrophoresis was carried out under the same conditions as in Example 3. From the results shown in FIG. 28, it was confirmed that “mSABA-DEL5” was cleaved with high yield and converted into “DS-mSABA-DEL5”. The samples of each Lane in FIG. 28 are as follows.
Lane 1: 20 bp DNA Ladder (manufactured by Lonza, Lonza 20 bp DNA Ladder, Catalog number: 50330)
Lane 2: mSABA-DEL5
Lane 3: Sample after subjecting to cleavage reaction of mSABA-DEL5 by USER® enzyme
Lane 4: 20 bp DNA ladder (manufactured by Lonza, Lonza 20 bp DNA Ladder, Catalog number: 50330)

### <Conversion of DEL Compound Having Double-Stranded Nucleic Acid “DS-mSABA-DEL5” to Single-Stranded DEL by Lambda Exonuclease>

The DEL compound having the double-stranded nucleic acid obtained above “DS-mSABA-DEL5” was treated by Lambda Exonuclease in the same manner as in Example 9 to prepare the DEL compound having the single-stranded DNA “SS-mSABA-DEL5”. “SS-mSABA-DEL5” is the oligonucleotide chain of SEQ ID NO: 154 in Table 43.

A part of the obtained supernatant was sampled and diluted with deionized water, and then mass spectrometry by ESI-MS was carried out under the conditions of Analytical condition 3 in Example 3, whereby the molecular weight of 23825.8 was observed and the DEL compound having the single-stranded DNA of interest “SS-mSABA-DEL5” was identified.

### <Synthesis of Photoreactive Cross Linker-Modified Primer “PXL-Pr5”>

The photoreactive cross linker-modified primer having the sequence shown in Table 44 “PXL-Pr5” was synthesized in the same procedures as in <Synthesis of photoreactive cross linker-modified primer “PXL-Pr2”> of Example 10. However, as a starting material, “L-Pr5” (synthesized in the same manner as in Example 1, and the sequence is shown in Table 45) was used instead of “L-Pr”. The sequence notation in Table 44 is the same as in Table 32. In addition, the sequence notation in Table 45 is the same as in Table 8.

TABLE 44

| | | | Expected | Observed MW. |
|---|---|---|---|---|
| | No. | Seq. | MW. | (deconvoluton) |
| PXL- | 155 | (2)GAATGACTCC | 7032.7 | 7032.0 |
| Pr5 | | CAAATCGATGTG | | |

TABLE 45

| | No. | Seq. |
|---|---|---|
| L-Pr5 | 156 | (amino-C6-L)GAATGACTCCCAAATCGATGTG |

A part of the obtained solution was sampled and diluted with deionized water, and then mass spectrometry by ESI-MS was carried out under the conditions of Analytical condition 3 in Example 3 to identify the photoreactive cross linker-modified primer of interest “PXL-Pr5” (the expected molecular weight and the observed molecular weight of each compound are shown in Table 44).

### <Synthesis of Photoreactive Cross Linker-Modified Double-Stranded DEL Compound (“PXL-DS-mSABA-DEL5”)>

The DEL compound having the single-stranded DNA obtained above “SS-mSABA-DEL5” was used as a template DNA, and a primer elongation reaction using “PXL-Pr5” was carried out in the same procedures as in Example 7 to synthesize the photoreactive cross linker-modified double-stranded DEL compound having the sequence shown in Table 46 “PXL-DS-mSABA-DEL5”. The sequence notations in Table 46 are the same as in Tables 36 and 44, and it means that the compound is formed by a double strand of the oligonucleotide chains of SEQ ID NOs: 157 and 154.

TABLE 46

| | | | Expected | Observed MW. |
|---|---|---|---|---|
| | No. | Seq. | MW. | (deconvoluton) |
| PXL-DS- | 157 | (X2)GAATGACTCCC | 23808.5 | 23872.7 |
| mSABA- | | AAATCGATGTGGTCA | | |
| DEL5 | | GGAAGCAAGGCAGTG | | |
| | | GCAAGTGACCGAGTA | | |
| | | TTCAGACAAGCTTCA | | |
| | | CCTGC | | |
| | 154 | GCAGGTGAAGCTTGT | 23798.4 | 23835.4 |
| | | CTGAATACTCGGTCA | | |
| | | CTTGCCACTGCCTTG | | |
| | | CTTCCTGACCACATC | | |
| | | GATTTGGGAGTCATT | | |
| | | [mSABA-mdC(TEG- | | |
| | | amino)](p) | | |

A part of the obtained solution was sampled and diluted with deionized water, and then mass spectrometry by ESI-MS was carried out under the conditions of Analytical condition 3 in Example 3 to identify the photoreactive cross linker-modified double-stranded DEL of interest “PXL-DS-mSABA-DEL5” (the expected molecular weights and the observed molecular weights of the compounds are shown in Table 46).

Also, a part of the obtained reaction solution was sampled and analysis by polyacrylamide gel electrophoresis was carried out under the conditions shown in Example 7. From the results shown in FIG. 29, it was confirmed that conversion to “PXL-DS-mSABA-DEL5” was accomplished with high yield by an elongation reaction of the primer. The samples of each Lane in FIG. 29 are as follows. The samples were prepared so that the loading amount of each DEL compound was about 40 ng.
Lane 1: 20 bp DNA Ladder (manufactured by Lonza, Lonza 20 bp DNA Ladder, Catalog number: 50330)
Lane 2: DS-mSABA-DEL5
Lane 3: SS-mSABA-DEL5
Lane 4: Sample (PXL-DS-mSABA-DEL5) after subjecting to primer elongation reaction of SS-mSABA-DEL5
Lane 5: 20 bp DNA ladder (manufactured by Lonza, Lonza 20 bp DNA Ladder, Catalog number: 50330)

### Example 15

### [Synthesis of Cross Linker-Modified Double-Stranded DEL]

### <Synthesis of 3 Kinds of Cross Linker-Modified Primers (PA-Pr, TPD-Pr, and ACA-Pr)>

Various cross linker-modified primers having the sequences shown in Table 47 were synthesized by the following procedures. In the sequence notation in Table 47, “(PA)” means the group represented by the following formula (22)

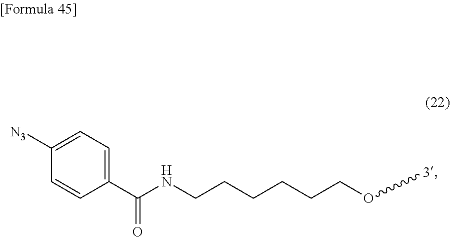

“(TPD)” means the group represented by the following formula (23)

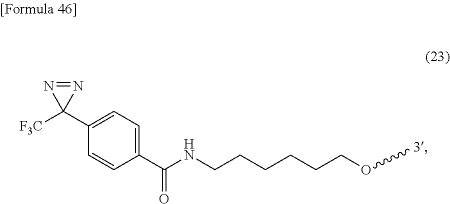

“(ACA)” means the group represented by the following formula (24)

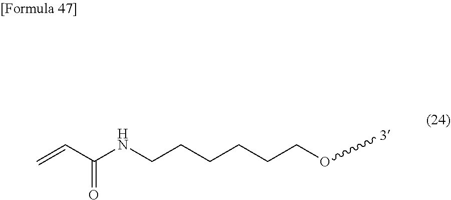

and other notations are the same as in Table 2.

TABLE 47

| | | | Expected | Observed MW. |
|---|---|---|---|---|
| | No. | Seq. | MW. | (deconvoluton) |
| PA-Pr | 158 | (PA)TTGACTCCCAAA | 6416.3 | 6417.7 |
| | | TCGATGTG | | |
| TPD-Pr | 159 | (TPD)TTGACTCCCAA | 6483.3 | 6483.8 |
| | | ATCGATGTG | | |
| ACA-Pr | 160 | (ACA)TTGACTCCCAA | 6325.2 | 6324.0 |
| | | ATCGATGTG | | |

The starting material active esters for synthesizing each compound are as follows.

| Compound: | Starting material active ester |
|---|---|
| PA-Pr: | N-succinimidyl 4-azidobenzoate |
| TPD-Pr: | N-succinimidyl |
| | 4-(3-(trifluoromethyl)-3H-diazilin-3-yl)benzoate |
| ACA-Pr: | N-succinimidyl acrylate |

To a PCR tube was added a solution (1 mM) of L-Pr (described in Example 7) in a sodium borate buffer (250 mM, pH 9.4) cooled to 10° C. To the tube was added 50 equivalents of the starting material active ester (25 μL, 0.2 M dimethylsulfoxide solution) and the formed mixture was shaken at 10° C. for 30 minutes.

The reaction solution was treated with 12 μL of 5 M aqueous sodium chloride solution and 396 μL of cooled (−20° C.) ethanol, and allowed to stand at −78° C. for 30 minutes. After centrifugation, the supernatant was removed and the obtained pellets were air-dried.

The obtained pellets were dissolved in 50 mM triethylammonium acetate buffer (pH 7.5) and purified by reverse phase HPLC using Phenomenex Gemini C18 column. Using a dual mobile phase gradient profile, the target product was eluted using 50 mM triethylammonium acetate buffer (pH 7.5) and acetonitrile/500 mM triethylammonium acetate buffer (9:1, v/v). Fractions containing the target product were collected, mixed, and concentrated. The resulting solution was desalted with the Amicon® Ultra Centrifugal filter (3 kD cutoff), ethanol precipitation was carried out, and then deionized water was added to the pellets to prepare a solution.

A part of the obtained solution was sampled and diluted with deionized water, and then mass spectrometry by ESI-MS was carried out under the conditions of Analytical condition 3 in Example 3 to identify the target product respectively (the expected molecular weights and the observed molecular weights of the compounds are shown in Table 47).

### <Synthesis of Compound “AOP-L-Pr”>

The compound having the sequence shown in Table 48 “AOP-L-Pr” was synthesized by the following procedures. In the sequence notation in Table 48, “(AOP-aminoC6-L)” means the group represented by the following formula (25)

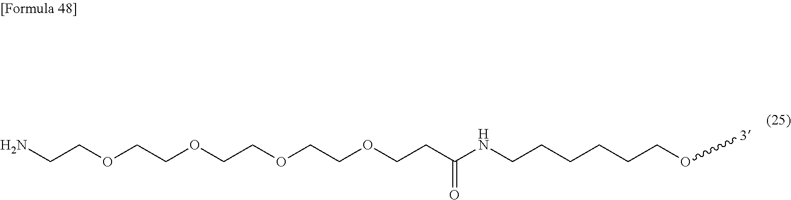

and other notations are the same as in Table 2.

TABLE 48

| | | | Expected | Observed MW. |
|---|---|---|---|---|
| | No. | Seq. | MW. | (deconvoluton) |
| AOP-L-Pr | 161 | (AOP-aminoC6-L) | 6518.5 | 6519.7 |
| | | TTGACTCCCAAATCG | | |
| | | ATGTG | | |

To a PCR tube was added a solution (200 μL, 1 mM) of L-Pr (synthesized in the same manner as in Example 1, and the sequence is shown in Table 28) in a sodium borate buffer (150 mM, pH 9.4) cooled to 10° C. To the tube were added 40 equivalents of N-Fmoc-15-amino-4,7,10,13-tetraoxaoctadecanoic acid (20 μL, 0.4 M N,N-dimethyl-acetamide solution), subsequently 40 equivalents of 4-(4,6-dimethoxy[1.3.5]triazin-2-yl)-4-methylmorpholinium chloride hydrate (DMTMM) (16 μL, 0.5 M aqueous solution), and the formed mixture was shaken at 10° C. for 4 hours.

The reaction solution was treated with 23.6 μL of 5 M aqueous sodium chloride solution and 778.8 μL of cooled (−20° C.) ethanol, and allowed to stand at −78° C. overnight. After centrifugation, the supernatant was removed and the obtained pellets were air-dried. To the pellets was added 180 μL of deionized water to prepare a solution, and then 20 μL of piperidine was added and the mixture was shaken at 10° C. for 3 hours.

The resulting solution was treated with 20 μL of 5 M aqueous sodium chloride solution and 660 μL of cooled (−20° C.) ethanol, and allowed to stand at −78° C. for 30 minutes. After centrifugation, the supernatant was removed, and to the obtained pellets was added 200 μL of deionized water to prepare a 1 mM solution.

A part of the obtained solution was sampled and diluted with deionized water, and then mass spectrometry by ESI-MS was carried out under the conditions of Analytical condition 2 in Example 1 to identify the target product (the expected molecular weight and the observed molecular weight of each sequence are shown in Table 48).

### <Synthesis of Cross Linker-Modified Primer (BMP-Pr)>

The cross linker-modified primer having the sequence shown in Table 49 (BMP-Pr) was synthesized by the following procedures. In the sequence notation in Table 49, “(BMP)” means the group represented by the following formula (26)

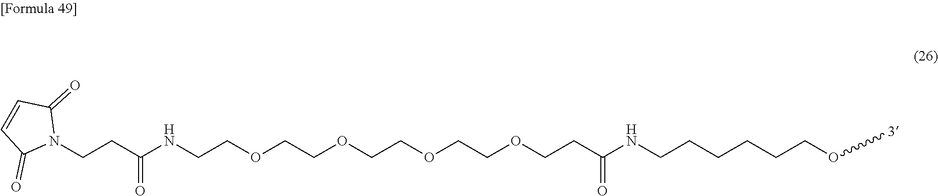

and other notations are the same as in Table 2.

TABLE 49

| | | | Expected | Observed MW. |
|---|---|---|---|---|
| | No. | Seq. | MW. | (deconvoluton) |
| BMP-Pr | 162 | (BMP)TTGACTCCCAAA | 6669.6 | 6670.8 |
| | | TCGATGTG | | |

To a PCR tube was added a solution (40 μL, 0.5 mM) of AOP-L-Pr in sodium phosphate buffer (125 mM, pH 9.4) cooled to 10° C. To the tube was added 50 equivalents of N-succinimidyl 3-maleimidopropionate (5 μL, 0.2 M dimethylsulfoxide solution) and the formed mixture was shaken at 10° C. for 40 minutes. Thereafter, 20 μL of dimethyl sulfoxide was added and the formed mixture was further shaken at 10° C. for 25 minutes.

The reaction solution was treated with 5 μL of 5 M aqueous sodium chloride solution and 215 μL of cooled (−20° C.) ethanol, and allowed to stand at −78° C. for 30 minutes. After centrifugation, the supernatant was removed and the obtained pellets were air-dried. The obtained pellets were dissolved in deionized water and the solution was desalted by the Amicon® Ultra Centrifugal filter (3 kD cutoff)

A part of the obtained solution was sampled and diluted with deionized water, and then mass spectrometry by ESI-MS was carried out under the conditions of Analytical condition 3 in Example 3 to identify the cross linker-modified primer of interest “BMP-Pr” (the expected molecular weight and the observed molecular weight of each compound are shown in Table 49).

### <Synthesis of Cross Linker-Modified Double-Stranded DEL (“TPD-DS-mSABA-DEL”)>

Using the DEL compound having the single-stranded DNA obtained in Example 10 “SS-mSABA-DEL” as a template DNA, a primer elongation reaction using the cross linker-modified primer “TPD-Pr” obtained in the above <Synthesis of 3 kinds of cross linker-modified primers> was carried out in the following procedures to synthesize the cross linker-modified double-stranded DEL compound having the sequence shown in Table 50 “TPD-DS-mSABA-DEL”. The sequence notations in Table 50 are the same as in Tables 26 and 47, and it show that “TPD-DS-mSABA-DEL”is formed by a double strand of the oligonucleotide chains of SEQ ID NOs: 163 and 131.

TABLE 50

| | | | Expected | Observed MW. |
|---|---|---|---|---|
| | No. | Seq. | MW. | (deconvoluton) |
| TPD-DS- | 163 | (TPD)TTGACTCCCAAATCGATGTGGTCAGGA | 23259.1 | 23285.9 |
| mSABA-DEL | | AGCAAGGCAGTGGCAAGTGACCGAGTATTCAG | | |
| | | ACAAGCTTCACCTGC | | |
| | 131 | GCAGGTGAAGCTTGTCTGAATACTCGGTCACT | 24173.7 | 24209.7 |
| | | TGCCACTGCCTTGCTTCCTGACCACATCGATT | | |
| | | TGGGAGTCAA(dSpacer)(dSpacer) | | |
| | | (mSABA-AOP-AminoC7)(dSpacer) | | |
| | | (dSpacer)(p) | | |

A part of the obtained solution was sampled and diluted with deionized water and then mass spectrometry by ESI-MS was carried out under the conditions of Analytical condition 3 in Example 3 to identify the cross linker-modified double-stranded DEL of interest “TPD-DS-mSABA-DEL” (the expected molecular weights and the observed molecular weights of the compounds are shown in Table 50).

### <Synthesis of Cross Linker-Modified Double-Stranded DEL (“ACA-DS-ClSABA-DEL”)>

Using the DEL compound having the single-stranded DNA obtained in Example 10 “SS-ClSABA-DEL” as a template DNA, a primer elongation reaction using the cross linker-modified primer “ACA-Pr” obtained above was carried out in the same procedures as in Example 7 to synthesize the cross linker-modified double-stranded DEL compound having the sequence shown in Table 51 “ACA-DS-ClSABA-DEL”. The sequence notations in Table 51 are the same as in Tables 26 and 47, and it shows that “ACA-DS-ClSABA-DEL” is formed by a double strand of the oligonucleotide chains of SEQ ID NO: 164 and SEQ ID NO: 129.

TABLE 51

| | | | Expected | Observed MW. |
|---|---|---|---|---|
| | No. | Seq. | MW. | (deconvoluton) |
| ACA-DS- | 164 | (ACA)TTGACTCCCAAATCGATGTGGTCAGG | 23101.0 | 23097.4 |
| CISABA-DEL | | AAGCAAGGCAGTGGCAAGTGACCGAGTATTC | | |
| | | AGACAAGCTTCACCTGC | | |
| | 129 | GCAGGTGAAGCTTGTCTGAATACTCGGTCAC | 24208.2 | 24235.7 |
| | | TTGCCACTGCCTTGCTTCCTGACCACATCGA | | |
| | | TTTGGGAGTCAA(dSpacer)(dSpacer) | | |
| | | (CISABA-AOP-AminoC7)(Spacer) | | |
| | | (dSpacer)(p) | | |

A part of the obtained solution was sampled and diluted with deionized water, and then mass spectrometry by ESI-MS was carried out under the conditions of Analytical condition 3 in Example 3 to identify the cross linker-modified double-stranded DEL of interest “ACA-DS-ClSABA-DEL” (the expected molecular weights and the observed molecular weights of the compounds are shown in Table 51).

### <Confirmation of Primer Elongation Reaction Using Gel Electrophoresis>

A part of the solutions obtained in the above <Synthesis of cross linker-modified double-stranded DEL (“TPD-DS-mSABA-DEL”)> and <Synthesis of cross linker-modified double-stranded DEL (“ACA-DS-ClSABA-DEL”)> was sampled, and analysis by polyacrylamide gel electrophoresis was carried out under the conditions shown in Example 7. From the results shown in FIG. 30, it was confirmed that conversion to “TPD-DS-mSABA-DEL” and “ACA-DS-ClSABA-DEL” was accomplished with high yield by an elongation reaction of the primer. The samples of each Lane in FIG. 30 are as follows. The samples were prepared so that the loading amount of each DEL compound was about 40 ng.
Lane 1: 20 bp DNA Ladder (manufactured by Lonza, Lonza 20 bp DNA Ladder, Catalog number: 50330)
Lane 2: SS-mSABA-DEL
Lane 3: Sample (TPD-DS-mSABA-DEL) after subjecting to primer elongation reaction of SS-mSABA-DEL
Lane 4: SS-ClSABA-DEL
Lane 5: Sample (ACA-DS-CISABA-DEL) after subjecting to primer elongation reaction of SS-ClSABA-DEL
Lane 6: 20 bp DNA ladder (manufactured by Lonza, Lonza 20 bp DNA Ladder, Catalog number: 50330)

### Example 16

### [Synthesis of Cross Linker-Modified Double-Stranded DEL Using Double-Stranded DEL Having Reactive Group for Cross Linker Modification]

### <Synthesis of Primer Having Reactive Group for Cross Linker Modification (“BCN-Pr”)>

The primer having the reactive group for cross linker modification of the sequence shown in Table 52 (BCN-Pr) was synthesized by the following procedures. In the sequence notation in Table 52, “(BCN)” means the group represented by the following formula (27)

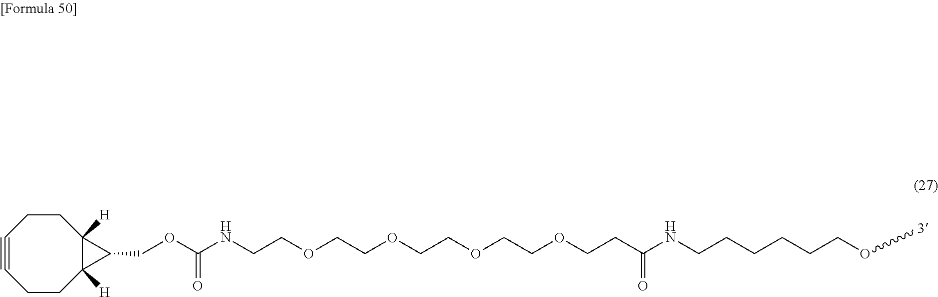

and other notations are the same as in Table 2.

TABLE 52

| | | | Expected | Observed MW. |
|---|---|---|---|---|
| | No. | Seq. | MW. | (deconvoluton) |
| BCN-Pr | 165 | (BCN)TTGACTCCCA | 6694.7 | 6695.1 |
| | | AATCGATGTG | | |

Synthesis of “BCN-Pr” was carried out in the same manner as in Example 15, using “AOP-L-Pr” as a starting material and (1R,8S,9s)-bicyclo[6.1.0]non-4-yn-9-ylmethyl succinimidyl carbonate as a starting material active ester.

A part of the obtained solution was sampled and diluted with deionized water, and then mass spectrometry by ESI-MS was carried out under the conditions of Analytical condition 3 in Example 3 to identify the “BCN-Pr” of interest (the expected molecular weight and the observed molecular weight of the compound are shown in Table 52).

### <Synthesis of Double-Stranded DEL Having Reactive Group for Cross Linker Modification>

Using “SS-mSABA-DEL” obtained in Example 10 as a template DNA, a primer elongation reaction using “BCN-Pr” was carried out in the same procedures as in Example 7 to synthesize the double-stranded DEL compound having the reactive group for cross linker modification having the sequence shown in Table 53 “BCN-DS-mSABA-DEL”. The sequence notations in Table 53 are the same as in Tables 26 and 52, and it shows that “BCN-DS-mSABA-DEL” is formed by a double strand of the oligonucleotide chains of SEQ ID NOs: 166 and 131.

TABLE 53

| | | | Expected | Observed MW. |
|---|---|---|---|---|
| | No. | Seq. | MW. | (deconvoluton) |
| BCN-DS- | 166 | (BCN)TTGACTCCCAAATCGATGTGGTCAGGA | 23470.5 | 23503.7 |
| mSABA-DEL | | AGCAAGGCAGTGGCAAGTGACCGAGTATTCAG | | |
| | | ACAAGCTTCACCTGC | | |
| | 131 | GCAGGTGAAGCTTGTCTGAATACTCGGTCACT | 24173.5 | 24198.2 |
| | | TGCCACTGCCTTGCTTCCTGACCACATCGATT | | |
| | | TGGGAGTCAA(dSpacer)(dSpacer) | | |
| | | (mSABA-AOP-AminoC7)(Spacer) | | |
| | | (dSpacer)(p) | | |

A part of the obtained solution was sampled and diluted with deionized water, and then mass spectrometry by ESI-MS was carried out under the conditions of Analytical condition 3 in Example 3 to identify the double-stranded DEL having the reactive group for cross linker modification of interest “BCN-DS-mSABA-DEL” (the expected molecular weights and the observed molecular weights of the compounds are shown in Table 53).

In addition, a part of the obtained reaction solution was sampled and analysis by polyacrylamide gel electrophoresis was carried out under the same conditions as in Example 7. From the results shown in FIG. 31, it was confirmed that conversion to “BCN-DS-mSABA-DEL” was accomplished with high yield by an elongation reaction of the primer. The samples of each Lane in FIG. 31 are as follows. The samples were prepared so that the loading amount of each DEL compound was about 40 ng.
Lane 1: 20 bp DNA Ladder (manufactured by Lonza, Lonza 20 bp DNA Ladder, Catalog number: 50330)
Lane 2: SS-mSABA-DEL
Lane 3: Sample (BCN-DS-mSABA-DEL) after subjecting to primer elongation reaction of SS-mSABA-DEL
Lane 4: 20 bp DNA ladder (manufactured by Lonza, Lonza 20 bp DNA Ladder, Catalog number: 50330)
   <Synthesis of Cross Linker-Modified Double-Stranded DEL (“PSF-DS-mSABA-DEL”) by Click Reaction with Double-Stranded DEL Having Reactive Group for Cross Linker Modification>

A cross linker was introduced to “BCN-DS-mSABA-DEL” obtained above by the click reaction to synthesize the cross linker-modified double-stranded DEL compound having the sequence shown in Table 54 “PSF-DS-mSABA-DEL”. The synthesis procedure is shown below. In the sequence notation in Table 54, “(PSF-t)” means a group represented by the following formula (28)

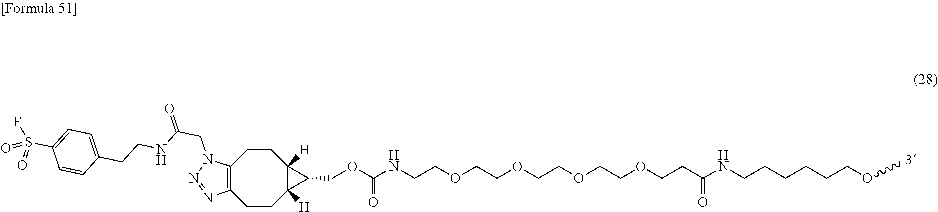

and other notations are the same as in Table 26, and it shows that “PSF-DS-mSABA-DEL” is formed by a double strand of the oligonucleotide chains of SEQ ID NOs: 131 and 167.

TABLE 54

| | | | Expected | Observed MW. |
|---|---|---|---|---|
| | No. | Seq. | MW. | (deconvoluton) |
| PSF-DS- | 167 | (PSF-t)TGACTCCCAAATCGATTGTGG | 23756.8 | 23798.4 |
| mSABA-DEL | | TCAGGAAGCAAGGCAGTGGCAAGTGACC | | |
| | | GAGTATTCAGACAAGCTTCACCTGC | | |
| | 131 | GCAGGTGAAGCTTGTCTGAATACTCGGT | 24173.5 | 24169.1 |
| | | CACTTGCCACTGCCTTGCTTCCTGACCA | | |
| | | CATCGATTTGGGAGTCAA(dSpacer) | | |
| | | (dSpacer)(mSABA-AOP-AminoC7) | | |
| | | (dSpacer)(dSpacer)(p) | | |

To a PCR tube was added N-succinimidyl 2-azidoacetate (125 μL, 0.2 M dimethylsulfoxide solution). To the tube were added N,N-diisopropylethylamine (5.2 μL), subsequently 4-(2-aminoethyl)benzenesulfonyl fluoride hydrochloride (30 mg), and the resulting mixture was shaken at 10° C. for 1 hour.

Dimethyl sulfoxide was added to the resulting mixture to be 10-fold diluted (diluted solution).

To a PCR tube were added a solution (2 μL, 0.1 mM) of BCN-DS-mSABA-DEL in a sodium phosphate buffer (250 mM, pH 7.0), subsequently 1.8 μL of dimethyl sulfoxide. Thereafter, 0.2 μL of the diluted solution obtained above was added and the obtained solution was shaken at 25° C. for 2 hours. [0457]1 μL of the obtained solution was sampled and diluted with deionized water, and then mass spectrometry by ESI-MS was carried out under the conditions of Analytical condition 3 in Example 3 to identify the cross linker-modified double-stranded DEL of interest “PSF-DS-mSABA-DEL” (the expected molecular weights and the observed molecular weights of the compounds are shown in Table 54).

<Synthesis of Cross Linker-Modified Double-Stranded DEL (“BMP-DS-mSABA-DEL”) by Click Reaction with Double-Stranded DEL Having Reactive Group for Cross Linker Modification>

A cross linker was introduced to “BCN-DS-mSABA-DEL” obtained above by the click reaction to synthesize the cross linker-modified double-stranded DEL compound having the sequence shown in Table 55 “BMP-DS-mSABA-DEL”. The synthesis procedure is shown below. In the sequence notation in Table 55, “(BMP-t)” means a group represented by the following formula (29)

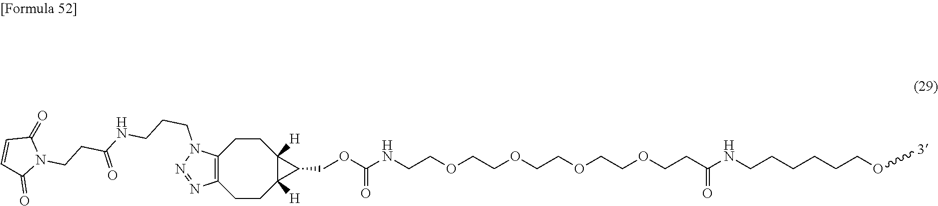

and other notations are the same as in Table 26, and it shows that “BMP-DS-mSABA-DEL” is formed by a double strand of the oligonucleotide chains of SEQ ID NOs: 131 and 168.

TABLE 55

| | | | Expected | Observed MW. |
|---|---|---|---|---|
| | No. | Seq. | MW. | (deconvoluton) |
| BMP-DS- | 168 | (BMP-t)TTGACTCCCAAATCGATG | 23721.8 | 23750.0 |
| mSABA-DEL | | TGGTCAGGAAGCAAGGCAGTGGC | | |
| | | AAGTGACCGAGTATTCAGACAAG | | |
| | | CTTCACCTGC | | |
| | 131 | GCAGGTGAAGCTTGTCTGAATAC | 24173.5 | 24177.9 |
| | | TCGGTCACTTGCCACTGCCTTGC | | |
| | | TTCCTGACCACATCGATTTGGGA | | |
| | | GTCAA(dSpacer)(dSpacer) | | |
| | | (mSABAAOP-AminoC7)(Spacer) | | |
| | | (dSpacer)(p) | | |

To a PCR tube was added 3-azidopropylamine (4.8 mg, 0.2 M dimethylsulfoxide solution). To the tube was added N-succinimidyl 3-maleimidopropionate (30 mg) and the resulting mixture was shaken at 10° C. for 1 hour.

Dimethyl sulfoxide was added to the resulting mixture to be 10-fold diluted (diluted solution).

To a PCR tube were added a solution (2 μL, 0.1 mM) of BCN-DS-mSABA-DEL in a sodium phosphate buffer (250 mM, pH 7.0), subsequently 1.8 μL of dimethyl sulfoxide. Thereafter, 0.2 μL of the diluted solution obtained above was added and the resulting solution was shaken at 25° C. for 2 hours.

1 μL of the obtained solution was sampled and diluted with deionized water, and then mass spectrometry by ESI-MS was carried out under the conditions of Analytical condition 3 in Example 3 to identify the cross linker-modified double-stranded DEL of interest “BMP-DS-mSABA-DEL” (the expected molecular weights and the observed molecular weights of the compounds are shown in Table 55).

### Example 17

[Conversion from Model Library Using U-DEL9-HP as Starting Material to Single-Stranded DNA and Addition of New Function]

### <Conversion of Model Library to DEL Compound Having Single-Stranded DNA Using Lambda Exonuclease>

Conversion to a DEL compound having a single-stranded DNA in the model library was carried out in the same procedures as in Example 9 using the sample of the model library (synthesized in Example 6) after carrying out a cleavage reaction by USER® enzyme. Then, the obtained solution was used as a starting material for the next step.

### <Conversion of Model Library Converted to DEL Having Single-Stranded DNA to Cross Linker-Modified Double-Stranded DEL>

Using the model library converted to the DEL having the single-stranded DNA obtained above as a template DNA, a primer elongation reaction using “PXL-Pr” (synthesized in Example 7) and “BCN-Pr” (synthesized in Example 16) was carried out in the same procedures as in Example 7.

### <Results>

A part of the obtained two kinds of the reaction solutions obtained by the primer elongation reaction was sampled and analysis by polyacrylamide gel electrophoresis was carried out under the same conditions as in Example 7. From the results shown in FIG. 32, it was confirmed that the model library converted to the single-stranded DEL was converted to a photoreactive cross linker-modified double-stranded DEL and a double-stranded DEL having a reactive group for cross linker modification with high yield by the elongation reaction of the primer. The samples of each Lane in FIG. 32 are as follows. The samples were prepared so that the loading amount of each DEL compound was about 40 ng.
Lane 1: 20 bp DNA Ladder (manufactured by Lonza, Lonza 20 bp DNA Ladder, Catalog number: 50330)
Lane 2: Sample after subjecting to cleavage reaction of model library by USER® enzyme
Lane 3: Sample after subjecting to conversion reaction of model library to single-stranded DEL by Lambda Exonuclease
Lane 4: Sample after subjecting to primer elongation reaction between model library after conversion to single-stranded DEL and PXL-Pr
Lane 5: Sample after subjecting to primer elongation reaction between model library after conversion to single-stranded DEL and BCN-Pr
Lane 6: 20 bp DNA ladder (manufactured by Lonza, Lonza 20 bp DNA Ladder, Catalog number: 50330)

### INDUSTRIAL APPLICABILITY

The present invention provides a method using a nucleic acid compound having selectively cleavable site(s). Further, the present invention provides a method of inducing a DEL containing a cleavable site in a DNA strand to a cross linker-modified double-stranded DEL and evaluating it, which makes it possible to screen compounds having both a “simple DEL synthesis method” and “expansion and improvement of the DEL evaluation method” as compared to conventional methods.

1. A method of evaluating a cross linker-modified double-stranded DNA-encoded library (DEL) induced from a hairpin type DEL having “selectively cleavable site(s)”, comprised of the following steps of:
(1) contacting the DEL with a biological target under conditions suitable for binding at least one library molecule of the DEL to the biological target;
(2) crosslinking a cross linker of the library molecule bound to the biological target with the biological target;
(3) separating a complex of the crosslinked library molecule and biological target from a non-crosslinked library molecule;
(4) identifying sequences of oligonucleotides that the library molecule in the recovered complex have; and
(5) using the sequences determined in (4) to identify a structure of one or more compounds that bind to the biological target.

2. The method according to claim 1, wherein the cross linker of the cross linker-modified double-stranded DEL is linked to an oligonucleotide having a coding sequence via a covalent bond.

3. The method according to claim 1, wherein the cross linker of the cross linker-modified double-stranded DEL binds to the 5′ end of the oligonucleotide directly or via a bifunctional spacer.

4.-15. (canceled)

16. The method according to claim 1, wherein the cross linker comprises at least one of an azide group, a diazirine group, a sulfonyl fluoride group, a diazo group, a cinnamoyl group, or an acrylate group.

17. (canceled)

18. The method according to claim 1, wherein the cross linker comprises
a structure of any of the formulae (AA) to (AE): 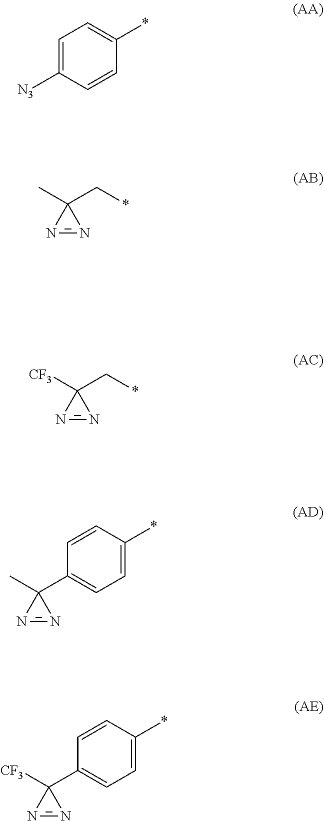
wherein * means a binding site with the 5′ end of the double-stranded DEL or with the bifunctional spacer side binding to the 5′ end of the double-stranded DEL
or
a structure of either formula (BA) or (BB): 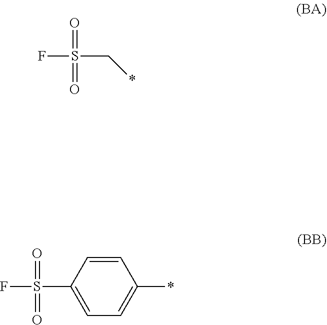 [SMILES: *CS(=O)(=O)F ; *c1ccc(S(=O)(=O)F)cc1]
wherein * means a binding site with the 5′ end of the double-stranded DEL or with the bifunctional spacer side binding to the 5′ end of the double-stranded DEL.

19.-24. (canceled)

25. The method according to claim 1, wherein the step of “crosslinking the cross linker of the library molecule bound to the biological target with the biological target” in (2) is a step of “crosslinking the cross linker of the library molecule bound to the biological target with the biological target by light irradiation” or a step of “crosslinking the cross linker of the library molecule bound to the biological target with the biological target by incubation”.

26.-30. (canceled)

31. The method according to claim 1, wherein the step of “separating the complex of the crosslinked library molecule and the biological target from the non-crosslinked library molecule” in (3) is a step of “separating the complex of the crosslinked library molecule and the biological target from the non-crosslinked library molecule by electrophoresis” or a step of “separating the complex of the crosslinked library molecule and the biological target by immobilizing the biological target on an immobilization carrier, and removing the non-crosslinked library molecule by washing”.

32.-34. (canceled)

35. The method according to claim 1, wherein the hairpin type DEL having the “selectively cleavable site(s)” is the DEL represented by the formula (I): 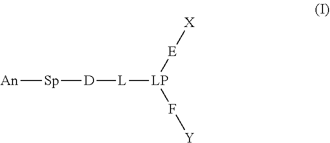
wherein
X and Y are oligonucleotide chains,
E and F are each independently an oligomer composed of nucleotides or nucleic acid analogues,
provided that E and F comprise base sequences complementary to each other and they form a duplex oligonucleotide,
LP is a loop site,
L is a linker,
D is a divalent group derived from a reactive functional group,
Sp is a bonding or a bifunctional spacer,
An is a partial structure comprised of at least one building block,
X and Y have a sequence capable of forming a duplex at least a part thereof,
X binds to E at the 5′ end,
Y binds to F at the 3′ end, and
at least any one site of E, F, or LP has at least one selectively cleavable site.

36. The method according to claim 35, wherein the hairpin type DEL having the “selectively cleavable site(s)” is the DEL represented by the formula (III):
An-Sp-C-Bn (III)
wherein
An and Sp represent the same meanings as defined in claim 35,
Bn represents a double-stranded oligonucleotide tag formed by an oligonucleotide chain X and an oligonucleotide chain Y,
C is the formula (I): 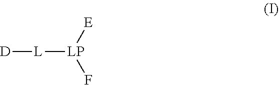
wherein E, LP, L, D, and F represent the same meanings as defined in claim 35, provided that D binds to An directly or via the bifunctional spacer, and E and F respectively bind to the corresponding terminal side of the double-stranded oligonucleotide tag Bn.

37. The method according to claim 35, wherein An is the same as defined in claim 35 and is a partial structure constructed by n building blocks α1 to an (n is an integer of 1 to 10), and Bn is the double-stranded oligonucleotide tag formed by an oligonucleotide chain X and an oligonucleotide chain Y and is a partial structure comprising an oligonucleotide which comprises a base sequence capable of identifying the structure of An.

38. The method according to claim 35, wherein LP is a loop site represented by (LP1)p-LS-(LP2)q, wherein
LS is a partial structure selected from the compound group described in the following (A) to (C):
(A) a nucleotide,
(B) a nucleic acid analogue, and
(C) a C1 to 14 trivalent group optionally having a substituent(s);
LP1 is each a partial structure independently or differently selected with the number of p from the compound group described in the following (1) and (2):
(1) a nucleotide, and
(2) a nucleic acid analogue;
LP2 is each a partial structure independently or differently selected with the number of q from the compound group described in the following (1) and (2):
(1) a nucleotide, and
(2) a nucleic acid analogue; and
the total number of p and q is 0 to 40.

39.-42. (canceled)

43. The method according to claim 38, wherein LP1, LP2, and LS are each a structure independently or differently selected from the following structures:
(A) a nucleotide or
(B) a nucleic acid analogue satisfying the following (B11) to (B15):
(B11) the nucleic acid analogue has phosphoric acid (or a corresponding site thereto) and a hydroxyl group (or a corresponding site thereto),
(B12) the nucleic acid analogue is comprised of carbon, hydrogen, oxygen, nitrogen, phosphorus or sulfur,
(B13) the nucleic acid analogue has a molecular weight from 142 to 1,500,
(B14) the nucleic acid analogue has the number of the atoms between the residues from 3 to 30, and
(B15) the nucleic acid analogue has the bonding mode of the atoms between the residues in which all bonds are single bonds or in which one or two double bonds are comprised and the remaining are single bonds.

44.-62. (canceled)

63. The method according to claim 36, wherein E and F are each independently an oligomer composed of nucleotides or nucleic acid analogues, and a chain length of E and F is each 3 to 40.

64.-76. (canceled)

77. The method according to claim 35, wherein L is
(1) a C1 to 20 aliphatic hydrocarbon optionally having a substituent(s) and may be replaced with 1 to 3 hetero atoms, or
(2) a C6 to 14 aromatic hydrocarbon optionally having a substituent(s).

78.-81. (canceled)

82. The method according to claim 35, wherein the reactive functional group for D is a reactive functional group which can constitute a C—C, amino, ether, carbonyl, amide, ester, urea, sulfide, disulfide, sulfoxide, sulfonamide, or sulfonyl bond.

83.-86. (canceled)

87. The method according to claim 35, wherein the selectively cleavable site(s) is/are deoxyribonucleoside which is not any one of deoxyadenosine, deoxyguanosine, thymidine, and deoxycytidine.

88. The method according to claim 35, wherein the selectively cleavable site(s) is/are deoxyuridine, bromodeoxyuridine, deoxyinosine, 8-hydroxydeoxyguanosine, 3-methyl-2′-deoxyadenosine, N6-etheno-2′-deoxyadenosine, 7-methyl-2′-deoxyguanosine, 2′-deoxyxanthosine, or 5,6-dihydroxy-5,6 dihydro-deoxythymidine.

89.-94. (canceled)

95. The method according to claim 35, wherein at least one cleavable site is comprised in E or (LP1)p, and at least one cleavable site is comprised in F or (LP2)q, and wherein the cleavable site comprised in E or (LP1)p and the cleavable site comprised in F or (LP2)q can be cleaved under different conditions.

96.-108. (canceled)

109. The method according to claim 1, wherein the bifunctional spacers are each SpD-SpL-SpX, wherein
SpD is a divalent group derived from a reactive group capable of constituting a C—C, amino, ether, carbonyl, amide, ester, urea, sulfide, disulfide, sulfoxide, sulfonamide, or sulfonyl bond,
SpL is polyalkylene glycol, polyethylene, a C1 to 20 aliphatic hydrocarbon which may be optionally replaced with a hetero atom(s), a peptide, an oligonucleotide, or a combination thereof, and
SpX is a divalent group derived from a reactive group which forms an amino, carbonyl, amide, ester, urea, or sulfonamide bond.

110.-120. (canceled)

121. The method according to claim 35, wherein a functional molecule is bound to any one of X and Y.

122.-123. (canceled)
